(19) United States
(12) Patent Application Publication
(10) Pub. No.: US 2023/0391739 A1
(43) Pub. Date: Dec. 7, 2023

(54) ARYL DERIVATIVES FOR TREATING TRPM3 MEDIATED DISORDERS

(71) Applicant: Katholieke Universiteit Leuven, Leuven (BE)

(72) Inventors: Sara ALLASIA, Leuven (BE); Philippe ARZEL, Gent (BE); Patrick CHALTIN, Zétrud-Lemay (BE); Amuri KILONDA, Boutersem (BE); Arnaud MARCHAND, Bierbeek (BE); Thomas VANHERCK, Heverlee (BE); Thomas VOETS, Kessel-Lo (BE); Joris VRIENS, Wakkerzeel (BE); Melanie REICH, Aachen (DE); André WELBERS, Koln (DE)

(21) Appl. No.: 18/250,798

(22) PCT Filed: Nov. 4, 2021
(86) PCT No.: PCT/EP2021/082853

§ 371 Date: Apr. 27, 2023

(30) Foreign Application Priority Data
Nov. 24, 2020 (EP) 20209570.9

(51) Int. Cl. C07D 307/83; C07D 405/12; C07D 407/12; C07D 413/12; C07D 405/14; C07D 487/04; C07D 409/12; C07D 471/08; C07D 498/08; A61P 25/04

(52) U.S. Cl. CPC C07D 307/83; C07D 405/12; C07D 407/12; C07D 413/12; C07D 405/14; C07D 487/04; C07D 409/12; C07D 471/08; C07D 498/08; A61P 25/04

(57) ABSTRACT

The invention relates to compounds that are useful for the prevention or treatment of TRPM3 mediated disorders, more in particular disorders selected from pain and inflammatory hypersensitivity. The invention also relates to a method for the prevention or treatment of said TRPM3 mediated disorders.

### FIELD OF THE INVENTION

The invention relates to compounds that are useful for the prevention or treatment of TRPM3 mediated disorders, more in particular disorders selected from pain and inflammatory hypersensitivity. The invention also relates to a method for the prevention or treatment of said TRPM3 mediated disorders.

### BACKGROUND OF THE INVENTION

The TRP superfamily consists of proteins with six transmembrane domains (6TM) that assemble as homo- or heterotetramers to form cation-permeable ion channels. The name TRP originates from the Drosophila trp (transient receptor potential) mutant, which is characterized by a transient receptor potential in the fly photoreceptors in the response to sustained light. In the last 15 years, trp-related channels have been identified in yeast, worms, insects, fish and mammals, including 27 TRPs in humans. Based on sequence homology, TRP channels can be divided into seven subfamilies: TRPC, TRPV, TRPM, TRPA, TRPP, TRPML and TRPN.

Members of the TRP superfamily are expressed in probably all mammalian organs and cell types, and in recent years great progress has been made in the understanding of their physiological role. The tailored selectivity of certain TRP channels enables them to play key roles in the cellular uptake and/or transepithelial transport of Ca²⁺, Mg²⁺ and trace metal ions. Moreover, the sensitivity of TRP channels to a broad array of chemical and physical stimuli, allows them to function as dedicated biological sensors involved in processes ranging from vision to taste, and tactile sensation. In particular, several members of the TRP superfamily exhibit a very high sensitivity to temperature. These so-called thermoTRPs are highly expressed in sensory neurons and/or skin keratinocytes, where they act as primary thermosensors for the detection of innocuous and noxious (painful) temperatures.

It is becoming increasingly clear that TRP channel dysfunction is directly involved in the etiology of various inherited and acquired diseases. Indeed, both loss-of-function and gain-of-function mutations in the TRP channel genes have been identified as the direct cause of inherited diseases, including brachyolmia, hypomagnesemia with secondary hypocalcemia, polycystic kidney disease, mucolipidosis type IV and familial focal segmental glomerulosclerosis. Moreover, TRP channel function/dysfunction has been directly linked to a wide range of pathological conditions, including chronic pain, hypertension, cancer and neurodegenerative disorders.

TRPM3 (Transient receptor potential melastatin 3) represents a promising pharmacological target. TRPM3 is expressed in a large subset of small-diameter sensory neurons from dorsal root and trigeminal ganglia, and is involved in heat sensing. The neurosteroid pregnenolone sulfate is a potent known activator of TRPM3 (Wagner et al., 2008). The neurosteroid pregnenolone sulfate evoked pain in wild type mice but not in knock-out TRPM3 mice. It was also recently shown that CFA induced inflammation and inflammatory pain are eliminated in TRPM3 knock-out mice. Therefore, TRPM3 antagonists could be used as analgesic drugs to counteract pain, such as inflammatory pain (Vriens J. et al. Neuron, May 2011).

A few TRPM3 antagonists are known, but none of them points towards the compounds of the current invention (Straub I et al. Mol Pharmacol, November 2013). For instance, Liquiritigenin, a postulated TRPM3 blocker has been described to decrease mechanical and cold hyperalgesia in a rat pain model (Chen L et al. Scientific reports, July 2014). There is still a great medical need for novel, alternative and/or better therapeutics for the prevention or treatment of TRPM3 mediated disorders, more in particular for pain such as inflammatory pain. Therapeutics with good potency on a certain type of pain, low level or no side-effects (such as no possibilities for addiction as with opiates, no toxicity) and/or good or better pharmacokinetic or -dynamic properties are highly needed.

The invention provides a class of novel compounds which are antagonists of TRPM3 and can be used as modulators of TRPM3 mediated disorders.

### SUMMARY OF THE INVENTION

The invention provides benzofuran derivatives and pharmaceutical compositions comprising such benzofuran derivatives. The invention also provides benzofuran derivatives for use as a medicament, more in particular for use in the prevention and/or treatment of TRPM3 mediated disorders, especially for use in the prevention and/or treatment of pain and/or inflammatory hypersensitivity; and/or for counteracting pain and/or inflammatory hypersensitivity.

The invention also provides the use of benzofuran derivatives for the manufacture of pharmaceutical compositions or medicaments for the prevention and/or treatment of TRPM3 mediated disorders, especially for the prevention and/or treatment of pain and/or inflammatory hypersensitivity; and/or for counteracting pain and/or inflammatory hypersensitivity.

The invention also provides a method for the prevention or treatment of a TRPM3 mediated disorder by administering the benzofuran derivatives according to the invention to a subject in need thereof. More in particular, the invention relates to such method for the prevention and/or treatment of pain and/or inflammatory hypersensitivity; and/or for counteracting pain and/or inflammatory hypersensitivity.

The invention further provides a method for the preparation of the benzofuran derivatives of the invention, comprising the steps of:
reacting a benzoquinone with a suitable 0-ketoester or an enamine derivative to obtain 5-hydroxybenzofuran-3-carboxylate ester derivatives,
substituting previously obtained 5-hydroxybenzofuran-3-carboxylate ester derivatives with suitable derivatives bearing a leaving group or alcohol derivatives under Mitsunobu conditions to obtain 5-O-substituted-benzofuran-3-carboxylate ester derivatives
converting the previously obtained 5-O-substituted-benzofuran-3-carboxylate ester derivatives in carboxylic acid to obtain the desired benzofuran derivatives of the invention, and
coupling the previously obtained 5-O-substituted-benzofuran-3-carboxylic acid derivatives with a suitable amine to obtain the desired amide derivatives of the invention.

### DETAILED DESCRIPTION OF THE INVENTION

The invention will be further described and in some instances with respect to particular embodiments, but the invention is not limited thereto.

The first aspect of the invention is the provision of a compound of formula (I) (also referred to as benzofuran derivative according to the invention), a stereo-isomeric form, a physiologically acceptable salt, solvate and/or polymorph thereof

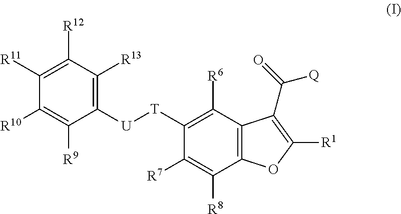

preferably for use in the treatment of pain,
wherein
R¹ represents —F, —Cl, —Br, —I, —CN, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
Q represents —OR² or —NR³R⁴; preferably Q represents —NR³R⁴;
R² represents —R^{Y};
R³ represents —OH or —R^{Y};
R⁴ represents —R^{Y} or —S(═O)₂R^{Y};
or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —CR⁵R⁵′—; or T represents —CR⁵R⁵′— and U represents —O—;
R⁵ and R⁵′ independently of one another represent —R^{Y};
or, provided that T represents —O— and U represents —CR⁵R⁵′—, alternatively R⁵ and R⁹ together form a 4-8-membered carbocycle, saturated or unsaturated, unsubstituted or mono- or polysubstituted; or a 4-8 membered heterocycle, saturated or unsaturated, containing 1 to 3 heteroatoms selected from N, O and S, unsubstituted or mono- or polysubstituted;
R⁶, R⁷ and R⁸ independently of one another represent —F, —Cl, —Br, —I, —CN, —NO₂, —SF₅, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
R⁹, R¹⁰, R¹¹, R¹² and R¹³ independently of one another represent —F, —Cl, —Br, —I, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}; preferably with the proviso that at least one of R⁹, R¹⁰, R¹¹, R¹² and R¹³ does not represent —H;
wherein
R^{W} and R^{X} independently of one another in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y} and R^{Z} independently of one another in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
and wherein “mono- or polysubstituted” in each case independently means substituted with one or more, e.g. 1, 2, 3, 4, or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, —S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, —S(═O)₂-(5 to 14-membered heteroaryl).

In preferred embodiments of the benzofuran derivative according to the invention
(a-1) Q represents —OR²; and R¹ represents —CH₂F, —CHF₂, —CF₃, or —CN; and/or
(a-2) Q represents —OR²; and at least one of R⁵ and R⁵′ does not represent —H; and at least one of R⁹, R¹⁰, R¹¹, R¹² and R¹³ does not represent —H; and/or
(a-3) Q represents —OR²; and R⁸ does not represent —H;
   or
(b-1) Q represents —NR³R⁴; and R¹ represents —CH₂F, —CHF₂, —CF₃, or —CN; and/or
(b-2) Q represents —NR³R⁴; and at least one of R⁹, R¹⁰, R¹¹, R¹² and R¹³ does not represent —H; and with the proviso that the following compounds are excluded:

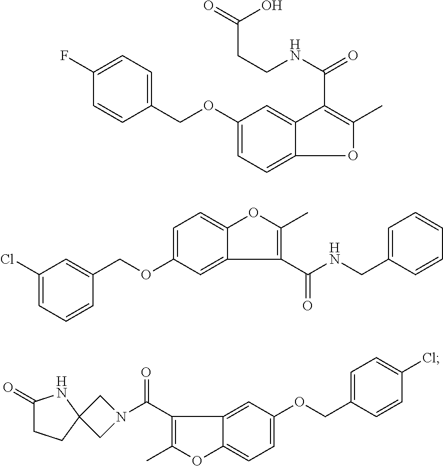

and/or
(b-3) Q represents —NR³R⁴; and at least one of R⁵ and R⁵′ does not represent —H; and/or
(b-4) Q represents —NR³R⁴; and at least one of R⁶, R⁷ and R⁸ does not represent —H; with the proviso that the following compound is excluded:

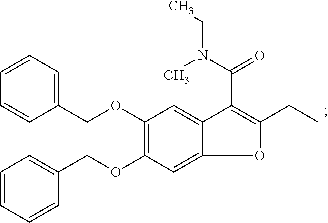 [SMILES: CCc1oc2cc(OCc3ccccc3)c(OCc3ccccc3)cc2c1C(=O)N(C)CC]

and/or
(b-5) Q represents —NR³R⁴; and R³ represent —H; and at least one of R⁹, R¹⁰, R¹¹, R¹² and R¹³ does not represent —H; and R⁴ represents
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted;
   wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.

In a preferred embodiment of the benzofuran derivative according to the invention T represents —O— and U represents —CR⁵R⁵′—. According to this embodiment, the benzofuran derivative according to the invention is a compound of formula (II), a stereo-isomeric form, a physiologically acceptable salt, solvate and/or polymorph thereof

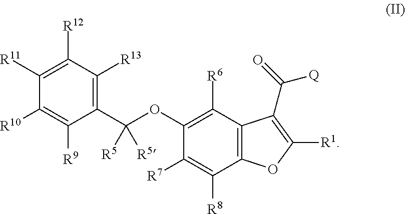

In a preferred embodiment of the benzofuran derivative according to the invention Q represents —NR³R⁴.

In another preferred embodiment of the benzofuran derivative according to the invention Q represents —OR².

In a preferred embodiment of the benzofuran derivative according to the invention R¹ represents
—H, —F, —Cl, —Br, —I;
—C₁₋₆-alkyl, saturated or unsaturated, unsubstituted, mono- or polysubstituted; —O—C₁₋₆-alkyl, saturated or unsaturated, unsubstituted, mono- or polysubstituted;
—C(═O)C₁₋₆-alkyl, saturated or unsaturated, unsubstituted, mono- or polysubstituted;
—C(═O)OC₁₋₆-alkyl, saturated or unsaturated, unsubstituted, mono- or polysubstituted;
—C(═O)NHC₁₋₆-alkyl, saturated or unsaturated, unsubstituted, mono- or polysubstituted;
—C(═O)N(C₁₋₆-alkyl)₂, saturated or unsaturated, unsubstituted, mono- or polysubstituted;
—S(═O)C₁₋₆-alkyl, saturated or unsaturated, unsubstituted, mono- or polysubstituted;
—S(═O)₂—C₁₋₆-alkyl, saturated or unsaturated, unsubstituted, mono- or polysubstituted;
—C₁-C₆-heteroalkyl, saturated or unsaturated, unsubstituted, mono- or polysubstituted; or
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.

Preferably, R¹ represents —H, —F, —Cl, —Br, —I, —C₁₋₆-alkyl, —O—C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NHC₁₋₆-alkyl, —C(═O)N(C₁₋₆-alkyl)₂, —S(═O)—C₁₋₆-alkyl, —S(═O)₂—C₁₋₆-alkyl, —O—C₁₋₆-alkyl, -cyclopropyl unsubstituted, cyclobutyl unsubstituted, cyclopentyl unsubstituted or cyclohexyl unsubstituted.

Preferably, R¹ represents —H, —C₁₋₆-alkyl, —C₁₋₆-alkylene-O—C₁₋₆-alkyl, —CH₂F, —CHF₂, —CF₃, or -cyclopentyl, unsubstituted. Preferably, R¹ represents —CH₃.

Preferably, R¹ represents —CH₂F, —CHF₂, —CF₃, or —CN. Preferably, R¹ represents —C(═O)NH₂, or —CHF₂.

In a preferred embodiment of the benzofuran derivative according to the invention R¹ is not —H.

Preferably, R¹ represents —H, —C₁₋₃-alkyl, —CF₃, —CF₂H, —CFH₂, —CF₂Cl, —CFCl₂, —C₁₋₃-alkylene-CF₃, —C₁₋₃-alkylene-CF₂H, or —C₁₋₃-alkylene-CFH₂; more preferably —CH₃.

In a preferred embodiment of the benzofuran derivative according to the invention R² represents
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted.

Preferably, R² represents —H, —C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.

Preferably, R² represents —H or —C₁₋₆-alkyl.

In a preferred embodiment of the benzofuran derivative according to the invention R³ represents
—H;
—OH;
—C₁-C₆-alkyl, saturated or unsaturated, unsubstituted, mono- or polysubstituted; or
—C₁-C₆-heteroalkyl, saturated or unsaturated, unsubstituted, mono- or polysubstituted.

Preferably, R³ represents —H, —OH, —C₁₋₆-alkyl, —C₁₋₆-alkylene-OH, —C₁₋₆-alkylene-C(═O)—NH₂, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃. More preferably, R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.

Preferably, R³ represents —H, —OH, or —C₁₋₆-alkyl, saturated, unsubstituted or monosubstituted with —OH. Preferably, R³ represents —H.

Preferably, R³ represents —H and R⁴ represents a residue other than —H.

In a preferred embodiment of the benzofuran derivative according to the invention R⁴ represents
—H;
—S(═O)C₁₋₆-alkyl, saturated or unsaturated, unsubstituted, mono- or polysubstituted;
—S(═O)₂—C₁₋₆-alkyl, saturated or unsaturated, unsubstituted, mono- or polysubstituted;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.

Preferably, R⁴ represents
—S(═O)₂C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted; —S(═O)₂(3-14-membered cycloalkyl), wherein said 3-14-membered cycloalkyl is selected from the group consisting of cyclopropyl, cyclobutyl, cyclopentyl, cyclohexyl, and cycloheptyl, in each case saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
—C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is selected from the group consisting of cyclopropyl, cyclobutyl, cyclopentyl, cyclohexyl, and cycloheptyl, in each case saturated or unsaturated, in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from the group consisting of azepane, 1,4-oxazepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofurane, tetrahydropyrane, tetrahydrothiopyrane, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo-[3.3.1]nonane, hexahydro-1H-pyrrolizine, hexahydrocyclopenta[c]pyrrole, octahydrocyclopenta[c]pyrrole, and octahydropyrrolo[1,2-a]pyrazin; in each case unsubstituted, mono- or polysubstituted with substituents independently of one another selected from the group consisting of —F, —Cl, —C₁₋₆-alkyl, —C₁₋₆-alkylene-CF₃, —OH, ═O, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
-phenyl unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of benzimidazole, benzisoxazole, benzoazole, benzodioxole, benzofuran, benzothiadiazole, benzothiazole, benzothiophene, carbazole, cinnoline, dibenzofuran, furane, furazane, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted.

Preferably, R⁴ represents
—H;
—S(═O)₂C₁₋₆-alkyl, saturated, unsubstituted, monosubstituted or polysubstituted with —F;
—S(═O)₂(3-14-membered cycloalkyl), saturated, unsubstituted;
—C₁₋₆-alkyl, saturated, unsubstituted, monosubstituted or disubstituted with substituents independently of one another selected from the group consisting of —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C(═O)NH₂, —C(═O)—NH—C₁₋₃-alkyl, —C(═O)—N(C₁₋₃-alkyl)₂, or -phenyl unsubstituted;
3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is saturated, unsubstituted, monosubstituted or disubstituted with substituents independently of one another selected from the group consisting of —C₁₋₆-alkyl, —C₁₋₆-alkylene-NH₂, —C₁₋₆-alkylene-NH—C₁₋₆-alkylene-CF₃, —C₁₋₆-alkylene-OH, —C₁₋₆-alkylene-NHC(═O)O—C₁₋₆-alkyl, —OH, —OC₁₋₆-alkyl, —NH₂, —N(C₁₋₆-alkyl)₂, —NHC(═O)O—C₁₋₆-alkyl;
3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from azetane, 1,4-oxazepane, pyrrolidine, piperidine, azepane, diazepane, tetrahydrofurane, tetrahydropyrane, oxetane, morpholine, piperazine, hexahydrocyclopenta[c]pyrrole, octahydrocyclopenta[c]pyrrole, octahydropyrrolo[1,2-a]pyrazin, 8-azabicyclo[3.2.1]octane, 9-azabicyclo[3.3.1]nonane, quinuclidine, hexahydro-1H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —OH, ═O, —C₁₋₆-alkyl, —C₁₋₆-alkylene-CF₃, —C₁₋₆-alkylene-OH, —C₁₋₆-alkylene-O—C₁₋₆-alkyl, —NH₂, —N(C₁₋₆-alkyl)₂, —C₁₋₆-alkylene-NH₂, —C₁₋₆-alkylene-N(C₁₋₆-alkyl)₂, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —S(═O)₂C₁₋₆-alkyl, oxetanyl, pyrimidinyl, —C₁₋₆-alkylene-phenyl;
-phenyl unsubstituted;
5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —C₁₋₆-alkyl, —OH.

In a preferred embodiment of the benzofuran derivative according to the invention R³ and R⁴ together form a 5- or 6-membered heterocycle containing 1 or 2 heteroatoms selected from N, O and S, saturated or unsaturated, unsubstituted or mono- or polysubstituted.

Preferably, R³ and R⁴ together form a heterocycle selected from the group consisting of pyrrolidine, piperidine, morpholine, and piperazine, in each case unsubstituted, mono- or polysubstituted with substituents independently of one another selected from the group consisting of —C₁₋₆-alkyl, —NH₂, —NHCH₃, —N(CH₃)₂, —C(═O)NH—C₁₋₆-alkyl, —C(═O)N(C₁₋₆-alkyl)₂, —C(═O)O—C₁₋₆-alkyl, —NHC(═O)O—C₁₋₆-alkyl, -pyridyl unsubstituted, and 1,2,4-oxadiazole unsubstituted or monosubstituted with —C₁₋₆-alkyl.

Preferably, R³ and R⁴ together form a
pyrrolidine ring, unsubstituted or monosubstituted with —N(CH₃)₂;
piperidine ring, unsubstituted or monosubstituted with a substituent selected from the group consisting of —C₁₋₆-alkyl, —NH₂, —N(CH₃)₂, —C(═O)NH—C₁₋₆-alkyl, —C(═O)O—C₁₋₆-alkyl, —NHC(═O)O—C₁₋₆-alkyl, and 1,2,4-oxadiazole unsubstituted or monosubstituted with —C₁₋₆-alkyl;
morpholine ring, unsubstituted; or
piperazine ring, unsubstituted or N-substituted with a substituent selected from the group consisting of —C₁₋₆-alkyl and -pyridyl unsubstituted.

In a preferred embodiment, R³ and R⁴ both do not represent —H. In preferred embodiments, R³ and R⁴ together with the nitrogen atom to which they are attached form a residue selected from the group consisting of:

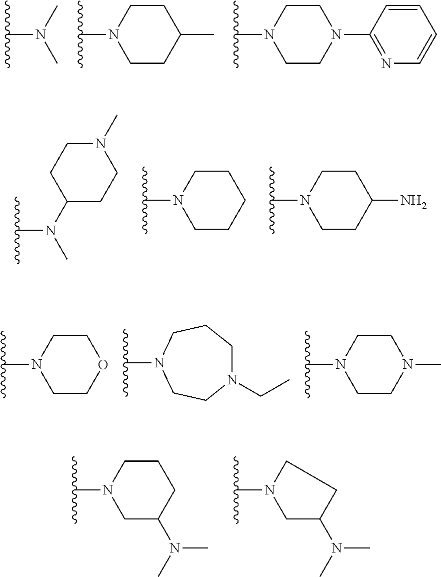

In other preferred embodiments, R³ represents —H and R⁴ does not represent —H.

In preferred embodiments, R³ represents —H and R⁴ represents —C₁-C₆-alkyl, saturated or unsaturated, unsubstituted, mono- or polysubstituted. In preferred embodiments, R³ represents —H and R⁴ represents a residue selected from the group consisting of:

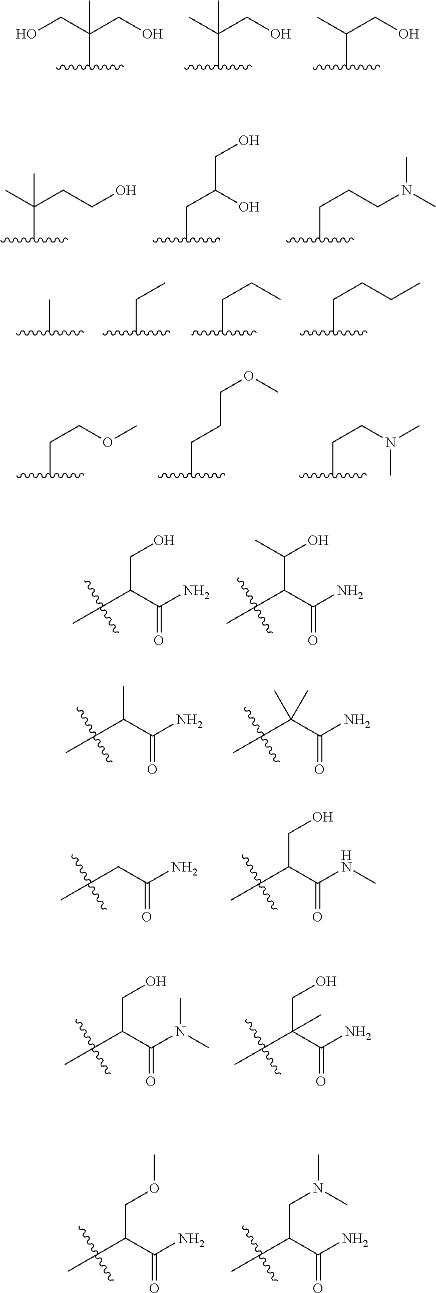

In preferred embodiments, R³ represents —H and R⁴ represents a residue —CR′R″—(CH₂)ₘ—OH, wherein m is an integer of from 1 to 6, preferably from 1 to 3; and wherein R′ and R″ independently of one another represent —H, —C₁₋₃-alkyl, —CF₃, —CF₂H, —CFH₂, —C₁₋₃-alkylene-CF₃, —C₁₋₃-alkylene-CF₂H, —C₁₋₃-alkylene-CFH₂, alkylene-O—C₁₋₃-alkyl, —C₁₋₃-alkylene-OH, —C(═O)—NH₂, —C(═O)—NH—C₁₋₃-alkyl, or —C(═O)—N(C₁₋₃-alkyl)₂; preferably —H, —CH₃, —C₁₋₃-alkylene-OH, C(═O)—NH₂, —C(═O)—NH—C₁₋₃-alkyl, or —C(═O)—N(C₁₋₃-alkyl)₂. In a preferred embodiment, at least R′ or R″ does not represent —H. In a preferred embodiment, neither R′ nor R″ represents —H.

In other preferred embodiments, R³ represents —H and R⁴ represents a 3-14-membered cycloalkyl, saturated or unsaturated, unsubstituted, mono- or polysubstituted; or a 3-14-membered heterocycloalkyl, saturated or unsaturated, unsubstituted, mono- or polysubstituted.

In preferred embodiments, R³ represents —H and R⁴ represents a 3-membered cycloalkyl, saturated or unsaturated, unsubstituted, mono- or polysubstituted; or a 3-membered heterocycloalkyl, saturated or unsaturated, unsubstituted, mono- or polysubstituted. In preferred embodiments, R³ represents —H and R⁴ represents a residue selected from the group consisting of:

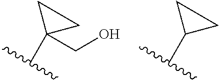

In preferred embodiments, R³ represents —H and R⁴ represents a 3-14-membered cycloalkyl (preferably a 4-membered cycloalkyl), saturated or unsaturated, unsubstituted, mono- or polysubstituted; or a 3-14-membered heterocycloalkyl (preferably a 4-membered heterocycloalkyl), saturated or unsaturated, unsubstituted, mono- or polysubstituted. In preferred embodiments, R³ represents —H and R⁴ represents a residue selected from the group consisting of:

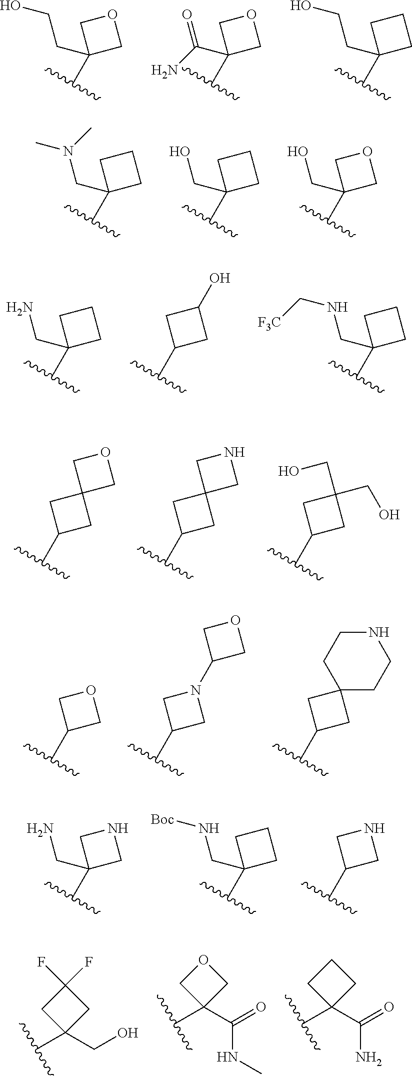

In preferred embodiments, R³ represents —H and R⁴ represents a residue according to general formula (A),

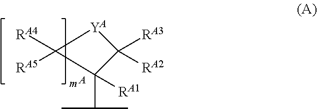

wherein
m^{A} is 0 or 1;
Y^{A} is selected from —O—, —NR^{A6}— and —CR^{A7}R^{A8}—; and
R^{A1}, R^{A2}, R^{A3}, R^{A4}, R^{A5}, R^{A6}, R^{A7}, and R^{A8} independently of one another represent —H, —F, —C₁₋₃-alkyl, —C₁₋₃-alkylene-OH, —C₁₋₃-alkylene-NH₂, —C₁₋₃-alkylene-NH(C₁₋₃-alkyl), —C₁₋₃-alkylene-N(C₁₋₃-alkyl)₂, —C₁₋₃-alkylene-NH(C₁₋₃-alkylene-CF₃), —C₁₋₃-alkylene-C(═O)NH₂, —C₁₋₃-alkylene-NH—C(═O)OC₁₋₄-alkyl, —C(═O)NH₂, —C(═O)—NH—C₁₋₃-alkyl, —C(═O)—N(C₁₋₃-alkyl)₂, or -3-oxetanyl; or R^{A7} and R^{A8} together with the carbon atom to which they are attached form a ring and represent —CH₂OCH₂—, —CH₂OCH₂CH₂— or —CH₂CH₂OCH₂CH₂—, —CH₂NHCH₂—, —CH₂NHCH₂CH₂— or —CH₂CH₂NHCH₂CH₂—.

In preferred embodiments, R³ represents —H and R⁴ represents a residue according to general formula (A) as defined above, wherein
m^{A} is 0 or 1;
Y^{A} is selected from —O— and —CR^{A7}R^{A8}—; and
R^{A1}, R^{A2}, R^{A3}, R^{A4}, R^{A5}, R^{A7}, and R^{A8} independently of one another represent —H, —C₁₋₃-alkylene-OH, —C₁₋₃-alkylene-N(C₁₋₃-alkyl)₂, or —C(═O)NH₂; preferably with the proviso that only one of R^{A1}, R^{A2}, R^{A3}, R^{A4}, R^{A5}, R^{A7}, and R^{A8} represents a residue that is not —H.

In preferred embodiments, R³ represents —H and R⁴ represents a residue according to general formula (A) as defined above, wherein
m^{A} is 0 or 1;
Y^{A} is selected from —O— and —CR^{A7}R^{A8}—; and
R^{A1} represents —C₁₋₃-alkylene-OH, —C₁₋₃-alkylene-N(C₁₋₃-alkyl)₂, or —C(═O)NH₂; and
R^{A2}, R^{A3}, R^{A4}, R^{A5}, R^{A7}, and R^{A8} represent —H.

In preferred embodiments, R³ represents —H and R⁴ represents a 3-14-membered cycloalkyl (preferably a cycloalkyl), saturated or unsaturated, unsubstituted, mono- or polysubstituted; or a 3-14-membered heterocycloalkyl (preferably a 5-membered heterocycloalkyl), saturated or unsaturated, unsubstituted, mono- or polysubstituted; or a 5-14-membered heteroaryl (preferably a 5-membered heteroaryl), unsubstituted, mono- or polysubstituted. In preferred embodiments, R³ represents —H and R⁴ represents a residue selected from the group consisting of:

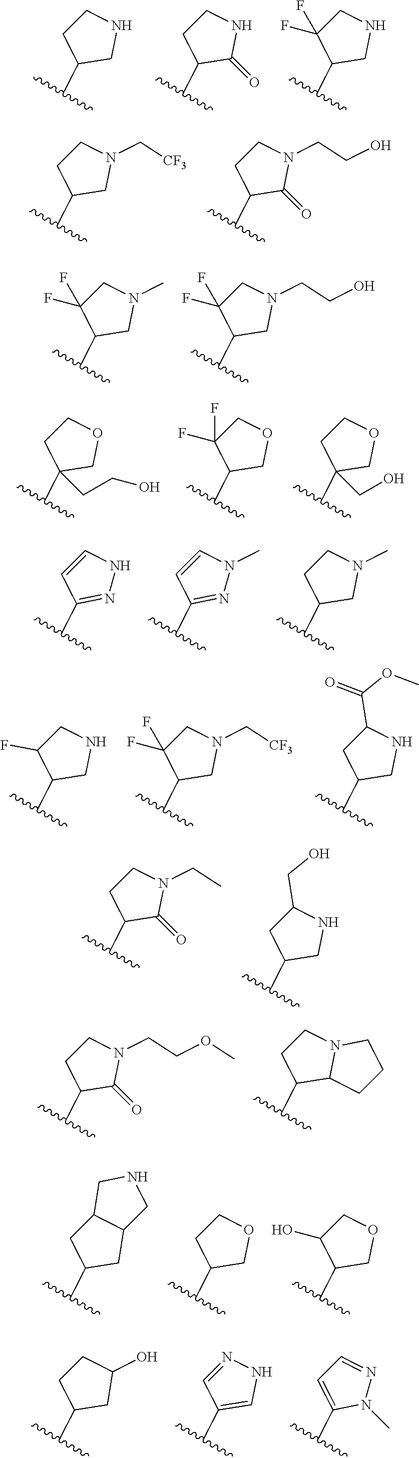

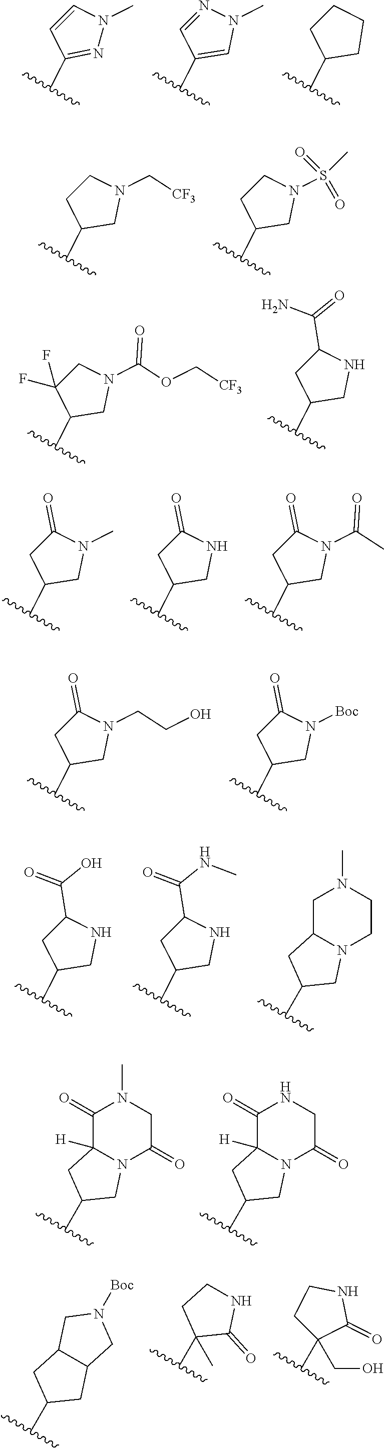

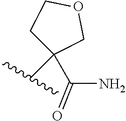 [SMILES: *C1(C(N)=O)CCOC1]

In preferred embodiments, R³ represents —H and R⁴ represents a residue according to general formula (B),

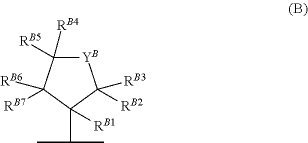

wherein
Y^{B} is selected from —O—, —NR^{B8}— and —CR^{B9}R^{B10}—; and
R^{B1}, R^{B2}, R^{B3}, R^{B4}, R^{B5}, R^{B6}, R^{B7}, R^{B8}, R^{B9} and R^{B10} independently of one another represent —H, —F, —OH, —C(═O)—NH₂, —C(═O)—NH—C₁₋₃-alkyl, —C(═O)—N(C₁₋₃-alkyl)₂, —C₁₋₃-alkyl, —C₁₋₃-alkylene-OH, —C₁₋₃-alkylene-O—C₁₋₃-alkyl, —C₁₋₃-alkylene-CF₃, —C₁₋₃-alkylene-CO₂H, or —C₁₋₃-alkylene-C(═O)O—C₁₋₃-alkyl; or R^{B2} and R^{B3} together represent ═O; or R^{B4} and R^{B5} together represent ═O.

In preferred embodiments, R³ represents —H and R⁴ represents a residue according to general formula (B) as defined above, wherein
Y^{B} is selected from —O— and —NR^{B8}—; and
R^{B1}, R^{B2}, R^{B3}, R^{B4}, R^{B5}, R^{B6}, R^{B7}, R^{B8} independently of one another represent —H, —F, —C₁₋₃-alkyl, —C₁₋₃-alkylene-OH, —C₁₋₃-alkylene-CF₃, or —C(═O)—NH₂; or R^{B2} and R^{B3} together represent ═O; or R^{B4} and R^{B5} together represent ═O; preferably with the proviso that only 1, 2 or 3 of R^{A1}, R^{A2}, R^{A3}, R^{A4}, R^{A5}, R^{A7}, and R^{A8} represent a residue that is not —H; preferably with the proviso that at least one of R^{A1}, R^{A2}, R^{A3}, R^{A4}, R^{A5}, R^{A7}, and R^{A8} represent a residue that is not —H.

In preferred embodiments, R³ represents —H and R⁴ represents a 3-14-membered cycloalkyl (preferably a 6-membered cycloalkyl), saturated or unsaturated, unsubstituted, mono- or polysubstituted; or a 3-14-membered heterocycloalkyl (preferably a 6-membered heterocycloalkyl), saturated or unsaturated, unsubstituted, mono- or poly substituted; or a 6-14-membered aryl (preferably a 6-membered aryl), unsubstituted, mono- or polysubstituted; or a 5-14-membered heteroaryl (preferably a 6-membered heteroaryl), unsubstituted, mono- or polysubstituted. In preferred embodiments, R³ represents —H and R⁴ represents a residue selected from the group consisting of:

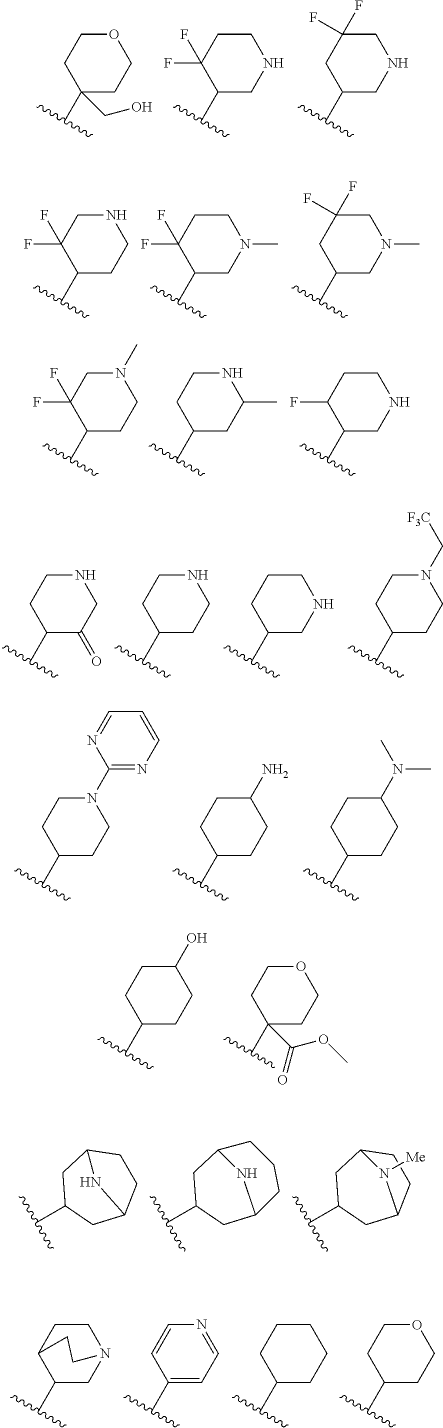

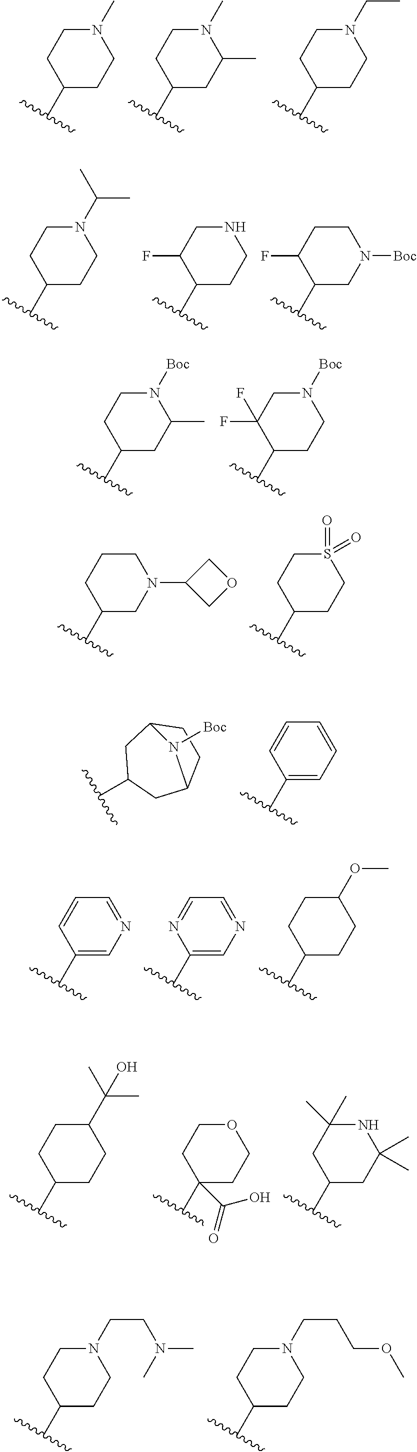

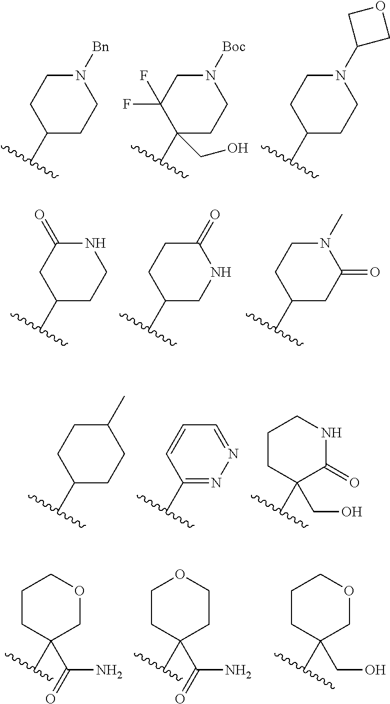

In preferred embodiments, R³ represents —H and R⁴ represents a residue according to general formula (C),

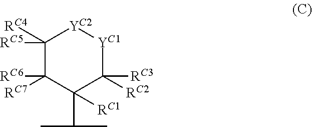

wherein
Y^{C1} is selected from —O—, —S(═O)₂—, —NR^{C8}— and —CR^{C9}R^{C10}— and Y^{C2} represents —CR^{C11}R^{C12}—; or Y^{C1} represents —CR^{C9}R^{C10}— and Y^{C2} is selected from —O—, —S(═O)₂—, and —NR^{C8}—;
R^{C1}, R^{C2}, R^{C3}, R^{C4}, R^{C5}, R^{C6}, R^{C7}, R^{C8}, R^{C9}, R^{C10}, R^{C11} and R^{C12} independently of one another represent —H, —F, —OH, —C(═O)OC₁₋₃-alkyl, —NH₂, —NH(C₁₋₃-alkyl), —N(C₁₋₃-alkyl)₂, —C₁₋₃-alkyl, —C₁₋₃-alkylene-OH, —C₁₋₃-alkylene-CF₃, —C(═O)—NH₂, —C(═O)—NH—C₁₋₃-alkyl, or —C(═O)N—(C₁₋₃-alkyl)₂; or R^{C2} and R^{C3} together represent ═O; or R^{C4} and R^{C5} together represent ═O; or R^{C9} and R^{C10} together represent ═O; or R^{C11} and R^{C12} together represent ═O.

In preferred embodiments, R³ represents —H and R⁴ represents a residue according to general formula (C) as defined above, wherein
Y^{C1} is selected from —O— or —NR^{C8}— and Y^{C2} represents —CR^{C11}R^{C12}—; or Y^{C1} represents —CR^{C9}R^{C10}— and Y^{C2} is selected from —O—, and —NR^{C8}—;
R^{C1}, R^{C2}, R^{C3}, R^{C4}, R^{C5}, R^{C6}, R^{C7}, R^{C8}, R^{C9}, R^{C10}, R^{C11} and R^{C12} independently of one another represent —H, —F, —C₁₋₃-alkyl, —C₁₋₃-alkylene-OH or —C(═O)—NH₂; preferably with the proviso that only 1, 2 or 3 of R^{C1}, R^{C2}, R^{C3}, R^{C4}, R^{C5}, R^{C6}, R^{C7}, R^{C8}, R^{C9}, R^{C10}, R^{C11} and R^{C12} represent a residue that is not —H; preferably with the proviso that at least one of R^{C1}, R^{C2}, R^{C3}, R^{C4}, R^{C5}, R^{C6}, R^{C7}, R^{C8}, R^{C9}, R^{C10}, R^{C11} and R^{C12} represent a residue that is not —H.

In preferred embodiments, R³ represents —H and R⁴ represents a 7-membered cycloalkyl, saturated or unsaturated, unsubstituted, mono- or polysubstituted; or a 7-membered heterocycloalkyl, saturated or unsaturated, unsubstituted, mono- or polysubstituted. In preferred embodiments, R³ represents —H and R⁴ represents a residue:

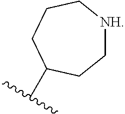 [SMILES: *C1CCCNCC1]

In preferred embodiments, R³ represents —H and R⁴ represents a 3-14-membered cycloalkyl (preferably a 4, 5 or 6-membered cycloalkyl), saturated or unsaturated, unsubstituted, mono- or polysubstituted; wherein said 3-14-membered cycloalkyl is connected through —C₁-C₆-alkylene-, saturated or unsaturated, unsubstituted, mono- or polysubstituted; or a 3-14-membered heterocycloalkyl (preferably a 4, 5 or 6-membered heterocycloalkyl), saturated or unsaturated, unsubstituted, mono- or polysubstituted; wherein said 3-14-membered heterocycloalkyl is connected through —C₁-C₆-alkylene-, saturated or unsaturated, unsubstituted, mono- or polysubstituted; or a 6-14-membered aryl (preferably a 6-membered aryl), unsubstituted, mono- or polysubstituted; wherein said 36-14-membered aryl is connected through —C₁-C₆-alkylene-, saturated or unsaturated, unsubstituted, mono- or polysubstituted; or a 5-14-membered heteroaryl (preferably a 5 or 6-membered heteroaryl), unsubstituted, mono- or polysubstituted; wherein said 5-14-membered heteroaryl is connected through —C₁-C₆-alkylene-, saturated or unsaturated, unsubstituted, mono- or polysubstituted. In preferred embodiments, R³ represents —H and R⁴ represents a residue selected from the group consisting of:

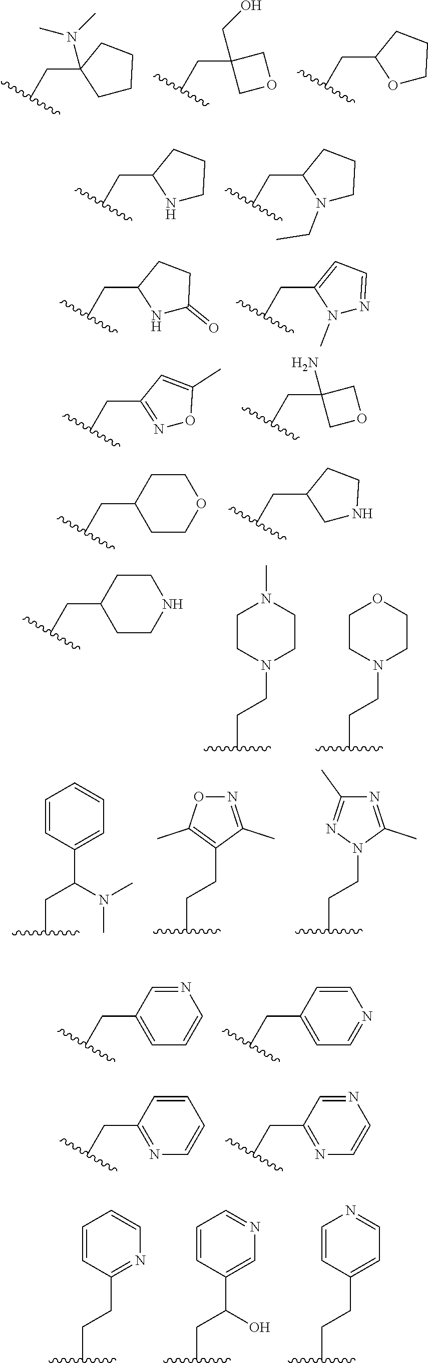

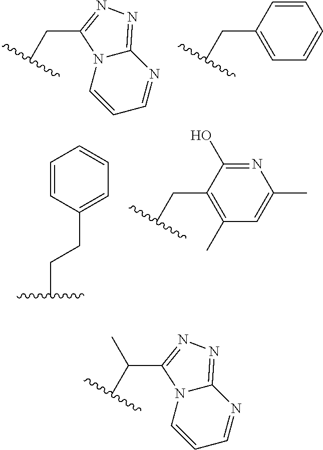

In preferred embodiments, R³ represents —H and R⁴ represents a 5-membered heterocycloalkyl, saturated or unsaturated, unsubstituted, mono- or polysubstituted; wherein said 5-membered heterocycloalkyl is connected through —C₁-C₆-alkylene-, saturated or unsaturated, unsubstituted, mono- or polysubstituted; or a 5-membered heteroaryl, unsubstituted, mono- or polysubstituted; wherein said 5-membered heteroaryl is connected through —C₁-C₆-alkylene-, saturated or unsaturated, unsubstituted, mono- or polysubstituted.

In preferred embodiments, R³ represents —H and R⁴ represents a residue selected from the group consisting of:

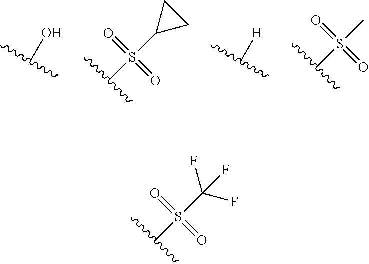

In preferred embodiments, R³ represents —H and R⁴ represents
(i) a residue —CR′R″—(CH₂)ₘ—OH, wherein m is an integer of from 1 to 6, preferably from 1 to 3; and wherein R′ and R″ independently of one another represent —H, —C₁₋₃-alkyl, —CF₃, —CF₂H, —CFH₂, —C₁₋₃-alkylene-CF₃, —C₁₋₃-alkylene-CF₂H, —C₁₋₃-alkylene-CFH₂, —C₁₋₃-alkylene-O—C₁₋₃-alkyl, or —C₁₋₃-alkylene-OH; preferably —H, —CH₃, or —C₁₋₃-alkylene-OH. In a preferred embodiment, at least R′ or R″ does not represent —H. In a preferred embodiment, neither R′ nor R″ represents —H; or
(ii) a residue according to general formula (D),

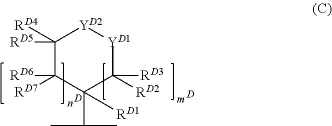

wherein
m^{D} and n^{D} independently of one another are 0, 1, 2, or 3; preferably with the proviso that m^{D}+n^{D}≤3;
Y_{D1} is selected from —O—, —S(═O)₂—, —NR^{D8}— and —CR^{D9}R^{D10}— and Y^{D2} represents —CR^{D11}R^{D12}—, or Y^{D1} represents —CR^{D9}R^{D10}— and Y^{D2} is selected from —O—, —S(═O)₂—, and —NR^{D8}—;
R^{D1}, R^{D2}, R^{D3}, R^{D4}, R^{D5}, R^{D6}, R^{D7}, R^{D8}, R^{D9}, R^{D10}, R^{D11} and R^{D12} independently of one another represent —H, —F, —OH, —C₁₋₃-alkylene-OH, —C(═O)NH₂, —C₁₋₃-alkylene-C(O)NH₂, —C(═O)O—C₁₋₃-alkyl, —NH₂, —C₁₋₃-alkylene-NH₂, —NH(C₁₋₃-alkyl), —N(C₁₋₃-alkyl)₂, —NH(C₁₋₃-alkylene-CF₃), —C₁₋₃-alkylene-OCH₃, —C₁₋₃-alkyl, —C₁₋₃-alkylene-CF₃: or R^{D2} and R^{D3} together represent ═O; or R^{D4} and R^{D5} together represent ═O; or R^{D9} and R^{D10} together represent ═O; or R^{D11} and R^{D12} together represent ═O;
preferably wherein
m^{D} and n^{D} independently of one another are 0, 1, 2 or 3; preferably with the proviso that m^{D}+n^{D}≤3;
Y^{D1} is selected from —O—, —NR^{D8}— and —CR^{D9}R^{D10}— and Y^{D2} represents —CR^{D11}R^{D12}—, or Y^{D1} represents —CR^{D9}R^{D10}— and Y^{D2} is selected from —O— and —NR^{D8}—;
R^{D1}, R^{D2}, R^{D3}, R^{D4}, R^{D5}, R^{D6}, R^{D7}, R^{D8}, R^{D9}, R^{D10}, R^{D11} and R^{D12} independently of one another represent —H, —F, —OH, —C₁₋₃-alkylene-OH, —C(═O)NH₂, —CH₂NH₂, —CH₂N(CH₃)₂, —NHCH₂CF₃, —CH₃, or —CH₂CF₃: or R^{D2} and R^{D3} together represent ═O; or R^{D4} and R^{D5} together represent ═O; or R^{D9} and R^{D10} together represent ═O; or R^{D11} and R^{D12} together represent ═O; preferably with the proviso that only 1, 2 or 3 of R^{D1}, R^{D2}, R^{D3}, R^{D4}, R^{D5}, R^{D6}, R^{D7}, R^{D8}, R^{D9}, R^{D10}, R^{D11} and R^{D12} represent a residue that is not —H; preferably with the proviso that at least one of R^{D1}, R^{D2}, R^{D3}, R^{D4}, R^{D5}, R^{D6}, R^{D7}, R^{D8}, R^{D9}, R^{D10}, R^{D11} and R^{D12} represent a residue that is not —H.

In a preferred embodiment of the benzofuran derivative according to the invention R⁵ and R⁵′ independently of one another represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.

Preferably, R⁵ and R⁵′ independently of one another represent —H, —C₁-C₆-alkyl, or —C₁-C₆-alkylene-N(C₁-C₆-alkyl)₂.

In a preferred embodiment of the benzofuran derivative according to the invention, at least one of R⁵ and R⁵′ is not —H.

In a preferred embodiment of the benzofuran derivative according to the invention, R⁵ and R⁵′ are both —H.

In preferred embodiments, T represents —O— and U represents —CR⁵R⁵′— and the resultant moiety —O—CR⁵R⁵′— represents a residue selected from the group consisting of:

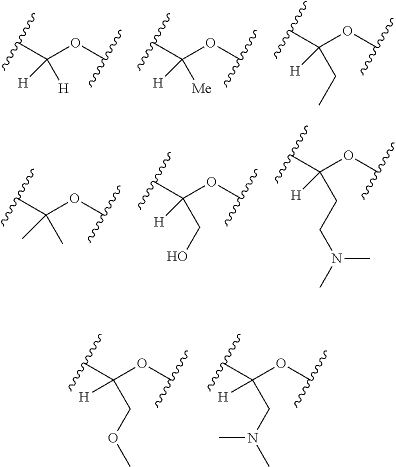

In preferred embodiments, T represents —CR⁵R⁵′— and U represents —O— and the resultant moiety —CR⁵R⁵′— represents a residue:

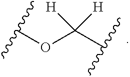 [SMILES: *CO*]

In preferred embodiments, R⁵ represents —H and R⁵′ represents a residue selected from the group consisting of —H, —C₁₋₃-alkyl, —CF₃, —CF₂H, —CFH₂, —C₁₋₃-alkylene-CF₃, —C₁₋₃-alkylene-CF₂H, —C₁₋₃-alkylene-CFH₂, and —C₁₋₃-alkylene-OH; preferably —H or C₁₋₃-alkyl.

In a preferred embodiment of the benzofuran derivative according to the invention R⁵ and R⁹ together form a 5-6-membered carbocycle, unsubstituted; or R⁵ and R⁹ together form a 5-6-membered heterocycle containing 1 heteroatom 0, unsubstituted.

In a preferred embodiment of the benzofuran derivative according to the invention R⁶, R⁷ and R⁸ independently of one another represent
—H;
—F, —Cl, —Br, —I, —OH, —SH, —SF₅, —CN, —NO₂, —C(═O)OH, —NH₂;
—C₁₋₆-alkyl, saturated or unsaturated, unsubstituted, mono- or polysubstituted;
—O—C₁₋₆-alkyl, saturated or unsaturated, unsubstituted, mono- or polysubstituted;
—NHC₁₋₆-alkyl, saturated or unsaturated, unsubstituted, mono- or polysubstituted;
—N(C₁₋₆-alkyl)₂, saturated or unsaturated, unsubstituted, mono- or polysubstituted;
—C(═O)OC₁₋₆-alkyl, saturated or unsaturated, unsubstituted, mono- or polysubstituted;
—OC(═O)C₁₋₆-alkyl, saturated or unsaturated, unsubstituted, mono- or polysubstituted;
—C₁₋₆-heteroalkyl, saturated or unsaturated, unsubstituted, mono- or polysubstituted.

Preferably, R⁶, R⁷ and R⁸ independently of one another represent
—H, —F, —Cl, —Br, —I, —OH, —SH, —SF₅, —CN, —NO₂, —C(═O)OH, —NH₂,
—C₁₋₆-alkyl, —CF₃, —CHF₂, —CH₂F,
—O—C₁₋₆-alkyl, —OCF₃, —OCHF₂, —OCH₂F,
—NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—C(═O)OC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—OC(═O)C₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂; or
—C₁₋₆-heteroalkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂.

In preferred embodiments, R⁶, R⁷ and R⁸ independently of one another represents a residue selected from the group consisting of —H, —F, —Cl, —Br, —I, —CN, C₁₋₃-alkyl, —CF₃, —CF₂H, and —CFH₂; preferably —H or —F.

In a preferred embodiment of the benzofuran derivative according to the invention R⁶ represents —H, —F, —Cl, —CN, or —C₁-C₆-alkyl.

In preferred embodiments, R⁶ represents a residue selected from the group consisting of —H, —F, —Cl, —CN or —CH₃; preferably —H, —F, —CN or —CH₃.

In a preferred embodiment of the benzofuran derivative according to the invention R⁶ does not represent —H.

In a preferred embodiment of the benzofuran derivative according to the invention R⁷ represents —H, —F, —Cl, —CN, or —C₁-C₆-alkyl.

In a preferred embodiment of the benzofuran derivative according to the invention R⁷ does not represent —H.

In preferred embodiments, especially when Q represents —NR³R⁴, R⁷ represents a residue selected from the group consisting of —H, —F, —Cl, —CN or CH₃; preferably —H, —F, —Cl or —CH₃.

In preferred embodiments, especially when Q represents —OR², R⁷ represents a residue selected from the group consisting of —H or

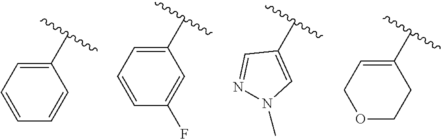

In a preferred embodiment of the benzofuran derivative according to the invention R⁸ represents —H, —F, —Cl, —CN, or —C₁-C₆-alkyl.

In a preferred embodiment of the benzofuran derivative according to the invention R⁸ does not represent —H.

In preferred embodiments, R⁸ represents a residue selected from the group consisting of —H, —F, —Cl, —CN or CH₃; preferably —F.

In preferred embodiments of the benzofuran derivative according to the invention
(i) R⁶, R⁷ and R⁸ each represent —H; or
(ii) two of R⁶, R⁷ and R⁸ represent —H and the other of R⁶, R⁷ and R⁸ represents —F, —Cl, —CN, or —CH₃; or
(iii) one of R⁶, R⁷ and R⁸ represents —H and the other of R⁶, R⁷ and R⁸ independently of one another represent —F, —Cl, —CN, or —CH₃.

In a particularly preferred embodiment, the compound is according to general formula (I), wherein
R¹ represents —CH₃; and/or
R³ represents —H; and/or
R⁴ represents

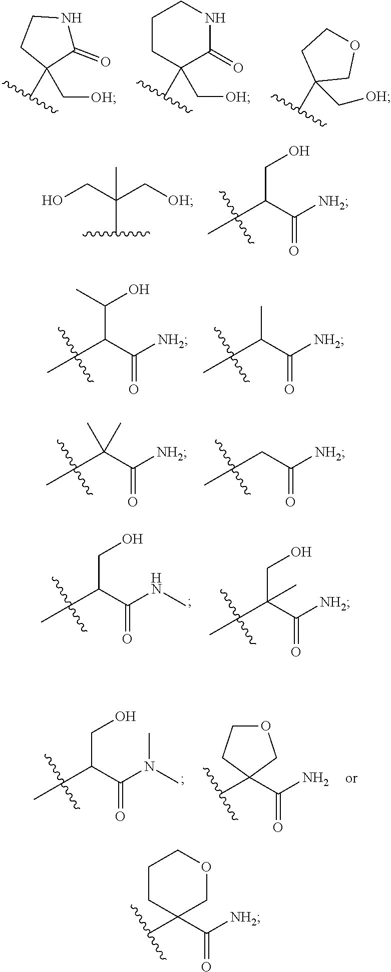

and/or
R⁶, R⁷ and R⁸ each represent —H; and/or
T represents —O—; and/or
U represents —CH₂—; and/or
R⁹, R¹⁰, R¹¹, R¹² and R¹³ independently of one another represent —H, —F, —Cl, —CF₃, —CN, —CH₃, or —O—CH₃.

In a preferred embodiment of the benzofuran derivative according to the invention R⁹, R¹⁰, R¹¹, R¹² and R¹³ independently of one another represent
—H, —F, —Cl, —Br, —I, —CN, —C(═O)OH, —NH₂, —NO₂, —OH, ═O, —SF₅;
—C₁₋₆-alkyl, saturated or unsaturated, unsubstituted, mono- or polysubstituted;
—C(═O)O—C₁₋₆-alkyl, saturated or unsaturated, unsubstituted, mono- or polysubstituted;
—NHC₁₋₆-alkyl, saturated or unsaturated, unsubstituted, mono- or polysubstituted;
—N(C₁₋₆-alkyl)₂, saturated or unsaturated, unsubstituted, mono- or polysubstituted;
—O—C₁₋₆-alkyl, saturated or unsaturated, unsubstituted, mono- or polysubstituted;
—S(═O)₂—C₁₋₆-alkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted.

Preferably, R⁹, R¹⁰, R¹¹, R¹² and R¹³ independently of one another represent
—H, —OH, —F, —Cl, —Br, —I, —SH, —CF₃, —CHF₂, —CH₂F, —OCF₃, —OCHF₂, —OCH₂F, SF₅, —CN, —NO₂, —C(═O)OH, —NH₂, or —N(CH₃)₂;
—C₁₋₆-alkyl, saturated or unsaturated, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
—C₁₋₆-heteroalkyl, saturated or unsaturated, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
—OC₁₋₆-alkyl, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
—O(C═O)C₁₋₆-alkyl, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
—C(═O)OC₁₋₆-alkyl, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
3-14-membered cycloalkyl selected from the group consisting of cyclopropyl, cyclobutyl, cyclopentyl, cyclohexyl and cycloheptyl; in each case unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
3-14-membered heterocycloalkyl selected from the group consisting of azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofurane, tetrahydropyrane, tetrahydrothiopyrane, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo-[3.3.1]nonane, hexahydro-1H-pyrrolizine, hexahydrocyclopenta[c]pyrrole, octahydrocyclopenta[c]pyrrole, and octahydropyrrolo[1,2-a]pyrazin, in each case unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂.

Preferably, R⁹, R¹¹, R¹¹, R¹² and R¹³ independently of one another represent —H, —F, —Cl, —CN, —OH, ═O, —C₁₋₆-alkyl, —CHF₂, —CF₃, —C₁₋₆-alkylene-NH₂, —C₁₋₆-alkylene-NHC(═O)O—C₁₋₆-alkyl, —C₁₋₆-alkylene-OH, —C₁₋₆-alkylene-NHC(═O)—O—C₁₋₆-alkyl, —C(═O)O—C₁₋₆-alkyl, —N(C₁₋₆-alkyl)₂, —OC₁₋₆-alkyl, —OCF₃, —O—C₁₋₆-alkylene-N(C₁₋₆-alkyl)₂, —S(═O)₂—C₁₋₆-alkyl, -azetidine, —C₁₋₆-alkylene-O-tetrahydropyran, or -piperazine substituted with —C₁₋₆-alkyl.

In preferred embodiments of the benzofuran derivative according to the invention
(i) R⁹, R¹⁰, R¹¹, R¹² and R¹³ represent —H;
(ii) four of R⁹, R¹⁰, R¹¹, R¹² and R¹³ represent —H and the other two of R⁹, R¹⁰, R¹¹, R¹² and R¹³ represent a substituent other than —H; or
(iii) three of R⁹, R¹⁰, R¹¹, R¹² and R¹³ represent —H and the other of R⁹, R¹⁰, R¹¹, R¹² and R¹³ represents a substituent other than —H.

In preferred embodiments, the phenyl moiety is unsubstituted or monosubstituted in ortho-position, i.e., R¹⁰, R¹¹, R¹², and R¹³ represent —H and R⁹ represents a residue that is not —H. Preferably, the phenyl moiety is unsubstituted or substituted in ortho position and selected from the group consisting of:

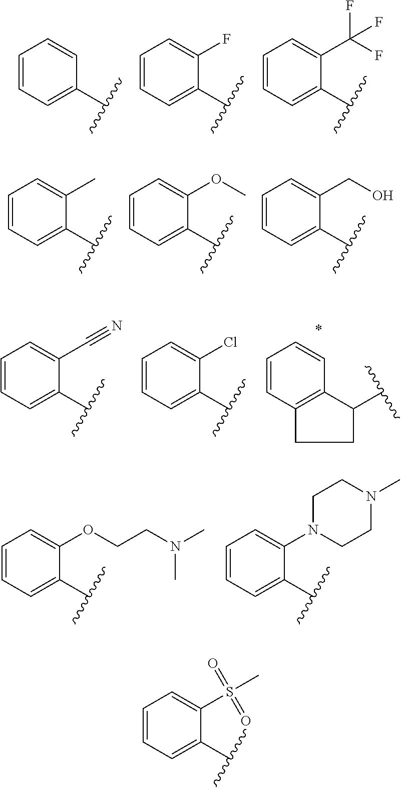

* the shown structure also includes U representing —CR⁵R⁵′—, wherein R⁵ and R⁹ together form a 5-membered saturated unsubstituted carbocycle

In preferred embodiments, the phenyl moiety is monosubstituted in ortho-position, i.e. R¹⁰, R¹¹, R¹², and R¹³ represent —H and R⁹ represents a residue selected from the group consisting of —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₁H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH; preferably —F, —Cl, —Br, —I, —CN, —CH₃, —CF₃, —CF₂H, —CFH₂, —OCF₃, and —OCH₃.

In preferred embodiments, the phenyl moiety is monosubstituted in meta-position, i.e., R⁹, R¹¹, R¹², and R¹³ represent —H and R¹⁰ represents a residue that is not —H. Preferably, the phenyl moiety that is substituted in meta position is selected from the group consisting of:

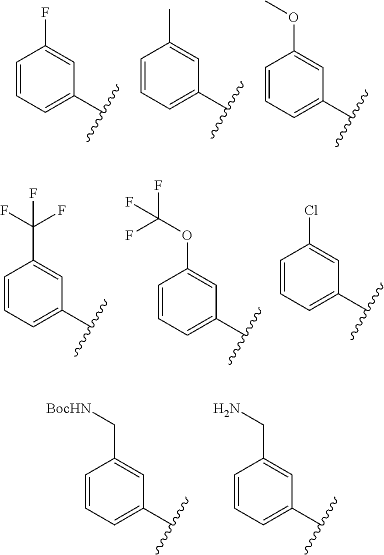

In preferred embodiments, the phenyl moiety is monosubstituted in meta-position, i.e. R⁹, R¹¹, R¹², and R¹³ represent —H and R¹⁰ represents a residue selected from the group consisting of —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH; preferably —F, —Cl, —Br, —I, —CN, —CH₃, —CF₃, —CF₂H, —CFH₂, —OCF₃, and —OCH₃.

In preferred embodiments, the phenyl moiety is monosubstituted in para-position, i.e., R⁹, R¹⁰, R¹², and R¹³ represent —H and R¹¹ represents a residue that is not —H. Preferably, the phenyl moiety that is substituted in para position is selected from the group consisting of:

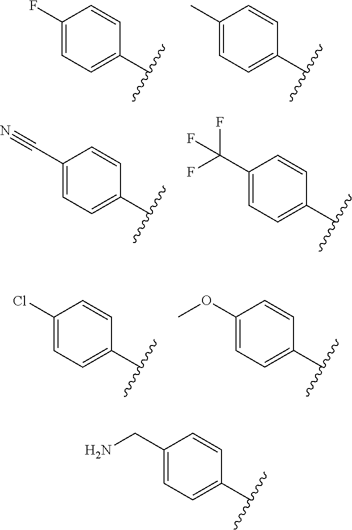

In preferred embodiments, the phenyl moiety is monosubstituted in para-position, i.e. R⁹, R¹⁰, R¹², and R¹³ represent —H and R¹¹ represents a residue selected from the group consisting of —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH; preferably —F.

In preferred embodiments, the phenyl moiety is disubstituted. Preferably, the phenyl moiety that is disubstituted is selected from the group consisting of:

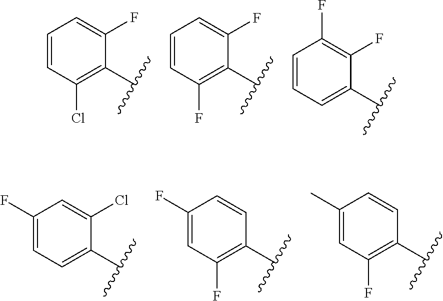

In preferred embodiments,
(i) one or two of R⁹, R¹⁰, R¹¹, R¹², and R¹³ are selected from —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, —C₁₋₃-alkylene-OH, while the other are hydrogen; or
(ii) one or two of R⁹, R¹⁰, R¹¹, R¹², and R¹³ are selected from —F, —Cl, —Br, —I, —CN, -Me, —CF₃, —CF₂H, —CFH₂, —OCF₃, —OCH₃.

In preferred embodiments, R⁹ or R¹³ are selected from —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH, while R″, R¹¹ and R¹² are hydrogen. In preferred embodiments, R⁹ or R¹³ are selected from —F, —Cl, —Br, —I, —CN, -Me, —CF₃, —CF₂H, —CFH₂, —OCF₃, and —OCH₃.

In preferred embodiments, R″ or R¹² are selected from —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH, while R⁹, R¹¹ and R¹³ are hydrogen. In preferred embodiments, R″ or R¹² are selected from —F, —Cl, —Br, —I, —CN, -Me, —CF₃, —CF₂H, —CFH₂, —OCF₃, and —OCH₃.

In preferred embodiments, R¹¹ is selected from —F, —Cl, —Br, and —I, while R⁹, R¹⁰, R¹² and R¹³ are hydrogen.

In preferred embodiments, R⁹ and R″ are selected from —F, —Cl, —Br, —I, —CN, C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, —OCF₃, —OCF₂H, —OCFH₂, —OCF₂Cl, —OCFCl₂, —O—C₁₋₃-alkyl, —C₁₋₃-alkylene-O—C₁₋₃-alkyl, and —C₁₋₃-alkylene-OH, while R¹¹, R¹² and R¹³ are hydrogen.

In a preferred embodiment of the invention, the benzofuran derivative is selected from the group consisting of
Cpd 001 5-(benzyloxy)benzofuran-3-carboxylic acid
Cpd 002 5-(benzyloxy)-2-methylbenzofuran-3-carboxylic acid
Cpd 003 2-methyl-5-((2-methylbenzyl)oxy)benzofuran-3-carboxylic acid
Cpd 004 2-methyl-5-(1-phenylethoxy)benzofuran-3-carboxylic acid
Cpd 005 2-methyl-5-((3-methylbenzyl)oxy)benzofuran-3-carboxylic acid
Cpd 006 2-methyl-5-((4-methylbenzyl)oxy)benzofuran-3-carboxylic acid
Cpd 007 5-((3-fluorobenzyl)oxy)-2-methylbenzofuran-3-carboxylic acid
Cpd 008 5-((2-fluorobenzyl)oxy)-2-methylbenzofuran-3-carboxylic acid
Cpd 009 5-((4-fluorobenzyl)oxy)-2-methylbenzofuran-3-carboxylic acid
Cpd 010 5-((2-cyanobenzyl)oxy)-2-methylbenzofuran-3-carboxylic acid
Cpd 011 5-((2,3-dihydro-1H-inden-1-yl)oxy)-2-methylbenzofuran-3-carboxylic acid
Cpd 012 5-((3-methoxybenzyl)oxy)-2-methylbenzofuran-3-carboxylic acid
Cpd 013 5-((2-methoxybenzyl)oxy)-2-methylbenzofuran-3-carboxylic acid
Cpd 014 5-((4-methoxybenzyl)oxy)-2-methylbenzofuran-3-carboxylic acid
Cpd 015 2-ethyl-5-((3-fluorobenzyl)oxy)benzofuran-3-carboxylic acid
Cpd 016 5-((4-chlorobenzyl)oxy)-2-methylbenzofuran-3-carboxylic acid
Cpd 017 5-((3-chlorobenzyl)oxy)-2-methylbenzofuran-3-carboxylic acid
Cpd 018 5-((2-chlorobenzyl)oxy)-2-methylbenzofuran-3-carboxylic acid
Cpd 019 5-((2,6-difluorobenzyl)oxy)-2-methylbenzofuran-3-carboxylic acid
Cpd 020 5-((3-fluorobenzyl)oxy)-2-(methoxymethyl)benzofuran-3-carboxylic acid
Cpd 021 5-((2-chloro-6-fluorobenzyl)oxy)-2-methylbenzofuran-3-carboxylic acid
Cpd 022 5-((2-chloro-4-fluorobenzyl)oxy)-2-methylbenzofuran-3-carboxylic acid
Cpd 023 5-(benzyloxy)-2-cyclopentylbenzofuran-3-carboxylic acid
Cpd 024 2-methyl-5-((2-(trifluoromethyl)benzyl)oxy)benzofuran-3-carboxylic acid
Cpd 025 2-methyl-5-((3-(trifluoromethyl)benzyl)oxy)benzofuran-3-carboxylic acid
Cpd 026 2-methyl-5-((4-(trifluoromethyl)benzyl)oxy)benzofuran-3-carboxylic acid
Cpd 027 5-((2,4-dichlorobenzyl)oxy)-2-methylbenzofuran-3-carboxylic acid
Cpd 028 2-methyl-5-((3-(trifluoromethoxy)benzyl)oxy)benzofuran-3-carboxylic acid
Cpd 029 ethyl 5-(benzyloxy)benzofuran-3-carboxylate
Cpd 030 methyl 2-ethyl-5-((3-fluorobenzyl)oxy)benzofuran-3-carboxylate
Cpd 031 methyl 5-((2-chlorobenzyl)oxy)-2-methylbenzofuran-3-carboxylate
Cpd 032 ethyl 5-((2,3-dihydro-1H-inden-1-yl)oxy)-2-methylbenzofuran-3-carboxylate
Cpd 033 ethyl 5-((2-chloro-6-fluorobenzyl)oxy)-2-methylbenzofuran-3-carboxylate
Cpd 034 5-((2,3-difluorobenzyl)oxy)-2-methylbenzofuran-3-carboxylic acid
Cpd 035 5-(benzyloxy)-2-methylbenzofuran-3-carboxamide
Cpd 036 5-(benzyloxy)-N-hydroxy-2-methylbenzofuran-3-carboxamide
Cpd 037 5-((3-fluorobenzyl)oxy)-2-methylbenzofuran-3-carboxamide
Cpd 038 5-(benzyloxy)-N-ethyl-2-methylbenzofuran-3-carboxamide
Cpd 039 5-(benzyloxy)-N-cyclopropyl-2-methylbenzofuran-3-carboxamide
Cpd 040 5-(benzyloxy)-2-methyl-N-propylbenzofuran-3-carboxamide
Cpd 041 5-((2-chloro-6-fluorobenzyl)oxy)-2-methylbenzofuran-3-carboxamide
Cpd 042 N-(azetidin-3-yl)-5-(benzyloxy)-2-methylbenzofuran-3-carboxamide
Cpd 043 5-(benzyloxy)-2-methyl-N-(oxetan-3-yl)benzofuran-3-carboxamide
Cpd 044 5-(benzyloxy)-N-(2-methoxyethyl)-2-methylbenzofuran-3-carboxamide
Cpd 045 5-((2-chloro-6-fluorobenzyl)oxy)-N,2-dimethylbenzofuran-3-carboxamide
Cpd 046 5-(benzyloxy)-N-cyclopentyl-2-methylbenzofuran-3-carboxamide
Cpd 047 (5-(benzyloxy)-2-methylbenzofuran-3-yl)(piperidin-1-yl)methanone
Cpd 048 (R)-5-(benzyloxy)-2-methyl-N-(pyrrolidin-3-yl)benzofuran-3-carboxamide
Cpd 049 (S)-5-(benzyloxy)-2-methyl-N-(pyrrolidin-3-yl)benzofuran-3-carboxamide
Cpd 050 (5-(benzyloxy)-2-methylbenzofuran-3-yl)(morpholino)methanone
Cpd 051 5-(benzyloxy)-2-methyl-N-(tetrahydrofuran-3-yl)benzofuran-3-carboxamide
Cpd 052 5-(benzyloxy)-N-(3-hydroxycyclobutyl)-2-methylbenzofuran-3-carboxamide
Cpd 053 5-(benzyloxy)-N-(trans-3-hydroxycyclobutyl)-2-methylbenzofuran-3-carboxamide
Cpd 054 5-(benzyloxy)-N-(cis-3-hydroxycyclobutyl)-2-methylbenzofuran-3-carboxamide
Cpd 055 5-(benzyloxy)-N-(2-(dimethylamino)ethyl)-2-methylbenzofuran-3-carboxamide
Cpd 056 5-(benzyloxy)-N-(3-methoxypropyl)-2-methylbenzofuran-3-carboxamide
Cpd 057 5-(benzyloxy)-N-(2,3-dihydroxypropyl)-2-methylbenzofuran-3-carboxamide
Cpd 058 5-(benzyloxy)-2-methyl-N-phenylbenzofuran-3-carboxamide
Cpd 059 5-(benzyloxy)-2-methyl-N-(pyridin-3-yl)benzofuran-3-carboxamide
Cpd 060 5-(benzyloxy)-2-methyl-N-(pyridin-4-yl)benzofuran-3-carboxamide
Cpd 061 5-(benzyloxy)-2-methyl-N-(pyrazin-2-yl)benzofuran-3-carboxamide
Cpd 062 5-(benzyloxy)-2-methyl-N-(pyridazin-3-yl)benzofuran-3-carboxamide
Cpd 063 5-(benzyloxy)-2-methyl-N-(methylsulfonyl)benzofuran-3-carboxamide
Cpd 064 5-(benzyloxy)-2-methyl-N-(1-methyl-1H-pyrazol-3-yl)benzofuran-3-carboxamide
Cpd 065 5-((2-chloro-6-fluorobenzyl)oxy)-N,N,2-trimethylbenzofuran-3-carboxamide
Cpd 066 5-(benzyloxy)-N-cyclohexyl-2-methylbenzofuran-3-carboxamide
Cpd 067 (5-(benzyloxy)-2-methylbenzofuran-3-yl)(4-methylpiperidin-1-yl)methanone
Cpd 068 5-(benzyloxy)-2-methyl-N-(5-oxopyrrolidin-3-yl)benzofuran-3-carboxamide
Cpd 069 (S)-5-(benzyloxy)-2-methyl-N-(2-oxopyrrolidin-3-yl)benzofuran-3-carboxamide
Cpd 070 (R)-5-(benzyloxy)-2-methyl-N-(2-oxopyrrolidin-3-yl)benzofuran-3-carboxamide
Cpd 071 5-(benzyloxy)-2-methyl-N-(2-oxopyrrolidin-3-yl)benzofuran-3-carboxamide
Cpd 072 (5-(benzyloxy)-2-methylbenzofuran-3-yl)(4-methylpiperazin-1-yl)methanone
Cpd 073 (4-aminopiperidin-1-yl)(5-(benzyloxy)-2-methylbenzofuran-3-yl)methanone
Cpd 074 5-(benzyloxy)-2-methyl-N-(piperidin-4-yl)benzofuran-3-carboxamide
Cpd 075 5-(benzyloxy)-2-methyl-N-(piperidin-3-yl)benzofuran-3-carboxamide
Cpd 076 5-(benzyloxy)-2-methyl-N-(1-methylpyrrolidin-3-yl)benzofuran-3-carboxamide
Cpd 077 5-(benzyloxy)-2-methyl-N-(pyrrolidin-3-ylmethyl)benzofuran-3-carboxamide
Cpd 078 5-(benzyloxy)-2-methyl-N-(pyrrolidin-2-ylmethyl)benzofuran-3-carboxamide
Cpd 079 (S)-5-(benzyloxy)-2-methyl-N-(1-methylpyrrolidin-3-yl)benzofuran-3-carboxamide
Cpd 080 (R)-5-(benzyloxy)-2-methyl-N-(1-methylpyrrolidin-3-yl)benzofuran-3-carboxamide
Cpd 081 5-(benzyloxy)-N-(1-methylpiperidin-4-yl)benzofuran-3-carboxamide
Cpd 082 N-(1-(aminomethyl)cyclobutyl)-5-(benzyloxy)-2-methylbenzofuran-3-carboxamide
Cpd 083 (S)-2-methyl-5-((2-methylbenzyl)oxy)-N-(pyrrolidin-3-yl)benzofuran-3-carboxamide
Cpd 084 (S)-2-methyl-5-((3-methylbenzyl)oxy)-N-(pyrrolidin-3-yl)benzofuran-3-carboxamide
Cpd 085 (S)-2-methyl-5-((4-methylbenzyl)oxy)-N-(pyrrolidin-3-yl)benzofuran-3-carboxamide
Cpd 086 5-(benzyloxy)-N-(3-hydroxycyclopentyl)-2-methylbenzofuran-3-carboxamide
Cpd 087 5-(benzyloxy)-2-methyl-N-((tetrahydrofuran-2-yl)methyl)benzofuran-3-carboxamide
Cpd 088 5-(benzyloxy)-2-methyl-N-(tetrahydro-2H-pyran-4-yl)benzofuran-3-carboxamide
Cpd 089 (R)-5-(benzyloxy)-2-methyl-N-((tetrahydrofuran-2-yl)methyl)benzofuran-3-carboxamide
Cpd 090 (S)-5-(benzyloxy)-2-methyl-N-((tetrahydrofuran-2-yl)methyl)benzofuran-3-carboxamide
Cpd 091 5-(benzyloxy)-N-(3-(dimethylamino)propyl)-2-methylbenzofuran-3-carboxamide
Cpd 092 5-(benzyloxy)-N-(3-(hydroxymethyl)oxetan-3-yl)-2-methylbenzofuran-3-carboxamide
Cpd 093 rac-5-(benzyloxy)-N-(trans-4-fluoropyrrolidin-3-yl)-2-methylbenzofuran-3-carboxamide
Cpd 094 rac-5-(benzyloxy)-N-(cis-4-fluoropyrrolidin-3-yl)-2-methylbenzofuran-3-carboxamide
Cpd 095 (S)-5-((2-fluorobenzyl)oxy)-2-methyl-N-(pyrrolidin-3-yl)benzofuran-3-carboxamide
Cpd 096 (S)-5-((3-fluorobenzyl)oxy)-2-methyl-N-(pyrrolidin-3-yl)benzofuran-3-carboxamide
Cpd 097 (S)-5-((4-fluorobenzyl)oxy)-2-methyl-N-(pyrrolidin-3-yl)benzofuran-3-carboxamide
Cpd 098 N-benzyl-5-(benzyloxy)-2-methylbenzofuran-3-carboxamide
Cpd 099 5-(benzyloxy)-2-methyl-N-(pyridin-2-ylmethyl)benzofuran-3-carboxamide
Cpd 100 5-(benzyloxy)-2-methyl-N-(pyridin-3-ylmethyl)benzofuran-3-carboxamide
Cpd 101 5-(benzyloxy)-2-methyl-N-(pyridin-4-ylmethyl)benzofuran-3-carboxamide
Cpd 102 5-(benzyloxy)-2-methyl-N-(pyrazin-2-ylmethyl)benzofuran-3-carboxamide
Cpd 103 (S)-5-((4-cyanobenzyl)oxy)-2-methyl-N-(pyrrolidin-3-yl)benzofuran-3-carboxamide
Cpd 104 5-(benzyloxy)-2-methyl-N-((1-methyl-1H-pyrazol-5-yl)methyl)benzofuran-3-carboxamide
Cpd 105 5-(benzyloxy)-2-methyl-N-((5-methylisoxazol-3-yl)methyl)benzofuran-3-carboxamide
Cpd 106 5-(benzyloxy)-2-methyl-N-(4-methylcyclohexyl)benzofuran-3-carboxamide
Cpd 107 (R)-5-(benzyloxy)-2-methyl-N-(2-oxopiperidin-3-yl)benzofuran-3-carboxamide
Cpd 108 5-(benzyloxy)-2-methyl-N-(2-oxopiperidin-4-yl)benzofuran-3-carboxamide
Cpd 109 (S)-5-(benzyloxy)-2-methyl-N-(2-oxopiperidin-3-yl)benzofuran-3-carboxamide
Cpd 110 5-(benzyloxy)-2-methyl-N-(1-methyl-5-oxopyrrolidin-3-yl)benzofuran-3-carboxamide
Cpd 111 5-(benzyloxy)-2-methyl-N-(6-oxopiperidin-3-yl)benzofuran-3-carboxamide
Cpd 112 5-(benzyloxy)-2-methyl-N-((5-oxopyrrolidin-2-yl)methyl)benzofuran-3-carboxamide
Cpd 113 5-(benzyloxy)-2-methyl-N-(2-oxopiperidin-3-yl)benzofuran-3-carboxamide
Cpd 114 rac-5-(benzyloxy)-2-methyl-N-(trans-2-methylpiperidin-4-yl)benzofuran-3-carboxamide
Cpd 115 rac-5-(benzyloxy)-2-methyl-N-(cis-2-methylpiperidin-4-yl)benzofuran-3-carboxamide
Cpd 116 N-(cis-4-aminocyclohexyl)-5-(benzyloxy)-2-methylbenzofuran-3-carboxamide
Cpd 117 5-(benzyloxy)-2-methyl-N-(1-methylpiperidin-4-yl)benzofuran-3-carboxamide
Cpd 118 (R)-(5-(benzyloxy)-2-methylbenzofuran-3-yl)(3-(dimethylamino)pyrrolidin-1-yl)methanone
Cpd 119 N-(azepan-4-yl)-5-(benzyloxy)-2-methylbenzofuran-3-carboxamide
Cpd 120 N-(trans-4-aminocyclohexyl)-5-(benzyloxy)-2-methylbenzofuran-3-carboxamide
Cpd 121 5-(benzyloxy)-2-methyl-N-(piperidin-4-ylmethyl)benzofuran-3-carboxamide
Cpd 122 5-(benzyloxy)-2-methyl-N-((tetrahydro-2H-pyran-4-yl)methyl)benzofuran-3-carboxamide
Cpd 123 5-(benzyloxy)-N-(4-hydroxycyclohexyl)-2-methylbenzofuran-3-carboxamide
Cpd 124 5-(benzyloxy)-N-(trans-4-hydroxycyclohexyl)-2-methylbenzofuran-3-carboxamide
Cpd 125 5-(benzyloxy)-N-(cis-4-hydroxycyclohexyl)-2-methylbenzofuran-3-carboxamide
Cpd 126 5-(benzyloxy)-N-((3S,5S)-5-(hydroxymethyl)pyrrolidin-3-yl)-2-methylbenzofuran-3-carboxamide
Cpd 127 (S)-5-((3-methoxybenzyl)oxy)-2-methyl-N-(pyrrolidin-3-yl)benzofuran-3-carboxamide
Cpd 128 5-(benzyloxy)-N-((3-(hydroxymethyl)oxetan-3-yl)methyl)-2-methylbenzofuran-3-carboxamide
Cpd 129 5-((2-fluorobenzyl)oxy)-2-methyl-N-(2-oxopyrrolidin-3-yl)benzofuran-3-carboxamide
Cpd 130 5-(benzyloxy)-N-(4-fluoropiperidin-3-yl)-2-methylbenzofuran-3-carboxamide
Cpd 131 5-(benzyloxy)-N-(3-fluoropiperidin-4-yl)-2-methylbenzofuran-3-carboxamide
Cpd 132 5-((3-fluorobenzyl)oxy)-2-methyl-N-((tetrahydrofuran-2-yl)methyl)benzofuran-3-carboxamide
Cpd 133 5-(benzyloxy)-N-(cyclopropylsulfonyl)-2-methylbenzofuran-3-carboxamide
Cpd 134 5-(benzyloxy)-2-methyl-N-phenethylbenzofuran-3-carboxamide
Cpd 135 (S)-5-((2,3-difluorobenzyl)oxy)-2-methyl-N-(pyrrolidin-3-yl)benzofuran-3-carboxamide
Cpd 136 (S)-5-((2,6-difluorobenzyl)oxy)-2-methyl-N-(pyrrolidin-3-yl)benzofuran-3-carboxamide
Cpd 137 5-(benzyloxy)-N-(4,4-difluoropyrrolidin-3-yl)-2-methylbenzofuran-3-carboxamide
Cpd 138 5-(benzyloxy)-2-methyl-N-(2-(pyridin-2-yl)ethyl)benzofuran-3-carboxamide
Cpd 139 5-(benzyloxy)-2-methyl-N-(2-(pyridin-4-yl)ethyl)benzofuran-3-carboxamide
Cpd 140 5-(benzyloxy)-2-methyl-N-(trans-octahydrocyclopenta[c]pyrrol-5-yl)benzofuran-3-carboxamide
Cpd 141 5-(benzyloxy)-N-((1R,3s,5S)-8-azabicyclo[3.2.1]octan-3-yl)-2-methylbenzofuran-3-carboxamide
Cpd 142 5-(benzyloxy)-2-methyl-N-(quinuclidin-3-yl)benzofuran-3-carboxamide
Cpd 143 5-(benzyloxy)-N-((1R,3r,5S)-8-azabicyclo[3.2.1]octan-3-yl)-2-methylbenzofuran-3-carboxamide
Cpd 144 5-(benzyloxy)-N-(hexahydro-1H-pyrrolizin-1-yl)-2-methylbenzofuran-3-carboxamide
Cpd 145 (S)—N-(1-acetylpyrrolidin-3-yl)-5-(benzyloxy)-2-methylbenzofuran-3-carboxamide
Cpd 146 5-(benzyloxy)-2-methyl-N-(1-methyl-2-oxopiperidin-4-yl)benzofuran-3-carboxamide
Cpd 147 5-(benzyloxy)-N-(1-ethyl-2-oxopyrrolidin-3-yl)-2-methylbenzofuran-3-carboxamide
Cpd 148 5-(benzyloxy)-2-methyl-N-(1-(oxetan-3-yl)azetidin-3-yl)benzofuran-3-carboxamide
Cpd 149 (5-(benzyloxy)-2-methylbenzofuran-3-yl)(4-ethyl-1,4-diazepan-1-yl)methanone
Cpd 150 (5-(benzyloxy)-2-methylbenzofuran-3-yl)(3-(dimethylamino)piperidin-1-yl)methanone
Cpd 151 5-(benzyloxy)-N-(1-ethylpiperidin-4-yl)-2-methylbenzofuran-3-carboxamide
Cpd 152 (S)-5-(benzyloxy)-N-((l-ethylpyrrolidin-2-yl)methyl)-2-methylbenzofuran-3-carboxamide
Cpd 153 (R)-5-(benzyloxy)-N-((l-ethylpyrrolidin-2-yl)methyl)-2-methylbenzofuran-3-carboxamide
Cpd 154 2-methyl-5-((4-methylbenzyl)oxy)-N-(1-methylpiperidin-4-yl)benzofuran-3-carboxamide
Cpd 155 5-(benzyloxy)-N-(1,2-dimethylpiperidin-4-yl)-2-methylbenzofuran-3-carboxamide
Cpd 156 5-(benzyloxy)-N,2-dimethyl-N-(1-methylpiperidin-4-yl)benzofuran-3-carboxamide
Cpd 157 5-((3-(aminomethyl)benzyl)oxy)-2-methyl-N-(2-oxopyrrolidin-3-yl)benzofuran-3-carboxamide
Cpd 158 (2S,4R)-4-(5-(benzyloxy)-2-methylbenzofuran-3-carboxamido)pyrrolidine-2-carboxamide
Cpd 159 5-(benzyloxy)-N-(4-methoxycyclohexyl)-2-methylbenzofuran-3-carboxamide
Cpd 160 (2S,4S)-4-(5-(benzyloxy)-2-methylbenzofuran-3-carboxamido)pyrrolidine-2-carboxylic acid
Cpd 161 (2S,4R)-4-(5-(benzyloxy)-2-methylbenzofuran-3-carboxamido)pyrrolidine-2-carboxylic acid
Cpd 162 (2R,4R)-4-(5-(benzyloxy)-2-methylbenzofuran-3-carboxamido)pyrrolidine-2-carboxylic acid
Cpd 163 (2R,4S)-4-(5-(benzyloxy)-2-methylbenzofuran-3-carboxamido)pyrrolidine-2-carboxylic acid
Cpd 164 5-((2-methoxybenzyl)oxy)-2-methyl-N-(2-oxopyrrolidin-3-yl)benzofuran-3-carboxamide
Cpd 165 5-(benzyloxy)-2-methyl-N-(2-morpholinoethyl)benzofuran-3-carboxamide
Cpd 166 5-(benzyloxy)-N-(3,3-bis(hydroxymethyl)cyclobutyl)-2-methylbenzofuran-3-carboxamide
Cpd 167 5-(benzyloxy)-N-(4-(hydroxymethyl)tetrahydro-2H-pyran-4-yl)-2-methylbenzofuran-3-carboxamide
Cpd 168 5-((3-fluorobenzyl)oxy)-2-methyl-N-(1-methylpiperidin-4-yl)benzofuran-3-carboxamide
Cpd 169 N-(cyclopropylsulfonyl)-2-methyl-5-((4-methylbenzyl)oxy)benzofuran-3-carboxamide
Cpd 170 N-(cyclopropylsulfonyl)-2-methyl-5-((3-methylbenzyl)oxy)benzofuran-3-carboxamide
Cpd 171 5-((2,3-difluorobenzyl)oxy)-2-methyl-N-(2-oxopyrrolidin-3-yl)benzofuran-3-carboxamide
Cpd 172 5-((2,6-difluorobenzyl)oxy)-2-methyl-N-(2-oxopyrrolidin-3-yl)benzofuran-3-carboxamide
Cpd 173 5-(benzyloxy)-N-(3,3-difluoropiperidin-4-yl)-2-methylbenzofuran-3-carboxamide
Cpd 174 5-(benzyloxy)-N-(2-hydroxy-2-(pyridin-3-yl)ethyl)-2-methylbenzofuran-3-carboxamide
Cpd 175 N-(cyclopropylsulfonyl)-5-((2-fluorobenzyl)oxy)-2-methylbenzofuran-3-carboxamide
Cpd 176 N-(cyclopropylsulfonyl)-5-((3-fluorobenzyl)oxy)-2-methylbenzofuran-3-carboxamide
Cpd 177 N-(cyclopropylsulfonyl)-5-((4-fluorobenzyl)oxy)-2-methylbenzofuran-3-carboxamide
Cpd 178 5-(benzyloxy)-N-(2-(3,5-dimethylisoxazol-4-yl)ethyl)-2-methylbenzofuran-3-carboxamide
Cpd 179 5-(benzyloxy)-N-(2-(3,5-dimethyl-1H-1,2,4-triazol-1-yl)ethyl)-2-methylbenzofuran-3-carboxamide
Cpd 180 5-(benzyloxy)-2-methyl-N-(7-azaspiro[3.5]nonan-2-yl)benzofuran-3-carboxamide
Cpd 181 5-(benzyloxy)-2-methyl-N-(8-methyl-8-azabicyclo[3.2.1]octan-3-yl)benzofuran-3-carboxamide
Cpd 182 5-(benzyloxy)-2-methyl-N-((1R,3s,5S)-8-methyl-8-azabicyclo[3.2.1]octan-3-yl)benzofuran-3-carboxamide
Cpd 183 5-(benzyloxy)-2-methyl-N-((1R,3r,5S)-8-methyl-8-azabicyclo[3.2.1]octan-3-yl)benzofuran-3-carboxamide
Cpd 184 5-(benzyloxy)-N-((1R,3s,5S)-9-azabicyclo[3.3.1]nonan-3-yl)-2-methylbenzofuran-3-carboxamide
Cpd 185 5-(benzyloxy)-N-(1-isopropylpiperidin-4-yl)-2-methylbenzofuran-3-carboxamide
Cpd 186 5-(benzyloxy)-N-(4-(dimethylamino)cyclohexyl)-2-methylbenzofuran-3-carboxamide
Cpd 187 5-(benzyloxy)-N-((1-(dimethylamino)cyclopentyl)methyl)-2-methylbenzofuran-3-carboxamide
Cpd 188 (2S,4R)-4-(5-(benzyloxy)-2-methylbenzofuran-3-carboxamido)-N-methylpyrrolidine-2-carboxamide
Cpd 189 5-(benzyloxy)-2-methyl-N-(2-(4-methylpiperazin-1-yl)ethyl)benzofuran-3-carboxamide
Cpd 190 5-((4-(aminomethyl)benzyl)oxy)-2-methyl-N-(1-methylpiperidin-4-yl)benzofuran-3-carboxamide
Cpd 191 methyl (2S,4S)-4-(5-(benzyloxy)-2-methylbenzofuran-3-carboxamido)pyrrolidine-2-carboxylate
Cpd 192 5-((2-(hydroxymethyl)benzyl)oxy)-2-methyl-N-(1-methylpiperidin-4-yl)benzofuran-3-carboxamide
Cpd 193 4-(5-(benzyloxy)-2-methylbenzofuran-3-carboxamido)tetrahydro-2H-pyran-4-carboxylic acid
Cpd 194 2-ethyl-5-((3-fluorobenzyl)oxy)-N-(1-methylpiperidin-4-yl)benzofuran-3-carboxamide
Cpd 195 5-(benzyloxy)-2-methyl-N-((trifluoromethyl)sulfonyl)benzofuran-3-carboxamide
Cpd 196 N-([1,2,4]triazolo[4,3-a]pyrimidin-3-ylmethyl)-5-(benzyloxy)-2-methylbenzofuran-3-carboxamide
Cpd 197 5-((2-fluorobenzyl)oxy)-N-(4-(hydroxymethyl)tetrahydro-2H-pyran-4-yl)-2-methylbenzofuran-3-carboxamide
Cpd 198 5-(benzyloxy)-N-(1,1-dioxidotetrahydro-2H-thiopyran-4-yl)-2-methylbenzofuran-3-carboxamide
Cpd 199 N-(cyclopropylsulfonyl)-5-((2-methoxybenzyl)oxy)-2-methylbenzofuran-3-carboxamide
Cpd 200 5-(benzyloxy)-N-((4,6-dimethyl-2-oxo-1,2-dihydropyridin-3-yl)methyl)-2-methylbenzofuran-3-carboxamide
Cpd 201 N-(3,3-difluoropiperidin-4-yl)-5-((2-fluorobenzyl)oxy)-2-methylbenzofuran-3-carboxamide
Cpd 202 (S)-2-methyl-N-(pyrrolidin-3-yl)-5-((4-(trifluoromethyl)benzyl)oxy)benzofuran-3-carboxamide
Cpd 203 (S)-2-methyl-N-(pyrrolidin-3-yl)-5-((2-(trifluoromethyl)benzyl)oxy)benzofuran-3-carboxamide
Cpd 204 (S)-2-methyl-N-(pyrrolidin-3-yl)-5-((3-(trifluoromethyl)benzyl)oxy)benzofuran-3-carboxamide
Cpd 205 5-(benzyloxy)-2-methyl-N-((7S,8aS)-2-methyloctahydropyrrolo[1,2-a]pyrazin-7-yl)benzofuran-3-carboxamide
Cpd 206 (S)-5-(benzyloxy)-2-methyl-N-(1-(oxetan-3-yl)piperidin-3-yl)benzofuran-3-carboxamide
Cpd 207 (R)-5-(benzyloxy)-2-methyl-N-(1-(oxetan-3-yl)piperidin-3-yl)benzofuran-3-carboxamide
Cpd 208 5-(benzyloxy)-2-methyl-N-(1-(oxetan-3-yl)piperidin-4-yl)benzofuran-3-carboxamide
Cpd 209 5-(benzyloxy)-2-methyl-N-(2,2,6,6-tetramethylpiperidin-4-yl)benzofuran-3-carboxamide
Cpd 210 5-(benzyloxy)-N-(trans-4-(2-hydroxypropan-2-yl)cyclohexyl)-2-methylbenzofuran-3-carboxamide
Cpd 211 5-((2,3-difluorobenzyl)oxy)-N-(4,4-difluoropyrrolidin-3-yl)-2-methylbenzofuran-3-carboxamide
Cpd 212 methyl 4-(5-(benzyloxy)-2-methylbenzofuran-3-carboxamido)tetrahydro-2H-pyran-4-carboxylate
Cpd 213 N-(1-([1,2,4]triazolo[4,3-a]pyrimidin-3-yl)ethyl)-5-(benzyloxy)-2-methylbenzofuran-3-carboxamide
Cpd 214 (5-(benzyloxy)-2-methylbenzofuran-3-yl)(4-(pyridin-2-yl)piperazin-1-yl)methanone
Cpd 215 (S)-5-(benzyloxy)-2-methyl-N-(1-(methylsulfonyl)pyrrolidin-3-yl)benzofuran-3-carboxamide
Cpd 216 5-(benzyloxy)-N-(2-(dimethylamino)-2-phenylethyl)-2-methylbenzofuran-3-carboxamide
Cpd 217 5-((2,3-difluorobenzyl)oxy)-N-(4-(hydroxymethyl)tetrahydro-2H-pyran-4-yl)-2-methylbenzo-furan-3-carboxamide
Cpd 218 5-((2,6-difluorobenzyl)oxy)-N-(4-(hydroxymethyl)tetrahydro-2H-pyran-4-yl)-2-methylbenzo-furan-3-carboxamide
Cpd 219 2-methyl-N-(2-oxopyrrolidin-3-yl)-5-((2-(trifluoromethyl)benzyl)oxy)benzofuran-3-carboxamide
Cpd 220 rac-2-methyl-N—((R)-pyrrolidin-3-yl)-5-(1-(2-(trifluoromethyl)phenyl)ethoxy)benzofuran-3-carboxamide
Cpd 221 5-(benzyloxy)-2-cyclopentyl-N-(1-methylpiperidin-4-yl)benzofuran-3-carboxamide
Cpd 222 5-(benzyloxy)-N-((7S,8aS)-1,4-dioxooctahydropyrrolo[1,2-a]pyrazin-7-yl)-2-methylbenzofuran-3-carboxamide
Cpd 223 (S)-2-methyl-N-(pyrrolidin-3-yl)-5-((3-(trifluoromethoxy)benzyl)oxy)benzofuran-3-carboxamide
Cpd 224 5-(3-(dimethylamino)-1-phenylpropoxy)-2-methyl-N-(2-oxopyrrolidin-3-yl)benzofuran-3-carboxamide
Cpd 225 5-(benzyloxy)-N-(1-(2-(dimethylamino)ethyl)piperidin-4-yl)-2-methylbenzofuran-3-carboxamide
Cpd 226 5-((2,6-difluorobenzyl)oxy)-N-(3,3-difluoropiperidin-4-yl)-2-methylbenzofuran-3-carboxamide
Cpd 227 5-((2,3-difluorobenzyl)oxy)-N-(3,3-difluoropiperidin-4-yl)-2-methylbenzofuran-3-carboxamide
Cpd 228 tert-butyl 3-(5-(benzyloxy)-2-methylbenzofuran-3-carboxamido)azetidine-1-carboxylate
Cpd 229 5-(benzyloxy)-N-(1-(3-methoxypropyl)piperidin-4-yl)-2-methylbenzofuran-3-carboxamide
Cpd 230 5-(benzyloxy)-2-methyl-N-(1-(pyrimidin-2-yl)piperidin-4-yl)benzofuran-3-carboxamide
Cpd 231 rac-2-methyl-N—((R)-pyrrolidin-3-yl)-5-(1-(2-(trifluoromethyl)phenyl)propoxy)benzofuran-3-carboxamide
Cpd 232 2-methyl-N-(1-methylpiperidin-4-yl)-5-((3-(trifluoromethyl)benzyl)oxy)benzofuran-3-carboxamide
Cpd 233 2-methyl-N-(1-methylpiperidin-4-yl)-5-((2-(trifluoromethyl)benzyl)oxy)benzofuran-3-carboxamide
Cpd 234 2-methyl-N-(1-methylpiperidin-4-yl)-5-((4-(trifluoromethyl)benzyl)oxy)benzofuran-3-carboxamide
Cpd 235 5-(benzyloxy)-2-methyl-N-((7S,8aS)-2-methyl-1,4-dioxooctahydropyrrolo[1,2-a]pyrazin-7-yl)benzofuran-3-carboxamide
Cpd 236 1-(5-(benzyloxy)-2-methylbenzofuran-3-carbonyl)-N-isobutylpiperidine-3-carboxamide
Cpd 237 tert-butyl 1-(5-(benzyloxy)-2-methylbenzofuran-3-carbonyl)piperidine-4-carboxylate
Cpd 238 tert-butyl (R)-3-(5-(benzyloxy)-2-methylbenzofuran-3-carboxamido)pyrrolidine-1-carboxylate
Cpd 239 tert-butyl (S)-3-(5-(benzyloxy)-2-methylbenzofuran-3-carboxamido)pyrrolidine-1-carboxylate
Cpd 240 N-(4,4-difluoropyrrolidin-3-yl)-2-methyl-5-((2-(trifluoromethyl)benzyl)oxy)benzofuran-3-carboxamide
Cpd 241 5-(benzyloxy)-N-(1-benzylpiperidin-4-yl)-2-methylbenzofuran-3-carboxamide
Cpd 242 2-methyl-N-(1-methylpiperidin-4-yl)-5-((2-(methylsulfonyl)benzyl)oxy)benzofuran-3-carboxamide
Cpd 243 (5-(benzyloxy)-2-methylbenzofuran-3-yl)(3-(3-isopropyl-1,2,4-oxadiazol-5-yl)piperidin-1-yl)-methanone
Cpd 244 2-methyl-N-(1-methylpiperidin-4-yl)-5-((3-(trifluoromethoxy)benzyl)oxy)benzofuran-3-carboxamide
Cpd 245 2-methyl-5-((2-(4-methylpiperazin-1-yl)benzyl)oxy)-N-(2-oxopyrrolidin-3-yl)benzofuran-3-carboxamide
Cpd 246 tert-butyl ((1-(5-(benzyloxy)-2-methylbenzofuran-3-carboxamido)cyclobutyl)methyl)carbamate
Cpd 247 tert-butyl 3-(5-(benzyloxy)-2-methylbenzofuran-3-carboxamido)piperidine-1-carboxylate
Cpd 248 tert-butyl (1-(5-(benzyloxy)-2-methylbenzofuran-3-carbonyl)piperidin-4-yl)carbamate
Cpd 249 tert-butyl 4-(5-(benzyloxy)-2-methylbenzofuran-3-carboxamido)piperidine-1-carboxylate
Cpd 250 5-((2-(2-(dimethylamino)ethoxy)benzyl)oxy)-2-methyl-N-(1-methylpiperidin-4-yl)benzofuran-3-carboxamide
Cpd 251 tert-butyl trans-4-(5-(benzyloxy)-2-methylbenzofuran-3-carboxamido)-2-methylpiperidine-1-carboxylate
Cpd 252 tert-butyl cis-4-(5-(benzyloxy)-2-methylbenzofuran-3-carboxamido)-2-methylpiperidine-1-carboxylate
Cpd 253 tert-butyl 4-((5-(benzyloxy)-2-methylbenzofuran-3-carboxamido)methyl)piperidine-1-carboxylate
Cpd 254 tert-butyl 4-(5-(benzyloxy)-2-methylbenzofuran-3-carboxamido)-3-fluoropiperidine-1-carboxylate
Cpd 255 tert-butyl 3-(5-(benzyloxy)-2-methylbenzofuran-3-carboxamido)-4-fluoropiperidine-1-carboxylate
Cpd 256 tert-butyl (S)-3-(5-((2,3-difluorobenzyl)oxy)-2-methylbenzofuran-3-carboxamido)pyrrolidine-1-carboxylate
Cpd 257 tert-butyl (S)-3-(5-((2,6-difluorobenzyl)oxy)-2-methylbenzofuran-3-carboxamido)pyrrolidine-1-carboxylate
Cpd 258 tert-butyl (1R,3s,5S)-3-(5-(benzyloxy)-2-methylbenzofuran-3-carboxamido)-8-azabicyclo-[3.2.1]octane-8-carboxylate
Cpd 259 tert-butyl trans-5-(5-(benzyloxy)-2-methylbenzofuran-3-carboxamido)hexahydrocyclopenta-[c]pyrrole-2(1H)-carboxylate
Cpd 260 2-methyl-N-(1-methylpiperidin-4-yl)-5-((2-(((tetrahydro-2H-pyran-2-yl)oxy)methyl)benzyl)oxy)-benzofuran-3-carboxamide
Cpd 261 tert-butyl (3-(((2-methyl-3-((2-oxopyrrolidin-3-yl)carbamoyl)benzofuran-5-yl)oxy)methyl)-benzyl)carbamate
Cpd 262 tert-butyl 4-(5-(benzyloxy)-2-methylbenzofuran-3-carboxamido)-4-(hydroxymethyl)piperidine-1-carboxylate
Cpd 263 tert-butyl 4-(5-(benzyloxy)-2-methylbenzofuran-3-carboxamido)-3,3-difluoropiperidine-1-carboxylate
Cpd 264 tert-butyl 2-(5-(benzyloxy)-2-methylbenzofuran-3-carboxamido)-7-azaspiro[3.5]nonane-7-carboxylate
Cpd 265 tert-butyl (1R,5S,7r)-7-(5-(benzyloxy)-2-methylbenzofuran-3-carboxamido)-3-oxa-9-azabicyclo-[3.3.1]nonane-9-carboxylate
Cpd 266 tert-butyl 4-(5-((2,3-difluorobenzyl)oxy)-2-methylbenzofuran-3-carboxamido)-3,3-difluoropyrrolidine-1-carboxylate
Cpd 267 rac-tert-butyl (3R)-3-(2-methyl-5-(1-(2-(trifluoromethyl)phenyl)ethoxy)benzofuran-3-carboxamido)pyrrolidine-1-carboxylate
Cpd 268 tert-butyl 4-(5-((2,6-difluorobenzyl)oxy)-2-methylbenzofuran-3-carboxamido)-3,3-difluoro-piperidine-1-carboxylate
Cpd 269 tert-butyl 34(5-(benzyloxy)-2-methylbenzofuran-3-carboxamido)methyl)pyrrolidine-1-carboxylate
Cpd 270 tert-butyl 2-((5-(benzyloxy)-2-methylbenzofuran-3-carboxamido)methyl)pyrrolidine-1-carboxylate
Cpd 271 tert-butyl trans-3-(5-(benzyloxy)-2-methylbenzofuran-3-carboxamido)-4-fluoropyrrolidine-1-carboxylate
Cpd 272 tert-butyl cis-3-(5-(benzyloxy)-2-methylbenzofuran-3-carboxamido)-4-fluoropyrrolidine-1-carboxylate
Cpd 273 tert-butyl (cis-4-(5-(benzyloxy)-2-methylbenzofuran-3-carboxamido)cyclohexyl)carbamate
Cpd 274 tert-butyl 4-(5-(benzyloxy)-2-methylbenzofuran-3-carboxamido)azepane-1-carboxylate
Cpd 275 tert-butyl (2R,4R)-4-(5-(benzyloxy)-2-methylbenzofuran-3-carboxamido)-2-(hydroxymethyl)-pyrrolidine-1-carboxylate
Cpd 276 tert-butyl 4-(5-(benzyloxy)-2-methylbenzofuran-3-carboxamido)-3,3-difluoropyrrolidine-1-carboxylate
Cpd 277 tert-butyl (1R,5R)-3-(5-(benzyloxy)-2-methylbenzofuran-3-carboxamido)-9-azabicyclo[3.3.1]-nonane-9-carboxylate
Cpd 278 1-(tert-butyl) 2-methyl (2S,4S)-4-(5-(benzyloxy)-2-methylbenzofuran-3-carboxamido)pyrrolidine-1,2-dicarboxylate
Cpd 279 tert-butyl 3,3-difluoro-4-(5-((2-fluorobenzyl)oxy)-2-methylbenzofuran-3-carboxamido)piperidine-1-carboxylate
Cpd 280 tert-butyl (R)-3-(2-methyl-5-((3-(trifluoromethoxy)benzyl)oxy)benzofuran-3-carboxamido)pyrrolidine-1-carboxylate
Cpd 281 tert-butyl 3,3-difluoro-4-(2-methyl-5-((2-(trifluoromethyl)benzyl)oxy)benzofuran-3-carboxamido)pyrrolidine-1-carboxylate
Cpd 282 tert-butyl (R)-3-(2-methyl-5-((3-methylbenzyl)oxy)benzofuran-3-carboxamido)pyrrolidine-1-carboxylate
Cpd 283 tert-butyl (R)-3-(2-methyl-5-((4-methylbenzyl)oxy)benzofuran-3-carboxamido)pyrrolidine-1-carboxylate
Cpd 284 rac-tert-butyl (R)-3-(2-methyl-5-((2-methylbenzyl)oxy)benzofuran-3-carboxamido)pyrrolidine-1-carboxylate
Cpd 285 tert-butyl (R)-3-(5-((2-fluorobenzyl)oxy)-2-methylbenzofuran-3-carboxamido)pyrrolidine-1-carboxylate
Cpd 286 tert-butyl (R)-3-(54(3-fluorobenzyl)oxy)-2-methylbenzofuran-3-carboxamido)pyrrolidine-1-carboxylate
Cpd 287 tert-butyl (R)-3-(54(4-fluorobenzyl)oxy)-2-methylbenzofuran-3-carboxamido)pyrrolidine-1-carboxylate
Cpd 288 tert-butyl (R)-3-(54(3-methoxybenzyl)oxy)-2-methylbenzofuran-3-carboxamido)pyrrolidine-1-carboxylate
Cpd 289 tert-butyl (1R,3r,5S)-3-(5-(benzyloxy)-2-methylbenzofuran-3-carboxamido)-8-azabicyclo-[3.2.1]octane-8-carboxylate
Cpd 290 tert-butyl (R)-3-(2-methyl-5-((4-(trifluoromethyl)benzyl)oxy)benzofuran-3-carboxamido)-pyrrolidine-1-carboxylate
Cpd 291 tert-butyl (R)-3-(2-methyl-5-((2-(trifluoromethyl)benzyl)oxy)benzofuran-3-carboxamido)-pyrrolidine-1-carboxylate
Cpd 292 tert-butyl (R)-3-(2-methyl-5-((3-(trifluoromethyl)benzyl)oxy)benzofuran-3-carboxamido)-pyrrolidine-1-carboxylate
Cpd 293 tert-butyl (R)-3-(54(4-cyanobenzyl)oxy)-2-methylbenzofuran-3-carboxamido)pyrrolidine-1-carboxylate
Cpd 294 rac-tert-butyl (3R)-3-(2-methyl-5-(1-(2-(trifluoromethyl)phenyl)propoxy)benzofuran-3-carboxamido)pyrrolidine-1-carboxylate
Cpd 295 tert-butyl (4-(((2-methyl-3-((1-methylpiperidin-4-yl)carbamoyl)benzofuran-5-yl)oxy)methyl)-benzyl)carbamate
Cpd 296 tert-butyl 4-(5-((2,3-difluorobenzyl)oxy)-2-methylbenzofuran-3-carboxamido)-3,3-difluoro-piperidine-1-carboxylate
Cpd 297 5-(benzyloxy)-4-cyano-N-(4,4-difluoropyrrolidin-3-yl)-2-methylbenzofuran-3-carboxamide
Cpd 298 5-(benzyloxy)-N-(4,4-difluoropyrrolidin-3-yl)-4-fluoro-2-methylbenzofuran-3-carboxamide
Cpd 299 5-(benzyloxy)-N-(4,4-difluoropyrrolidin-3-yl)-2,4-dimethylbenzofuran-3-carboxamide
Cpd 300 5-(benzyloxy)-6-chloro-N-(4,4-difluoropyrrolidin-3-yl)-2-methylbenzofuran-3-carboxamide
Cpd 301 5-(benzyloxy)-6-cyano-N-(4,4-difluoropyrrolidin-3-yl)-2-methylbenzofuran-3-carboxamide
Cpd 302 5-(benzyloxy)-N-(4,4-difluoropyrrolidin-3-yl)-6-fluoro-2-methylbenzofuran-3-carboxamide
Cpd 303 5-(benzyloxy)-N-(4,4-difluoropyrrolidin-3-yl)-2,6-dimethylbenzofuran-3-carboxamide
Cpd 304 5-(benzyloxy)-7-chloro-N-(4,4-difluoropyrrolidin-3-yl)-2-methylbenzofuran-3-carboxamide
Cpd 305 5-(benzyloxy)-7-cyano-N-(4,4-difluoropyrrolidin-3-yl)-2-methylbenzofuran-3-carboxamide
Cpd 306 5-(benzyloxy)-N-(4,4-difluoropyrrolidin-3-yl)-7-fluoro-2-methylbenzofuran-3-carboxamide
Cpd 307 5-(benzyloxy)-N-(4,4-difluoropyrrolidin-3-yl)-2,7-dimethylbenzofuran-3-carboxamide
Cpd 308 N-(4-(hydroxymethyl)tetrahydro-2H-pyran-4-yl)-2-methyl-5-(1-phenylethoxy)benzofuran-3-carboxamide
Cpd 309 N-(3-carbamoyloxetan-3-yl)-5-((2-fluorobenzyl)oxy)-2-methylbenzofuran-3-carboxamide
Cpd 310 5-((2-fluorobenzyl)oxy)-N-(3-(hydroxymethyl)oxetan-3-yl)-2-methylbenzofuran-3-carboxamide
Cpd 311 5-((2-fluorobenzyl)oxy)-N-(1-(2-hydroxyethyl)-2-oxopyrrolidin-3-yl)-2-methylbenzofuran-3-carboxamide
Cpd 312 N-(1,3-dihydroxy-2-methylpropan-2-yl)-5-((2-fluorobenzyl)oxy)-2-methylbenzofuran-3-carboxamide
Cpd 313 5-((2-fluorobenzyl)oxy)-N-(1-hydroxy-2-methylpropan-2-yl)-2-methylbenzofuran-3-carboxamide
Cpd 314 N-(1-amino-3-hydroxy-1-oxopropan-2-yl)-5-((2-fluorobenzyl)oxy)-2-methylbenzofuran-3-carboxamide
Cpd 315 N-(1-amino-2-methyl-1-oxopropan-2-yl)-5-((2-fluorobenzyl)oxy)-2-methylbenzofuran-3-carboxamide
Cpd 316 N-(4-(hydroxymethyl)tetrahydro-2H-pyran-4-yl)-2-methyl-5-((2-phenylpropan-2-yl)oxy)benzofuran-3-carboxamide
Cpd 317 5-(2-hydroxy-1-phenylethoxy)-N-(4-(hydroxymethyl)tetrahydro-2H-pyran-4-yl)-2-methylbenzofuran-3-carboxamide
Cpd 318 N-(1-amino-2-methyl-1-oxopropan-2-yl)-5-((2-fluorophenoxy)methyl)-2-methylbenzofuran-3-carboxamide
Cpd 319 5-((2-fluorophenoxy)methyl)-N-(1-hydroxy-2-methylpropan-2-yl)-2-methylbenzofuran-3-carboxamide
Cpd 320 N-(1-amino-1-oxopropan-2-yl)-5-((2-fluorobenzyl)oxy)-2-methylbenzofuran-3-carboxamide
Cpd 321 N-(1-amino-3-hydroxy-1-oxopropan-2-yl)-5-((2-fluorophenoxy)methyl)-2-methylbenzofuran-3-carboxamide
Cpd 322 N-(1-carbamoylcyclobutyl)-5-((2-fluorobenzyl)oxy)-2-methylbenzofuran-3-carboxamide
Cpd 323 N-(3-carbamoyltetrahydrofuran-3-yl)-5-((2-fluorobenzyl)oxy)-2-methylbenzofuran-3-carboxamide
Cpd 324 5-((2-fluorobenzyl)oxy)-N-(1-(hydroxymethyl)cyclobutyl)-2-methylbenzofuran-3-carboxamide
Cpd 325 5-((2-fluorobenzyl)oxy)-N-(1-(hydroxymethyl)cyclopropyl)-2-methylbenzofuran-3-carboxamide
Cpd 326 N-(1-carbamoylcyclopropyl)-5-((2-fluorobenzyl)oxy)-2-methylbenzofuran-3-carboxamide
Cpd 327 5-((2-fluorobenzyl)oxy)-N-(3-(hydroxymethyl)tetrahydrofuran-3-yl)-2-methylbenzofuran-3-carboxamide
Cpd 328 5-((2-fluorobenzyl)oxy)-N-(1-hydroxypropan-2-yl)-2-methylbenzofuran-3-carboxamide
Cpd 329 5-((2-fluorobenzyl)oxy)-N-(cis-4-hydroxytetrahydrofuran-3-yl)-2-methylbenzofuran-3-carboxamide
Cpd 330 5-((2-fluorobenzyl)oxy)-N-(trans-4-hydroxytetrahydrofuran-3-yl)-2-methylbenzofuran-3-carboxamide
Cpd 331 N-(4,4-difluorotetrahydrofuran-3-yl)-5-((2-fluorobenzyl)oxy)-2-methylbenzofuran-3-carboxamide
Cpd 332 N-(4-(hydroxymethyl)tetrahydro-2H-pyran-4-yl)-2-methyl-5-((2-(methylsulfonyl)benzyl)oxy)benzofuran-3-carboxamide
Cpd 333 N-(3,3-difluoropiperidin-4-yl)-2-methyl-5-((2-(methylsulfonyl)benzyl)oxy)benzofuran-3-carboxamide
Cpd 334 5-((2-fluorobenzyl)oxy)-N-(3-(hydroxymethyl)-2-oxopyrrolidin-3-yl)-2-methylbenzofuran-3-carboxamide
Cpd 335 N—((S)-1-amino-3-hydroxy-1-oxopropan-2-yl)-5-(1-(2-fluorophenyl)-2-hydroxyethoxy)-2-methylbenzofuran-3-carboxamide
Cpd 336 N—((S)-1-amino-3-hydroxy-1-oxopropan-2-yl)-2-methyl-5-(1-phenylethoxy)benzofuran-3-carboxamide
Cpd 337 (S)—N-(1-amino-3-hydroxy-1-oxopropan-2-yl)-5-(benzyloxy)-2-methylbenzofuran-3-carboxamide
Cpd 338 (S)—N-(1-Amino-3-hydroxy-1-oxopropan-2-yl)-5-((2-chlorobenzyl)oxy)-2-methylbenzofuran-3-carboxamide
Cpd 339 (S)—N-(1-Amino-3-hydroxy-1-oxopropan-2-yl)-5-((2-methoxybenzyl) oxy)-2-methylbenzofuran-3-carboxamide
Cpd 340 (S)—N-(1-amino-3-hydroxy-1-oxopropan-2-yl)-5-((2,4-difluorobenzyl)oxy)-2-methylbenzofuran-3-carboxamide
Cpd 341 (S)—N-(1-Amino-3-hydroxy-1-oxopropan-2-yl)-5-((2-cyanobenzyl)oxy)-2-methylbenzofuran-3-carboxamide
Cpd 342 (S)—N-(1-amino-3-hydroxy-1-oxopropan-2-yl)-5-((2-fluoro-4-methylbenzyl)oxy)-2-methylbenzofuran-3-carboxamide
Cpd 343 N-(1-carbamoylcyclobutyl)-2-methyl-5-(1-phenylethoxy)benzofuran-3-carboxamide
Cpd 344 5-(benzyloxy)-N-(1-carbamoylcyclobutyl)-2-methylbenzofuran-3-carboxamide
Cpd 345 N-(1-carbamoylcyclobutyl)-5-(1-(2-fluorophenyl)-2-hydroxyethoxy)-2-methylbenzofuran-3-carboxamide
Cpd 346 N—((S)-1-Amino-3-hydroxy-1-oxopropan-2-yl)-5-(2-methoxy-1-phenylethoxy)-2-methylbenzofuran-3-carboxamide
Cpd 347 N—((S)-1-Amino-3-hydroxy-1-oxopropan-2-yl)-5-(2-(dimethylamino)-1-phenylethoxy)-2-methylbenzofuran-3-carboxamide
   and the physiologically acceptable salts thereof.

The benzofuran derivative according to the invention is for use in the treatment of pain which is preferably selected from nociceptive pain, inflammatory pain, and neuropathic pain. More preferably, the pain is post-operative pain.

Another aspect of the invention relates to a compound of formula (I)

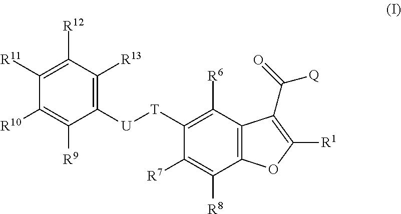

a stereo-isomeric form, a physiologically acceptable salt, solvate and/or polymorph thereof, as defined above;
preferably wherein
(a-1) Q represents —OR²; and R¹ represents —CH₂F, —CHF₂, or —CF₃; and/or
(a-2) Q represents —OR²; and at least one of R⁵ and R⁵′ does not represent —H; and at least one of R⁹, R¹⁰, R¹¹, R¹² and R¹³ does not represent —H; and/or
(a-3) Q represents —OR²; and R⁸ does not represent —H;
   or
(b-1) Q represents —NR³R⁴; and R¹ represents —CH₂F, —CHF₂, or —CF₃; and/or
(b-2) Q represents —NR³R⁴; and at least one of R⁹, R¹⁰, R¹¹, R¹² and R¹³ does not represent —H; and with the proviso that the following compounds are excluded:

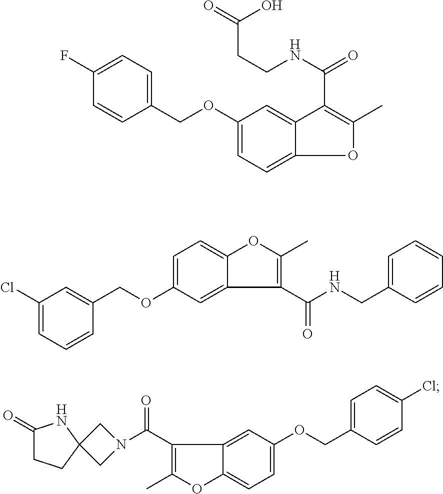 [SMILES: Cc1oc2ccc(OCc3ccc(F)cc3)cc2c1C(=O)NCCC(=O)O ; Cc1oc2ccc(OCc3cccc(Cl)c3)cc2c1C(=O)NCc1ccccc1 ; Cc1oc2ccc(OCc3ccc(Cl)cc3)cc2c1C(=O)N1CC2(CCC(=O)N2)C1]

and/or
(b-3) Q represents —NR³R⁴; and at least one of R⁵ and R⁵′ does not represent —H; and/or
(b-4) Q represents —NR³R⁴; and at least one of R⁶, R⁷ and R⁸ does not represent —H; with the proviso that the following compound is excluded:

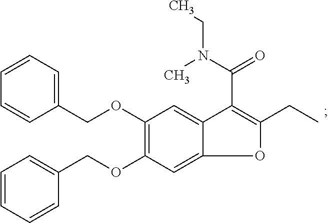 [SMILES: CCc1oc2cc(OCc3ccccc3)c(OCc3ccccc3)cc2c1C(=O)N(C)CC]

and/or
(b-5) Q represents —NR³R⁴; and R³ represent —H; and at least one of R⁹, R¹⁰, R¹¹, R¹² and R¹³ does not represent —H; and R⁴ represents
   —C₁-C₆-alkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.

In preferred embodiments of the benzofuran derivatives according to the invention (a-1), (a-2), (a-3), (b-1), (b-2), (b-3), (b-4), and (b-5) T represents —O— and U represents —CR⁵R⁵′— (i.e., the benzofuran derivatives is of formula (II)).

All definitions, preferred embodiments and preferred meanings of Q, T, U, R¹, R², R³, R⁴, R⁵, R⁵′, R⁶, R⁷, R⁸, R⁹, R¹⁰, R¹¹, R¹² and R¹³ including the preferred substituents also analogously apply the benzofuran derivatives according to the invention, including but not limited to (a-1), (a-2), (a-3), (b-1), (b-2), (b-3), (b-4), and (b-5), which are not necessarily restricted for use in the treatment of pain. Thus, this aspect of the invention relates to the benzofuran derivatives as such, compositions comprising the benzofuran derivatives, medicaments comprising the benzofuran derivatives, and the benzofuran derivatives for use in the prevention and/or treatment of TRPM3 mediated disorders such as pain and/or inflammatory hypersensitivity; and/or for counteracting pain and/or inflammatory hypersensitivity. Preferably, the pain is selected from nociceptive pain, inflammatory pain, and neuropathic pain. More preferably, the pain is post-operative pain.

In a preferred embodiment of the benzofuran derivative according to the invention Q represents —NR³R⁴ and with the proviso that at least one of R⁹, R¹⁰, R¹¹, R¹² and R¹³ represents neither —H, nor —F, nor —Cl.

In preferred embodiments of the invention, the benzofuran derivative is selected from the group consisting of Cpd 001 to Cpd 308 as mentioned above and the physiologically acceptable salts thereof.

Another aspect of the invention relates to a pharmaceutical composition or a medicament comprising a benzofuran derivative according to the invention as described above.

Reference throughout this specification to “one embodiment” or “an embodiment” means that a particular feature, structure or characteristic described in connection with the embodiment is included in at least one embodiment of the invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments. Also, embodiments described for an aspect of the invention may be used for another aspect of the invention and can be combined. Where an indefinite or definite article is used when referring to a singular noun e.g., “a” or “an”, “the”, this includes a plural of that noun unless something else is specifically stated.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects.

In each of the following definitions, the number of carbon atoms represents the maximum number of carbon atoms generally optimally present in the substituent or linker; it is understood that where otherwise indicated in the present application, the number of carbon atoms represents the optimal maximum number of carbon atoms for that particular substituent or linker.

The term “leaving group” or “LG” as used herein means a chemical group which is susceptible to be displaced by a nucleophile or cleaved off or hydrolyzed in basic or acidic conditions. In a particular embodiment, a leaving group is selected from a halogen atom (e.g., Cl, Br, I) or a sulfonate (e.g., mesylate, tosylate, triflate).

The term “protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

The term “heteroatom(s)” as used herein means an atom selected from nitrogen, which can be quaternized; oxygen; and sulfur, including sulfoxide and sulfone.

The term “alkyl, saturated or unsaturated” as used herein encompasses saturated alkyl as well as unsaturated alkyl such as alkenyl, alkynyl, and the like. The term “alkyl” as used herein means normal, secondary, or tertiary, linear or branched hydrocarbon with no site of unsaturation. Examples are methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. The term “alkenyl” as used herein means normal, secondary or tertiary, linear or branched hydrocarbon with at least one site (usually 1 to 3, preferably 1) of unsaturation, namely a carbon-carbon, sp2 double bond. Examples include, but are not limited to: ethylene or vinyl (—CH═CH₂), allyl (—CH₂CH═CH₂), and 5-hexenyl (—CH₂CH₂CH₂CH₂CH═CH₂). The double bond may be in the cis or trans configuration. The term “alkynyl” as used herein means normal, secondary, tertiary, linear or branched hydrocarbon with at least one site (usually 1 to 3, preferably 1) of unsaturation, namely a carbon-carbon, sp triple bond. Examples include, but are not limited to: ethynyl (—C≡CH), and 1-propynyl (propargyl, —CH₂C≡CH).

The term “alkylene, saturated or unsaturated” as used herein encompasses saturated alkylene as well as unsaturated alkylene such as alkenylene, alkynylene, alkenylene and the like. The term “alkylene” as used herein means saturated, linear or branched chain hydrocarbon radical having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like. The term “alkenylene” as used herein means linear or branched chain hydrocarbon radical with at least one site (usually 1 to 3, preferably 1) of unsaturation, namely a carbon-carbon, sp2 double bond, and having two monovalent radical centers derived by the removal of two hydrogen atoms from the same or two different carbon atoms of a parent alkene. The term “alkynylene” as used herein means linear or branched chain hydrocarbon radical with at least one site (usually 1 to 3, preferably 1) of unsaturation, namely a carbon-carbon, sp triple bond, and having two monovalent radical centers derived by the removal of two hydrogen atoms from the same or two different carbon atoms of a parent alkyne.

The term “heteroalkyl, saturated or unsaturated” as used herein encompasses saturated heteroalkyl as well as unsaturated heteroalkyl such as heteroalkenyl, heteroalkynyl, heteroalkenyl and the like. The term “heteroalkyl” as used herein means linear or branched chain alkyl wherein one or more carbon atoms (usually 1, 2 or 3) are replaced by a heteroatom, i.e., an oxygen, nitrogen or sulfur atom, with the proviso that said chain may not contain two adjacent O atoms or two adjacent S atoms. This means that one or more —CH₃ of said alkyl can be replaced by —NH₂ and/or that one or more —CH₂— of said alkyl can be replaced by —NH—, —O— or —S—. The S atoms in said chains may be optionally oxidized with one or two oxygen atoms, to afford sulfoxides and sulfones, respectively. Furthermore, the heteroalkyl groups in the benzofuran derivatives of the invention can contain an oxo or thio group at any carbon or heteroatom that will result in a stable compound. Exemplary heteroalkyl groups include, but are not limited to, alcohols, alkyl ethers (such as for example -methoxy, -ethoxy, -butoxy, . . . ), primary, secondary, and tertiary alkyl amines, amides, ketones, esters, alkyl sulfides, and alkyl sulfones. The term “heteroalkenyl” means linear or branched chain alkenyl wherein one or more carbon atoms (usually 1, 2 or 3) are replaced by an oxygen, nitrogen or sulfur atom, with the proviso that said chain may not contain two adjacent O atoms or two adjacent S atoms. The term heteroalkenyl thus comprises imines, —O-alkenyl, —NH-alkenyl, —N(alkenyl)₂, —N(alkyl)(alkenyl), and —S-alkenyl. The term “heteroalkynyl” as used herein means linear or branched chain alkynyl wherein one or more carbon atoms (usually 1, 2 or 3) are replaced by an oxygen, nitrogen or sulfur atom, with the proviso that said chain may not contain two adjacent O atoms or two adjacent S atoms. The term heteroalkynyl thus comprises -cyano, —O-alkynyl, —NH-alkynyl, —N(alkynyl)₂, —N(alkyl)(alkynyl), —N(alkenyl)(alkynyl), and —S-alkynyl.

The term “heteroalkylene, saturated or unsaturated” as used herein encompasses saturated heteroalkylene as well as unsaturated heteroalkylene such as heteroalkenylene, heteroalkynylene, heteroalkenylene and the like. The term “heteroalkylene” as used herein means linear or branched chain alkylene wherein one or more carbon atoms (usually 1, 2 or 3) are replaced by a heteroatom, i.e., an oxygen, nitrogen or sulfur atom, with the proviso that said chain may not contain two adjacent O atoms or two adjacent S atoms. The term “heteroalkenylene” as used herein means linear or branched chain alkenylene wherein one or more carbon atoms (usually 1, 2 or 3) are replaced by an oxygen, nitrogen or sulfur atom, with the proviso that said chain may not contain two adjacent O atoms or two adjacent S atoms. The term “heteroalkynylene” as used herein means linear or branched chain alkynylene wherein one or more carbon atoms (usually 1, 2 or 3) are replaced by an oxygen, nitrogen or sulfur atom, with the proviso that said chain may not contain two adjacent O atoms or two adjacent S atoms.

The term “cycloalkyl, saturated or unsaturated” as used herein encompasses saturated cycloalkyl as well as unsaturated cycloalkyl such as cycloalkenyl, cycloalkynyl and the like. The term “cycloalkyl” as used herein and unless otherwise stated means a saturated cyclic hydrocarbon radical, such as for instance cyclopropyl, cyclobutyl, cyclopentyl, cyclohexyl, cycloheptyl, cyclooctyl, norbornyl, fenchyl, decalinyl, adamantyl and the like. The term “cycloalkenyl” as used herein means a non-aromatic cyclic hydrocarbon radical with at least one site (usually 1 to 3, preferably 1) of unsaturation, namely a carbon-carbon, sp2 double bond. Examples include, but are not limited to cyclopentenyl and cyclohexenyl. The double bond may be in the cis or trans configuration. The term “cycloalkynyl” as used herein means a non-aromatic cyclic hydrocarbon radical with at least one site (usually 1 to 3, preferably 1) of unsaturation, namely a carbon-carbon, sp triple. An example is cyclohept-1-yne. Fused systems of a cycloalkyl ring with a heterocycloalkyl ring are considered as heterocycloalkyl irrespective of the ring that is bound to the core structure. Fused systems of a cycloalkyl ring with an aryl ring are considered as aryl irrespective of the ring that is bound to the core structure. Fused systems of a cycloalkyl ring with a heteroaryl ring are considered as heteroaryl irrespective of the ring that is bound to the core structure.

The term “heterocycloalkyl, saturated or unsaturated” as used herein encompasses saturated heterocycloalkyl as well as unsaturated non-aromatic heterocycloalkyl including at least one heteroatom, i.e., an N, O, or S as ring member. The term “heterocycloalkyl” as used herein and unless otherwise stated means “cycloalkyl” wherein one or more carbon atoms (usually 1, 2 or 3) are replaced by an oxygen, nitrogen or sulfur atom, with the proviso that said chain may not contain two adjacent O atoms or two adjacent S atoms. The term “heterocycloalkenyl” as used herein and unless otherwise stated means “cycloalkenyl” wherein one or more carbon atoms (usually 1, 2 or 3) are replaced by an oxygen, nitrogen or sulfur atom, with the proviso that said chain may not contain two adjacent O atoms or two adjacent S atoms. The term “heterocycloalkynyl” as used herein and unless otherwise stated means “cycloalkynyl” wherein one or more carbon atoms (usually 1, 2 or 3) are replaced by an oxygen, nitrogen or sulfur atom, with the proviso that said chain may not contain two adjacent O atoms or two adjacent S atoms. Examples of saturated and unsaturated heterocycloalkyl include but are not limited to azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofurane, tetrahydropyrane, tetrahydrothiopyrane, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo-[3.2.1]octane, 9-azabicyclo[3.3.1]nonane, hexahydro-1H-pyrrolizine, hexahydrocyclopenta[c]pyrrole, octahydrocyclopenta[c]pyrrole, and octahydropyrrolo[1,2-a]pyrazin. Further heterocycloalkyls in the meaning of the invention are described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566. When the heterocycloalkyl contains no nitrogen as ring member, it is typically bonded through carbon. When the heterocycloalkyl contains nitrogen as ring member, it may be bonded through nitrogen or carbon. Fused systems of heterocycloalkyl ring with a cycloalkyl ring are considered as heterocycloalkyl irrespective of the ring that is bound to the core structure. Fused systems of a heterocycloalkyl ring with an aryl ring are considered as heterocycloalkyl irrespective of the ring that is bound to the core structure. Fused systems of a heterocycloalkyl ring with a heteroaryl ring are considered as heteroaryl irrespective of the ring that is bound to the core structure.

The term “aryl” as used herein means an aromatic hydrocarbon. Typical aryl groups include, but are not limited to 1 ring, or 2 or 3 rings fused together, radicals derived from benzene, naphthalene, anthracene, biphenyl, and the like. Fused systems of an aryl ring with a cycloalkyl ring are considered as aryl irrespective of the ring that is bound to the core structure. Fused systems of an aryl ring with a heterocycloalkyl ring are considered as heterocycloalkyl irrespective of the ring that is bound to the core structure. Thus, indoline, dihydrobenzofuran, dihydrobenzothiophene and the like are considered as heterocycloalkyl according to the invention. Fused systems of an aryl ring with a heteroaryl ring are considered as heteroaryl irrespective of the ring that is bound to the core structure.

The term “heteroaryl” as used herein means an aromatic ring system including at least one heteroatom, i.e., N, O, or S as ring member of the aromatic ring system. Examples of heteroaryl include but are not limited to benzimidazole, benzisoxazole, benzoazole, benzodioxole, benzofuran, benzothiadiazole, benzothiazole, benzothiophene, carbazole, cinnoline, dibenzofuran, furane, furazane, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine.

By further way of example, carbon bonded heterocyclic rings are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 6, 7, or 8 of an isoquinoline.

Preferred carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl. By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of an isoindole, or isoindoline, position 4 of a morpholine, and position 9 of a carbazole, or ß-carboline. Preferred nitrogen bonded heterocycles include 1-aziridyl, 1-azetedyl, 1-pyrrolyl, 1-imidazolyl, 1-pyrazolyl, and 1-piperidinyl. Further heteroaryls in the meaning of the invention are described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

As used herein with respect to a substituting group, and unless otherwise stated, the terms “monosubstituted” “disubstituted”, “trisubstituted”, “polysubstituted” and the like means chemical structures defined herein, wherein the respective moiety is substituted with one or more substituents, meaning that one or more hydrogen atoms of said moiety are each independently replaced with a substituent. For example, —C₁₋₆-alkyl that may be polysubstituted with —F covers —CH₂F, —CHF₂, —CF₃, —CH₂CF₃, CF₂CF₃, and the like. Likewise, —C₁₋₆-alkyl that may be polysubstituted with substituents independently of one another selected from —F and —Cl covers —CH₂F, —CHF₂, —CF₃, —CH₂CF₃, CF₂CF₃, —CH₂Cl, —CHCl₂, —CCl₃, —CH₂CCl₃, CCl₂CCl₃, —CHClF, —CClF₂, —CCl₂CF₃, —CF₂CCl₃, —CClFCCl₂F, and the like. Any substituent designation that is found in more than one site in a compound of this invention shall be independently selected.

As used herein and unless otherwise stated, the term “solvate” includes any combination which may be formed by a derivative of this invention with a suitable inorganic solvent (e.g., hydrates) or organic solvent, such as but not limited to alcohols, ketones, esters, ethers, nitriles and the like.

The term “subject” as used herein, refers to an animal including humans, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation or partial alleviation of the symptoms of the disease or disorder being treated.

The term “composition” as used herein is intended to encompass a product comprising the specified ingredients in the therapeutically effective amounts, as well as any product which results, directly or indirectly, from combinations of the specified ingredients in the specified amounts.

The term “antagonist” or “inhibitor” as used herein refers to a compound capable of producing, depending on the circumstance, a functional antagonism of the TRPM3 ion channel, including competitive antagonists, non-competitive antagonists, desensitizing agonists, and partial agonists.

For purposes of the invention, the term “TRPM3-modulated” is used to refer to the condition of being affected by the modulation of the TRPM3 ion channel, including the state of being mediated by the TRPM3 ion channel.

The term “TRPM3 mediated disorder” as used herein refers to disorders or diseases for which the use of an antagonist of TRPM3 would prevent, treat, (partially) alleviate or improve the symptoms and consist of pain and inflammatory hypersensitivity condition. According to the International Association for the Study of Pain and for the purpose of the invention, pain is an unpleasant sensory and emotional experience associated with actual or potential tissue damage, or described in terms of such damage. Preferably, the TRPM3 mediated disorder is pain which is preferably selected from nociceptive pain, inflammatory pain, and neuropathic pain. More preferably, the pain is post-operative pain. For the purpose of the invention, the term “inflammatory hypersensitivity” is used to refer to a condition that is characterized by one or more hallmarks of inflammation, including edema, erythema, hyperthermia and pain, and/or by an exaggerated physiologic or pathophysiologic response to one or more than one type of stimulation, including thermal, mechanical and/or chemical stimulation.

The benzofuran derivatives of the invention have been shown to be antagonists of TRPM3 and the invention therefore provides the compounds as such, the compounds for use as a medicine, more specifically for use as a medicine in the prevention or treatment of TRPM3 mediated disorders in a subject with a therapeutically effective amount of a benzofuran derivative of the invention.

In a preferred embodiment of the invention, the benzofuran derivative of the invention is the sole pharmacologically active compound to be administered for therapy. In another preferred embodiment of the invention, the benzofuran derivative of the invention may be employed in combination with other therapeutic agents for the treatment or prophylaxis of TRPM3 mediated disorders. The invention therefore also relates to the use of a composition comprising:
one or more compounds of the formulae and embodiments herein, and
one or more further therapeutic or preventive agents that are used for the prevention or treatment of TRPM3 mediated disorders as biologically active agents in the form of a combined preparation for simultaneous, separate or sequential use.

The pharmaceutical composition or combined preparation according to this invention may contain benzofuran derivatives of the invention over a broad content range depending on the contemplated use and the expected effect of the preparation. Generally, the content of the benzofuran derivatives of the invention of the combined preparation is within the range of 0.1 to 99.9% by weight, preferably from 1 to 99% by weight, more preferably from 5 to 95% by weight.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g., one of them may be in the form of an oral or parenteral formulation whereas the other is in the form of an ampoule for intravenous injection or an aerosol.

Those of skill in the art will also recognize that the benzofuran derivatives of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The term “pharmaceutically acceptable salts” as used herein means the therapeutically active non-toxic salt forms which the compounds of formulae herein are able to form. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The benzofuran derivatives of the invention may bear multiple positive or negative charges. The net charge of the benzofuran derivatives of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalogen acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein comprise benzofuran derivatives of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The benzofuran derivatives of the invention also include physiologically acceptable salts thereof. Examples of physiologically acceptable salts of the benzofuran derivatives of the invention include salts derived from an appropriate base, such as an alkali metal (for example, sodium), an alkaline earth (for example, magnesium), ammonium and NX4⁺ (wherein X is —C₁₋₆-alkyl). Physiologically acceptable salts of a hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX4⁺ (wherein X typically is independently selected from —H or a —C₁₋₄-alkyl group). However, salts of acids or bases which are not physiologically acceptable may also find use, for example, in the preparation or purification of a physiologically acceptable compound. All salts, whether or not derived form a physiologically acceptable acid or base, are within the scope of the invention.

As used herein and unless otherwise stated, the term “enantiomer” means each individual optically active form of a benzofuran derivative of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “isomers” as used herein means all possible isomeric forms, including tautomeric and stereochemical forms, which the compounds of formulae herein may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e., at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of a benzofuran derivative of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compound. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel® CA, OA, OB5, OC5, OD, OF, OG, OJ and OK, and Chiralpak® AD, AS, OP(+) and OT(+). Appropriate eluents or mobile phases for use in combination with said polysaccharide chiral stationary phases are hexane and the like, modified with an alcohol such as ethanol, isopropanol and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

The terms cis and trans are used herein in accordance with Chemical Abstracts nomenclature and include reference to the position of the substituents on a ring moiety. The absolute stereochemical configuration of the compounds of the formulae described herein may easily be determined by those skilled in the art while using well-known methods such as, for example, X-ray diffraction.

When a compound is crystallized from a solution or slurry, it can be crystallized in a different arrangement lattice of spaces (this property is called “polymorphism”) to form crystals with different crystalline forms, each of which is known as “polymorphs”. The term “Polymorph” as used herein therefore, refers to a crystal form of a compound of Formula (I), where the molecules are localized in the three-dimensional lattice sites. Different polymorphs of the compound of Formula (I) may be different from each other in one or more physical properties, such as solubility and dissolution rate, true specific gravity, crystal form, accumulation mode, flowability and/or solid-state stability. etc.

Benzofuran derivatives of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intranasal, intravenous, intraarterial, intradermal, intrathecal and epidural). The preferred route of administration may vary with for example the condition of the recipient.

The therapeutically effective amount of the preparation of the compound(s), especially for the treatment of TRPM3 mediated disorders in humans and other mammals or in animals, preferably is a TRPM3 ion channel inhibiting amount of the compounds as defined herein and corresponds to an amount which ensures a plasma level of between 1 μg/ml and 100 mg/ml, optionally of 10 mg/ml.

Suitable dosages of the compounds or compositions of the invention should be used to treat or prevent the TRPM3 mediated disorders in a subject. Depending upon the pathologic condition to be treated and the patient's condition, the said effective amount may be divided into several sub-units per day or may be administered at more than one day intervals.

The invention further provides (pharmaceutical) compositions comprising one or more benzofuran derivatives of the invention, more in particular of all the Formula (I) and other formulas and embodiments described herein and the more particular aspects or embodiments thereof. Furthermore, the invention provides the compounds or (pharmaceutical) compositions of the invention, more in particular of all the Formula (I) and other formulas and embodiments described herein and the more particular aspects or embodiments thereof, for use as a medicine, more in particular for use in the treatment of pain. The TRPM3 mediated disorders are selected from pain and an inflammatory hypersensitivity condition.

The benzofuran derivatives of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986).

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e., the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the invention. They may also include additives such as wetting agents, dispersing agents, stickers, adhesives, emulsifying agents, surface-active agents, solvents, coatings, antibacterial and antifungal agents, isotonic agents and the like, provided the same are consistent with pharmaceutical practice, i.e., carriers and additives which do not create permanent damage to mammals. The pharmaceutical compositions of the invention may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by micronisation, for instance in view to obtain them in the form of microspheres usually having a diameter of about 1 to 10 gm, namely for the manufacture of microcapsules for controlled or sustained release of the active ingredients.

While it is possible for the benzofuran derivatives to be administered alone it is preferable to present them as pharmaceutical formulations. The formulations, both for veterinary and for human use, of the invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g. mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus, the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc.), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Benzofuran derivatives of the invention can be used to provide controlled release pharmaceutical formulations containing as active ingredient one or more benzofuran derivatives of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more benzofuran derivatives of the invention can be prepared according to conventional methods.

Another embodiment of this invention relates to various precursor or “prodrug” forms of the benzofuran derivatives of the invention. It may be desirable to formulate the benzofuran derivatives of the invention in the form of a chemical species which itself is not significantly biologically-active, but which when delivered to the animal, mammal or human will undergo a chemical reaction catalyzed by the normal function of the body, inter alia, enzymes present in the stomach or in blood serum, said chemical reaction having the effect of releasing a compound as defined herein. The term “prodrug” thus relates to these species which are converted in vivo into the active pharmaceutical ingredient.

The prodrugs of the benzofuran derivatives of the invention can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used. The counterpart of the active pharmaceutical ingredient in the pro-drug can have different structures such as an amino acid or peptide structure, alkyl chains, sugar moieties and others as known in the art.

For the purpose of the invention the term “therapeutically suitable pro-drug” is defined herein as “a compound modified in such a way as to be transformed in vivo to the therapeutically active form, whether by way of a single or by multiple biological transformations, when in contact with the tissues of the animal, mammal or human to which the pro-drug has been administered, and without undue toxicity, irritation, or allergic response, and achieving the intended therapeutic outcome”.

More specifically the term “prodrug” as used herein, relates to an inactive or significantly less active derivative of a compound such as represented by the structural formulae herein described, which undergoes spontaneous or enzymatic transformation within the body in order to release the pharmacologically active form of the compound. For a comprehensive review, reference is made to Rautio J. et al. (“Prodrugs: design and clinical applications” Nature Reviews Drug Discovery, 2008, doi: 10.1038/nrd2468).

Representative benzofuran derivatives of the invention can be synthesized in accordance with the general synthetic methods described below and illustrated in the schemes that follow. Since the schemes are an illustration, the invention should not be construed as being limited by the specific chemical reaction and specific conditions described in the schemes and examples. The various starting material used in the schemes are commercially available or may be prepared by methods well within the skill persons versed in the art. The variables are as defined herein and within the skill of persons verses in the art.

Preferred embodiments of the invention are summarized as clauses 1 to 42 hereinafter:
1. A compound of formula (I), a stereo-isomeric form, a physiologically acceptable salt, solvate and/or polymorph thereof

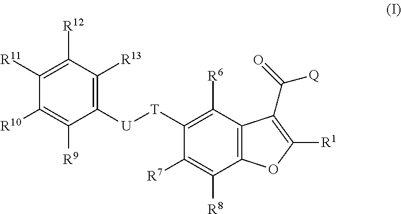

preferably the compound of formula (I)), a stereo-isomeric form, a physiologically acceptable salt, solvate and/or polymorph thereof, for use in the treatment of pain,
wherein
R¹ represents —F, —Cl, —Br, —I, —CN, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
Q represents —OR² or —NR³R⁴;
R² represents —R^{Y};
R³ represents —OH or —R^{Y};
R⁴ represents —R^{Y} or —S(═O)₂R^{Y};
   or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —CR⁵R^{5′}—; or T represents —CR⁵R⁵′— and U represents —O—;
R⁵ and R^{5′} independently of one another represent —R^{Y};
   or, provided that T represents —O— and U represents —CR⁵R^{5′}—, alternatively R⁵ and R⁹ together form a 4-8-membered carbocycle, saturated or unsaturated, unsubstituted or mono- or polysubstituted; or a 4-8 membered heterocycle, saturated or unsaturated, containing 1 to 3 heteroatoms selected from N, O and S, unsubstituted or mono- or polysubstituted;
R⁶, R⁷ and R⁸ independently of one another represent —F, —Cl, —Br, —I, —CN, —NO₂, —SF₅, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
R⁹, R¹⁰, R¹² and R¹³ independently of one another represent —F, —Cl, —Br, —I, —CN, —NO, —NO₂, ═O, ═S, —SF₅, —R^{Y}, —OR^{Y}, —OC(═O)R^{Y}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z};
wherein
R^{W} and R^{X} independently of one another in each case independently represent
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or 3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y} and R^{Z} independently of one another in each case independently represent
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, 6-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, —S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, —S(═O)₂-(5 to 14-membered heteroaryl).
2. The compound per se, or for use according to Clause 1, wherein
   Q represents —NR³R⁴;
   T represents —O— and U represents —CR⁵R^{5′}—;
   at least one of R⁹, R¹⁰, R¹¹, R¹² and R¹³ does not represent —H; and
   with the proviso that the following compounds are excluded:

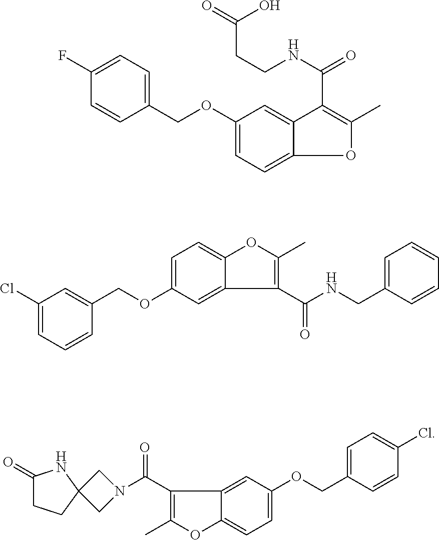 [SMILES: Cc1oc2ccc(OCc3ccc(F)cc3)cc2c1C(=O)NCCC(=O)O ; Cc1oc2ccc(OCc3cccc(Cl)c3)cc2c1C(=O)NCc1ccccc1 ; Cc1oc2ccc(OCc3ccc(Cl)cc3)cc2c1C(=O)N1CC2(CCC(=O)N2)C1]

3. The compound per se, or for use according to Clause 1 or 2, wherein T represents —O— and U represents —CR⁵R^{5′}—.
4. The compound per se, or for use according to any one of Clauses 1 to 3, wherein Q represents —NR³R⁴.
5. The compound per se, or for use according to any one of Clauses 1 to 3, wherein Q represents —OR².
6. The compound per se, or for use according to any one of the preceding Clauses, wherein R¹ represents
   —H, —F, —Cl, —Br, —I;
   —C₁₋₆-alkyl, saturated or unsaturated, unsubstituted, mono- or polysubstituted; —O—C₁₋₆-alkyl, saturated or unsaturated, unsubstituted, mono- or polysubstituted;
   —C(═O)C₁₋₆-alkyl, saturated or unsaturated, unsubstituted, mono- or polysubstituted;
   —C(═O)OC₁₋₆-alkyl, saturated or unsaturated, unsubstituted, mono- or polysubstituted;
   —C(═O)NHC₁₋₆-alkyl, saturated or unsaturated, unsubstituted, mono- or polysubstituted;
   —C(═O)N(C₁₋₆-alkyl)₂, saturated or unsaturated, unsubstituted, mono- or polysubstituted;
   —S(═O)C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   —C₁-C₆-heteroalkyl, saturated or unsaturated, unsubstituted, mono- or polysubstituted; or
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.
7. The compound per se, or for use according to any one of the preceding Clauses, wherein R¹ represents —H, —F, —Cl, —Br, —I, —C₁₋₆-alkyl, —O—C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NHC₁₋₆-alkyl, —C(═O)N(C₁₋₆-alkyl)₂, —S(═O)—C₁₋₆-alkyl, —S(═O)₂—C₁₋₆-alkyl, —O—C₁₋₆-alkyl, -cyclopropyl unsubstituted, cyclobutyl unsubstituted, cyclopentyl unsubstituted or cyclohexyl unsubstituted.
8. The compound per se, or for use according to any one of the preceding Clauses, wherein R¹ represents —H, —C₁₋₆-alkyl, —C₁₋₆-alkylene-O—C₁₋₆-alkyl, —CHF₂, —CF₃, or -cyclopentyl, unsubstituted.
9. The compound per se, or for use according to any one of the preceding Clauses, wherein R² represents
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted.
10. The compound per se, or for use according to any one of the preceding Clauses, wherein R² represents —H, —C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.
11. The compound per se, or for use according to any one of the preceding Clauses, wherein R² represents —H or —C₁₋₆-alkyl.
12. The compound per se, or for use according to any one of the preceding Clauses, wherein R³ represents
   —H;
   —OH;
   —C₁-C₆-alkyl, saturated or unsaturated, unsubstituted, mono- or polysubstituted; or
   —C₁-C₆-heteroalkyl, saturated or unsaturated, unsubstituted, mono- or polysubstituted.
13. The compound per se, or for use according to any one of the preceding Clauses, wherein R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.
14. The compound per se, or for use according to any one of the preceding Clauses, wherein R³ represents —H, —OH, or —C₁₋₆-alkyl, saturated, unsubstituted or monosubstituted with —OH.
15. The compound per se, or for use according to any one of the preceding Clauses, wherein R⁴ represents
   —H;
   —S(═O)C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.
16. The compound per se, or for use according to any one of the preceding Clauses, wherein R⁴ represents —S(═O)₂C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   —S(═O)₂(3-14-membered cycloalkyl), wherein said 3-14-membered cycloalkyl is selected from the group consisting of cyclopropyl, cyclobutyl, cyclopentyl, cyclohexyl, and cycloheptyl, in each case saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   —C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is selected from the group consisting of cyclopropyl, cyclobutyl, cyclopentyl, cyclohexyl, and cycloheptyl, in each case saturated or unsaturated, in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from the group consisting of azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofurane, tetrahydropyrane, tetrahydrothiopyrane, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexahydrocyclopenta[c]pyrrole, octahydrocyclopenta[c]pyrrole, and octahydropyrrolo[1,2-a]pyrazin; in each case unsubstituted, mono- or polysubstituted with substituents independently of one another selected from the group consisting of —F, —Cl, —C₁₋₆-alkyl, —C₁₋₆-alkylene-CF₃, —OH, ═O, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl,
   -phenyl, —C₁₋₆-alkylene-phenyl, 3-14-membered heterocycloalkyl, saturated or unsaturated, unsubstituted; and 5-14-membered heteroaryl, unsubstituted;
   -phenyl unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of benzimidazole, benzisoxazole, benzoazole, benzodioxole, benzofuran, benzothiadiazole, benzothiazole, benzothiophene, carbazole, cinnoline, dibenzofuran, furane, furazane, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted.
17. The compound per se, or for use according to any one of the preceding Clauses, wherein R⁴ represents
   —H;
   —S(═O)₂C₁₋₆-alkyl, saturated, unsubstituted, monosubstituted or polysubstituted with —F;
   —S(═O)₂(3-14-membered cycloalkyl), saturated, unsubstituted;
   —C₁₋₆-alkyl, saturated, unsubstituted, monosubstituted or disubstituted with substituents independently of one another selected from the group consisting of —OH, —OC₁₋₆-alkyl, —N(C₁₋₆-alkyl)₂, —C₁₋₆-alkylene-NH₂, —C₁₋₆-alkylene-NH—C₁₋₆-alkyl, -phenyl unsubstituted;
   3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is saturated, unsubstituted, monosubstituted or disubstituted with substituents independently of one another selected from the group consisting of —C₁₋₆-alkyl, —C₁₋₆-alkylene-NH₂, —C₁₋₆-alkylene-NH—C₁₋₆-alkylene-CF₃, —C₁₋₆-alkylene-OH, —C₁₋₆-alkylene-NHC(═O)O—C₁₋₆-alkyl, —OH, —OC₁₋₆-alkyl, —NH₂, —N(C₁₋₆-alkyl)₂, —NHC(═O)O—C₁₋₆-alkyl;
   3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from azetane, 1,4-oxazepane, pyrrolidine, piperidine, azepane, diazepane, tetrahydrofurane, tetrahydropyrane, oxetane, morpholine, piperazine, hexahydrocyclopenta[c]pyrrole, octahydrocyclopenta[c]pyrrole, octahydropyrrolo[1,2-a]pyrazin, 8-azabicyclo[3.2.1]octane, 9-azabicyclo-[3.3.1]nonane, quinuclidine, hexahydro-1H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —OH, ═O, —C₁₋₆-alkyl, —C₁₋₆-alkylene-CF₃, —C₁₋₆-alkylene-OH, —C₁₋₆-alkylene-O—C₁₋₆-alkyl, —NH₂, —N(C₁₋₆-alkyl)₂, —C₁₋₆-alkylene-NH₂, —C₁₋₆-alkylene-N(C₁₋₆-alkyl)₂, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —S(═O)₂C₁₋₆-alkyl, oxetanyl, pyrimidinyl, —C₁₋₆-alkylene-phenyl;
   -phenyl unsubstituted;
   5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —C₁₋₆-alkyl, —OH.
18. The compound per se, or for use according to any one of the preceding Clauses, wherein R³ and R⁴ together form a 5- or 6-membered heterocycle containing 1 or 2 heteroatoms selected from N, O and S, saturated or unsaturated, unsubstituted or mono- or polysubstituted.
19. The compound per se, or for use according to any one of the preceding Clauses, wherein R³ and R⁴ together form a heterocycle selected from the group consisting of pyrrolidine, piperidine, morpholine, and piperazine, in each case unsubstituted, mono- or polysubstituted with substituents independently of one another selected from the group consisting of —C₁₋₆-alkyl, —NH₂, —NHCH₃, —N(CH₃)₂, —C(═O)NH—C₁₋₆-alkyl, —C(═O)N(C₁₋₆-alkyl)₂, —C(═O)O—C₁₋₆-alkyl, —NHC(═O)O—C₁₋₆-alkyl, -pyridyl unsubstituted, and 1,2,4-oxadiazole unsubstituted or monosubstituted with —C₁₋₆-alkyl.
20. The compound per se, or for use according to any one of the preceding Clauses, wherein R³ and R⁴ together form a
   pyrrolidine ring, unsubstituted or monosubstituted with —N(CH₃)₂;
   piperidine ring, unsubstituted or monosubstituted with a substituent selected from the group consisting of —C₁₋₆-alkyl, —NH₂, —N(CH₃)₂, —C(═O)NH—C₁₋₆-alkyl, —C(═O)O—C₁₋₆-alkyl, —NHC(═O)O—C₁₋₆-alkyl, and 1,2,4-oxadiazole unsubstituted or monosubstituted with —C₁₋₆-alkyl;
   morpholine ring, unsubstituted; or
   piperazine ring, unsubstituted or N-substituted with a substituent selected from the group consisting of —C₁₋₆-alkyl and -pyridyl unsubstituted.
21. The compound per se, or for use according to any one of the preceding Clauses, wherein R⁵ and R⁵′ independently of one another represent
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.
22. The compound per se, or for use according to any one of the preceding Clauses, wherein R⁵ and R⁵′ independently of one another represent —H, —C₁-C₆-alkyl, or —C₁-C₆-alkylene-N(C₁-C₆-alkyl)₂.
23. The compound per se, or for use according to any one of the preceding Clauses, wherein R⁵ and R⁹ together form a 5-6-membered carbocycle, unsubstituted; or R⁵ and R⁹ together form a 5-6-membered heterocycle containing 1 heteroatom 0, unsubstituted.
24. The compound per se, or for use according to any one of the preceding Clauses, wherein R⁶, R⁷ and R⁸ independently of one another represent
   —H;
   —F, —Cl, —Br, —I, —OH, —SH, —SF₅, —CN, —NO₂, —C(═O)OH, —NH₂;
   —C₁₋₆-alkyl, saturated or unsaturated, unsubstituted, mono- or polysubstituted;
   —O—C₁₋₆-alkyl, saturated or unsaturated, unsubstituted, mono- or polysubstituted;
   —NHC₁₋₆-alkyl, saturated or unsaturated, unsubstituted, mono- or polysubstituted;
   —N(C₁₋₆-alkyl)₂, saturated or unsaturated, unsubstituted, mono- or polysubstituted;
   —C(═O)OC₁₋₆-alkyl, saturated or unsaturated, unsubstituted, mono- or polysubstituted;
   —OC(═O)C₁₋₆-alkyl, saturated or unsaturated, unsubstituted, mono- or polysubstituted;
   —C₁₋₆-heteroalkyl, saturated or unsaturated, unsubstituted, mono- or polysubstituted.
25. The compound per se, or for use according to any one of the preceding Clauses, wherein R⁶, R⁷ and R⁸ independently of one another represent
   —H, —F, —Cl, —Br, —I, —OH, —SH, —SF₅, —CN, —NO₂, —C(═O)OH, —NH₂,
   —C₁₋₆-alkyl, —CF₃, —CHF₂, —CH₂F,
   —O—C₁₋₆-alkyl, —OCF₃, —OCHF₂, —OCH₂F,
   —NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —C(═O)OC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —OC(═O)C₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂; or
   —C₁₋₆-heteroalkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂.
26. The compound per se, or for use according to any one of the preceding Clauses, wherein R⁶ represents —H, —F, —Cl, —CN, or —C₁-C₆-alkyl.
27. The compound per se, or for use according to any one of the preceding Clauses, wherein R⁶ does not represent —H.
28. The compound per se, or for use according to any one of the preceding Clauses, wherein R⁷ represents —H, —F, —Cl, —CN, or —C₁-C₆-alkyl.
29. The compound per se, or for use according to any one of the preceding Clauses, wherein R⁷ does not represent —H.
30. The compound per se, or for use according to any one of the preceding Clauses, wherein R⁸ represents —H, —F, —Cl, —CN, or —C₁-C₆-alkyl.
31. The compound per se, or for use according to any one of the preceding Clauses, wherein R⁸ does not represent —H.
32. The compound per se, or for use according to any one of the preceding Clauses, wherein
   (i) R⁶, R⁷ and R⁸ each represent —H;
   (ii) two of R⁶, R⁷ and R⁸ represent —H and the other of R⁶, R⁷ and R⁸ represents —F, —Cl, —CN, or —CH₃;
   (iii) one of R⁶, R⁷ and R⁸ represents —H and the other of R⁶, R⁷ and R⁸ independently of one another represent —F, —Cl, —CN, or —CH₃.
33. The compound per se, or for use according to any one of the preceding Clauses, wherein R⁹, R¹⁰, R¹¹, R¹² and R¹³ independently of one another represent
   —H, —F, —Cl, —Br, —I, —CN, —C(═O)OH, —NH₂, —NO₂, —OH, ═O, —SF₅;
   —C₁₋₆-alkyl, saturated or unsaturated, unsubstituted, mono- or polysubstituted;
   —C(═O)O—C₁₋₆-alkyl, saturated or unsaturated, unsubstituted, mono- or polysubstituted;
   —NHC₁₋₆-alkyl, saturated or unsaturated, unsubstituted, mono- or polysubstituted;
   —N(C₁₋₆-alkyl)₂, saturated or unsaturated, unsubstituted, mono- or polysubstituted;
   —O—C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
   3-14-membered heterocycloalkyl, saturated or unsaturated, unsubstituted, mono- or polysubstituted;
   wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted.
34. The compound per se, or for use according to any one of the preceding Clauses, wherein R⁹, R¹⁰, R¹¹, R¹² and R¹³ independently of one another represent
   —H, —OH, —F, —Cl, —Br, —I, —SH, —CF₃, —CHF₂, —CH₂F, —OCF₃, —OCHF₂, —OCH₂F, SF₅, —CN, —NO₂, —C(═O)OH, —NH₂, or —N(CH₃)₂;
   —C₁₋₆-alkyl, saturated or unsaturated, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   —C₁₋₆-heteroalkyl, saturated or unsaturated, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   —OC₁₋₆-alkyl, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   —O(C═O)C₁₋₆-alkyl, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   —C(═O)OC₁₋₆-alkyl, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   3-14-membered cycloalkyl selected from the group consisting of cyclopropyl, cyclobutyl, cyclopentyl, cyclohexyl and cycloheptyl; in each case unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   3-14-membered heterocycloalkyl selected from the group consisting of azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofurane, tetrahydropyrane, tetrahydrothiopyrane, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]-nonane, 8-azabicyclo[3.2.1]octane, 9-azabicyclo[3.3.1]nonane, hexahydro-1H-pyrrolizine, hexahydrocyclopenta[c]pyrrole, octahydrocyclopenta[c]pyrrole, and octahydropyrrolo[1,2-a]pyrazin, in each case unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂.
35. The compound per se, or for use according to any one of the preceding Clauses, wherein R⁹, R¹⁰, R¹¹, R¹² and R¹³ independently of one another represent —H, —F, —Cl, —CN, —OH, ═O, —C₁₋₆-alkyl, —CHF₂, —CF₃, —C₁₋₆-alkylene-NH₂, —C₁₋₆-alkylene-NHC(═O)O—C₁₋₆-alkyl, —C₁₋₆-alkylene-OH, —C₁₋₆-alkylene-NHC(═O)—O—C₁₋₆-alkyl, —C(═O)O—C₁₋₆-alkyl, —N(C₁₋₆-alkyl)₂, —OC₁₋₆-alkyl, —OCF₃, —O—C₁₋₆-alkylene-N(C₁₋₆-alkyl)₂, —S(═O)₂—C₁₋₆-alkyl, -azetidine, —C₁₋₆-alkylene-O-tetrahydropyran, or -piperazine substituted with —C₁₋₆-alkyl.
36. The compound per se, or for use according to any one of the preceding Clauses, wherein
   (i) R⁹, R¹⁰, R¹¹, R¹² and R¹³ represent —H;
   (ii) four of R⁹, R¹⁰, R¹¹, R¹² and R¹³ represent —H and the other two of R⁹, R¹⁰, R¹¹, R¹² and R¹³ represent a substituent other than —H; or
   (iii) three of R⁹, R¹⁰, R¹¹, R¹² and R¹³ represent —H and the other of R⁹, R¹⁰, R¹¹, R¹² and R¹³ represents a substituent other than —H.
37. The compound per se, or for use according to any one of the preceding Clauses, which is selected from the group consisting of Cpd 001 to Cpd 308 as mentioned above and the physiologically acceptable salts thereof.
38. The compound per se, or for use according to any one of the preceding Clauses, wherein the pain is selected from nociceptive pain, inflammatory pain, and neuropathic pain.
39. The compound per se, or for use according to any one of the preceding Clauses, wherein the pain is post-operative pain.
40. A compound of formula (I), a stereo-isomeric form, a physiologically acceptable salt, solvate and/or polymorph thereof, as defined in any one of the preceding Clauses, wherein
   (a) Q represents —OR²; and
      (a-1) R¹ represents —CH₂F, —CHF₂, or —CF₃; and/or
      (a-2) at least one of R⁵ and R⁵′ does not represent —H; and at least one of R⁹, R¹⁰, R¹¹, R¹² and R¹³ does not represent —H; and/or
      (a-3) R⁸ does not represent —H;
   or
   (b) Q represents —NR³R⁴; and
      (b-1) R¹ represents —CH₂F, —CHF₂, or —CF₃; and/or
      (b-2) at least one of R⁹, R¹⁰, R¹¹, R¹² and R¹³ does not represent —H; and with the proviso that the following compounds are excluded:

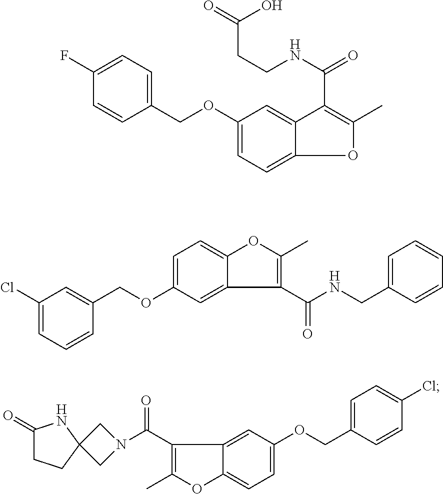 [SMILES: Cc1oc2ccc(OCc3ccc(F)cc3)cc2c1C(=O)NCCC(=O)O ; Cc1oc2ccc(OCc3cccc(Cl)c3)cc2c1C(=O)NCc1ccccc1 ; Cc1oc2ccc(OCc3ccc(Cl)cc3)cc2c1C(=O)N1CC2(CCC(=O)N2)C1]

and/or
(b-3) at least one of R⁵ and R⁵′ does not represent —H; and/or
(b-4) at least one of R⁶, R⁷ and R⁸ does not represent —H; with the proviso that the following compound is excluded:

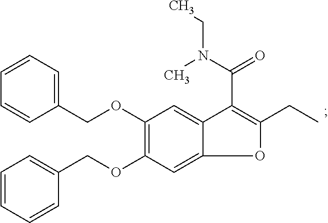 [SMILES: CCc1oc2cc(OCc3ccccc3)c(OCc3ccccc3)cc2c1C(=O)N(C)CC]

and/or
(b-5) R³ represent —H; and at least one of R⁹, R¹⁰, R¹¹, R¹² and R¹³ does not represent —H; and R⁴ represents
   —C₁-C₆-alkyl, saturated or unsaturated, unsubstituted, mono- or polysubstituted; 3-14-membered cycloalkyl, saturated or unsaturated, unsubstituted, mono- or
   3-14-membered polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.
41. The compound according to Clause 40, wherein Q represents —NR³R⁴ and with the proviso that at least one of R⁹, R¹⁰, R¹¹, R¹² and R¹³ represents neither —H, nor —F, nor —Cl.
42. A pharmaceutical composition or a medicament comprising a compound according to any one of the preceding Clauses.

### Examples

The following examples are provided for the purpose of illustrating the invention and by no means should be interpreted to limit the scope of the invention.

Representative compounds of the present invention can be synthesized in accordance with the general synthetic methods described below and are illustrated in the schemes that follow. Since the schemes are an illustration, the invention should not be construed as being limited by the specific chemical reaction and specific conditions described in the schemes and examples. The various starting materials used in the schemes are commercially available or may be prepared by methods well within the skill of persons versed in the art. The variables are as defined herein and within the skill of persons versed in the art.

Abbreviations used in the instant specification, particularly in the schemes and examples, are as follows: AcOH—Acetic acid, ADDP—1,1′-(Azodicarbonyl)dipiperidine, aq.—Aqueous, COMU—(1-Cyano-2-ethoxy-2-oxoethylidenaminooxy)dimethylamino-morpholino-carbenium hexafluorophosphate, DBU—1,8-Diazabicyclo-[5.4.0]undec-7-ene, DCC—N,N′-dicyclohexylcathodiimide, DCM—Dichloromethane, DEAD—Diethyl azodicarboxylate, DEA—Diethylamine, DIPEA—Diisopropyl-ethyl amine, DIA—Diastereomer, DIAD—Diisopropyl azodicarboxylate, DME—1,2-Dimethoxyethane, DMF—N,N-Dimethylformamide, DMSO—Dimethylsulfoxide, DTBAD—tert-Butylazodicarboxylate, EDCI—1-Ethyl-3-(3-dimethylaminopropyl)carbodiimide, En—Enantiomer, Et2O—Diethyl ether, EtOAc—Ethyl acetate, EtOH—Ethanol, Eq.—Equivalent, FA—Formic acid, FCC—Flash column chromatography, h—Hour, HATU—O-(7-Azabenzotriazol-1-yl)-N,N,N′,N′-tetramethyluronium hexafluorophosphate, HPLC—High performance liquid chromatography, IPA—isopropyl alcohol, LG—Leaving group, MgSO4—Magnesium sulfate, min.—Minute, Na2SO4—Sodium sulfate, NBS—N-Bromosuccinimide, NMP—1-Methyl-2-pyrrolidinone, Pd(PPh3)4—Tetrakis-(triphenylphosphine)-palladium(0), Pd2(dba)3—Tris(dibenzylideneacetone)dipalladium, PPh3—Triphenylphospine, PS-DIEA Diisopropyl-ethyl amine supported on PolyStyrene, PS—PPh3—Triphenylphospine supported on PolyStyrene, PyBop—Benzotriazol-1-yl-oxytripyrrolidinophosphonium hexafluorophosphate, RP—Reverse phase, RT—Room temperature, RM—Reaction mixture, sat.—Saturated, SFC—Supercritical fluid chromatography, SPE—Solid Phase Extraction, TBAI—Tetrabutylammonium iodide, TEA—Triethylamine, THF—Tetrahydrofuran, TFA—Trifluoroacetic acid.

The compounds of interest have a structure according to the general formula (A) and all other formulas described herein and embodiments thereof can be prepared as outlined in the general chemical scheme 1.

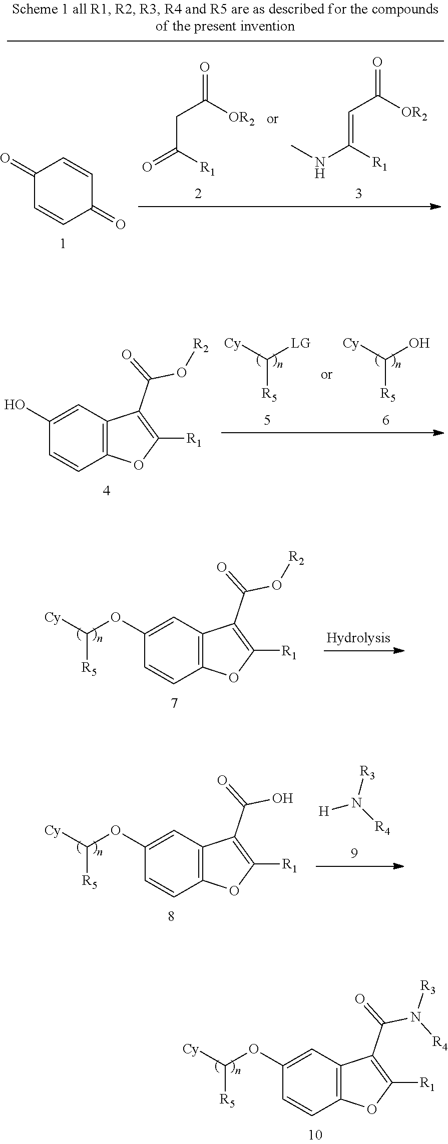

para-Benzoquinone of formula 1, may be condensed with a ketoester of formula 2 (commercially available or synthesized by procedures known to the person skilled in the art), wherein R₂ is an ester protecting group (e.g. methyl, ethyl, t-Bu and the like), in the presence of a Lewis acid (e.g., Titanium(IV) chloride, zinc(II) chloride and the like) in a polar solvent (e.g., DCM, MeOH, EtOH, and the like) at a temperature ranging from 0 to 100° C. to provide intermediates of formula 4. More detailed information can be found in the following references (Bioorg. Med. Chem. 2012, 20, 4237-4244 and FR 1319594). Alternatively, para-benzoquinone of formula 1 may be reacted with an enamine of formula 3 (commercially available or synthesized by procedures known to the person skilled in the art), in the presence of a protic acid (e.g., trifluoroacetic acid, para-toluenesulfonic acid, and the like) in a polar solvent (e.g., DCM, MeOH, EtOH, and the like) at a temperature ranging from 0 to 100° C. to provide intermediates of formula 4. More detailed information can be found in the following reference (J. Heterocyclic Chem. 2006, 43, 873). Intermediates of formula 4 may then be converted into the desired compounds of formula 7 via nucleophilic substitution using intermediates of formula 5 (commercially available or synthesized), wherein LG is a leaving group, in the presence of a base (e.g., DIPEA, DBU, triethylamine, Cs₂CO₃, and the like) in a polar solvent (e.g., acetonitrile, DMF, NMP, and the like), with or without a chelating agent (e.g., 18-crown-6, cis-anti-cis-dicyclohexano-18-crown-6, and the like) at a temperature ranging from 0 to 100° C. Alternatively, intermediates of formula 4 may also be reacted with intermediates of formula 6 (commercially available or synthesized) in the presence of an azodicarboxylate reagent (e.g., DEAD, DIAD, ADDP, and the like) and a phosphine (e.g., tributylphosphine, triphenylphosphine and the like) in a solvent (e.g., THF, toluene, and the like) at a temperature ranging from 0 to 100° C., to provide the desired compounds of formula 7. Ester derivatives 7 may then be converted into the desired compounds of formula 8 via standard saponification reactions. The desired compounds of formula 10 may be obtained from acid derivatives of formula 8 by reaction with amine derivatives of formula 9 (commercially available or synthesized by procedures known in the art or as set forth in the examples below) under standard peptide coupling conditions (e.g., DCC, EDCI, HATU, PyBop and the like) in a polar aprotic solvent (e.g., DCM, DMF and the like). Alternatively, carboxylic acid derivatives of formula 8, may be converted into acid chloride derivatives by procedures known to those skilled in the art or as set forth in the examples below, and then reacted with amines of formula 9 to obtain the desired compounds of formula 10 by procedures known to those skilled in the art or as set forth in the examples below.

In a more particular embodiment, the compounds of the present invention may be synthesized as depicted in scheme 2.

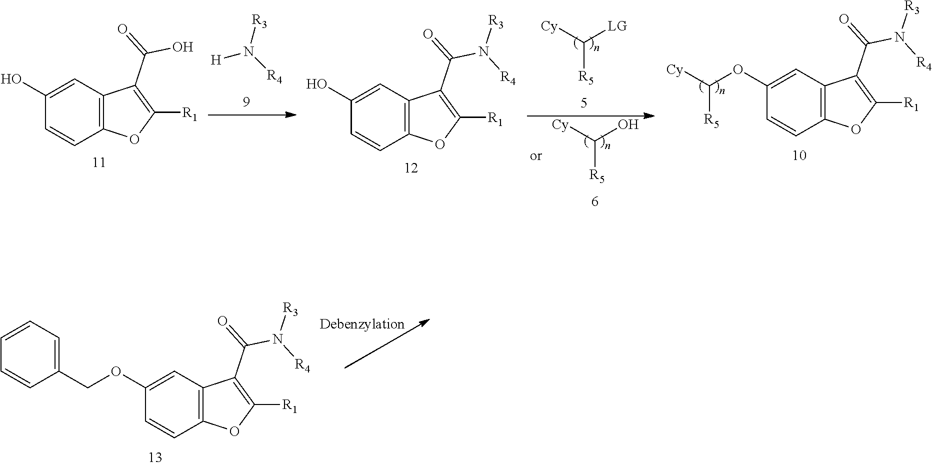

Scheme 2: all R1, R2, R3, R4 and R5 are as described for the compounds of the present invention.

5-Hydroxy-benzofuran-3-carboxylic acid derivatives 11 (commercially available or synthesized by procedures known in the art or as set forth in the examples below) may be reacted with amine derivatives of formula 9 (commercially available or synthesized by procedures known in the art or as set forth in the examples below) under standard peptide coupling conditions (e.g. DCC, EDCI, HATU, PyBop and the like) in a polar aprotic solvent (e.g. DCM, DMF and the like) to provide intermediates of formula 12. Alternatively, compounds of formula 13 (synthetized as described in scheme 1) may be converted into intermediates of formula 12 via hydrogenation reactions with a reducing agent (e.g., hydrogen gas, ammonium formate, cyclohexadiene and the like) using a catalyst (more preferably Pd or Pt) in a solvent (e.g., THF, EtOH, and the like). Intermediates of formula 12 may then be converted into the desired compounds of formula 10 via nucleophilic substitution using intermediates of formula 5 (commercially available or synthesized), wherein LG is a leaving group, in the presence of a base (e.g., DIPEA, DBU, triethylamine, Cs₂CO₃, and the like) in a polar solvent (e.g., acetonitrile, NMP, and the like), with or without a chelating agent (e.g., 18-crown-6, cis-anti-cis-dicyclohexano-18-crown-6, and the like) at a temperature ranging from 0 to 100° C. Alternatively, compounds of formula 12 may also be reacted with intermediates of formula 6 (commercially available or synthesized) in the presence of an azodicarboxylate reagent (e.g., DEAD, ADDP, DIAD, tert-butylazodicarboxylate, and the like) and a phosphine (e.g., tributylphosphine, triphenylphosphine and the like) in a solvent (e.g., THF, toluene, and the like) at a temperature ranging from 0 to 100° C., to provide the desired compounds of formula 10.

In a more particular embodiment, the compounds of the present invention may be synthesized as depicted in scheme 3.

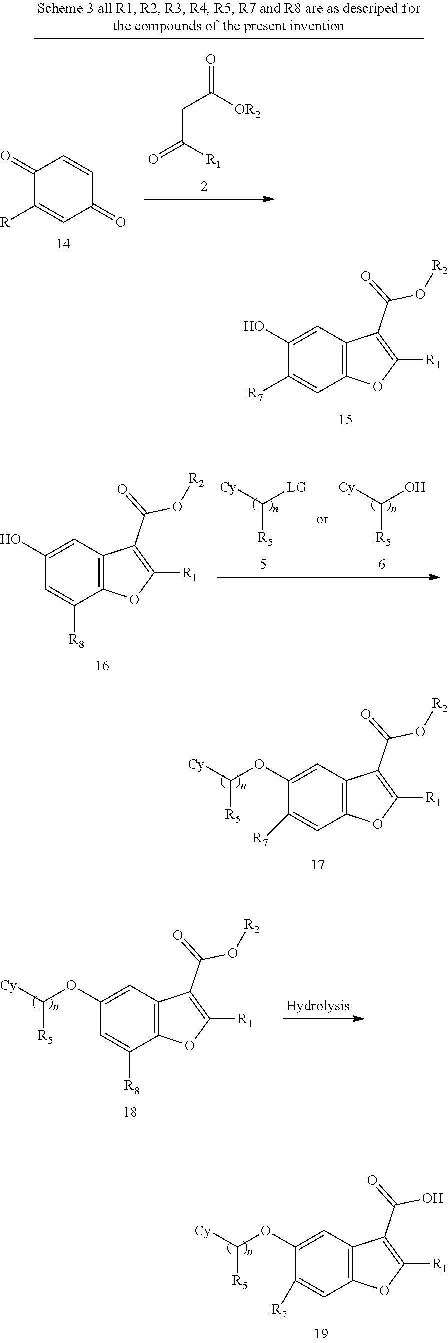

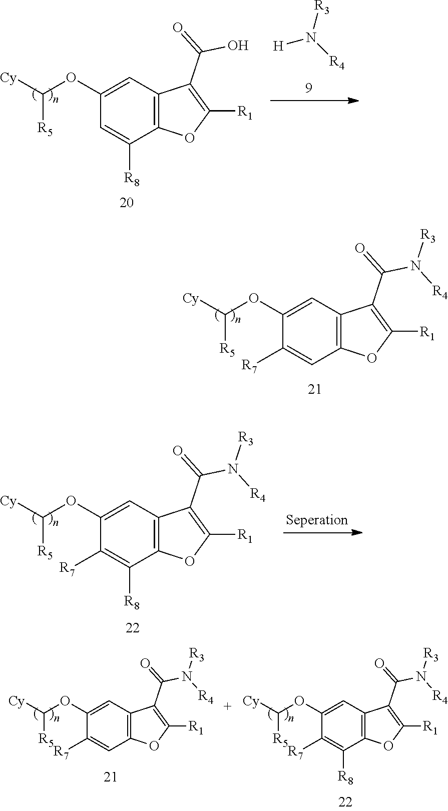

A substituted para-Benzoquinone derivatives of formula 14, may be condensed with a ketoester of formula 2 (commercially available or synthesized by procedures known to those skilled in the art), wherein R₂ is an ester protecting group (e.g. methyl, ethyl, t-Bu and the like), in the presence of a Lewis acid (e.g., titanium(IV) chloride, zinc(II) chloride and the like) in a polar solvent (e.g., DCM, MeOH, EtOH, and the like) at a temperature ranging from 0 to 100° C. to provide a mixture of substituted intermediates of formula 15 and 16. More detailed information can be found in the following references (Bioorg. Med. Chem. 2012, 20, 4237-4244 and FR 1319594). Intermediates of formula 15 and/or 16 may then be converted into the desired compounds of formula 17 and/or 18 via nucleophilic substitution using intermediates of formula 5 (commercially available or synthesized), wherein LG is a leaving group, in the presence of a base (e.g., DIPEA, DBU, triethylamine, Cs₂CO₃, and the like) in a polar solvent (e.g., acetonitrile, DMF, NMP, and the like), with or without a chelating agent (e.g., 18-crown-6, cis-anti-cis-dicyclohexano-18-crown-6, and the like) at a temperature ranging from 0 to 100° C. Alternatively, intermediates of formula 15 and/or 16 may also be reacted with intermediates of formula 6 (commercially available or synthesized) in the presence of an azodicarboxylate reagent (e.g., DEAD, DIAD, ADDP, and the like) and a phosphine (e.g., tributylphosphine, triphenylphosphine and the like) in a solvent (e.g., THF, toluene, and the like) at a temperature ranging from 0 to 100° C., to provide the desired compounds of formula 17 and/or 18. Ester derivatives 17 and/or 18 may then be converted into the desired carboxylic acid of formula 19 and/or 20 via standard saponification reactions. The desired compounds of formula 21 and/or 22 may be obtained from acid derivatives of formula 19 and/or 20 by reaction with amine derivatives of formula 9 (commercially available or synthesized by procedures known in the art or as set forth in the examples below) under standard peptide coupling conditions (e.g., DCC, EDCI, HATU, PyBop and the like) in a polar aprotic solvent (e.g., DCM, DMF and the like). A mixture of compounds 21 and 22 may be separated (e.g., silica gel, HPLC, SFC or preparative CFC) to provide the desired compounds of formula 21 or 22.

In a more particular embodiment, the compounds of the present invention may be synthesized as depicted in scheme 4

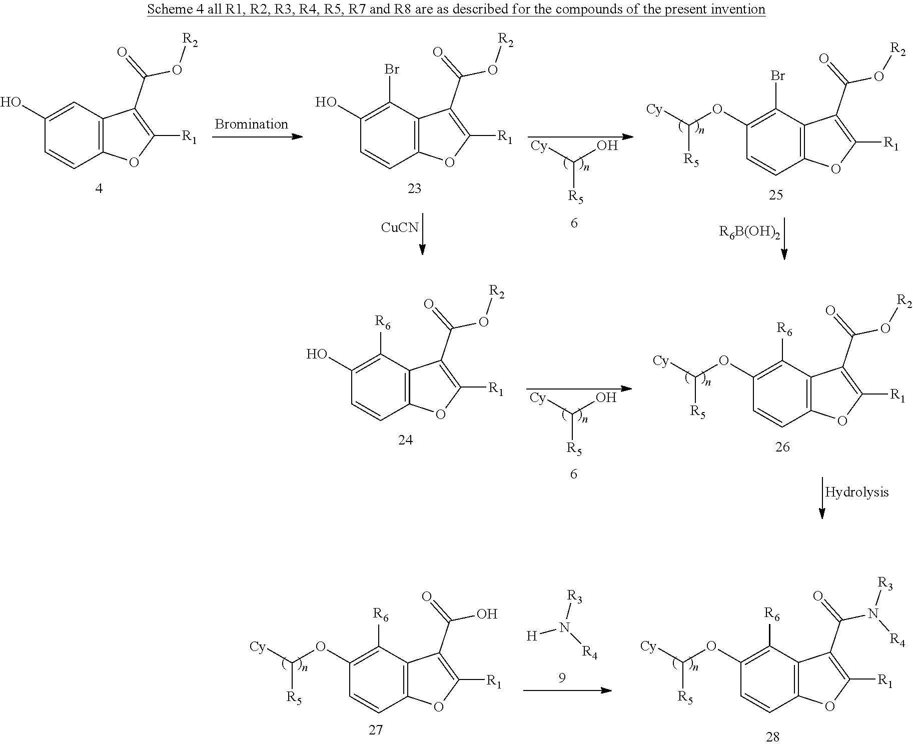

Intermediates of formula 4 may be halogenated with a suitable halogenating agent (e.g., bromine, N-bromosuccinimide and the like) in a solvent (e.g., chloroform, water and the like) to provide the desired intermediates 23. The desired compounds of formula 26 may be obtained by an Ullmann type reaction with CuCN followed by a Mitsunobu type reaction with intermediates of formula 6 (commercially available or synthesized). Alternatively, the desired compounds of formula 26 may be obtained via a Mitsunobu type reaction with intermediates of formula 6 (commercially available or synthesized) followed by a Suzuki reaction. Ester derivatives 26 may then be converted into the desired compounds of formula 27 via standard saponification reactions. The desired compounds of formula 28 may be obtained from acid derivatives of formula 27 by reaction with amine derivatives of formula 9 (commercially available or synthesized by procedures known in the art or as set forth in the examples below) under standard peptide coupling conditions (e.g., DCC, EDCI, HATU, PyBop and the like) in a polar aprotic solvent (e.g., DCM, DMF and the like).

In a more particular embodiment, the compounds of the present invention may be synthesized as depicted in scheme 5.

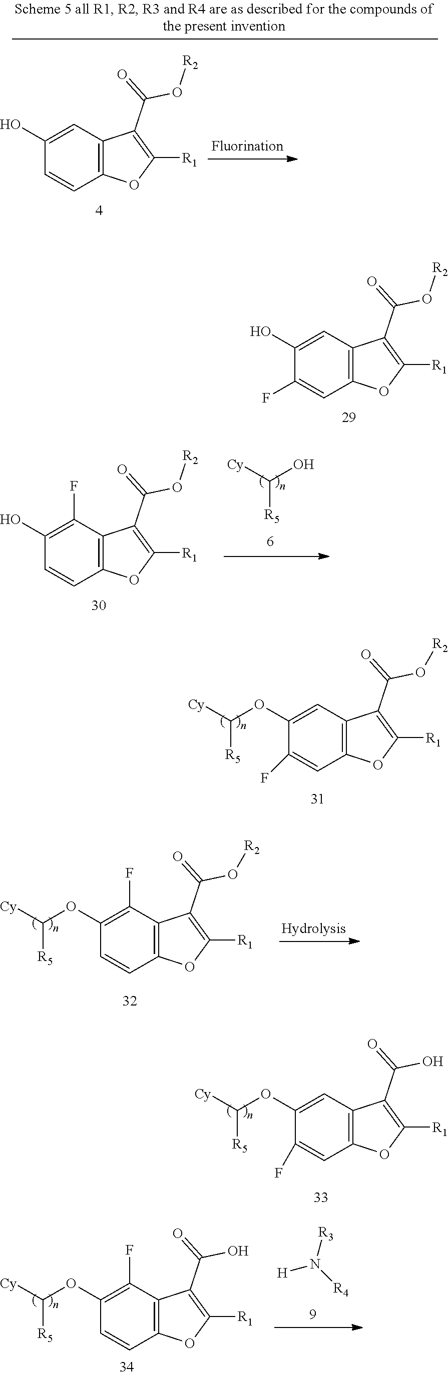

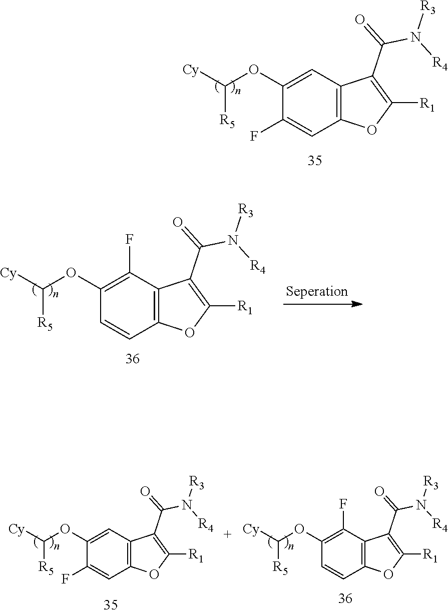

Intermediates of formula 4 may be halogenated with a suitable halogenating agent (e.g. select fluor and the like) in a solvent (e.g. chloroform, acetonitrile and the like) to provide the desired intermediates 29 and 30. Intermediates of formula 29 and/or 30 may be reacted with intermediates of formula 6 (commercially available or synthesized) in the presence of an azodicarboxylate reagent (e.g., DEAD, DIAD, ADDP, and the like) and a phosphine (e.g., triphenylphosphine and the like) in a solvent (e.g., THF, toluene, and the like) at a temperature ranging from 0 to 100° C., to provide the desired compounds of formula 31 and/or 32. Ester derivatives 31 and/or 32 may then be converted into the corresponding carboxylic acid of formula 33 and/or 34 via standard saponification reactions. The desired compounds of formula 35 and/or 36 may be obtained from acid derivatives of formula 33 and/or 34 by reaction with amine derivatives of formula 9 (commercially available or synthesized by procedures known in the art or as set forth in the examples below) under standard peptide coupling conditions (e.g., DCC, EDCI, HATU, PyBop and the like) in a polar aprotic solvent (e.g., DCM, DMF and the like). A mixture of compounds 35 and 36 may be separated (e.g., silica gel, HPLC, SFC or preparative CFC) to provide the desired compounds of formula 35 or 36.

### Examples

| Structure | CODE |
|---|---|
| 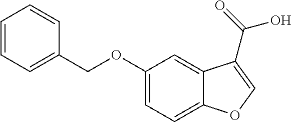 [SMILES: O=C(O)c1coc2ccc(OCc3ccccc3)cc12] | Cpd 001 |
| 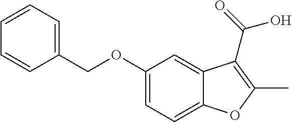 [SMILES: Cc1oc2ccc(OCc3ccccc3)cc2c1C(=O)O] | Cpd 002 |
| 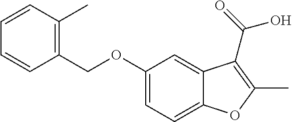 [SMILES: Cc1ccccc1COc1ccc2oc(C)c(C(=O)O)c2c1] | Cpd 003 |
| 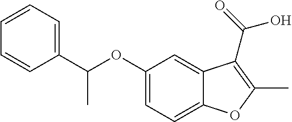 [SMILES: Cc1oc2ccc(OC(C)c3ccccc3)cc2c1C(=O)O] | Cpd 004 |
| 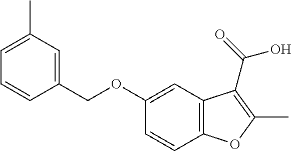 [SMILES: Cc1cccc(COc2ccc3oc(C)c(C(=O)O)c3c2)c1] | Cpd 005 |
| 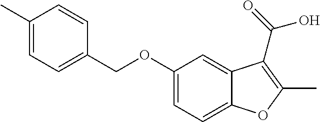 | Cpd 006 |
| 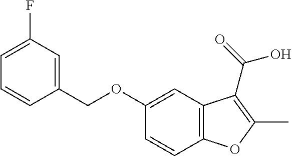 [SMILES: Cc1oc2ccc(OCc3cccc(F)c3)cc2c1C(=O)O] | Cpd 007 |
| 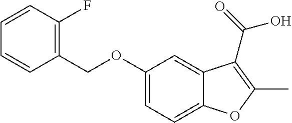 [SMILES: Cc1oc2ccc(OCc3ccccc3F)cc2c1C(=O)O] | Cpd 008 |
| 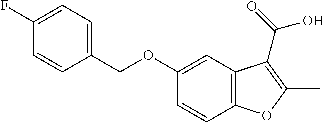 [SMILES: Cc1oc2ccc(OCc3ccc(F)cc3)cc2c1C(=O)O] | Cpd 009 |
| 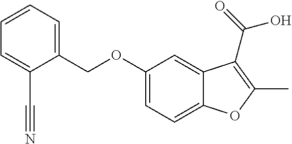 [SMILES: Cc1oc2ccc(OCc3ccccc3C#N)cc2c1C(=O)O] | Cpd 010 |
| 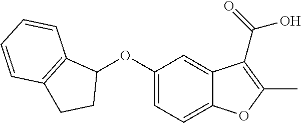 [SMILES: Cc1oc2ccc(OC3CCc4ccccc43)cc2c1C(=O)O] | Cpd 011 |
| 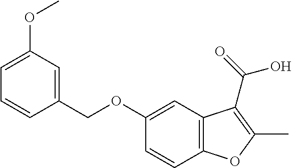 [SMILES: COc1cccc(COc2ccc3oc(C)c(C(=O)O)c3c2)c1] | Cpd 012 |
| 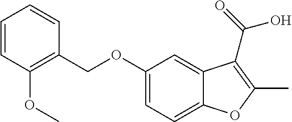 [SMILES: COc1ccccc1COc1ccc2oc(C)c(C(=O)O)c2c1] | Cpd 013 |
| 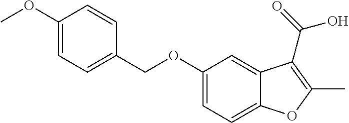 | Cpd 014 |
| 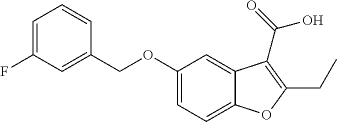 [SMILES: CCc1oc2ccc(OCc3cccc(F)c3)cc2c1C(=O)O] | Cpd 015 |
| 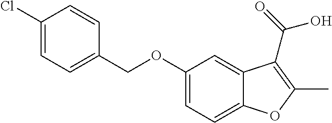 [SMILES: Cc1oc2ccc(OCc3ccc(Cl)cc3)cc2c1C(=O)O] | Cpd 016 |
| 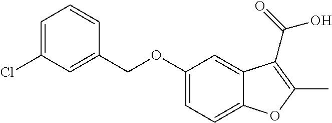 [SMILES: Cc1oc2ccc(OCc3cccc(Cl)c3)cc2c1C(=O)O] | Cpd 017 |
| 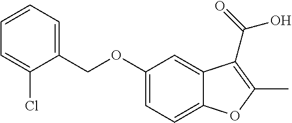 [SMILES: Cc1oc2ccc(OCc3ccccc3Cl)cc2c1C(=O)O] | Cpd 018 |
| 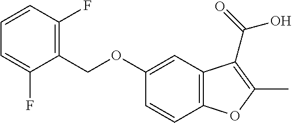 [SMILES: Cc1oc2ccc(OCc3c(F)cccc3F)cc2c1C(=O)O] | Cpd 019 |
| 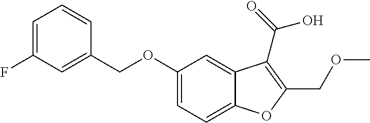 [SMILES: COCc1oc2ccc(OCc3cccc(F)c3)cc2c1C(=O)O] | Cpd 020 |
| 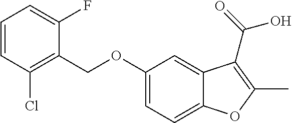 [SMILES: Cc1oc2ccc(OCc3c(F)cccc3Cl)cc2c1C(=O)O] | Cpd 021 |
| 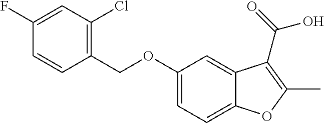 [SMILES: Cc1oc2ccc(OCc3ccc(F)cc3Cl)cc2c1C(=O)O] | Cpd 022 |
| 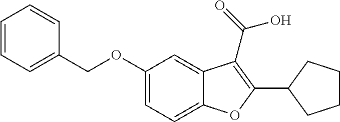 [SMILES: O=C(O)c1c(C2CCCC2)oc2ccc(OCc3ccccc3)cc12] | Cpd 023 |
| 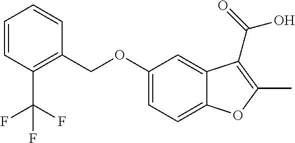 [SMILES: Cc1oc2ccc(OCc3ccccc3C(F)(F)F)cc2c1C(=O)O] | Cpd 024 |
| 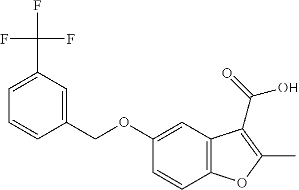 [SMILES: Cc1oc2ccc(OCc3cccc(C(F)(F)F)c3)cc2c1C(=O)O] | Cpd 025 |
| 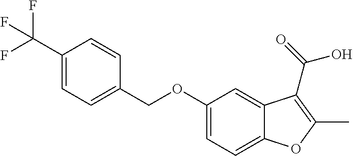 [SMILES: Cc1oc2ccc(OCc3ccc(C(F)(F)F)cc3)cc2c1C(=O)O] | Cpd 026 |
| 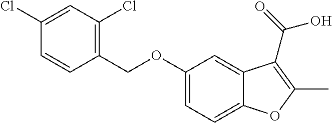 [SMILES: Cc1oc2ccc(OCc3ccc(Cl)cc3Cl)cc2c1C(=O)O] | Cpd 027 |
| 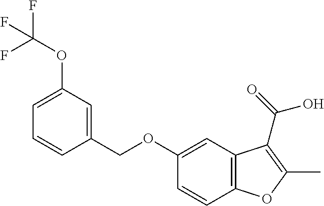 [SMILES: Cc1oc2ccc(OCc3cccc(OC(F)(F)F)c3)cc2c1C(=O)O] | Cpd 028 |
| 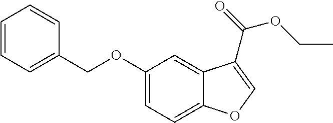 [SMILES: CCOC(=O)c1coc2ccc(OCc3ccccc3)cc12] | Cpd 029 |
| 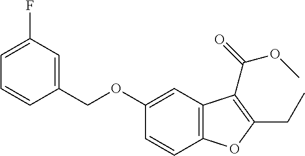 [SMILES: CCc1oc2ccc(OCc3cccc(F)c3)cc2c1C(=O)OC] | Cpd 030 |
| 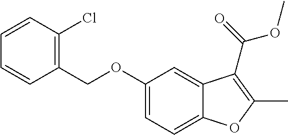 [SMILES: COC(=O)c1c(C)oc2ccc(OCc3ccccc3Cl)cc12] | Cpd 031 |
| 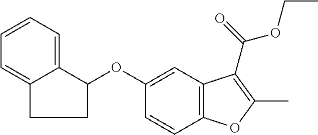 [SMILES: CCOC(=O)c1c(C)oc2ccc(OC3CCc4ccccc43)cc12] | Cpd 032 |
| 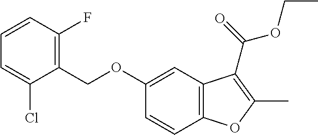 [SMILES: CCOC(=O)c1c(C)oc2ccc(OCc3c(F)cccc3Cl)cc12] | Cpd 033 |
| 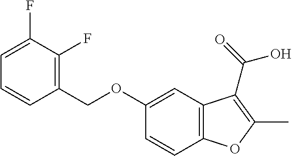 [SMILES: Cc1oc2ccc(OCc3cccc(F)c3F)cc2c1C(=O)O] | Cpd 034 |
| 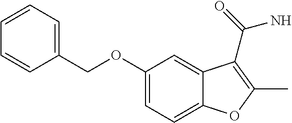 [SMILES: Cc1oc2ccc(OCc3ccccc3)cc2c1C(N)=O] | Cpd 035 |
| 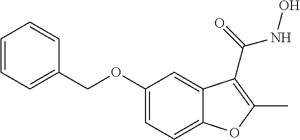 [SMILES: Cc1oc2ccc(OCc3ccccc3)cc2c1C(=O)NO] | Cpd 036 |
| 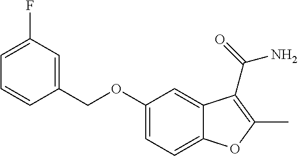 [SMILES: Cc1oc2ccc(OCc3cccc(F)c3)cc2c1C(N)=O] | Cpd 037 |
| 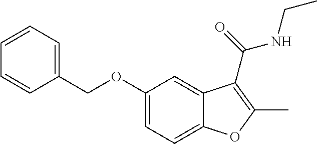 [SMILES: CCNC(=O)c1c(C)oc2ccc(OCc3ccccc3)cc12] | Cpd 038 |
| 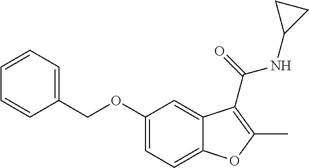 [SMILES: Cc1oc2ccc(OCc3ccccc3)cc2c1C(=O)NC1CC1] | Cpd 039 |
| 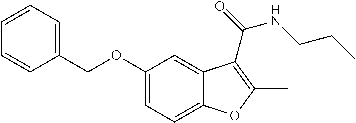 [SMILES: CCCNC(=O)c1c(C)oc2ccc(OCc3ccccc3)cc12] | Cpd 040 |
| 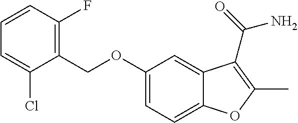 [SMILES: Cc1oc2ccc(OCc3c(F)cccc3Cl)cc2c1C(N)=O] | Cpd 041 |
| 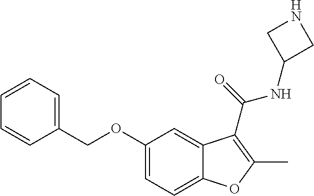 [SMILES: Cc1oc2ccc(OCc3ccccc3)cc2c1C(=O)NC1CNC1] | Cpd 042 |
| 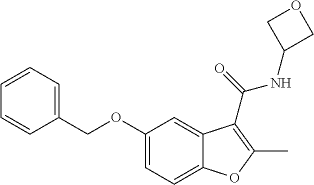 [SMILES: Cc1oc2ccc(OCc3ccccc3)cc2c1C(=O)NC1COC1] | Cpd 043 |
| 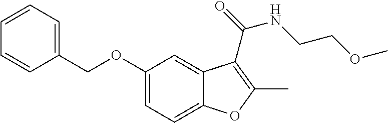 [SMILES: COCCNC(=O)c1c(C)oc2ccc(OCc3ccccc3)cc12] | Cpd 044 |
| 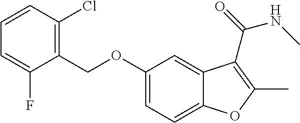 [SMILES: CNC(=O)c1c(C)oc2ccc(OCc3c(F)cccc3Cl)cc12] | Cpd 045 |
| 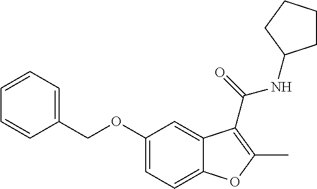 [SMILES: Cc1oc2ccc(OCc3ccccc3)cc2c1C(=O)NC1CCCC1] | Cpd 046 |
| 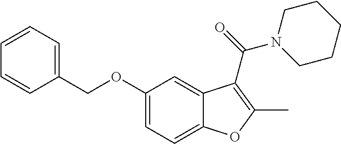 [SMILES: Cc1oc2ccc(OCc3ccccc3)cc2c1C(=O)N1CCCCC1] | Cpd 047 |
| 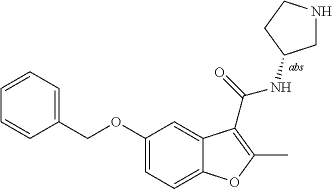 [SMILES: Cc1oc2ccc(OCc3ccccc3)cc2c1C(=O)N[C@@H]1CCNC1] | Cpd 048 |
| 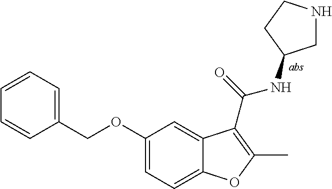 [SMILES: Cc1oc2ccc(OCc3ccccc3)cc2c1C(=O)N[C@H]1CCNC1] | Cpd 049 |
| 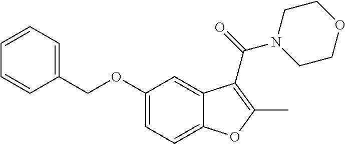 [SMILES: Cc1oc2ccc(OCc3ccccc3)cc2c1C(=O)N1CCOCC1] | Cpd 050 |
| 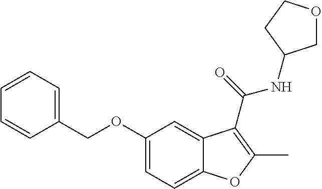 [SMILES: Cc1oc2ccc(OCc3ccccc3)cc2c1C(=O)NC1CCOC1] | Cpd 051 |
| 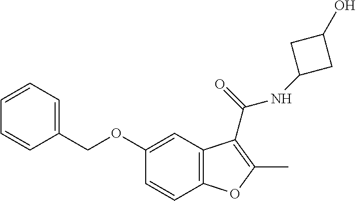 [SMILES: Cc1oc2ccc(OCc3ccccc3)cc2c1C(=O)NC1CC(O)C1] | Cpd 052 |
| 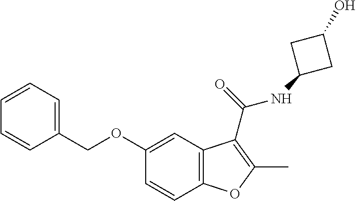 [SMILES: Cc1oc2ccc(OCc3ccccc3)cc2c1C(=O)N[C@H]1C[C@H](O)C1] | Cpd 053 |
| 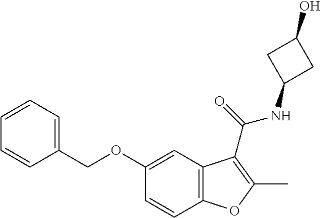 [SMILES: Cc1oc2ccc(OCc3ccccc3)cc2c1C(=O)N[C@H]1C[C@@H](O)C1] | Cpd 054 |
| 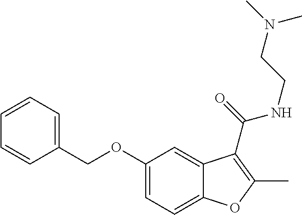 [SMILES: Cc1oc2ccc(OCc3ccccc3)cc2c1C(=O)NCCN(C)C] | Cpd 055 |
| 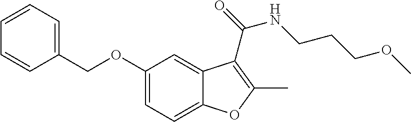 [SMILES: COCCCNC(=O)c1c(C)oc2ccc(OCc3ccccc3)cc12] | Cpd 056 |
| 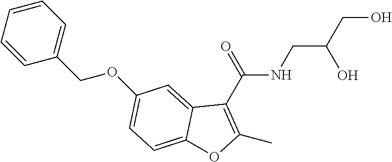 [SMILES: Cc1oc2ccc(OCc3ccccc3)cc2c1C(=O)NCC(O)CO] | Cpd 057 |
| 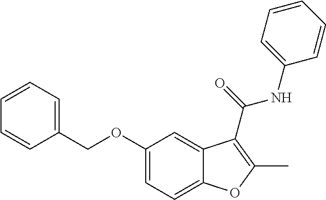 [SMILES: Cc1oc2ccc(OCc3ccccc3)cc2c1C(=O)Nc1ccccc1] | Cpd 058 |
| 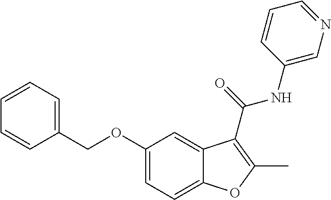 [SMILES: Cc1oc2ccc(OCc3ccccc3)cc2c1C(=O)Nc1cccnc1] | Cpd 059 |
| 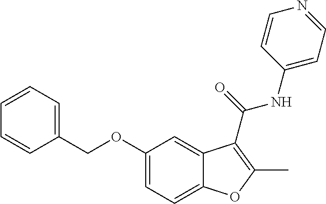 [SMILES: Cc1oc2ccc(OCc3ccccc3)cc2c1C(=O)Nc1ccncc1] | Cpd 060 |
| 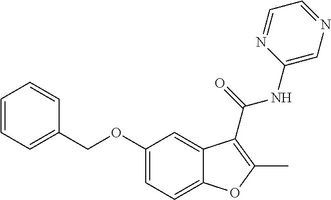 [SMILES: Cc1oc2ccc(OCc3ccccc3)cc2c1C(=O)Nc1cnccn1] | Cpd 061 |
| 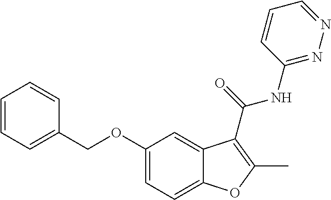 [SMILES: Cc1oc2ccc(OCc3ccccc3)cc2c1C(=O)Nc1cccnn1] | Cpd 062 |
| 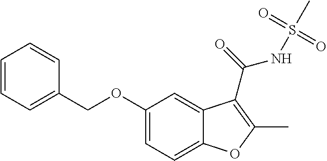 [SMILES: Cc1oc2ccc(OCc3ccccc3)cc2c1C(=O)NS(C)(=O)=O] | Cpd 063 |
| 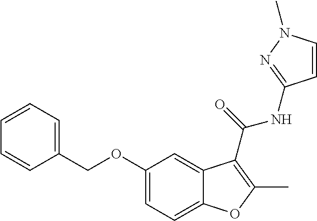 [SMILES: Cc1oc2ccc(OCc3ccccc3)cc2c1C(=O)Nc1ccn(C)n1] | Cpd 064 |
| 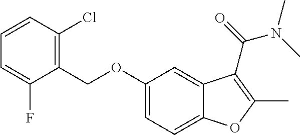 [SMILES: Cc1oc2ccc(OCc3c(F)cccc3Cl)cc2c1C(=O)N(C)C] | Cpd 065 |
| 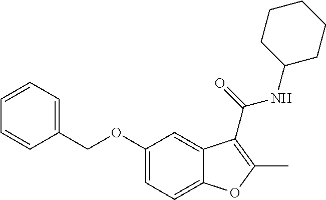 [SMILES: Cc1oc2ccc(OCc3ccccc3)cc2c1C(=O)NC1CCCCC1] | Cpd 066 |
| 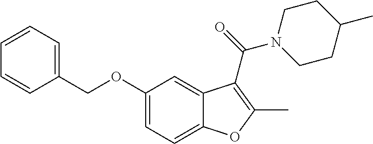 [SMILES: Cc1oc2ccc(OCc3ccccc3)cc2c1C(=O)N1CCC(C)CC1] | Cpd 067 |
| 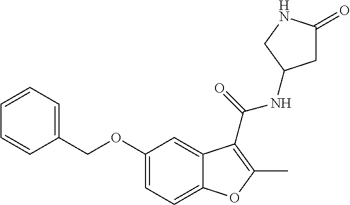 [SMILES: Cc1oc2ccc(OCc3ccccc3)cc2c1C(=O)NC1CNC(=O)C1] | Cpd 068 |
| 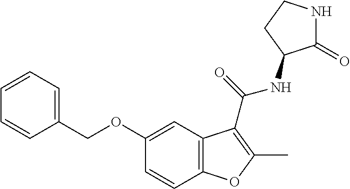 [SMILES: Cc1oc2ccc(OCc3ccccc3)cc2c1C(=O)N[C@H]1CCNC1=O] | Cpd 069 |
| 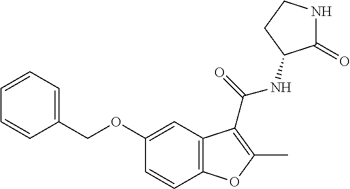 [SMILES: Cc1oc2ccc(OCc3ccccc3)cc2c1C(=O)N[C@@H]1CCNC1=O] | Cpd 070 |
| 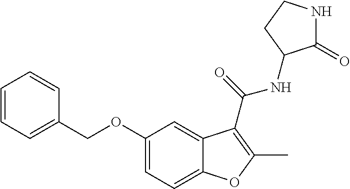 [SMILES: Cc1oc2ccc(OCc3ccccc3)cc2c1C(=O)NC1CCNC1=O] | Cpd 071 |
| 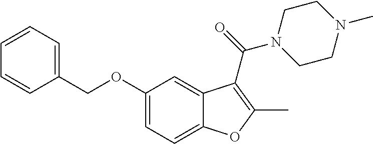 [SMILES: Cc1oc2ccc(OCc3ccccc3)cc2c1C(=O)N1CCN(C)CC1] | Cpd 072 |
| 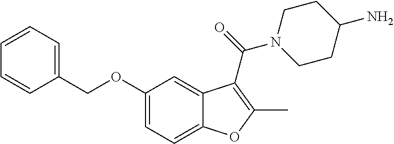 [SMILES: Cc1oc2ccc(OCc3ccccc3)cc2c1C(=O)N1CCC(N)CC1] | Cpd 073 |
| 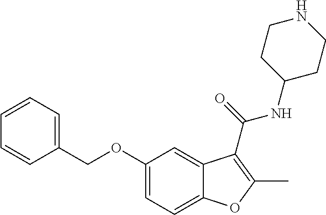 [SMILES: Cc1oc2ccc(OCc3ccccc3)cc2c1C(=O)NC1CCNCC1] | Cpd 074 |
| 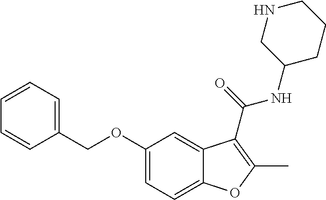 [SMILES: Cc1oc2ccc(OCc3ccccc3)cc2c1C(=O)NC1CCCNC1] | Cpd 075 |
| 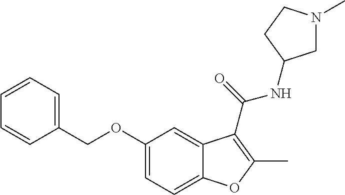 [SMILES: Cc1oc2ccc(OCc3ccccc3)cc2c1C(=O)NC1CCN(C)C1] | Cpd 076 |
| 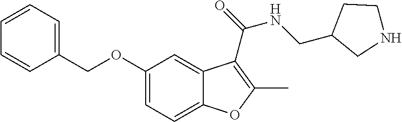 [SMILES: Cc1oc2ccc(OCc3ccccc3)cc2c1C(=O)NCC1CCNC1] | Cpd 077 |
| 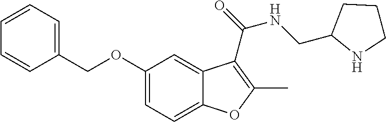 [SMILES: Cc1oc2ccc(OCc3ccccc3)cc2c1C(=O)NCC1CCCN1] | Cpd 078 |
| 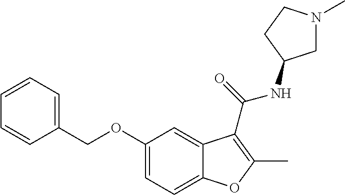 [SMILES: Cc1oc2ccc(OCc3ccccc3)cc2c1C(=O)N[C@H]1CCN(C)C1] | Cpd 079 |
| 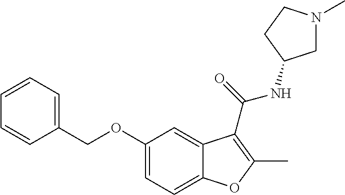 [SMILES: Cc1oc2ccc(OCc3ccccc3)cc2c1C(=O)N[C@@H]1CCN(C)C1] | Cpd 080 |
| 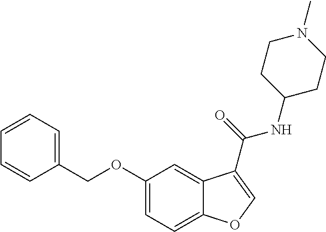 [SMILES: CN1CCC(NC(=O)c2coc3ccc(OCc4ccccc4)cc23)CC1] | Cpd 081 |
| 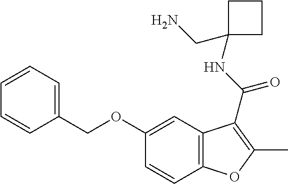 [SMILES: Cc1oc2ccc(OCc3ccccc3)cc2c1C(=O)NC1(CN)CCC1] | Cpd 082 |
| 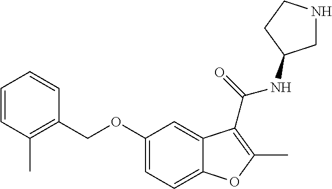 [SMILES: Cc1ccccc1COc1ccc2oc(C)c(C(=O)N[C@H]3CCNC3)c2c1] | Cpd 083 |
| 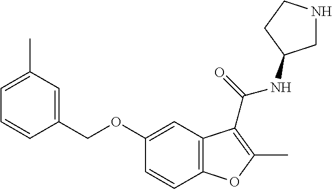 [SMILES: Cc1cccc(COc2ccc3oc(C)c(C(=O)N[C@H]4CCNC4)c3c2)c1] | Cpd 084 |
| 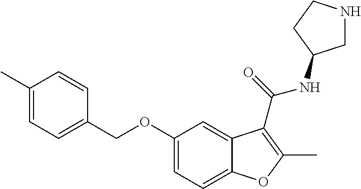 [SMILES: Cc1ccc(COc2ccc3oc(C)c(C(=O)N[C@H]4CCNC4)c3c2)cc1] | Cpd 085 |
| 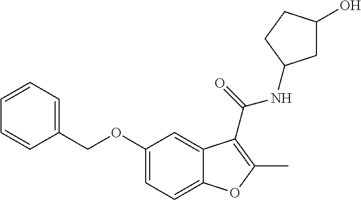 [SMILES: Cc1oc2ccc(OCc3ccccc3)cc2c1C(=O)NC1CCC(O)C1] | Cpd 086 |
| 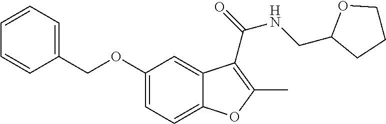 [SMILES: Cc1oc2ccc(OCc3ccccc3)cc2c1C(=O)NCC1CCCO1] | Cpd 087 |
| 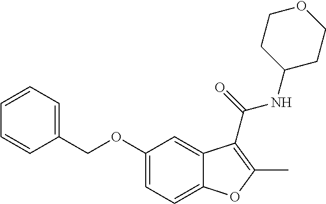 [SMILES: Cc1oc2ccc(OCc3ccccc3)cc2c1C(=O)NC1CCOCC1] | Cpd 088 |
| 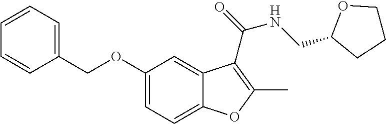 [SMILES: Cc1oc2ccc(OCc3ccccc3)cc2c1C(=O)NC[C@H]1CCCO1] | Cpd 089 |
| 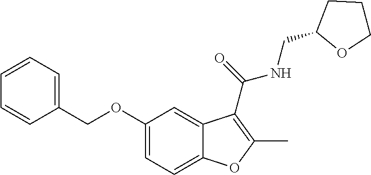 [SMILES: Cc1oc2ccc(OCc3ccccc3)cc2c1C(=O)NC[C@@H]1CCCO1] | Cpd 090 |
| 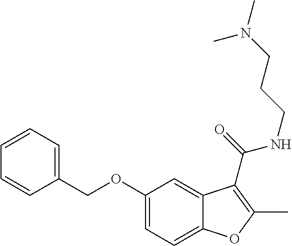 [SMILES: Cc1oc2ccc(OCc3ccccc3)cc2c1C(=O)NCCCN(C)C] | Cpd 091 |
| 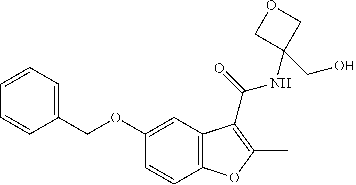 [SMILES: Cc1oc2ccc(OCc3ccccc3)cc2c1C(=O)NC1(CO)COC1] | Cpd 092 |
| 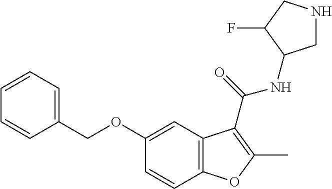 [SMILES: Cc1oc2ccc(OCc3ccccc3)cc2c1C(=O)NC1CNCC1F] | Cpd 093 |
| 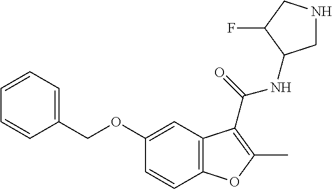 [SMILES: Cc1oc2ccc(OCc3ccccc3)cc2c1C(=O)NC1CNCC1F] | Cpd 094 |
| 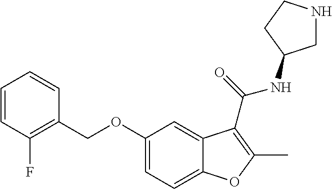 [SMILES: Cc1oc2ccc(OCc3ccccc3F)cc2c1C(=O)N[C@H]1CCNC1] | Cpd 095 |
| 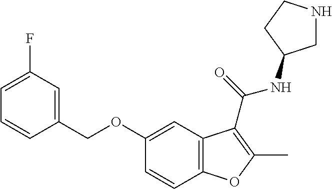 [SMILES: Cc1oc2ccc(OCc3cccc(F)c3)cc2c1C(=O)N[C@H]1CCNC1] | Cpd 096 |
| 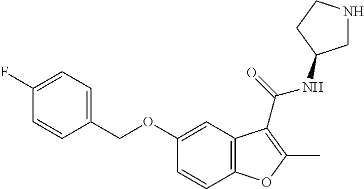 [SMILES: Cc1oc2ccc(OCc3ccc(F)cc3)cc2c1C(=O)N[C@H]1CCNC1] | Cpd 097 |
| 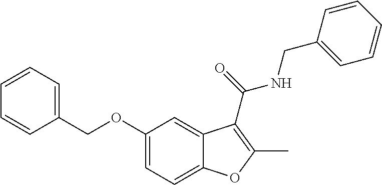 [SMILES: Cc1oc2ccc(OCc3ccccc3)cc2c1C(=O)NCc1ccccc1] | Cpd 098 |
| 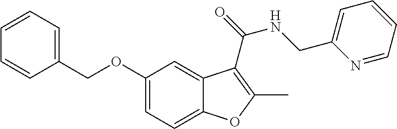 [SMILES: Cc1oc2ccc(OCc3ccccc3)cc2c1C(=O)NCc1ccccn1] | Cpd 099 |
| 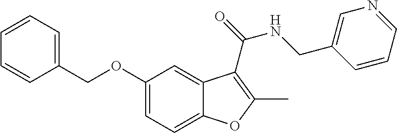 [SMILES: Cc1oc2ccc(OCc3ccccc3)cc2c1C(=O)NCc1cccnc1] | Cpd 100 |
| 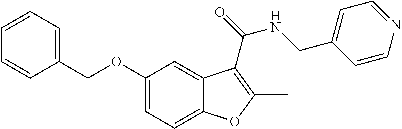 [SMILES: Cc1oc2ccc(OCc3ccccc3)cc2c1C(=O)NCc1ccncc1] | Cpd 101 |
| 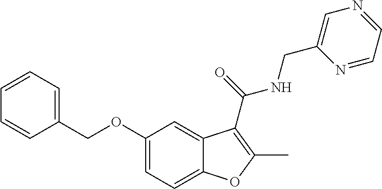 [SMILES: Cc1oc2ccc(OCc3ccccc3)cc2c1C(=O)NCc1cnccn1] | Cpd 102 |
| 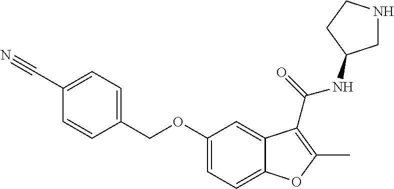 [SMILES: Cc1oc2ccc(OCc3ccc(C#N)cc3)cc2c1C(=O)N[C@H]1CCNC1] | Cpd 103 |
| 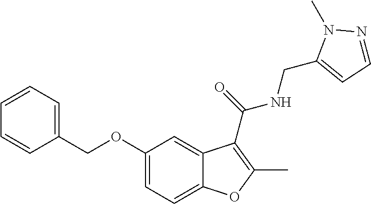 [SMILES: Cc1oc2ccc(OCc3ccccc3)cc2c1C(=O)NCc1ccnn1C] | Cpd 104 |
| 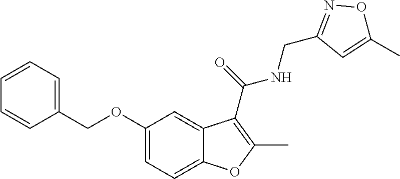 [SMILES: Cc1cc(CNC(=O)c2c(C)oc3ccc(OCc4ccccc4)cc23)no1] | Cpd 105 |
| 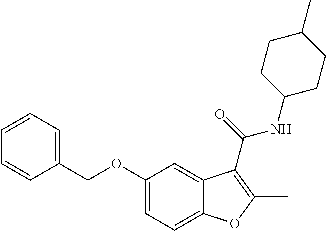 [SMILES: Cc1oc2ccc(OCc3ccccc3)cc2c1C(=O)NC1CCC(C)CC1] | Cpd 106 |
| 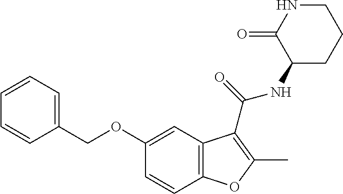 [SMILES: Cc1oc2ccc(OCc3ccccc3)cc2c1C(=O)N[C@@H]1CCCNC1=O] | Cpd 107 |
| 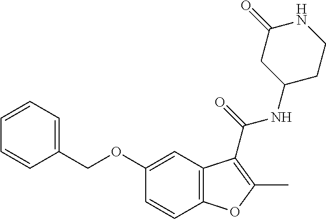 [SMILES: Cc1oc2ccc(OCc3ccccc3)cc2c1C(=O)NC1CCNC(=O)C1] | Cpd 108 |
| 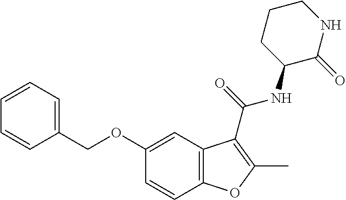 [SMILES: Cc1oc2ccc(OCc3ccccc3)cc2c1C(=O)N[C@H]1CCCNC1=O] | Cpd 109 |
| 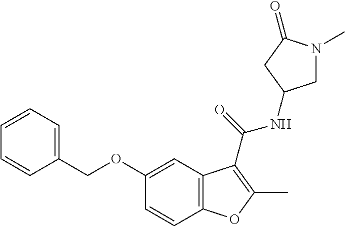 [SMILES: Cc1oc2ccc(OCc3ccccc3)cc2c1C(=O)NC1CC(=O)N(C)C1] | Cpd 110 |
| 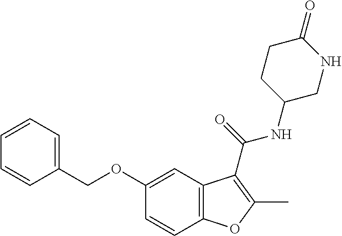 [SMILES: Cc1oc2ccc(OCc3ccccc3)cc2c1C(=O)NC1CCC(=O)NC1] | Cpd 111 |
| 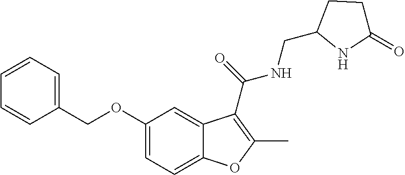 [SMILES: Cc1oc2ccc(OCc3ccccc3)cc2c1C(=O)NCC1CCC(=O)N1] | Cpd 112 |
| 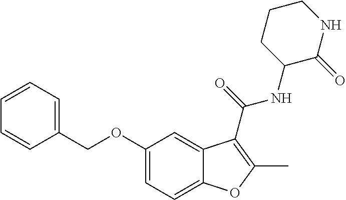 [SMILES: Cc1oc2ccc(OCc3ccccc3)cc2c1C(=O)NC1CCCNC1=O] | Cpd 113 |
| 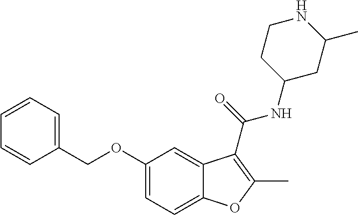 [SMILES: Cc1oc2ccc(OCc3ccccc3)cc2c1C(=O)NC1CCNC(C)C1] | Cpd 114 |
| 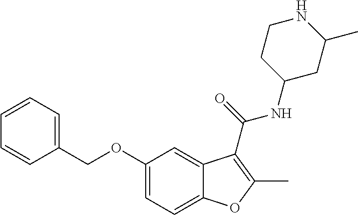 [SMILES: Cc1oc2ccc(OCc3ccccc3)cc2c1C(=O)NC1CCNC(C)C1] | Cpd 115 |
| 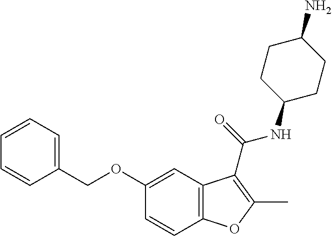 [SMILES: Cc1oc2ccc(OCc3ccccc3)cc2c1C(=O)N[C@H]1CC[C@@H](N)CC1] | Cpd 116 |
| 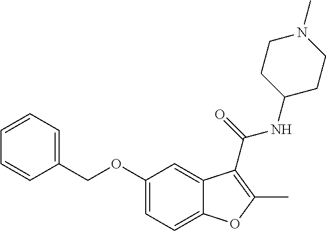 [SMILES: Cc1oc2ccc(OCc3ccccc3)cc2c1C(=O)NC1CCN(C)CC1] | Cpd 117 |
| 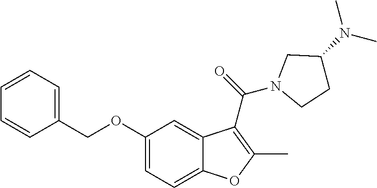 [SMILES: Cc1oc2ccc(OCc3ccccc3)cc2c1C(=O)N1CC[C@@H](N(C)C)C1] | Cpd 118 |
| 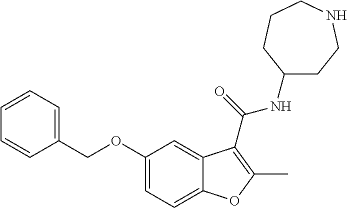 [SMILES: Cc1oc2ccc(OCc3ccccc3)cc2c1C(=O)NC1CCCNCC1] | Cpd 119 |
| 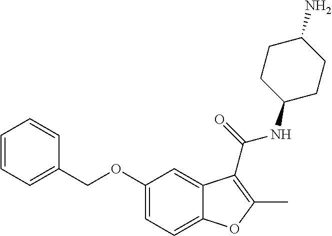 [SMILES: Cc1oc2ccc(OCc3ccccc3)cc2c1C(=O)N[C@H]1CC[C@H](N)CC1] | Cpd 120 |
| 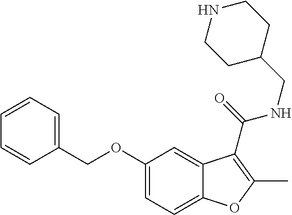 [SMILES: Cc1oc2ccc(OCc3ccccc3)cc2c1C(=O)NCC1CCNCC1] | Cpd 121 |
| 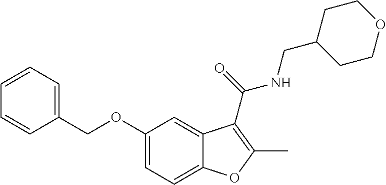 [SMILES: Cc1oc2ccc(OCc3ccccc3)cc2c1C(=O)NCC1CCOCC1] | Cpd 122 |
| 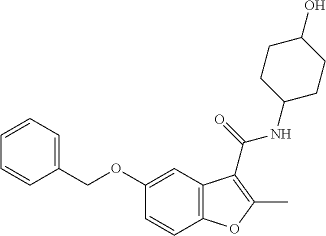 [SMILES: Cc1oc2ccc(OCc3ccccc3)cc2c1C(=O)NC1CCC(O)CC1] | Cpd 123 |
| 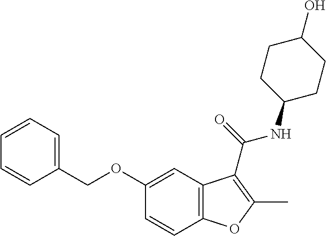 | Cpd 124 |
| 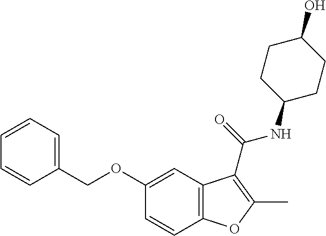 [SMILES: Cc1oc2ccc(OCc3ccccc3)cc2c1C(=O)N[C@H]1CC[C@@H](O)CC1] | Cpd 125 |
| 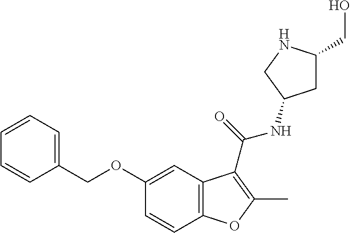 [SMILES: Cc1oc2ccc(OCc3ccccc3)cc2c1C(=O)N[C@@H]1CN[C@H](CO)C1] | Cpd 126 |
| 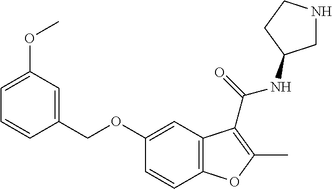 [SMILES: COc1cccc(COc2ccc3oc(C)c(C(=O)N[C@H]4CCNC4)c3c2)c1] | Cpd 127 |
| 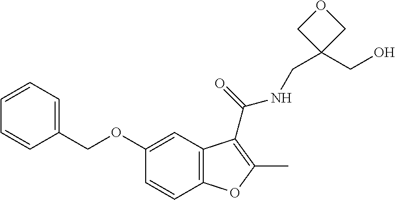 [SMILES: Cc1oc2ccc(OCc3ccccc3)cc2c1C(=O)NCC1(CO)COC1] | Cpd 128 |
| 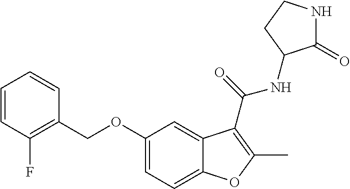 [SMILES: Cc1oc2ccc(OCc3ccccc3F)cc2c1C(=O)NC1CCNC1=O] | Cpd 129 |
| 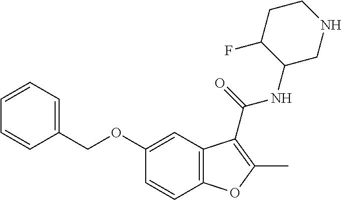 [SMILES: Cc1oc2ccc(OCc3ccccc3)cc2c1C(=O)NC1CNCCC1F] | Cpd 130 |
| 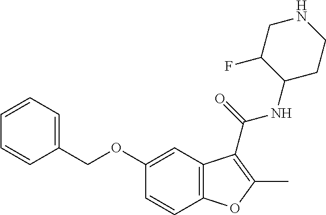 [SMILES: Cc1oc2ccc(OCc3ccccc3)cc2c1C(=O)NC1CCNCC1F] | Cpd 131 |
| 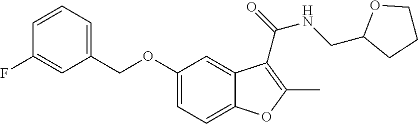 [SMILES: Cc1oc2ccc(OCc3cccc(F)c3)cc2c1C(=O)NCC1CCCO1] | Cpd 132 |
| 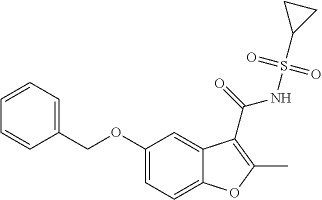 [SMILES: Cc1oc2ccc(OCc3ccccc3)cc2c1C(=O)NS(=O)(=O)C1CC1] | Cpd 133 |
| 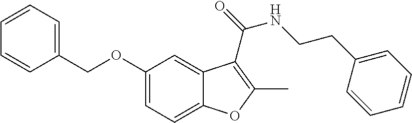 [SMILES: Cc1oc2ccc(OCc3ccccc3)cc2c1C(=O)NCCc1ccccc1] | Cpd 134 |
| 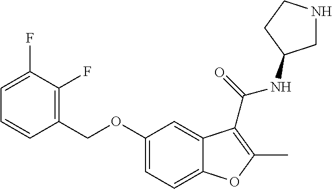 [SMILES: Cc1oc2ccc(OCc3cccc(F)c3F)cc2c1C(=O)N[C@H]1CCNC1] | Cpd 135 |
| 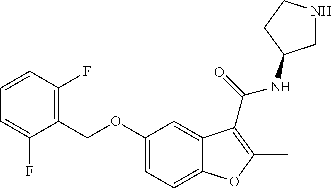 [SMILES: Cc1oc2ccc(OCc3c(F)cccc3F)cc2c1C(=O)N[C@H]1CCNC1] | Cpd 136 |
| 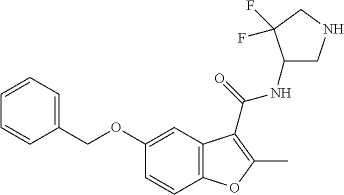 [SMILES: Cc1oc2ccc(OCc3ccccc3)cc2c1C(=O)NC1CNCC1(F)F] | Cpd 137 |
| 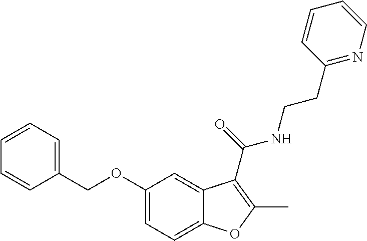 [SMILES: Cc1oc2ccc(OCc3ccccc3)cc2c1C(=O)NCCc1ccccn1] | Cpd 138 |
| 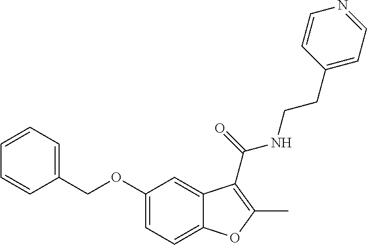 [SMILES: Cc1oc2ccc(OCc3ccccc3)cc2c1C(=O)NCCc1ccncc1] | Cpd 139 |
| 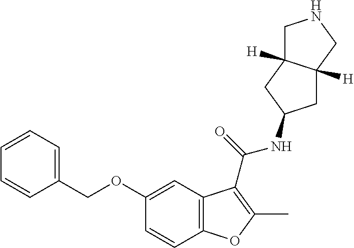 [SMILES: Cc1oc2ccc(OCc3ccccc3)cc2c1C(=O)N[C@@H]1C[C@H]2CNC[C@H]2C1] | Cpd 140 |
| 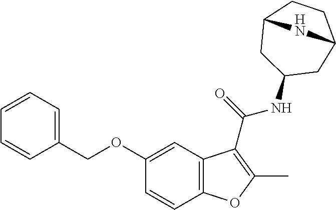 [SMILES: Cc1oc2ccc(OCc3ccccc3)cc2c1C(=O)N[C@@H]1C[C@H]2CC[C@@H](C1)N2] | Cpd 141 |
| 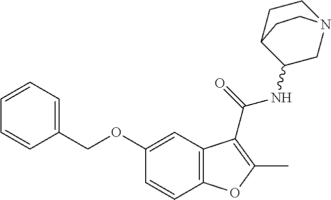 [SMILES: Cc1oc2ccc(OCc3ccccc3)cc2c1C(=O)NC1CN2CCC1CC2] | Cpd 142 |
| 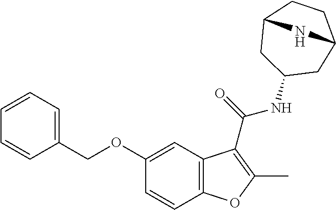 [SMILES: Cc1oc2ccc(OCc3ccccc3)cc2c1C(=O)N[C@H]1C[C@H]2CC[C@@H](C1)N2] | Cpd 143 |
| 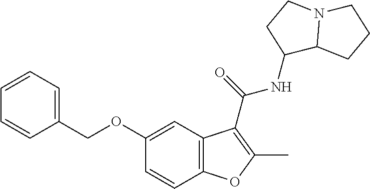 [SMILES: Cc1oc2ccc(OCc3ccccc3)cc2c1C(=O)NC1CCN2CCCC12] | Cpd 144 |
| 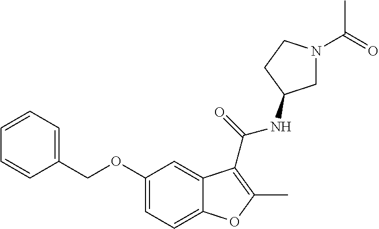 [SMILES: CC(=O)N1CC[C@H](NC(=O)c2c(C)oc3ccc(OCc4ccccc4)cc23)C1] | Cpd 145 |
| 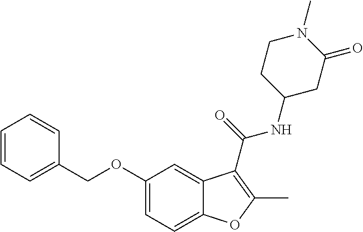 [SMILES: Cc1oc2ccc(OCc3ccccc3)cc2c1C(=O)NC1CCN(C)C(=O)C1] | Cpd 146 |
| 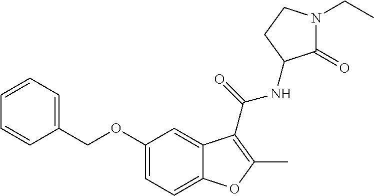 [SMILES: CCN1CCC(NC(=O)c2c(C)oc3ccc(OCc4ccccc4)cc23)C1=O] | Cpd 147 |
| 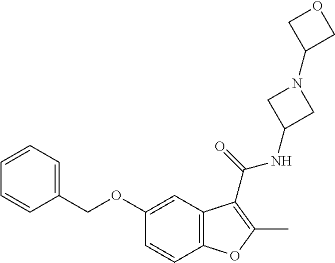 [SMILES: Cc1oc2ccc(OCc3ccccc3)cc2c1C(=O)NC1CN(C2COC2)C1] | Cpd 148 |
| 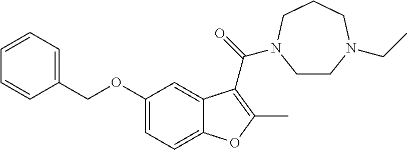 [SMILES: CCN1CCCN(C(=O)c2c(C)oc3ccc(OCc4ccccc4)cc23)CC1] | Cpd 149 |
| 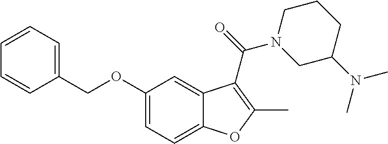 [SMILES: Cc1oc2ccc(OCc3ccccc3)cc2c1C(=O)N1CCCC(N(C)C)C1] | Cpd 150 |
| 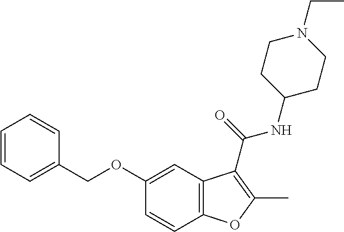 [SMILES: CCN1CCC(NC(=O)c2c(C)oc3ccc(OCc4ccccc4)cc23)CC1] | Cpd 151 |
| 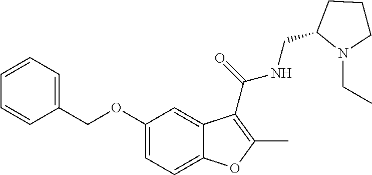 [SMILES: CCN1CCC[C@H]1CNC(=O)c1c(C)oc2ccc(OCc3ccccc3)cc12] | Cpd 152 |
| 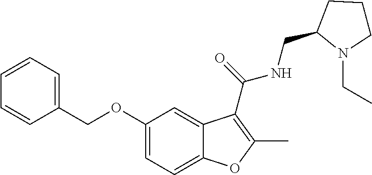 [SMILES: CCN1CCC[C@@H]1CNC(=O)c1c(C)oc2ccc(OCc3ccccc3)cc12] | Cpd 153 |
| 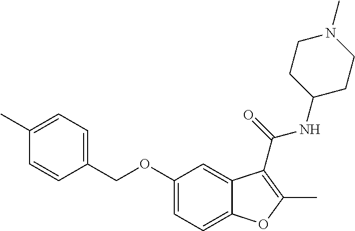 [SMILES: Cc1ccc(COc2ccc3oc(C)c(C(=O)NC4CCN(C)CC4)c3c2)cc1] | Cpd 154 |
| 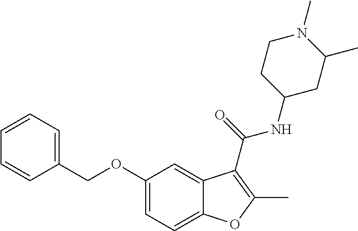 [SMILES: Cc1oc2ccc(OCc3ccccc3)cc2c1C(=O)NC1CCN(C)C(C)C1] | Cpd 155 |
| 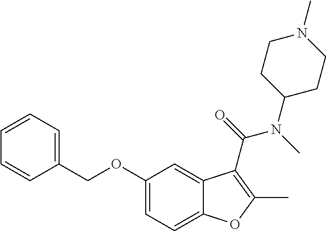 [SMILES: Cc1oc2ccc(OCc3ccccc3)cc2c1C(=O)N(C)C1CCN(C)CC1] | Cpd 156 |
| 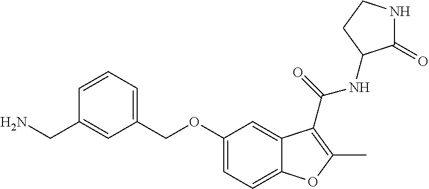 [SMILES: Cc1oc2ccc(OCc3cccc(CN)c3)cc2c1C(=O)NC1CCNC1=O] | Cpd 157 |
| 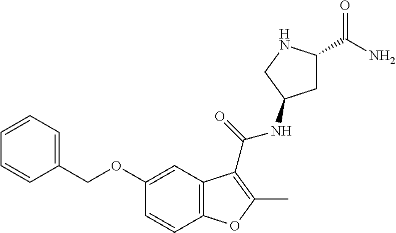 [SMILES: Cc1oc2ccc(OCc3ccccc3)cc2c1C(=O)N[C@H]1CN[C@H](C(N)=O)C1] | Cpd 158 |
| 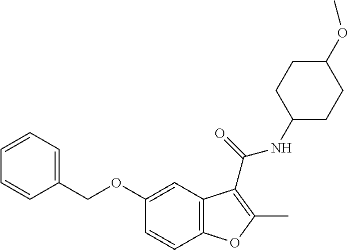 [SMILES: COC1CCC(NC(=O)c2c(C)oc3ccc(OCc4ccccc4)cc23)CC1] | Cpd 159 |
| 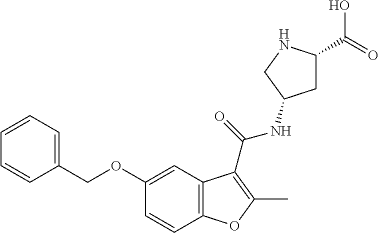 [SMILES: Cc1oc2ccc(OCc3ccccc3)cc2c1C(=O)N[C@@H]1CN[C@H](C(=O)O)C1] | Cpd 160 |
| 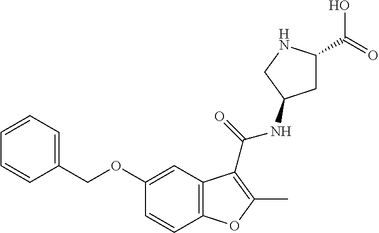 [SMILES: Cc1oc2ccc(OCc3ccccc3)cc2c1C(=O)N[C@H]1CN[C@H](C(=O)O)C1] | Cpd 161 |
| 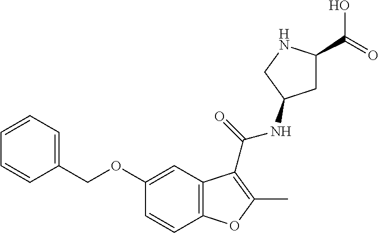 [SMILES: Cc1oc2ccc(OCc3ccccc3)cc2c1C(=O)N[C@H]1CN[C@@H](C(=O)O)C1] | Cpd 162 |
| 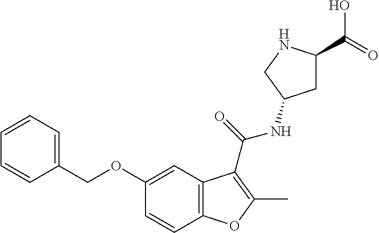 [SMILES: Cc1oc2ccc(OCc3ccccc3)cc2c1C(=O)N[C@@H]1CN[C@@H](C(=O)O)C1] | Cpd 163 |
| 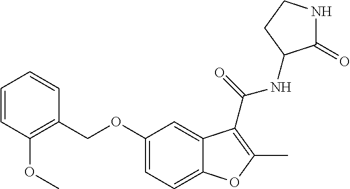 [SMILES: COc1ccccc1COc1ccc2oc(C)c(C(=O)NC3CCNC3=O)c2c1] | Cpd 164 |
| 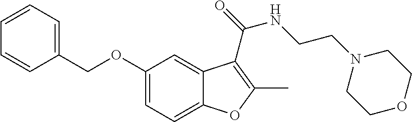 [SMILES: Cc1oc2ccc(OCc3ccccc3)cc2c1C(=O)NCCN1CCOCC1] | Cpd 165 |
| 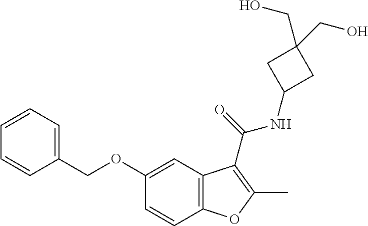 [SMILES: Cc1oc2ccc(OCc3ccccc3)cc2c1C(=O)NC1CC(CO)(CO)C1] | Cpd 166 |
| 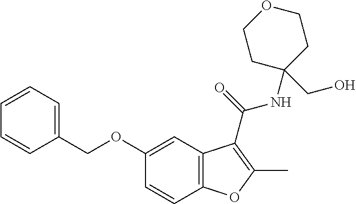 [SMILES: Cc1oc2ccc(OCc3ccccc3)cc2c1C(=O)NC1(CO)CCOCC1] | Cpd 167 |
| 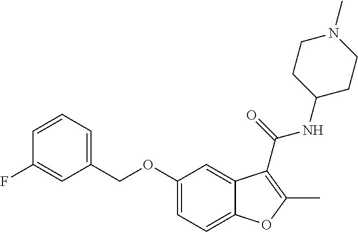 [SMILES: Cc1oc2ccc(OCc3cccc(F)c3)cc2c1C(=O)NC1CCN(C)CC1] | Cpd 168 |
| 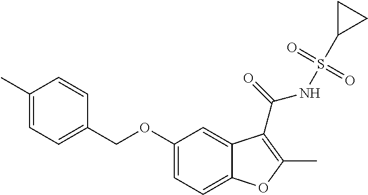 [SMILES: Cc1ccc(COc2ccc3oc(C)c(C(=O)NS(=O)(=O)C4CC4)c3c2)cc1] | Cpd 169 |
| 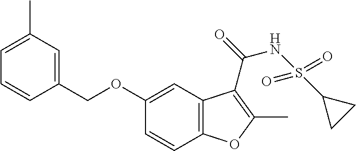 [SMILES: Cc1cccc(COc2ccc3oc(C)c(C(=O)NS(=O)(=O)C4CC4)c3c2)c1] | Cpd 170 |
| 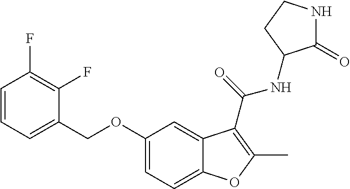 [SMILES: Cc1oc2ccc(OCc3cccc(F)c3F)cc2c1C(=O)NC1CCNC1=O] | Cpd 171 |
| 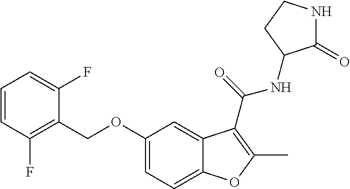 [SMILES: Cc1oc2ccc(OCc3c(F)cccc3F)cc2c1C(=O)NC1CCNC1=O] | Cpd 172 |
| 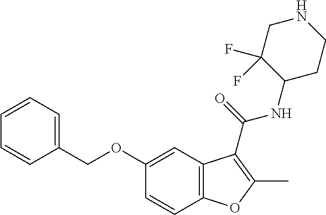 [SMILES: Cc1oc2ccc(OCc3ccccc3)cc2c1C(=O)NC1CCNCC1(F)F] | Cpd 173 |
| 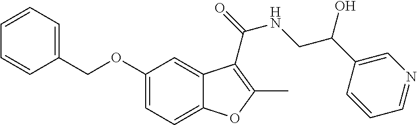 [SMILES: Cc1oc2ccc(OCc3ccccc3)cc2c1C(=O)NCC(O)c1cccnc1] | Cpd 174 |
| 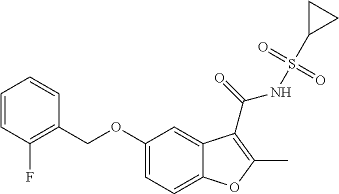 [SMILES: Cc1oc2ccc(OCc3ccccc3F)cc2c1C(=O)NS(=O)(=O)C1CC1] | Cpd 175 |
| 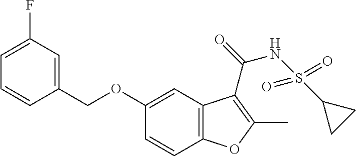 [SMILES: Cc1oc2ccc(OCc3cccc(F)c3)cc2c1C(=O)NS(=O)(=O)C1CC1] | Cpd 176 |
| 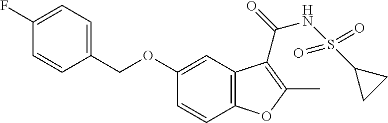 [SMILES: Cc1oc2ccc(OCc3ccc(F)cc3)cc2c1C(=O)NS(=O)(=O)C1CC1] | Cpd 177 |
| 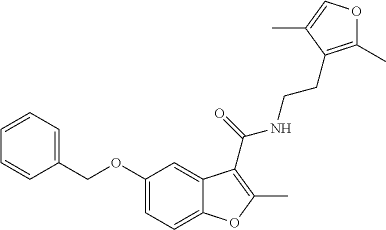 [SMILES: Cc1coc(C)c1CCNC(=O)c1c(C)oc2ccc(OCc3ccccc3)cc12] | Cpd 178 |
| 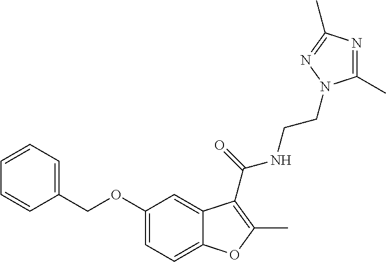 [SMILES: Cc1nc(C)n(CCNC(=O)c2c(C)oc3ccc(OCc4ccccc4)cc23)n1] | Cpd 179 |
| 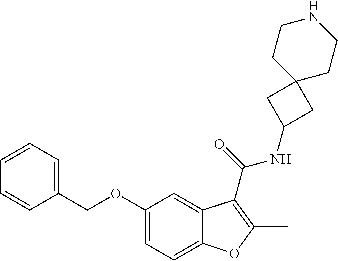 [SMILES: Cc1oc2ccc(OCc3ccccc3)cc2c1C(=O)NC1CC2(CCNCC2)C1] | Cpd 180 |
| 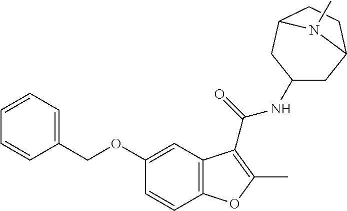 [SMILES: Cc1oc2ccc(OCc3ccccc3)cc2c1C(=O)NC1CC2CCC(C1)N2C] | Cpd 181 |
| 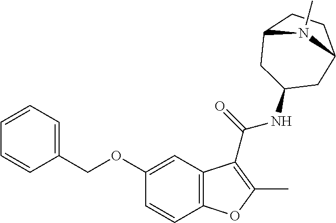 | Cpd 182 |
| 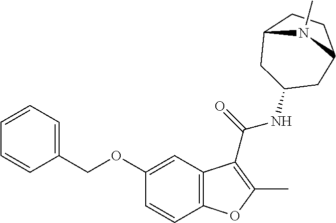 | Cpd 183 |
| 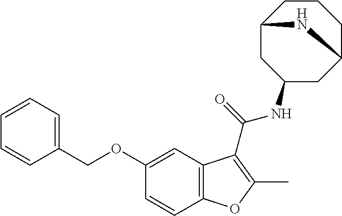 [SMILES: Cc1oc2ccc(OCc3ccccc3)cc2c1C(=O)N[C@@H]1C[C@H]2CCC[C@@H](C1)N2] | Cpd 184 |
| 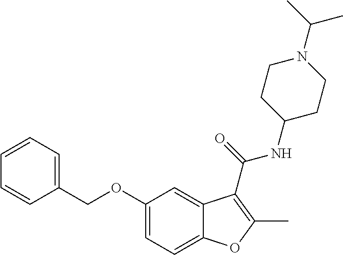 [SMILES: Cc1oc2ccc(OCc3ccccc3)cc2c1C(=O)NC1CCN(C(C)C)CC1] | Cpd 185 |
| 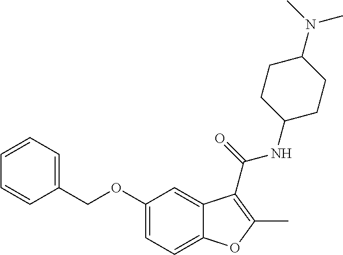 [SMILES: Cc1oc2ccc(OCc3ccccc3)cc2c1C(=O)NC1CCC(N(C)C)CC1] | Cpd 186 |
| 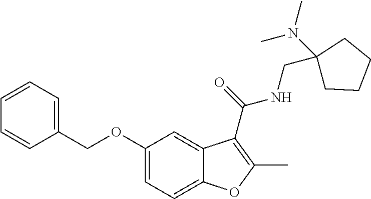 [SMILES: Cc1oc2ccc(OCc3ccccc3)cc2c1C(=O)NCC1(N(C)C)CCCC1] | Cpd 187 |
| 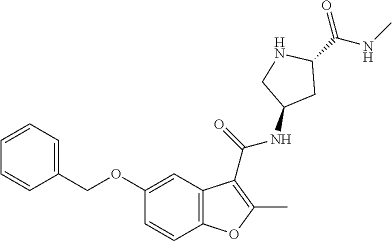 [SMILES: CNC(=O)[C@@H]1C[C@@H](NC(=O)c2c(C)oc3ccc(OCc4ccccc4)cc23)CN1] | Cpd 188 |
| 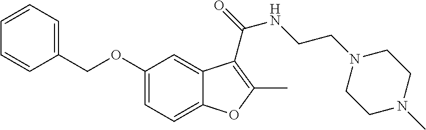 [SMILES: Cc1oc2ccc(OCc3ccccc3)cc2c1C(=O)NCCN1CCN(C)CC1] | Cpd 189 |
| 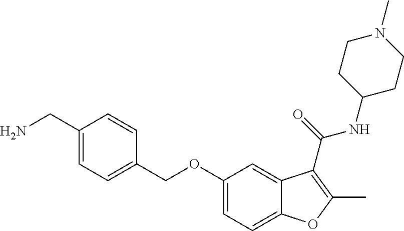 [SMILES: Cc1oc2ccc(OCc3ccc(CN)cc3)cc2c1C(=O)NC1CCN(C)CC1] | Cpd 190 |
| 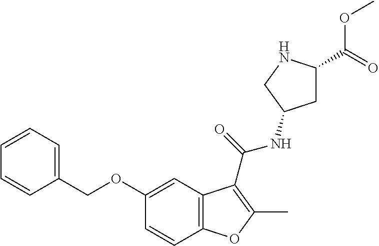 [SMILES: COC(=O)[C@@H]1C[C@H](NC(=O)c2c(C)oc3ccc(OCc4ccccc4)cc23)CN1] | Cpd 191 |
| 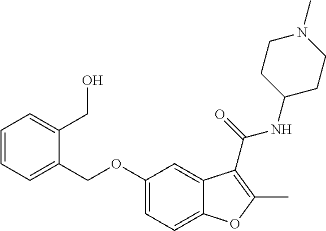 [SMILES: Cc1oc2ccc(OCc3ccccc3CO)cc2c1C(=O)NC1CCN(C)CC1] | Cpd 192 |
| 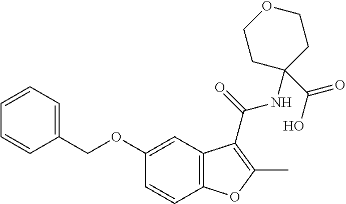 [SMILES: Cc1oc2ccc(OCc3ccccc3)cc2c1C(=O)NC1(C(=O)O)CCOCC1] | Cpd 193 |
| 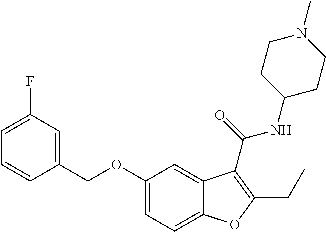 [SMILES: CCc1oc2ccc(OCc3cccc(F)c3)cc2c1C(=O)NC1CCN(C)CC1] | Cpd 194 |
| 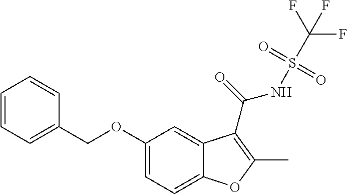 [SMILES: Cc1oc2ccc(OCc3ccccc3)cc2c1C(=O)NS(=O)(=O)C(F)(F)F] | Cpd 195 |
| 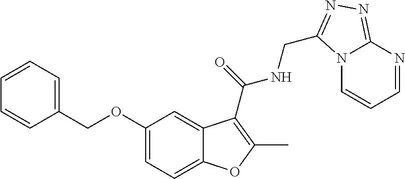 [SMILES: Cc1oc2ccc(OCc3ccccc3)cc2c1C(=O)NCc1nnc2ncccn12] | Cpd 196 |
| 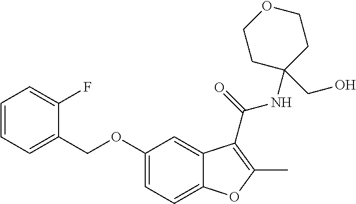 [SMILES: Cc1oc2ccc(OCc3ccccc3F)cc2c1C(=O)NC1(CO)CCOCC1] | Cpd 197 |
| 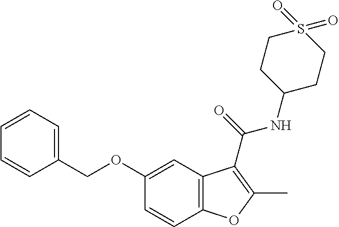 [SMILES: Cc1oc2ccc(OCc3ccccc3)cc2c1C(=O)NC1CCS(=O)(=O)CC1] | Cpd 198 |
| 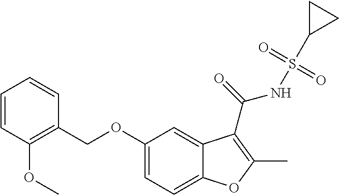 [SMILES: COc1ccccc1COc1ccc2oc(C)c(C(=O)NS(=O)(=O)C3CC3)c2c1] | Cpd 199 |
| 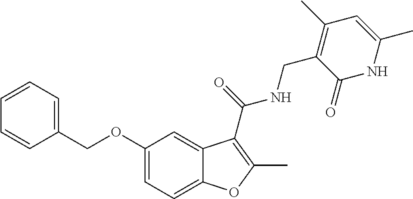 [SMILES: Cc1cc(C)c(CNC(=O)c2c(C)oc3ccc(OCc4ccccc4)cc23)c(=O)[nH]1] | Cpd 200 |
| 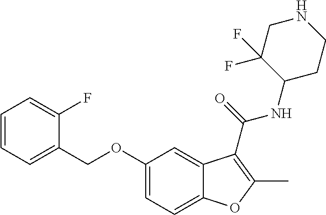 [SMILES: Cc1oc2ccc(OCc3ccccc3F)cc2c1C(=O)NC1CCNCC1(F)F] | Cpd 201 |
| 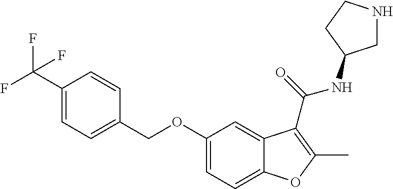 [SMILES: Cc1oc2ccc(OCc3ccc(C(F)(F)F)cc3)cc2c1C(=O)N[C@H]1CCNC1] | Cpd 202 |
| 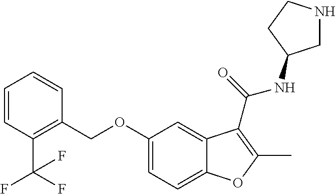 [SMILES: Cc1oc2ccc(OCc3ccccc3C(F)(F)F)cc2c1C(=O)N[C@H]1CCNC1] | Cpd 203 |
| 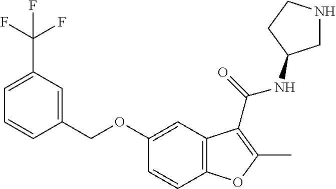 [SMILES: Cc1oc2ccc(OCc3cccc(C(F)(F)F)c3)cc2c1C(=O)N[C@H]1CCNC1] | Cpd 204 |
| 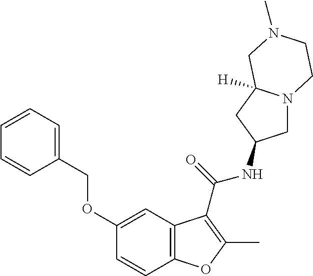 [SMILES: Cc1oc2ccc(OCc3ccccc3)cc2c1C(=O)N[C@H]1C[C@H]2CN(C)CCN2C1] | Cpd 205 |
| 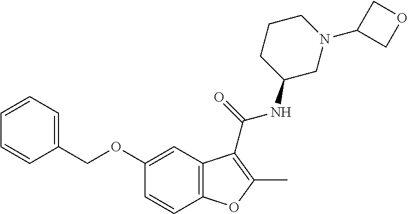 [SMILES: Cc1oc2ccc(OCc3ccccc3)cc2c1C(=O)N[C@H]1CCCN(C2COC2)C1] | Cpd 206 |
| 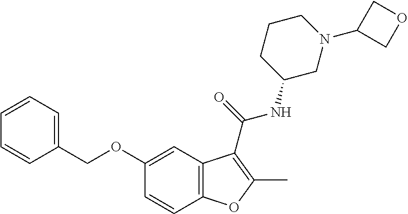 [SMILES: Cc1oc2ccc(OCc3ccccc3)cc2c1C(=O)N[C@@H]1CCCN(C2COC2)C1] | Cpd 207 |
| 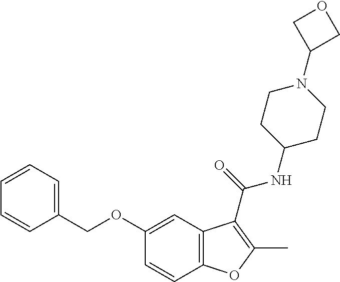 [SMILES: Cc1oc2ccc(OCc3ccccc3)cc2c1C(=O)NC1CCN(C2COC2)CC1] | Cpd 208 |
| 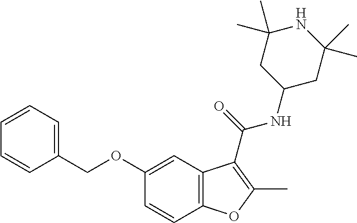 [SMILES: Cc1oc2ccc(OCc3ccccc3)cc2c1C(=O)NC1CC(C)(C)NC(C)(C)C1] | Cpd 209 |
| 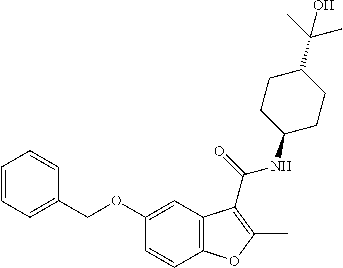 | Cpd 210 |
| 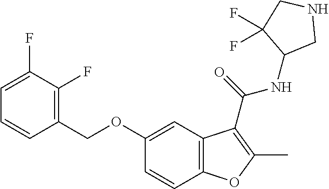 [SMILES: Cc1oc2ccc(OCc3cccc(F)c3F)cc2c1C(=O)NC1CNCC1(F)F] | Cpd 211 |
| 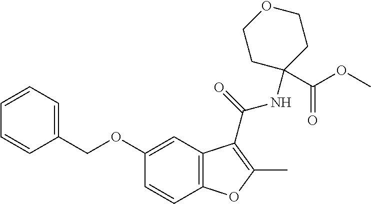 [SMILES: COC(=O)C1(NC(=O)c2c(C)oc3ccc(OCc4ccccc4)cc23)CCOCC1] | Cpd 212 |
| 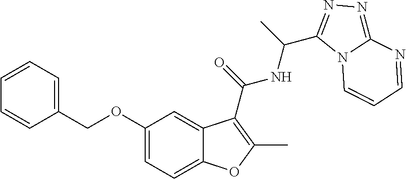 [SMILES: Cc1oc2ccc(OCc3ccccc3)cc2c1C(=O)NC(C)c1nnc2ncccn12] | Cpd 213 |
| 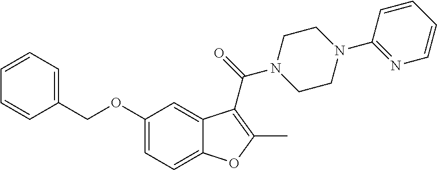 [SMILES: Cc1oc2ccc(OCc3ccccc3)cc2c1C(=O)N1CCN(c2ccccn2)CC1] | Cpd 214 |
| 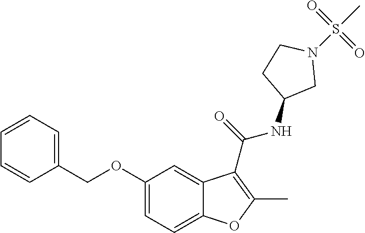 [SMILES: Cc1oc2ccc(OCc3ccccc3)cc2c1C(=O)N[C@H]1CCN(S(C)(=O)=O)C1] | Cpd 215 |
| 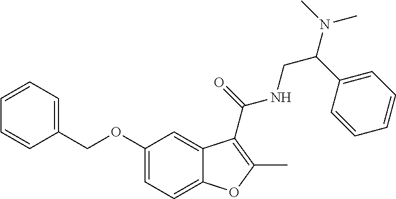 [SMILES: Cc1oc2ccc(OCc3ccccc3)cc2c1C(=O)NCC(c1ccccc1)N(C)C] | Cpd 216 |
| 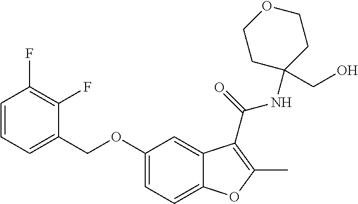 [SMILES: Cc1oc2ccc(OCc3cccc(F)c3F)cc2c1C(=O)NC1(CO)CCOCC1] | Cpd 217 |
| 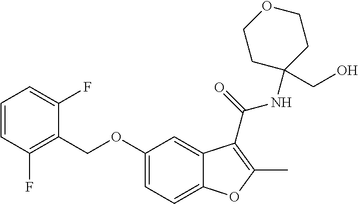 [SMILES: Cc1oc2ccc(OCc3c(F)cccc3F)cc2c1C(=O)NC1(CO)CCOCC1] | Cpd 218 |
| 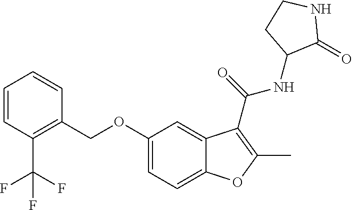 [SMILES: Cc1oc2ccc(OCc3ccccc3C(F)(F)F)cc2c1C(=O)NC1CCNC1=O] | Cpd 219 |
| 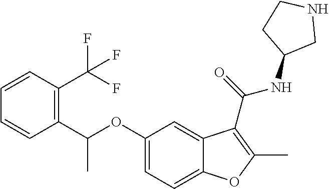 [SMILES: Cc1oc2ccc(OC(C)c3ccccc3C(F)(F)F)cc2c1C(=O)N[C@H]1CCNC1] | Cpd 220 |
| 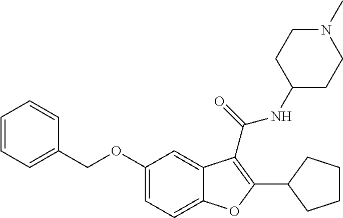 [SMILES: CN1CCC(NC(=O)c2c(C3CCCC3)oc3ccc(OCc4ccccc4)cc23)CC1] | Cpd 221 |
| 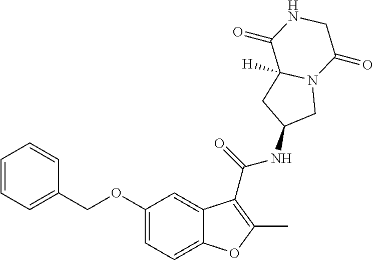 [SMILES: Cc1oc2ccc(OCc3ccccc3)cc2c1C(=O)N[C@H]1C[C@H]2C(=O)NCC(=O)N2C1] | Cpd 222 |
| 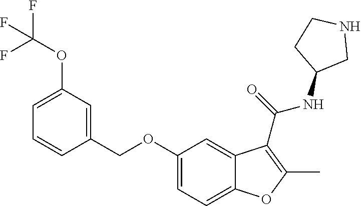 [SMILES: Cc1oc2ccc(OCc3cccc(OC(F)(F)F)c3)cc2c1C(=O)N[C@H]1CCNC1] | Cpd 223 |
| 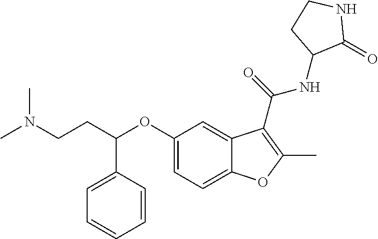 [SMILES: Cc1oc2ccc(OC(CCN(C)C)c3ccccc3)cc2c1C(=O)NC1CCNC1=O] | Cpd 224 |
| 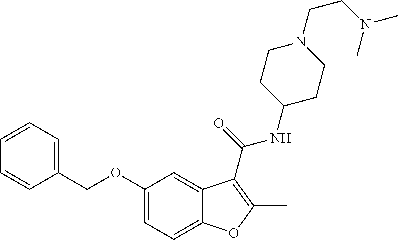 [SMILES: Cc1oc2ccc(OCc3ccccc3)cc2c1C(=O)NC1CCN(CCN(C)C)CC1] | Cpd 225 |
| 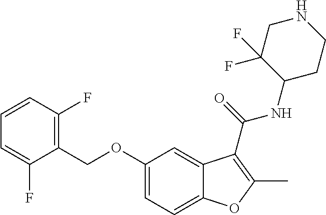 [SMILES: Cc1oc2ccc(OCc3c(F)cccc3F)cc2c1C(=O)NC1CCNCC1(F)F] | Cpd 226 |
| 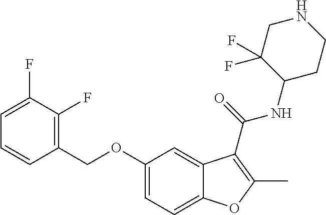 [SMILES: Cc1oc2ccc(OCc3cccc(F)c3F)cc2c1C(=O)NC1CCNCC1(F)F] | Cpd 227 |
| 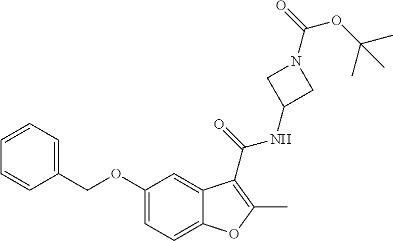 [SMILES: Cc1oc2ccc(OCc3ccccc3)cc2c1C(=O)NC1CN(C(=O)OC(C)(C)C)C1] | Cpd 228 |
| 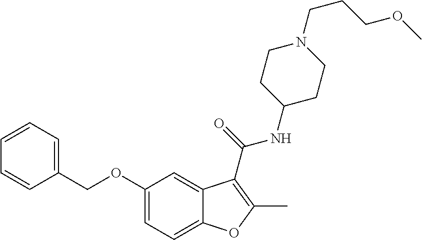 [SMILES: COCCCN1CCC(NC(=O)c2c(C)oc3ccc(OCc4ccccc4)cc23)CC1] | Cpd 229 |
| 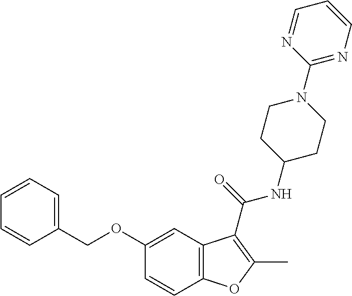 [SMILES: Cc1oc2ccc(OCc3ccccc3)cc2c1C(=O)NC1CCN(c2ncccn2)CC1] | Cpd 230 |
| 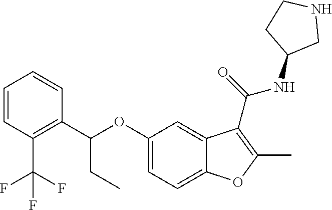 [SMILES: CCC(Oc1ccc2oc(C)c(C(=O)N[C@H]3CCNC3)c2c1)c1ccccc1C(F)(F)F] | Cpd 231 |
| 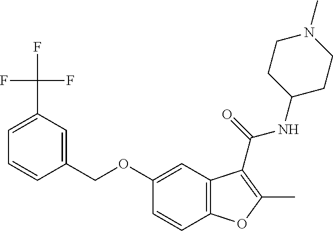 [SMILES: Cc1oc2ccc(OCc3cccc(C(F)(F)F)c3)cc2c1C(=O)NC1CCN(C)CC1] | Cpd 232 |
| 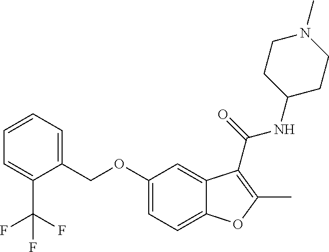 [SMILES: Cc1oc2ccc(OCc3ccccc3C(F)(F)F)cc2c1C(=O)NC1CCN(C)CC1] | Cpd 233 |
| 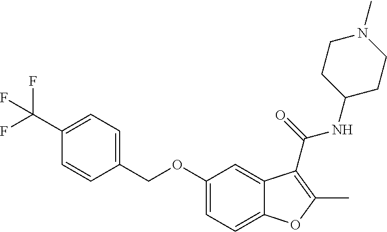 [SMILES: Cc1oc2ccc(OCc3ccc(C(F)(F)F)cc3)cc2c1C(=O)NC1CCN(C)CC1] | Cpd 234 |
| 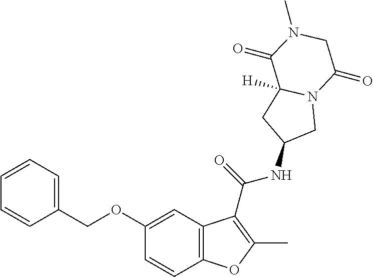 [SMILES: Cc1oc2ccc(OCc3ccccc3)cc2c1C(=O)N[C@H]1C[C@H]2C(=O)N(C)CC(=O)N2C1] | Cpd 235 |
| 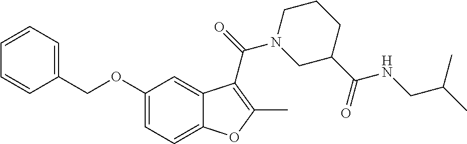 [SMILES: Cc1oc2ccc(OCc3ccccc3)cc2c1C(=O)N1CCCC(C(=O)NCC(C)C)C1] | Cpd 236 |
| 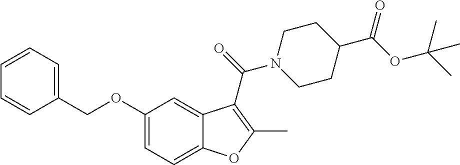 [SMILES: Cc1oc2ccc(OCc3ccccc3)cc2c1C(=O)N1CCC(C(=O)OC(C)(C)C)CC1] | Cpd 237 |
| 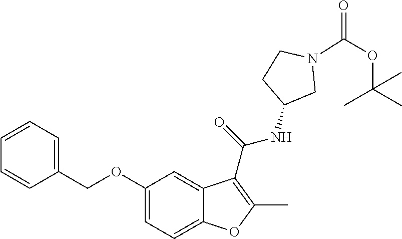 [SMILES: Cc1oc2ccc(OCc3ccccc3)cc2c1C(=O)N[C@@H]1CCN(C(=O)OC(C)(C)C)C1] | Cpd 238 |
| 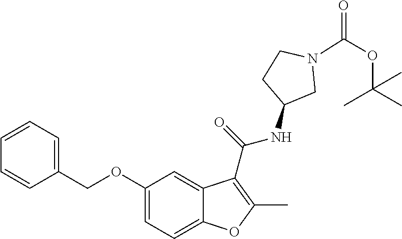 [SMILES: Cc1oc2ccc(OCc3ccccc3)cc2c1C(=O)N[C@H]1CCN(C(=O)OC(C)(C)C)C1] | Cpd 239 |
| 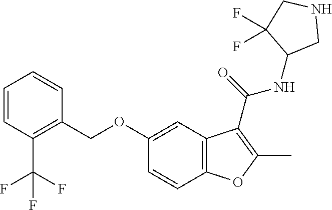 [SMILES: Cc1oc2ccc(OCc3ccccc3C(F)(F)F)cc2c1C(=O)NC1CNCC1(F)F] | Cpd 240 |
| 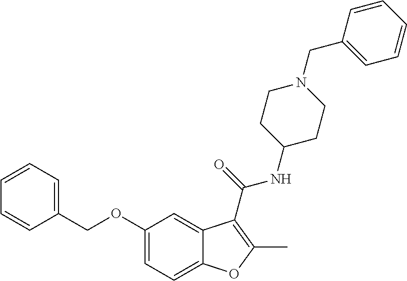 [SMILES: Cc1oc2ccc(OCc3ccccc3)cc2c1C(=O)NC1CCN(Cc2ccccc2)CC1] | Cpd 241 |
| 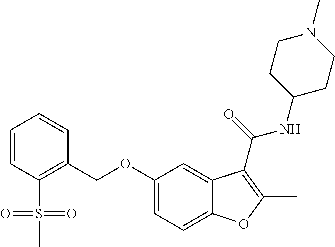 [SMILES: Cc1oc2ccc(OCc3ccccc3S(C)(=O)=O)cc2c1C(=O)NC1CCN(C)CC1] | Cpd 242 |
| 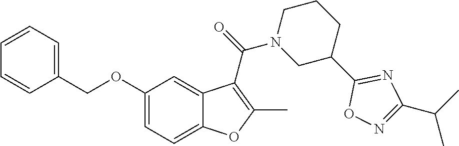 [SMILES: Cc1oc2ccc(OCc3ccccc3)cc2c1C(=O)N1CCCC(c2nc(C(C)C)no2)C1] | Cpd 243 |
| 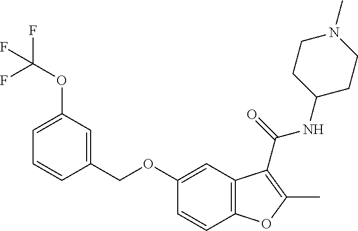 [SMILES: Cc1oc2ccc(OCc3cccc(OC(F)(F)F)c3)cc2c1C(=O)NC1CCN(C)CC1] | Cpd 244 |
| 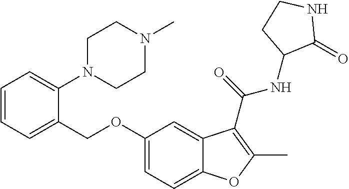 [SMILES: Cc1oc2ccc(OCc3ccccc3N3CCN(C)CC3)cc2c1C(=O)NC1CCNC1=O] | Cpd 245 |
| 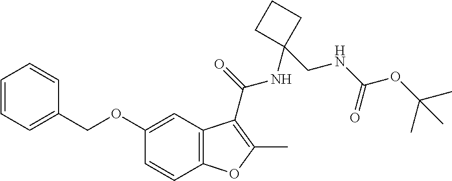 [SMILES: Cc1oc2ccc(OCc3ccccc3)cc2c1C(=O)NC1(CNC(=O)OC(C)(C)C)CCC1] | Cpd 246 |
| 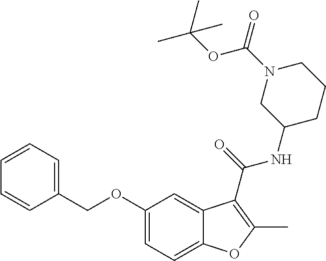 [SMILES: Cc1oc2ccc(OCc3ccccc3)cc2c1C(=O)NC1CCCN(C(=O)OC(C)(C)C)C1] | Cpd 247 |
| 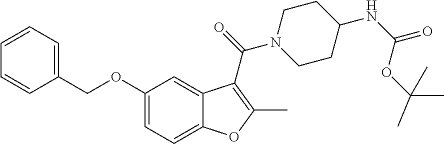 [SMILES: Cc1oc2ccc(OCc3ccccc3)cc2c1C(=O)N1CCC(NC(=O)OC(C)(C)C)CC1] | Cpd 248 |
| 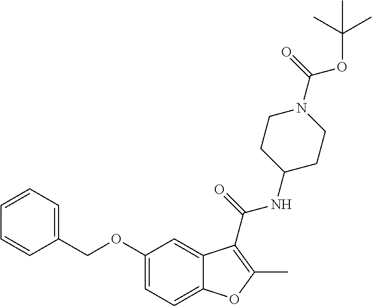 [SMILES: Cc1oc2ccc(OCc3ccccc3)cc2c1C(=O)NC1CCN(C(=O)OC(C)(C)C)CC1] | Cpd 249 |
| 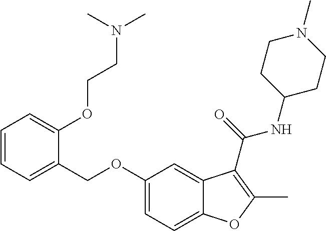 [SMILES: Cc1oc2ccc(OCc3ccccc3OCCN(C)C)cc2c1C(=O)NC1CCN(C)CC1] | Cpd 250 |
| 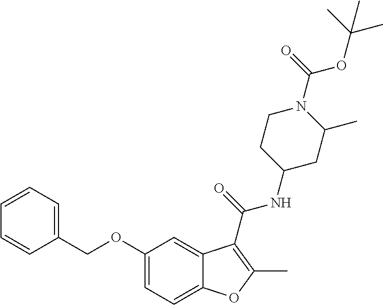 [SMILES: Cc1oc2ccc(OCc3ccccc3)cc2c1C(=O)NC1CCN(C(=O)OC(C)(C)C)C(C)C1] | Cpd 251 |
| 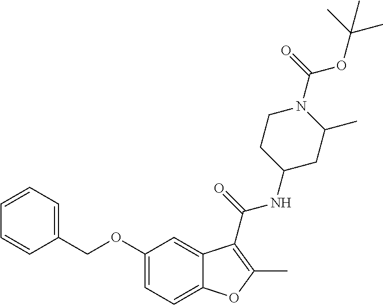 [SMILES: Cc1oc2ccc(OCc3ccccc3)cc2c1C(=O)NC1CCN(C(=O)OC(C)(C)C)C(C)C1] | Cpd 252 |
| 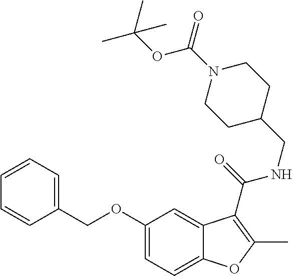 [SMILES: Cc1oc2ccc(OCc3ccccc3)cc2c1C(=O)NCC1CCN(C(=O)OC(C)(C)C)CC1] | Cpd 253 |
| 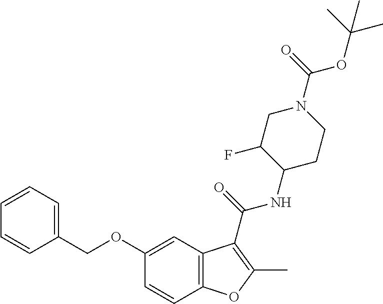 [SMILES: Cc1oc2ccc(OCc3ccccc3)cc2c1C(=O)NC1CCN(C(=O)OC(C)(C)C)CC1F] | Cpd 254 |
| 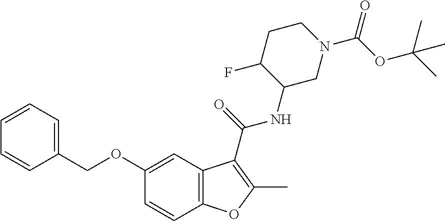 [SMILES: Cc1oc2ccc(OCc3ccccc3)cc2c1C(=O)NC1CN(C(=O)OC(C)(C)C)CCC1F] | Cpd 255 |
| 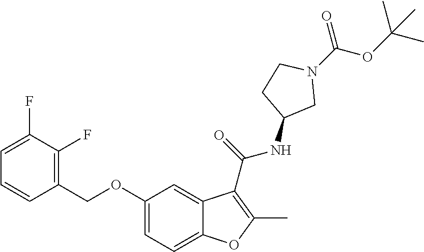 [SMILES: Cc1oc2ccc(OCc3cccc(F)c3F)cc2c1C(=O)N[C@H]1CCN(C(=O)OC(C)(C)C)C1] | Cpd 256 |
| 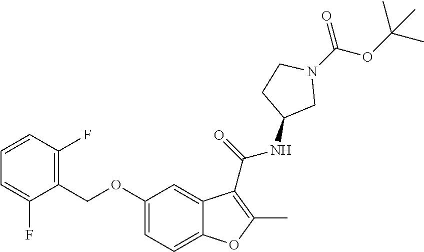 [SMILES: Cc1oc2ccc(OCc3c(F)cccc3F)cc2c1C(=O)N[C@H]1CCN(C(=O)OC(C)(C)C)C1] | Cpd 257 |
| 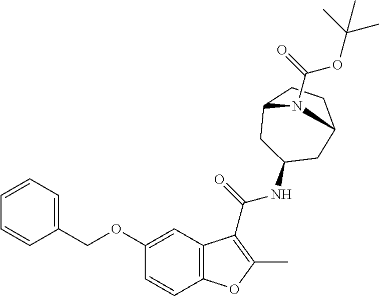 | Cpd 258 |
| 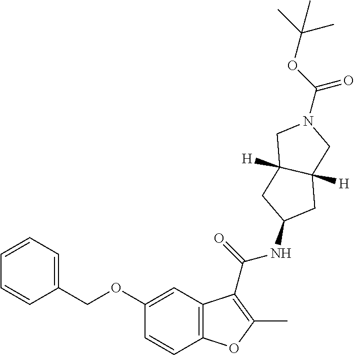 [SMILES: Cc1oc2ccc(OCc3ccccc3)cc2c1C(=O)N[C@H]1C[C@@H]2CN(C(=O)OC(C)(C)C)C[C@@H]2C1] | Cpd 259 |
| 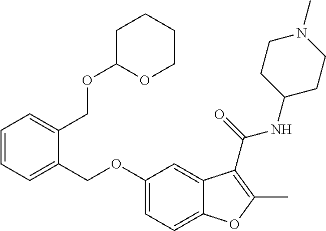 [SMILES: Cc1oc2ccc(OCc3ccccc3COC3CCCCO3)cc2c1C(=O)NC1CCN(C)CC1] | Cpd 260 |
| 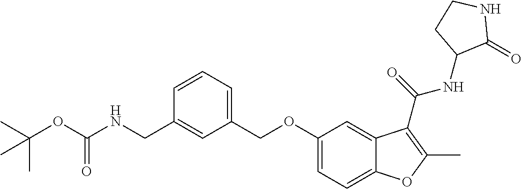 [SMILES: Cc1oc2ccc(OCc3cccc(CNC(=O)OC(C)(C)C)c3)cc2c1C(=O)NC1CCNC1=O] | Cpd 261 |
| 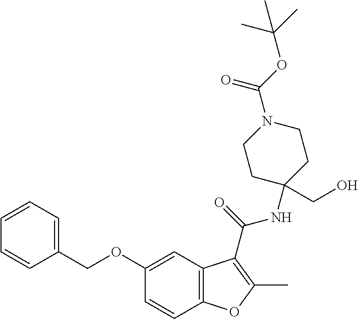 [SMILES: Cc1oc2ccc(OCc3ccccc3)cc2c1C(=O)NC1(CO)CCN(C(=O)OC(C)(C)C)CC1] | Cpd 262 |
| 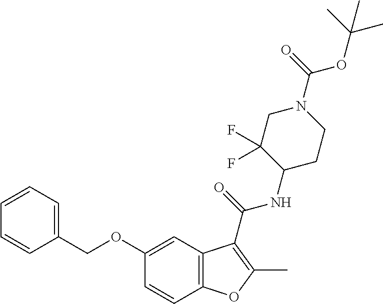 [SMILES: Cc1oc2ccc(OCc3ccccc3)cc2c1C(=O)NC1CCN(C(=O)OC(C)(C)C)CC1(F)F] | Cpd 263 |
| 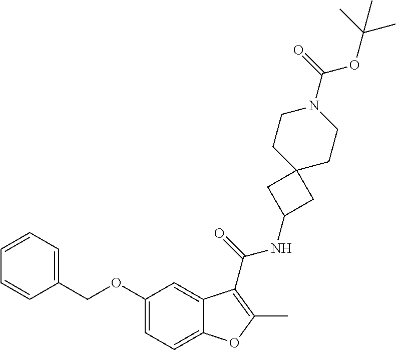 [SMILES: Cc1oc2ccc(OCc3ccccc3)cc2c1C(=O)NC1CC2(CCN(C(=O)OC(C)(C)C)CC2)C1] | Cpd 264 |
| 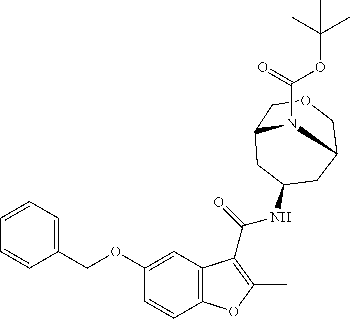 | Cpd 265 |
| 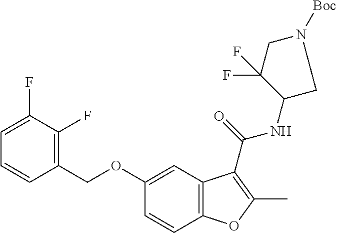 [SMILES: Cc1oc2ccc(OCc3cccc(F)c3F)cc2c1C(=O)NC1CN(C(=O)OC(C)(C)C)CC1(F)F] | Cpd 266 |
| 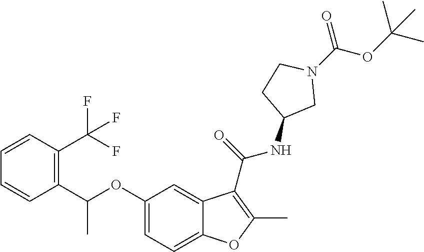 [SMILES: Cc1oc2ccc(OC(C)c3ccccc3C(F)(F)F)cc2c1C(=O)N[C@H]1CCN(C(=O)OC(C)(C)C)C1] | Cpd 267 |
| 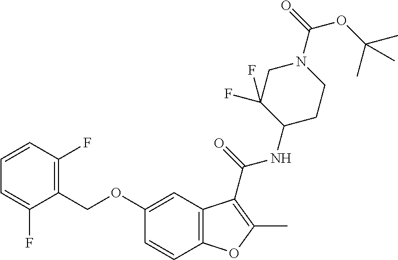 [SMILES: Cc1oc2ccc(OCc3c(F)cccc3F)cc2c1C(=O)NC1CCN(C(=O)OC(C)(C)C)CC1(F)F] | Cpd 268 |
| 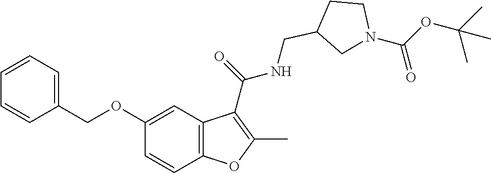 [SMILES: Cc1oc2ccc(OCc3ccccc3)cc2c1C(=O)NCC1CCN(C(=O)OC(C)(C)C)C1] | Cpd 269 |
| 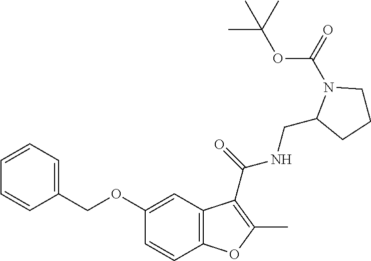 [SMILES: Cc1oc2ccc(OCc3ccccc3)cc2c1C(=O)NCC1CCCN1C(=O)OC(C)(C)C] | Cpd 270 |
| 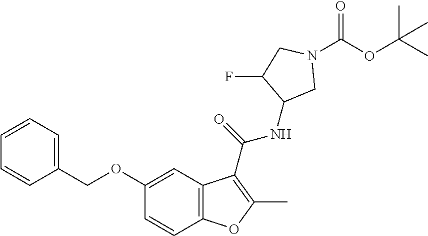 [SMILES: Cc1oc2ccc(OCc3ccccc3)cc2c1C(=O)NC1CN(C(=O)OC(C)(C)C)CC1F] | Cpd 271 |
| 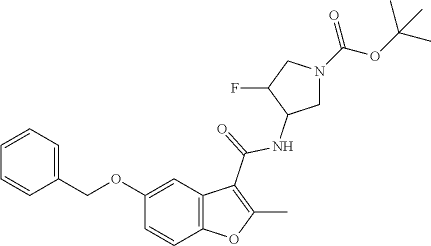 [SMILES: Cc1oc2ccc(OCc3ccccc3)cc2c1C(=O)NC1CN(C(=O)OC(C)(C)C)CC1F] | Cpd 272 |
| 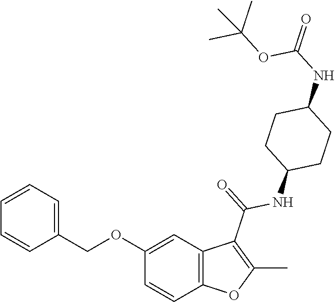 [SMILES: Cc1oc2ccc(OCc3ccccc3)cc2c1C(=O)N[C@H]1CC[C@@H](NC(=O)OC(C)(C)C)CC1] | Cpd 273 |
| 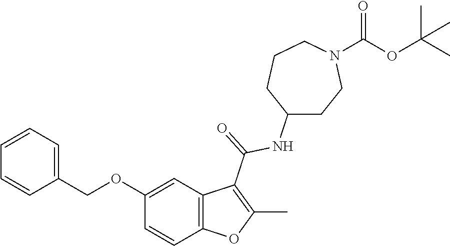 [SMILES: Cc1oc2ccc(OCc3ccccc3)cc2c1C(=O)NC1CCCN(C(=O)OC(C)(C)C)CC1] | Cpd 274 |
| 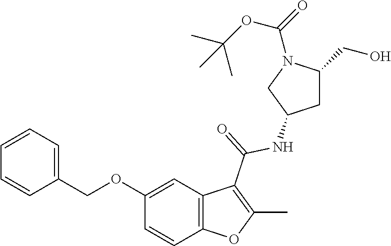 [SMILES: Cc1oc2ccc(OCc3ccccc3)cc2c1C(=O)N[C@H]1C[C@@H](CO)N(C(=O)OC(C)(C)C)C1] | Cpd 275 |
| 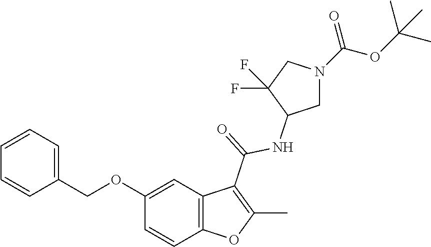 [SMILES: Cc1oc2ccc(OCc3ccccc3)cc2c1C(=O)NC1CN(C(=O)OC(C)(C)C)CC1(F)F] | Cpd 276 |
| 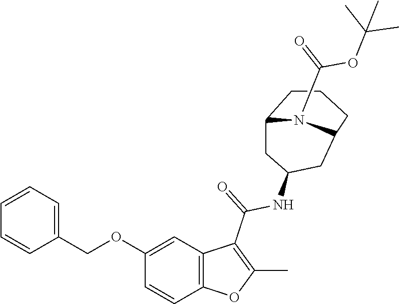 | Cpd 277 |
| 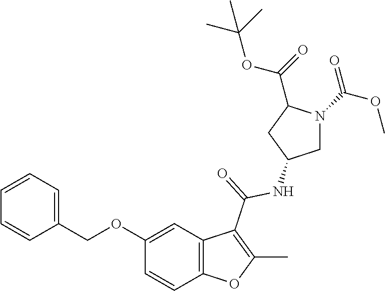 | Cpd 278 |
| 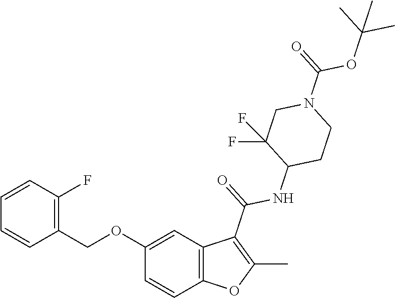 [SMILES: Cc1oc2ccc(OCc3ccccc3F)cc2c1C(=O)NC1CCN(C(=O)OC(C)(C)C)CC1(F)F] | Cpd 279 |
| 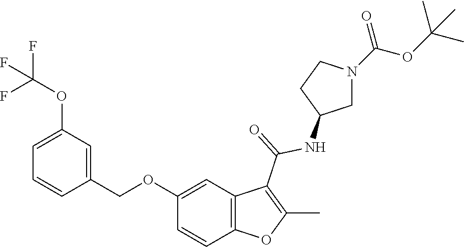 [SMILES: Cc1oc2ccc(OCc3cccc(OC(F)(F)F)c3)cc2c1C(=O)N[C@H]1CCN(C(=O)OC(C)(C)C)C1] | Cpd 280 |
| 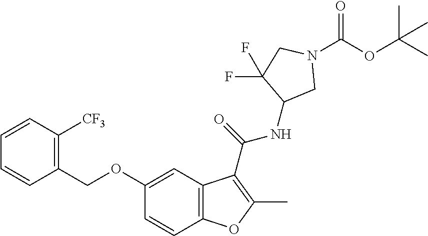 [SMILES: Cc1oc2ccc(OCc3ccccc3C(F)(F)F)cc2c1C(=O)NC1CN(C(=O)OC(C)(C)C)CC1(F)F] | Cpd 281 |
| 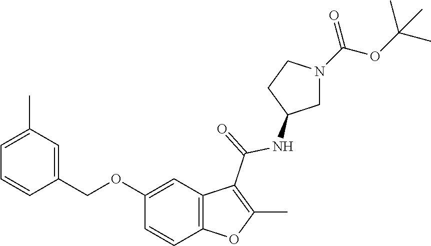 [SMILES: Cc1cccc(COc2ccc3oc(C)c(C(=O)N[C@H]4CCN(C(=O)OC(C)(C)C)C4)c3c2)c1] | Cpd 282 |
| 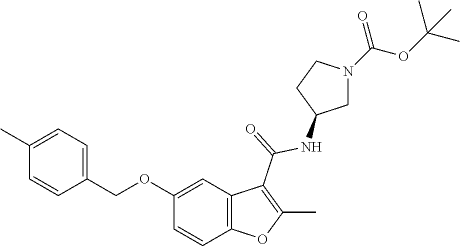 [SMILES: Cc1ccc(COc2ccc3oc(C)c(C(=O)N[C@H]4CCN(C(=O)OC(C)(C)C)C4)c3c2)cc1] | Cpd 283 |
| 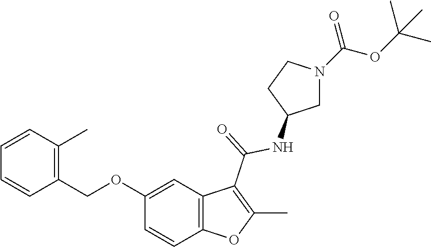 [SMILES: Cc1ccccc1COc1ccc2oc(C)c(C(=O)N[C@H]3CCN(C(=O)OC(C)(C)C)C3)c2c1] | Cpd 284 |
| 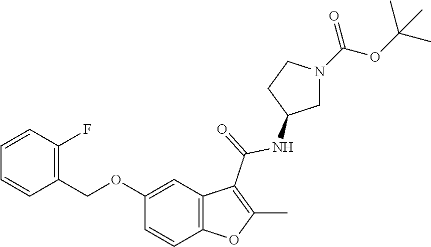 [SMILES: Cc1oc2ccc(OCc3ccccc3F)cc2c1C(=O)N[C@H]1CCN(C(=O)OC(C)(C)C)C1] | Cpd 285 |
| 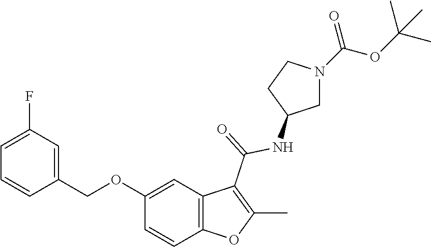 [SMILES: Cc1oc2ccc(OCc3cccc(F)c3)cc2c1C(=O)N[C@H]1CCN(C(=O)OC(C)(C)C)C1] | Cpd 286 |
| 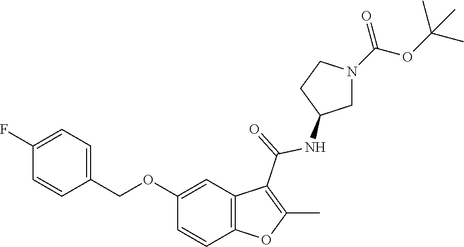 [SMILES: Cc1oc2ccc(OCc3ccc(F)cc3)cc2c1C(=O)N[C@H]1CCN(C(=O)OC(C)(C)C)C1] | Cpd 287 |
| 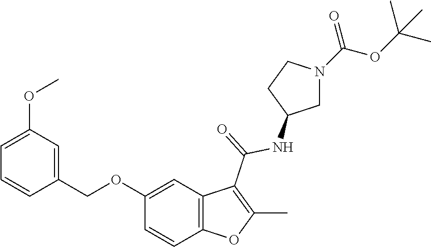 [SMILES: COc1cccc(COc2ccc3oc(C)c(C(=O)N[C@H]4CCN(C(=O)OC(C)(C)C)C4)c3c2)c1] | Cpd 288 |
| 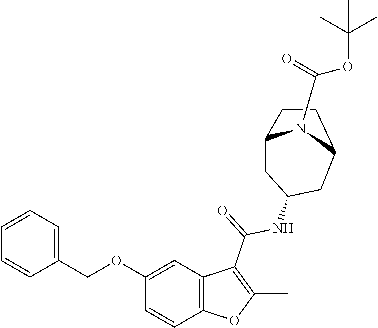 | Cpd 289 |
| 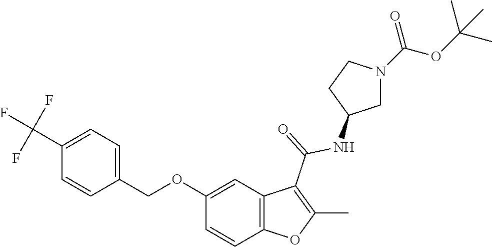 [SMILES: Cc1oc2ccc(OCc3ccc(C(F)(F)F)cc3)cc2c1C(=O)N[C@H]1CCN(C(=O)OC(C)(C)C)C1] | Cpd 290 |
| 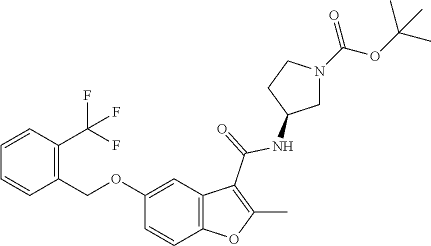 [SMILES: Cc1oc2ccc(OCc3ccccc3C(F)(F)F)cc2c1C(=O)N[C@H]1CCN(C(=O)OC(C)(C)C)C1] | Cpd 291 |
| 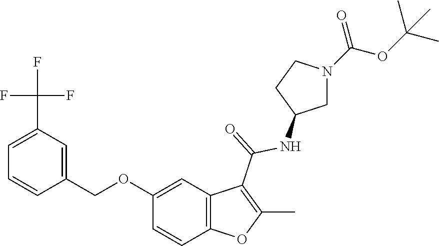 [SMILES: Cc1oc2ccc(OCc3cccc(C(F)(F)F)c3)cc2c1C(=O)N[C@H]1CCN(C(=O)OC(C)(C)C)C1] | Cpd 292 |
| 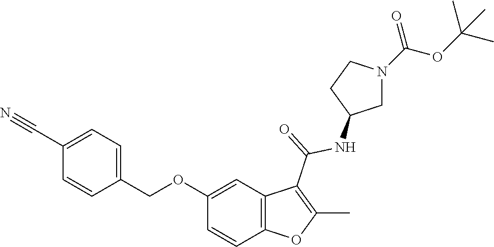 [SMILES: Cc1oc2ccc(OCc3ccc(C#N)cc3)cc2c1C(=O)N[C@H]1CCN(C(=O)OC(C)(C)C)C1] | Cpd 293 |
| 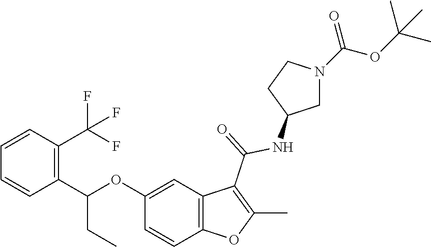 [SMILES: CCC(Oc1ccc2oc(C)c(C(=O)N[C@H]3CCN(C(=O)OC(C)(C)C)C3)c2c1)c1ccccc1C(F)(F)F] | Cpd 294 |
| 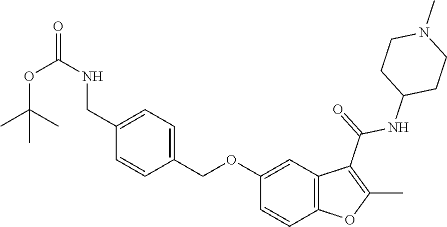 [SMILES: Cc1oc2ccc(OCc3ccc(CNC(=O)OC(C)(C)C)cc3)cc2c1C(=O)NC1CCN(C)CC1] | Cpd 295 |
| 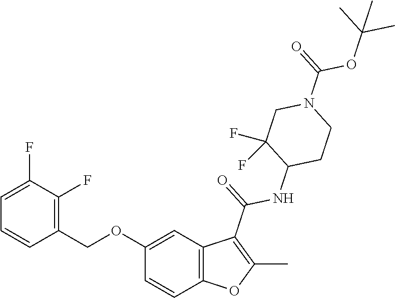 [SMILES: Cc1oc2ccc(OCc3cccc(F)c3F)cc2c1C(=O)NC1CCN(C(=O)OC(C)(C)C)CC1(F)F] | Cpd 296 |
| 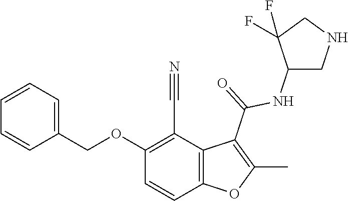 [SMILES: Cc1oc2ccc(OCc3ccccc3)c(C#N)c2c1C(=O)NC1CNCC1(F)F] | Cpd 297 |
| 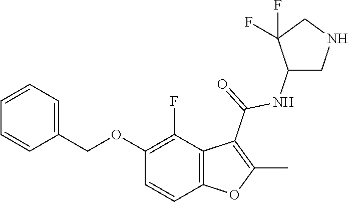 [SMILES: Cc1oc2ccc(OCc3ccccc3)c(F)c2c1C(=O)NC1CNCC1(F)F] | Cpd 298 |
| 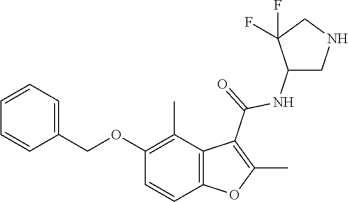 [SMILES: Cc1oc2ccc(OCc3ccccc3)c(C)c2c1C(=O)NC1CNCC1(F)F] | Cpd 299 |
| 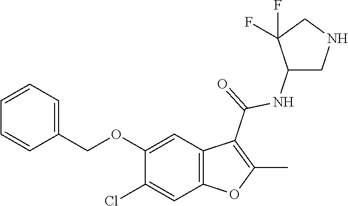 [SMILES: Cc1oc2cc(Cl)c(OCc3ccccc3)cc2c1C(=O)NC1CNCC1(F)F] | Cpd 300 |
| 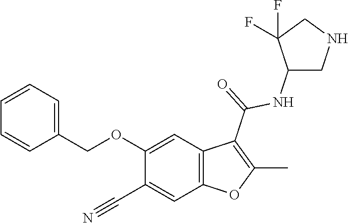 [SMILES: Cc1oc2cc(C#N)c(OCc3ccccc3)cc2c1C(=O)NC1CNCC1(F)F] | Cpd 301 |
| 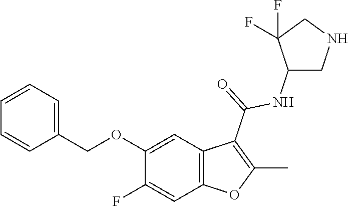 [SMILES: Cc1oc2cc(F)c(OCc3ccccc3)cc2c1C(=O)NC1CNCC1(F)F] | Cpd 302 |
| 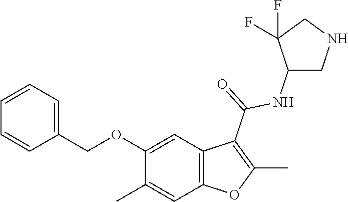 [SMILES: Cc1cc2oc(C)c(C(=O)NC3CNCC3(F)F)c2cc1OCc1ccccc1] | Cpd 303 |
| 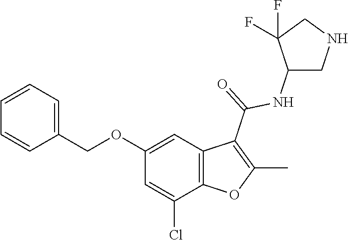 [SMILES: Cc1oc2c(Cl)cc(OCc3ccccc3)cc2c1C(=O)NC1CNCC1(F)F] | Cpd 304 |
| 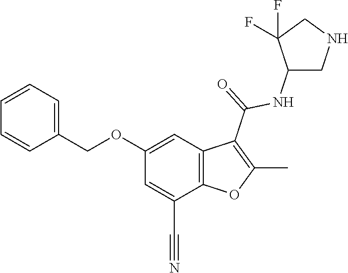 [SMILES: Cc1oc2c(C#N)cc(OCc3ccccc3)cc2c1C(=O)NC1CNCC1(F)F] | Cpd 305 |
| 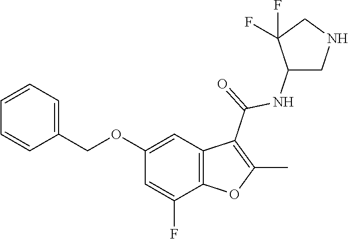 [SMILES: Cc1oc2c(F)cc(OCc3ccccc3)cc2c1C(=O)NC1CNCC1(F)F] | Cpd 306 |
| 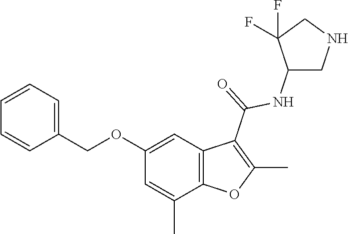 [SMILES: Cc1oc2c(C)cc(OCc3ccccc3)cc2c1C(=O)NC1CNCC1(F)F] | Cpd 307 |
| 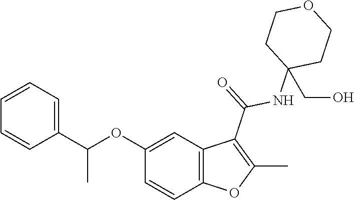 [SMILES: Cc1oc2ccc(OC(C)c3ccccc3)cc2c1C(=O)NC1(CO)CCOCC1] | Cpd 308 |
| 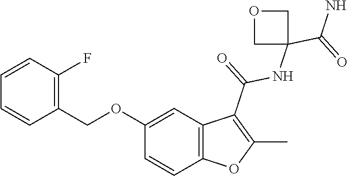 [SMILES: Cc1oc2ccc(OCc3ccccc3F)cc2c1C(=O)NC1(C(N)=O)COC1] | Cpd 309 |
| 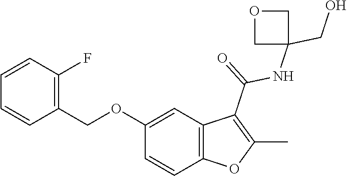 [SMILES: Cc1oc2ccc(OCc3ccccc3F)cc2c1C(=O)NC1(CO)COC1] | Cpd 310 |
| 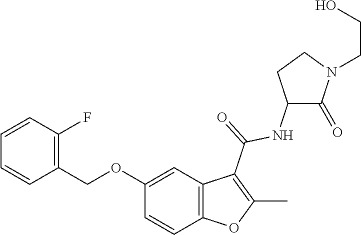 [SMILES: Cc1oc2ccc(OCc3ccccc3F)cc2c1C(=O)NC1CCN(CCO)C1=O] | Cpd 311 |
| 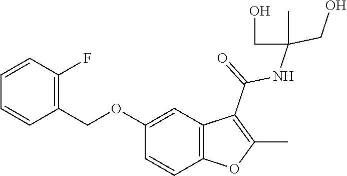 [SMILES: Cc1oc2ccc(OCc3ccccc3F)cc2c1C(=O)NC(C)(CO)CO] | Cpd 312 |
| 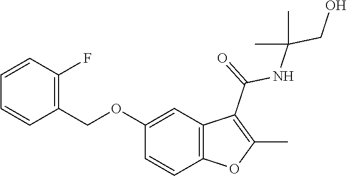 [SMILES: Cc1oc2ccc(OCc3ccccc3F)cc2c1C(=O)NC(C)(C)CO] | Cpd 313 |
| 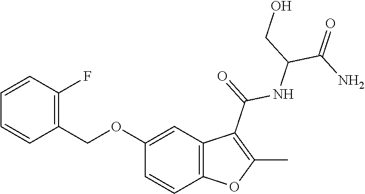 [SMILES: Cc1oc2ccc(OCc3ccccc3F)cc2c1C(=O)NC(CO)C(N)=O] | Cpd 314 |
| 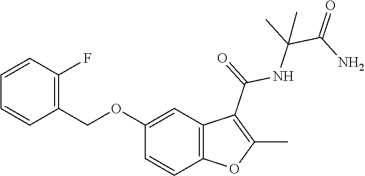 [SMILES: Cc1oc2ccc(OCc3ccccc3F)cc2c1C(=O)NC(C)(C)C(N)=O] | Cpd 315 |
| 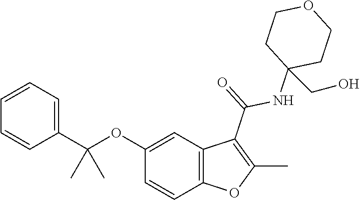 [SMILES: Cc1oc2ccc(OC(C)(C)c3ccccc3)cc2c1C(=O)NC1(CO)CCOCC1] | Cpd 316 |
| 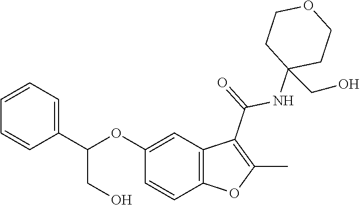 [SMILES: Cc1oc2ccc(OC(CO)c3ccccc3)cc2c1C(=O)NC1(CO)CCOCC1] | Cpd 317 |
| 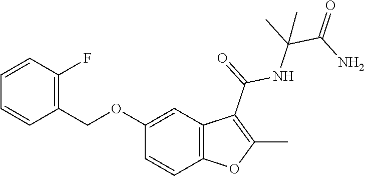 [SMILES: Cc1oc2ccc(OCc3ccccc3F)cc2c1C(=O)NC(C)(C)C(N)=O] | Cpd 318 |
| 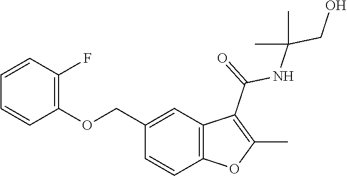 [SMILES: Cc1oc2ccc(COc3ccccc3F)cc2c1C(=O)NC(C)(C)CO] | Cpd 319 |
| 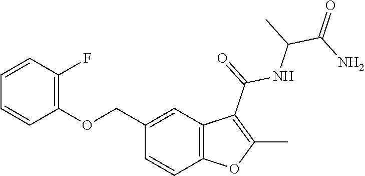 [SMILES: Cc1oc2ccc(COc3ccccc3F)cc2c1C(=O)NC(C)C(N)=O] | Cpd 320 |
| 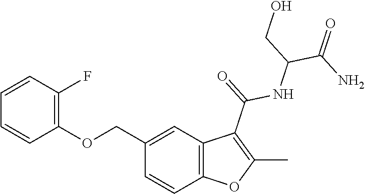 [SMILES: Cc1oc2ccc(COc3ccccc3F)cc2c1C(=O)NC(CO)C(N)=O] | Cpd 321 |
| 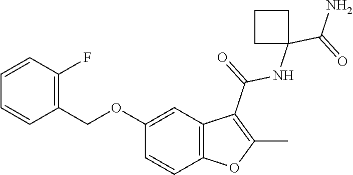 [SMILES: Cc1oc2ccc(OCc3ccccc3F)cc2c1C(=O)NC1(C(N)=O)CCC1] | Cpd 322 |
| 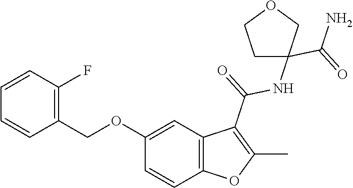 [SMILES: Cc1oc2ccc(OCc3ccccc3F)cc2c1C(=O)NC1(C(N)=O)CCOC1] | Cpd 323 |
| 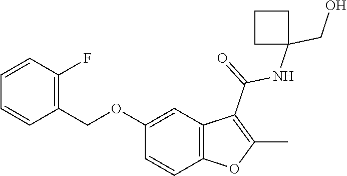 [SMILES: Cc1oc2ccc(OCc3ccccc3F)cc2c1C(=O)NC1(CO)CCC1] | Cpd 324 |
| 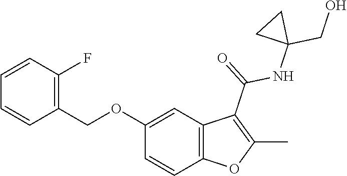 [SMILES: Cc1oc2ccc(OCc3ccccc3F)cc2c1C(=O)NC1(CO)CC1] | Cpd 325 |
| 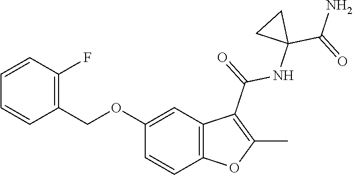 [SMILES: Cc1oc2ccc(OCc3ccccc3F)cc2c1C(=O)NC1(C(N)=O)CC1] | Cpd 326 |
| 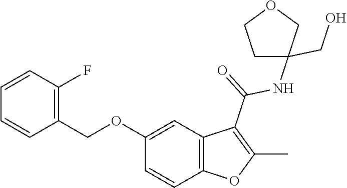 [SMILES: Cc1oc2ccc(OCc3ccccc3F)cc2c1C(=O)NC1(CO)CCOC1] | Cpd 327 |
| 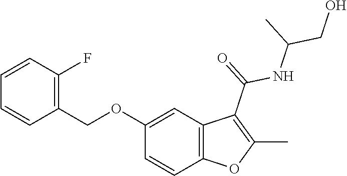 [SMILES: Cc1oc2ccc(OCc3ccccc3F)cc2c1C(=O)NC(C)CO] | Cpd 328 |
| 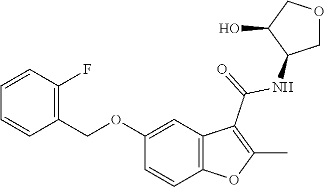 [SMILES: Cc1oc2ccc(OCc3ccccc3F)cc2c1C(=O)N[C@@H]1COC[C@@H]1O] | Cpd 329 |
| 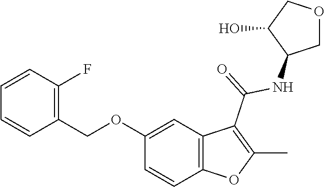 [SMILES: Cc1oc2ccc(OCc3ccccc3F)cc2c1C(=O)N[C@@H]1COC[C@H]1O] | Cpd 330 |
| 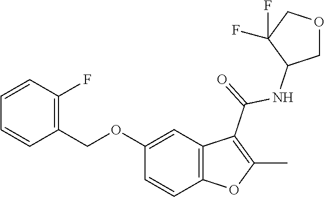 [SMILES: Cc1oc2ccc(OCc3ccccc3F)cc2c1C(=O)NC1COCC1(F)F] | Cpd 331 |
| 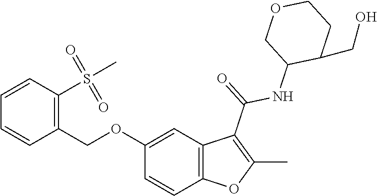 [SMILES: Cc1oc2ccc(OCc3ccccc3S(C)(=O)=O)cc2c1C(=O)NC1COCCC1CO] | Cpd 332 |
| 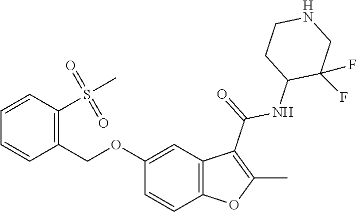 [SMILES: Cc1oc2ccc(OCc3ccccc3S(C)(=O)=O)cc2c1C(=O)NC1CCNCC1(F)F] | Cpd 333 |
| 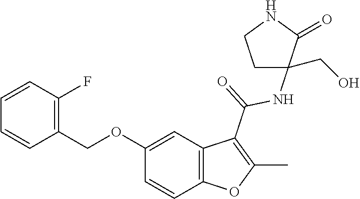 [SMILES: Cc1oc2ccc(OCc3ccccc3F)cc2c1C(=O)NC1(CO)CCNC1=O] | Cpd 334 |
| 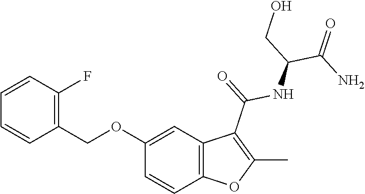 [SMILES: Cc1oc2ccc(OCc3ccccc3F)cc2c1C(=O)N[C@@H](CO)C(N)=O] | Cpd 335 |
| 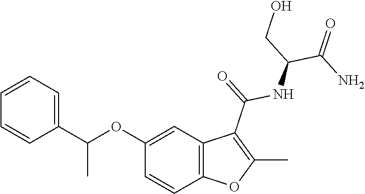 [SMILES: Cc1oc2ccc(OC(C)c3ccccc3)cc2c1C(=O)N[C@@H](CO)C(N)=O] | Cpd 336 |
| 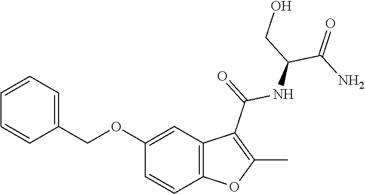 [SMILES: Cc1oc2ccc(OCc3ccccc3)cc2c1C(=O)N[C@@H](CO)C(N)=O] | Cpd 337 |
| 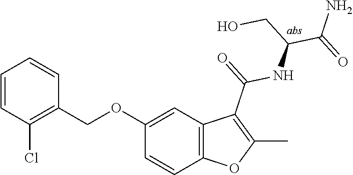 [SMILES: Cc1oc2ccc(OCc3ccccc3Cl)cc2c1C(=O)N[C@@H](CO)C(N)=O] | Cpd 338 |
| 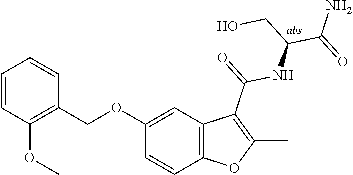 [SMILES: COc1ccccc1COc1ccc2oc(C)c(C(=O)N[C@@H](CO)C(N)=O)c2c1] | Cpd 339 |
| 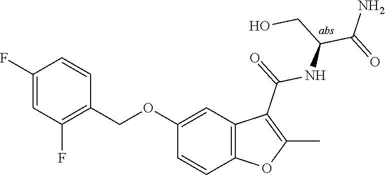 [SMILES: Cc1oc2ccc(OCc3ccc(F)cc3F)cc2c1C(=O)N[C@@H](CO)C(N)=O] | Cpd 340 |
| 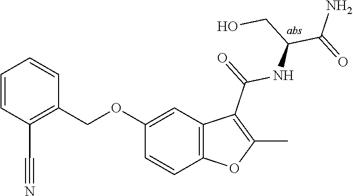 [SMILES: Cc1oc2ccc(OCc3ccccc3C#N)cc2c1C(=O)N[C@@H](CO)C(N)=O] | Cpd 341 |
| 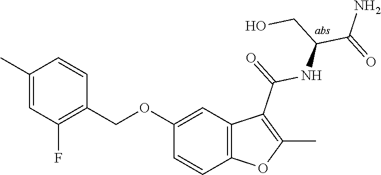 [SMILES: Cc1ccc(COc2ccc3oc(C)c(C(=O)N[C@@H](CO)C(N)=O)c3c2)c(F)c1] | Cpd 342 |
| 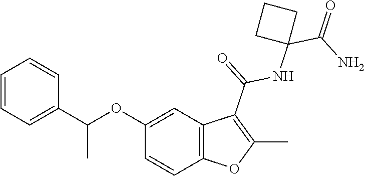 [SMILES: Cc1oc2ccc(OC(C)c3ccccc3)cc2c1C(=O)NC1(C(N)=O)CCC1] | Cpd 343 |
| 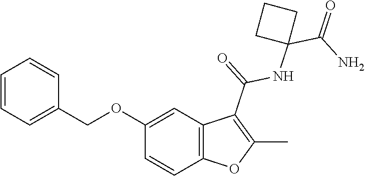 [SMILES: Cc1oc2ccc(OCc3ccccc3)cc2c1C(=O)NC1(C(N)=O)CCC1] | Cpd 344 |
| 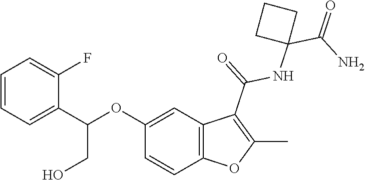 [SMILES: Cc1oc2ccc(OC(CO)c3ccccc3F)cc2c1C(=O)NC1(C(N)=O)CCC1] | Cpd 345 |
| 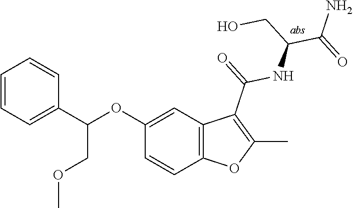 [SMILES: COCC(Oc1ccc2oc(C)c(C(=O)N[C@@H](CO)C(N)=O)c2c1)c1ccccc1] | Cpd 346 |
| 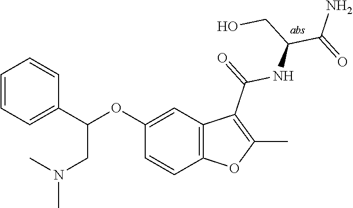 [SMILES: Cc1oc2ccc(OC(CN(C)C)c3ccccc3)cc2c1C(=O)N[C@@H](CO)C(N)=O] | Cpd 347 |

The examples depicted above are provided for the purpose of illustrating the present invention and by no means should be interpreted to limit the scope of the present invention.

Part A represents the preparation of the compounds whereas Part B represents the pharmacological examples.

Part A

All starting materials which are not explicitly described were either commercially available (the details of suppliers such as for example Acros, Avocado, Aldrich, Fluka, FluoroChem, MatrixScientific, Maybridge, Merck, Sigma, etc. can be found in the SciFinder® Database for example) or the synthesis thereof has already been described precisely in the specialist literature (experimental guidelines can be found in the Reaxys® Database or the SciFinder® Database respectively, for example) or can be prepared using the conventional methods known to the person skilled in the art.

The reactions were, if necessary, carried out under an inert atmosphere (mostly argon and N₂). The number of equivalents of reagents and the amounts of solvents employed as well as the reaction temperatures and times can vary slightly between different reactions carried out by analogous methods. The work-up and purification methods were adapted according to the characteristic properties of each compound and can vary slightly for analogous methods. The yields of the compounds prepared are not optimized.

The indication “equivalents” (“eq.” or “eq” or “equiv.”) means molar equivalents, “RT” or “rt” means room temperature T (23±7° C.), “M” are indications of concentration in mol/l, “sol.” means solution, “conc.” means concentrated. The mixing ratios of solvents are usually stated in the volume/volume ratio.

Key analytical characterization was carried out by means of ¹H-NMR spectroscopy and/or mass spectrometry (MS, m/z for [M+H]⁺ and/or for [M−H]⁻) for all the exemplary compounds and selected intermediate products. In certain cases, where e.g., regioisomers and/or diastereomers could be/were formed, additional analytics, such as, e.g., ¹³C NMR and NOE (nuclear overhauser effect) NMR experiments were in some cases performed.

Analytical instruments employed were e.g., for NMR analysis a BRUKER 400 MHz or a BRUKER 500 MHz machine (Software Topspin), alternatively a BRUKER AVANCE 300 MHz and 400 Mhz was employed. For LC/MS analysis e.g., an Agilent 1290 infinity, Mass: 6150 SQD(ESI/APCI) or an Agilent 1200 SERIES, Mass: 6130 SQD(ESI/APCI) (Software Chemistation) was employed. Analytical HPLCs were measured e.g., on Waters (Software Empower), an Agilent-1200-ELSD (Software Chemistation) or an Agilent-1260 (Software OpenLAB). Analytical SFC were performed e.g., on a PIC solution (Software: SFC PICLAB ONLINE), a WAFERS-X5 (Software MASSLYNX) or a WA IERS-UPC2 (Empower).

Preparative HPLC were performed e.g., on a Waters 2998 (Software Empower) or a YMC (Software K-Prep). Preparative SFC were performed e.g., on a Waters, SFC-200 (Software Chromscope or Super chrome), a Waters, SFC-80 (Super chrome) or a PIC, PIC-175 (Software S10-100).

Structures of example compounds that contain stereocenters are drawn and named with absolute stereochemistry, if known. In case of unknown absolute stereochemistry, the compounds can be either racemic, a mixture of diastereomers, a pure diastereomer of unknown stereochemistry, or a pure enantiomer of unknown stereochemistry. Dia 1 and Dia 2 means that diastereoisomers were separated but the stereochemistry is unknown. En 1 and En 2 means that both enantiomers were separated but the absolute configuration is unknown. No suffix given after the compound code means that a compound containing stereocenters was obtained as a racemic mixture or a mixture of diastereomers, respectively, unless the chemical name of the compound specifies the exact stereochemistry.

The LC/MS analysis were also performed on a Dionex Ultimate 3000 HPLC system (equipped with a PDA detector) connected to a mass spectrometer Brucker Esquire 6000 (equipped with a multimode source, ESI/APCI) (Method L in the table below). Or the LC/MS analysis mentioned in the experimental part were performed on a Waters system combining an Acquity UPLC H-Class equipped with a Acquity UPLC PDA Detector and an Acquity TQ Detector (ESI) (Method U in the table below).

The separations were e.g., performed with a SunFireC18, 3.5 μm 3.0×100 mm, column equipped with a SunFire C18, 3.5 μm, 3.0×20 mm Guard column or a X-Bridge C18 100×3.0 mm column equipped with a X-Bridge C18, 3.5 μm, 3.0×20 mm Guard column thermostated to 30° C. and the DAD acquisition wavelength was set in the range of 190-420 nm (Method L in the table below). Or the separations were performed with an Acquity UPLC HSS C18, 2.1×50 mm, 1.8 μM column equipped with a prefilter and thermostated at 40° C. or an Acquity UPLC BEH C18, 2.1×50 mm, 1.7 μM column equipped with a prefilter and thermostated at 40° C. and the PAD acquisition wavelength was set in the range of 210-420 nm (Method U in the table below). Elutions were carried out with the methods described in the following tables.

| | | | Solvents | | | Flow | |
|---|---|---|---|---|---|---|---|
| LC/MS | | Time | A | B | C | (mL/ | |
| Method | System | (min) | (%) | (%) | (%) | min) | Column |
| L1 | Dionex Ultimate | 0 | 80 | — | 20 | 1 | SunFire C18 |
| | 3000 HPLC | 0.2 | 80 | — | 20 | 1 | |
| | | 7 | 40 | — | 60 | 1 | |
| | | 8 | 10 | — | 90 | 1 | |
| | | 10.8 | 10 | — | 90 | 1 | |
| | | 11 | 80 | — | 20 | 1 | |
| | | 14 | 80 | — | 20 | 1 | |
| L2 | Dionex Ultimate | 0 | 50 | — | 50 | 1 | SunFire C18 |
| | 3000 HPLC | 0.2 | 50 | — | 50 | 1 | |
| | | 6 | 10 | — | 90 | 1 | |
| | | 10.8 | 10 | — | 90 | 1 | |
| | | 11 | 50 | — | 50 | 1 | |
| | | 14 | 50 | — | 50 | 1 | |
| L3 | Dionex Ultimate | 0 | — | 80 | 20 | 1 | X-Bridge C18 |
| | 3000 HPLC | 0.2 | — | 80 | 20 | 1 | |
| | | 7 | — | 40 | 60 | 1 | |
| | | 8 | — | 10 | 90 | 1 | |
| | | 10.8 | — | 10 | 90 | 1 | |
| | | 11 | — | 80 | 20 | 1 | |
| | | 14 | — | 80 | 20 | 1 | |
| L4 | Dionex Ultimate | 0 | — | 50 | 50 | 1 | X-Bridge C18 |
| | 3000 HPLC | 0.2 | — | 50 | 50 | 1 | |
| | | 6 | — | 10 | 90 | 1 | |
| | | 10.8 | — | 10 | 90 | 1 | |
| | | 11 | — | 50 | 50 | 1 | |
| | | 14 | — | 50 | 50 | 1 | |
| L5 | Dionex Ultimate | 0 | 95 | — | 5 | 1 | SunFire C18 |
| | 3000 HPLC | 1 | 95 | — | 5 | 1 | |
| | | 7 | 50 | — | 50 | 1 | |
| | | 8 | 10 | — | 90 | 1 | |
| | | 10.8 | 10 | — | 90 | 1 | |
| | | 11 | 95 | — | 5 | 1 | |
| | | 14 | 95 | — | 5 | 1 | |
| L6 | Dionex Ultimate | 0 | — | 95 | 5 | 1 | X-Bridge C18 |
| | 3000 HPLC | 1 | — | 95 | 5 | 1 | |
| | | 7 | — | 50 | 50 | 1 | |
| | | 8 | — | 10 | 90 | 1 | |
| | | 10.8 | — | 10 | 90 | 1 | |
| | | 11 | — | 95 | 5 | 1 | |
| | | 14 | — | 95 | 5 | 1 | |

Solvent A:: Formic Acid LC-MS grade 0.1% in milliQ water
Solvent B: NH4OAc (LC-MS grade) 10 mMol in milliQ water, adjusted at pH10 with an aqueous solution of NH3, LC-MS grade
Solvent C: Acetonitrile LC-MS grade

| | | | Solvents | | | | |
|---|---|---|---|---|---|---|---|
| LC/MS | | Time | A | B | C | Flow | |
| Method | System | (min) | (%) | (%) | (%) | (mL/min) | Column |
| U1 | Waters | 0 | 95 | — | 5 | 0.5 | Acquity UPLC |
| | UPLC | 3.18 | 50 | — | 50 | 0.5 | HSS C18 |
| | | 4 | 10 | — | 90 | 0.5 | |
| | | 5 | 10 | — | 90 | 0.5 | |
| U2 | Waters | 0 | 80 | — | 20 | 0.5 | Acquity UPLC |
| | UPLC | 3.4 | 40 | — | 60 | 0.5 | HSS C18 |
| | | 4 | 10 | — | 90 | 0.5 | |
| | | 5 | 10 | — | 90 | 0.5 | |
| U3 | Waters | 0 | 50 | — | 50 | 0.5 | Acquity UPLC |
| | UPLC | 3.5 | 10 | — | 90 | 0.5 | HSS C18 |
| | | 5 | 10 | — | 90 | 0.5 | |
| U4 | Waters | 0 | — | 95 | 5 | 0.5 | Acquity UPLC |
| | UPLC | 3.18 | — | 50 | 50 | 0.5 | BEH C18 |
| | | 4 | — | 10 | 90 | 0.5 | |
| | | 5 | — | 10 | 90 | 0.5 | |
| U5 | Waters | 0 | — | 80 | 20 | 0.5 | Acquity UPLC |
| | UPLC | 3.4 | — | 40 | 60 | 0.5 | BEH C18 |
| | | 4 | — | 10 | 90 | 0.5 | |
| | | 5 | — | 10 | 90 | 0.5 | |
| U6 | Waters | 0 | — | 50 | 50 | 0.5 | Acquity UPLC |
| | UPLC | 3.5 | — | 10 | 90 | 0.5 | BEH C18 |
| | | 5 | — | 10 | 90 | 0.5 | |

Solvent A:: Formic Acid LC-MS grade 0.1% in milliQ water
Solvent B: NH4OAc (LC-MS grade) 10 mMol in milliQ water, adjusted at pH10 with an aqueous solution of NH3, LC-MS grade
Solvent C: Acetonitrile LC-MS grade

Preparative HPLC purifications have also been carried out with the following system: a Waters 2489 UV/Visible Detector, a Waters 2545 Binary Gradient Module, a Waters Fraction Collector III and a Waters Dual Flex Injector.

The separations were performed with a X-Bridge Prep C18 column, 100×19 mm, 5 μm column equipped with a X-Bridge C18, 19×10 mm, 5 μm Guard column or with a SunFire Prep C18 ODB column (5 jam; 19×100 mm) equipped with a SunFire C18 guard column (5 μm; 19×10 mm).

Elutions were carried out with the methods described in the following tables, and detection wavelengths were fixed at 210 and 254 nm.

| HPLC | Time | Solvent | | Flow | Column |
|---|---|---|---|---|---|
| Method | (min) | A (%) | B (%) | (mL/min) | |
| H1 | 0 | 80 | 20 | 20 | X-Bridge |
| | 2 | 80 | 20 | 20 | Prep C18 |
| | 8 | 10 | 90 | 20 | |
| | 10.8 | 10 | 90 | 20 | |
| | 11 | 80 | 20 | 20 | |
| | 16 | 80 | 20 | 20 | |
| H2 | 0 | 95 | 5 | 20 | SunFire |
| | 2 | 95 | 5 | 20 | Prep C18 |
| | 8 | 50 | 50 | 20 | ODB |
| | 9 | 10 | 90 | 20 | |
| | 13 | 10 | 90 | 20 | |
| | 14 | 95 | 5 | 20 | |
| | 16 | 95 | 5 | 20 | |

Solvent A: Ammonium Acetate puriss p.a. for HPLC 10 mM in milliQ water, adjusted at pH10 with Ammonium Hydroxyde puriss p.a. for HPLC
Solvent B: Acetonitrile HPLC grade.

### Synthesis of methyl 2-ethyl-5-((3-fluorobenzyl)oxy)benzofuran-3-carboxylate (Cpd 030) and 2-ethyl-5-((3-fluorobenzyl)oxy)benzofuran-3-carboxylic Acid (Cpd 015)

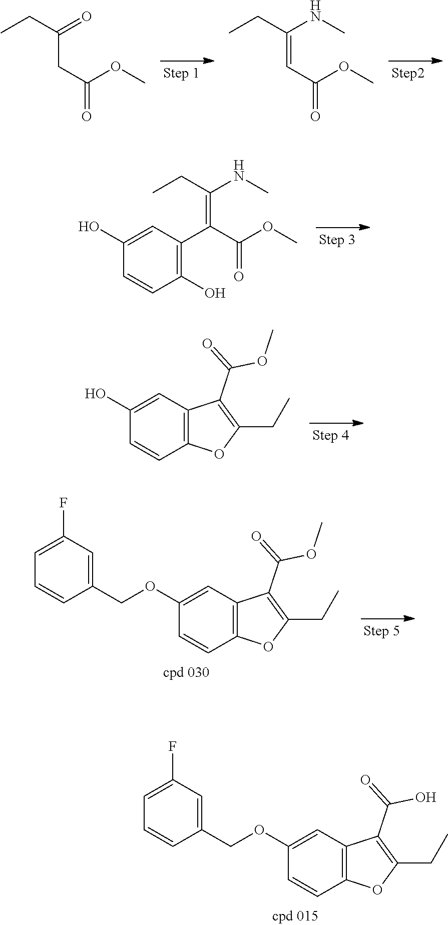

Step 1: Methylamine (2 M in THF; 2.9 mL) was added dropwise at RT to a solution of methyl 3-oxopentanoate (1.3 M in MeOH; 2.9 mL) and the RM was stirred for 3 h at RT. The volatiles were removed under reduced pressure to afford 550 mg (quantitative yield) of methyl 3-(methylamino)pent-2-enoate which was used in the next step without further purification.

Step 2: A solution of methyl 3-(methylamino)pent-2-enoate (1.3 M in DCM; 7.7 mL) was added dropwise over 10 min to a cold (−45° C.) mixture of TFA (0.1 mL; 1.31 mmol) and p-benzoquinone (1.08 g; 9.99 mmol) in DCM (7.5 mL). The RM was stirred for 6 h at −30° C. and then kept without stirring at −25° C. overnight. The RM was allowed to warm to RT and the volatiles were removed under reduced pressure to afford 2.14 g (90%) of the desired methyl 2-(2,5-dihydroxyphenyl)-3-(methylamino)pent-2-enoate which was used in the next step without further purification.

Step 3: TFA (1 mL; 13.06 mmol) was added to a suspension of methyl 2-(2,5-dihydroxyphenyl)-3-(methylamino)pent-2-enoate (0.55 g; 2.19 mmol) in toluene (25 mL). The RM was stirred at 85° C. for 7 h and was allowed to stir at RT overnight. Then, the volatiles were removed under reduced pressure and the residue was purified by FCC on silica gel using a gradient of EtOAc (0% to 100%) in heptane to afford 0.482 g (22%) of methyl 2-ethyl-5-hydroxybenzofuran-3-carboxylate as a beige solid. ¹H NMR (CDCl³, 300 MHz): δ ppm 9.36 (s, 1H); 7.40 (d, 1H); 7.26 (d, 1H); 6.75 (dd, 1H); 3.87 (s, 3H); 3.12 (q, 2H); 1.26 (t, 3H).

Step 4: Cesium carbonate (323 mg; 0.990 mmol) was added to a solution of methyl 2-ethyl-5-hydroxybenzofuran-3-carboxylate (109 mg; 0.495 mmol) in THF (3 mL). The RM was stirred for 25 min. at RT. 1-(Bromomethyl)-3-fluorobenzene (0.091 mL; 0.742 mmol) was then added and the solution was stirred for 18 h at 95° C. The mixture was cooled down to RT, diluted with EtOAc and washed two times with water and with brine. The organic layer was dried over magnesium sulfate and filtered. The solvent was removed under reduced pressure and the resulting oil was purified by FCC on silica gel using a gradient of EtOAc (0% to 100%) in heptane to afford 110 mg (54%) of methyl 2-ethyl-5-((3-fluorobenzyl)oxy)benzofuran-3-carboxylate (cpd 030).

Step 5: In a sealed tube, KOH (75 mg; 1.34 mmol.) was added to a solution of methyl 2-ethyl-5-((3-fluorobenzyl)oxy)benzofuran-3-carboxylate (cpd 030) (110 mg; 0.335 mmol) in a mixture of water/EtOH/MeOH/THF (6/3/3/1; 3.25 mL) and the RM was stirred overnight at 85° C. The RM was cooled down to RT, diluted with water, washed with EtOAc, acidified with a 1N HCl solution and extracted with EtOAc. The resulting organic layer was washed with water and brine, dried over magnesium sulfate and filtered. The solvent was removed under reduced pressure. The compound was purified by SPE on C18 gel using a gradient of acetonitrile (0% to 80%) in water to afford 49.8 mg (46%) of 2-ethyl-5-((3-fluorobenzyl)oxy)benzofuran-3-carboxylic acid (cpd 015).

Cpd 007, cpd 020 and cpd 023 were prepared in a manner similar (use of appropriate reagents and purification methods known to the person skilled in the art) to cpd 030 and cpd 015.

### Synthesis of ethyl 5-((2-chloro-6-fluorobenzyl)oxy)-2-methylbenzofuran-3-carboxylate (Cpd 033) and 5-((2-chloro-6-fluorobenzyl)oxy)-2-methylbenzofuran-3-carboxylic Acid (Cpd 021)

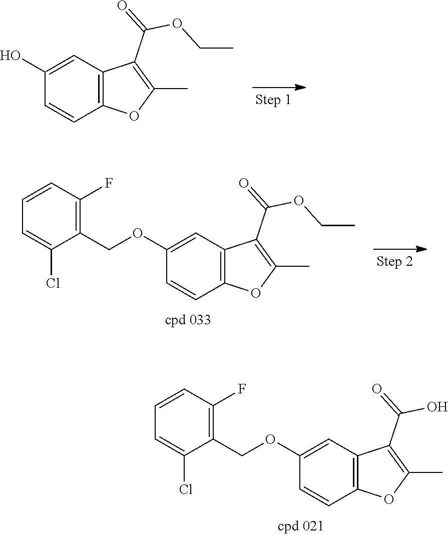 [SMILES: CCOC(=O)c1c(C)oc2ccc(O)cc12 ; CCOC(=O)c1c(C)oc2ccc(OCc3c(F)cccc3Cl)cc12 ; Cc1oc2ccc(OCc3c(F)cccc3Cl)cc2c1C(=O)O]

Step 1: To a solution of ethyl 5-hydroxy-2-methylbenzofuran-3-carboxylate (0.220 g; 1 mmol) in THF (8 mL) was added cesium carbonate (0.652 g; 2 mmol) and the RM was stirred at RT for 10 min. Then, 2-(bromomethyl)-1-chloro-3-fluorobenzene (0.338 mL; 2.5 mmol) was added and the stirred solution was heated at 95° C. until the consumption of 5-hydroxy-2-methylbenzofuran-3-carboxylate. The mixture was cooled down to RT, poured in water and extracted twice with EtOAc. The combined organic layers were dried over sodium sulfate, filtered and concentrated under reduced pressure. The residue was purified by FCC on silica gel using a gradient of DCM (0% to 100%) in heptane to afford 0.282 g (78%) of ethyl 5-((2-chloro-6-fluorobenzyl)oxy)-2-methylbenzofuran-3-carboxylate (cpd 033).

Step 2: To a solution of ethyl 5-((2-chloro-6-fluorobenzyl)oxy)-2-methylbenzofuran-3-carboxylate (cpd 033) (0.282 g; 0.777 mmol) in a mixture of MeOH-EtOH (2:1, 23 mL) was added an aq. solution of sodium hydroxide (1 N; 23 mL) and the RM was heated under reflux until the consumption of ethyl 5-((2-chloro-6-fluorobenzyl)oxy)-2-methylbenzofuran-3-carboxylate (cpd 033). After cooling, the volatiles were removed under reduced pressure and the remaining residue was dissolved in water. The mixture was acidified with an aq. solution of hydrochloric acid (6 N) until pH-2. The white precipitate was washed with water and dried under reduced pressure to afford 0.259 g (99%) of 5-((2-chloro-6-fluorobenzyl)oxy)-2-methylbenzofuran-3-carboxylic acid (cpd 21).

Cpd 001, cpd 004, cpd 005, cpd 006, cpd 009, cpd 010, cpd 012, cpd 024, cpd 025, cpd 026 and cpd 028 were prepared in a manner similar (use of appropriate reagents and purification methods known to the person skilled in the art) to cpd 33 and cpd 21.

### Synthesis of ethyl 5-((2,3-dihydro-1H-inden-1-yl)oxy)-2-methylbenzofuran-3-carboxylate (Cpd 032) and 5-((2,3-dihydro-1H-inden-1-yl)oxy)-2-methylbenzofuran-3-carboxylic Acid (Cpd 011)

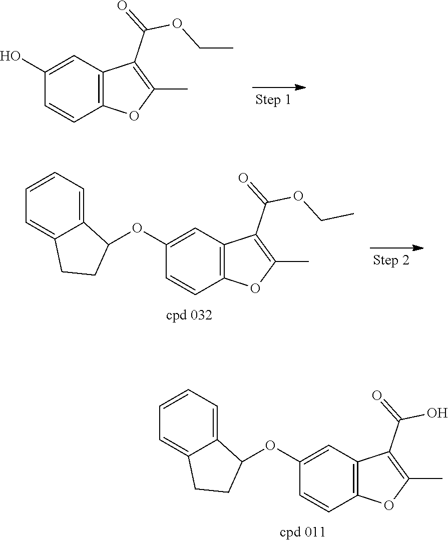 [SMILES: CCOC(=O)c1c(C)oc2ccc(O)cc12 ; CCOC(=O)c1c(C)oc2ccc(OC3CCc4ccccc43)cc12 ; Cc1oc2ccc(OC3CCc4ccccc43)cc2c1C(=O)O]

Step 1: Ethyl 5-hydroxy-2-methylbenzofuran-3-carboxylate (100 mg; 0.454 mmol), 2,3-dihydro-1H-inden-1-ol (73.1 mg; 0.545 mmol) and PPh₃ (119 mg; 0.454 mmol) were dissolved in toluene (5 mL) and cooled down to 0° C. A solution of DEAD (2 M in THF; 1.2 eq.) was then added dropwise and the RM was stirred 18 h at RT. The volatiles were removed under reduced pressure and the residue was diluted with DCM and washed successively with an aq. solution of NaOH (1 N), water and brine. The organic layer was dried over magnesium sulfate, filtered and the solvent was removed under reduced pressure. The compound was purified by SPE on C18 gel using a gradient of acetonitrile (10% to 80%) in water to afford 130 mg (77%) of ethyl 5-((2,3-dihydro-1H-inden-1-yl)oxy)-2-methylbenzofuran-3-carboxylate (cpd 032).

Step 2: An aq. solution of NaOH (1 N; 7.83 mL; 40 mmol) was added to a solution of ethyl 5-((2,3-dihydro-1H-inden-1-yl)oxy)-2-methylbenzofuran-3-carboxylate (cpd 032) (130 mg; 0.386 mmol) in MeOH (2 mL) and the mixture was stirred for 4 h at 80° C. After cooling, the mixture was diluted with water and washed with DCM. The aq. layer was acidified till pH=1 with an aq. solution of HCl (6 N) and extracted with EtOAc. The resulting organic layer was dried over magnesium sulfate, filtered and the solvent was removed under reduced pressure. The crude was purified by SPE on C18 gel using a gradient of acetonitrile (10% to 80%) in water to afford 26.6 mg (21%) of 5-((2,3-dihydro-1H-inden-1-yl)oxy)-2-methylbenzofuran-3-carboxylic acid (cpd 011).

Cpd 013 and cpd 014 were prepared in a manner similar (use of appropriate reagents and purification methods known to the person skilled in the art) to cpd 32 and cpd 11.

### Synthesis of tert-butyl 3-(5-(benzyloxy)-2-methylbenzofuran-3-carboxamido)piperidine-1-carboxylate (Cpd 247)

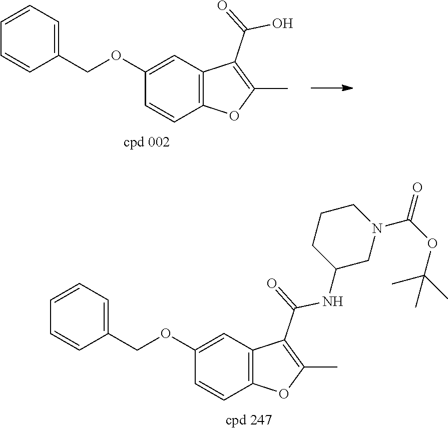 [SMILES: Cc1oc2ccc(OCc3ccccc3)cc2c1C(=O)O ; Cc1oc2ccc(OCc3ccccc3)cc2c1C(=O)NC1CCCN(C(=O)OC(C)(C)C)C1]

Step 1: HATU (121 mg; 0.319 mmol) was added to a solution of 5-(benzyloxy)-2-methylbenzofuran-3-carboxylic acid (cpd 002) (60 mg; 0.213 mmol) and DIPEA (82 mg; 0.638 mmol) in DCM (3 mL). After 3 h at RT, tert-butyl 3-aminopiperidine-1-carboxylate (53.2 mg; 0.266 mmol) was added and the RM was stirred overnight. The RM was then diluted with DCM and washed successively with a sat. solution of sodium hydrogen carbonate and brine. The organic layer was dried over magnesium sulfate and filtered. The volatiles were removed under reduced pressure and the crude material was purified by FCC on silica gel using a gradient of EtOAc (10% to 100%) in heptane to afford 86.0 mg (84%) of tert-butyl 3-(5-(benzyloxy)-2-methylbenzofuran-3-carboxamido)piperidine-1-carboxylate (cpd 247).

The following compounds were prepared in a manner similar (use of appropriate reagents and purification methods known to the person skilled in the art) to cpd 247:
Cpd 035, 037, 038, 039, 040, 041, 043, 044, 045, 046, 047, 050, 051, 052, 053, 054, 055, 056, 057, 060, 064, 065, 066, 067, 068, 071, 072, 076, 079, 080, 081, 086, 087, 088, 089, 090, 091, 092, 098, 099, 100, 101, 102, 104, 105, 106, 109, 111, 117, 118, 122, 124, 125, 128, 132, 134, 138, 139, 142, 144, 148, 149, 150, 151, 152, 153, 154, 156, 165, 166, 168, 171, 172, 179, 182, 183, 185, 186, 187, 189, 194, 196, 197, 198, 200, 205, 106, 107, 210, 212, 213, 214, 217, 218, 221, 222, 225, 228, 229, 230, 232, 233, 234, 235, 236, 237, 238, 239, 241, 243, 244, 248, 249, 251, 252, 253, 258, 262, 264, 265, 266, 268, 269, 270, 271, 272, 273, 274, 275, 276, 277, 278, 279, 280, 281, 309, 310, 312, 313, 315, 326, 328—En 1 (R), 328—En 2 (S) and 337.

### Synthesis of tert-butyl ((1-(5-(benzyloxy)-2-methylbenzofuran-3-carboxamido)cyclobutyl)methyl)carbamate (Cpd 246)

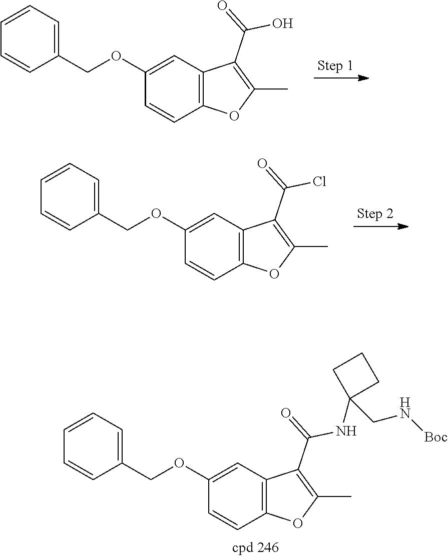 [SMILES: Cc1oc2ccc(OCc3ccccc3)cc2c1C(=O)O ; Cc1oc2ccc(OCc3ccccc3)cc2c1C(=O)Cl ; Cc1oc2ccc(OCc3ccccc3)cc2c1C(=O)NC1(CNC(=O)OC(C)(C)C)CCC1]

Step 1: To a solution of 5-(benzyloxy)-2-methylbenzofuran-3-carboxylic acid (450 mg; 1.59 mmol) in toluene (30 mL) was added thionyl chloride (0.578 mL; 7.97 mmol) at RT. The RM was refluxed overnight, cooled to room temperature, and concentrated under reduced pressure to provide the desired acid chloride which was used without further purification.

Step 2: 5-(benzyloxy)-2-methylbenzofuran-3-carbonyl chloride (200 mg; 0.66 mmol) in DCM (2 mL) was added to a solution of tert-butyl ((1-aminocyclobutyl)methyl)carbamate (160 mg; 0.8 mmol) and DIPEA (0.5 mL; 2.7 mmol) in DCM (2 mL). The RM was stirred 36 h at RT. The RM was concentrated under reduced pressure. The residue was purified by FCC on silica gel using a gradient of EtOAc (4% to 40%) in heptane to afford 208 mg (67%) of tert-butyl(0-(5-(benzyloxy)-2-methylbenzofuran-3-carboxamido)cyclobutyl)-methyl)carbamate (cpd 246).

The following compounds were prepared in a manner similar (use of appropriate reagents and purification methods known to the person skilled in the art) to cpd 246:
Cpd 036, 058, 059, 061, 062, 069, 070, 107, 108, 110, 112, 112, 123, 129, 146, 155, 159, 164, 167, 174, 178, 181, 208, 209, 216, 219, 254, 259 and 263, 282, 283, 284, 285, 286, 287, 288, 289, 290, 291 and 292.

### Synthesis of tert-butyl (S)-3-(5-((2,3-difluorobenzyl)oxy)-2-methylbenzofuran-3-carboxamido)pyrrolidine-1-carboxylate (Cpd 256)

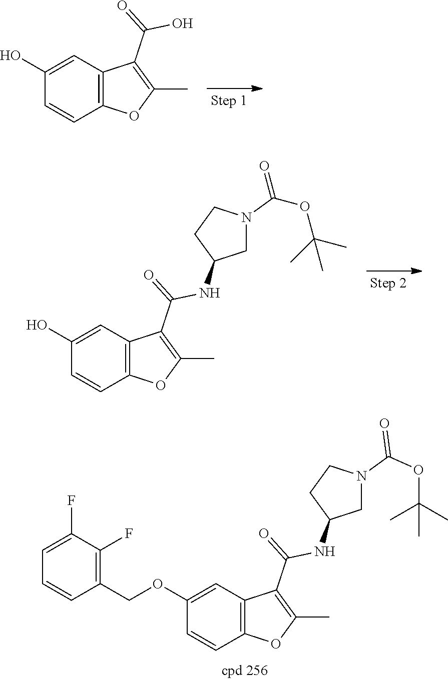 [SMILES: Cc1oc2ccc(O)cc2c1C(=O)O ; Cc1oc2ccc(O)cc2c1C(=O)N[C@H]1CCN(C(=O)OC(C)(C)C)C1 ; Cc1oc2ccc(OCc3cccc(F)c3F)cc2c1C(=O)N[C@H]1CCN(C(=O)OC(C)(C)C)C1]

Step 1: Tert-butyl (S)-3-aminopyrrolidine-1-carboxylate (1.1 g; 5.7 mmol) was added to a solution of 5-hydroxy-2-methylbenzofuran-3-carboxylic acid (1 g; 5.2 mmol), HATU (1.98 g; 5.2 mmol) and DIPEA (2.7 mL; mmol) in DMF (10 mL). After 60 h, the reaction was concentrated under reduced pressure. The residue was partitioned between water and EtOAc. After separation, the aq. layer was extracted twice with EtOAc. Combined EtOAc extracts were dried over magnesium sulphate, filtered, and concentrated under reduced pressure. The residue was purified by FCC on silica gel using a gradient of MeOH (0 to 6%) in DCM to afford 0.94 g (50%) of the desired compound as a white solid. M/Z(+): 361 (M+H). M/Z(−): 359 (M−H). ¹H NMR (DMSO-d₆, 300 MHz) δ ppm: 9.26 (s, 1H), 8.17 (d, J=6.4 Hz, 1H), 7.32 (d, J=9.0 Hz, 1H), 6.99 (d, J=1.88 Hz, 1H), 6.70 (dd, J=8.9, 2.1 Hz, 1H), 4.35-4.50 (m, 1H), 3.51-3.3.65 (m, 1H), 3.35-3.49 (m, 1H), 3.15-3.31 (m, 1H), 2.53 (s., 3H), 2.02-2.19 (m, 1H), 1.84-1.99 (m, 1H), 1.41 (s, 9H).

Step 2: Tributylphosphine (0.103 mL; 0.39 mmol) was added dropwise to a stirred mixture of tert-butyl-(S)-3-(5-hydroxy-2-methylbenzofuran-3-carboxamido)pyrrolidine-1-carboxylate (0.100 g; 0.28 mmol), (2,3-difluorophenyl)methanol (0.063 g; 0.42 mmol) and ADDP (0.100 g; 0.39 mmol) in dry THF (5 mL) under argon. The mixture was stirred for 2 h and then the reaction was concentrated under reduced pressure. The residue was purified by FCC on silica gel using a gradient of EtOAc (7-100%) in heptane to afford 0.119 (83%) of tert-butyl (S)-3-(5-((2,3-difluorobenzyl)oxy)-2-methylbenzofuran-3-carboxamido)pyrrolidine-1-carboxylate (cpd 256).

Cpd 224, cpd 242, cpd 245, cpd 250, cpd 257, cpd 260, cpd 261 cpd 267, cpd 293, cpd 294, cpd 295 and cpd 296 were prepared in a manner similar (use of appropriate reagents and purification methods known to the person skilled in the art) to cpd 256.

### Synthesis of 5-((2,6-difluorobenzyl)oxy)-2-methylbenzofuran-3-carboxylic Acid (Cpd 019)

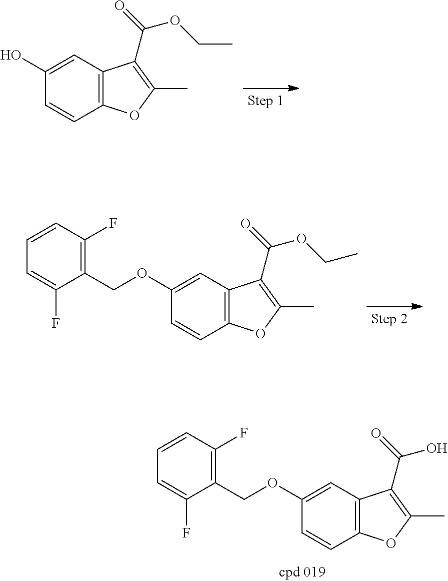 [SMILES: CCOC(=O)c1c(C)oc2ccc(O)cc12 ; CCOC(=O)c1c(C)oc2ccc(OCc3c(F)cccc3F)cc12 ; Cc1oc2ccc(OCc3c(F)cccc3F)cc2c1C(=O)O]

Step 1: Tributylphosphine (0.8 mL; 3 mmol) was added dropwise to a stirred mixture of ethyl 5-hydroxy-2-methylbenzofuran-3-carboxylate (0.5 g; 2.2 mmol), (2,6-difluorophenyl)methanol (0.48 g; 3.2 mmol) and ADDP (0.78 g; 3 mmol) in dry THF (30 mL) under argon. The mixture was stirred for 2 h and was then concentrated under reduced pressure. The residue was purified by FCC on silica gel using a gradient of EtOAc (3-30%) in heptane to afford 0.73 g (97%) of ethyl 5-((2,6-difluorobenzyl)oxy)-2-methylbenzofuran-3-carboxylate. M/Z(+): 347 (M+H).

Step 2: To a solution of ethyl 5-((2,6-difluorobenzyl)oxy)-2-methylbenzofuran-3-carboxylate (0.73 g; 2.1 mmol) in a mixture of MeOH-THF (1:1, 6 mL) was added a solution of sodium hydroxide (2 N; 4.5 mL; 9 mmol) and the RM was heated at 75° C. overnight. After cooling, the volatiles were removed under reduced pressure and the remaining residue was dissolved in water. The mixture was acidified with a solution of HCl (6 N) until pH-5. The white precipitate was filtered off, washed with water and dried under reduced pressure to afford 0.64 g (96%) of the desired compound (cpd 019).

Cpd 034 was prepared in a manner similar (use of appropriate reagents and purification methods known to the person skilled in the art) to cpd 019.

### Synthesis of tert-butyl 3-(5-(benzyloxy)-2-methylbenzofuran-3-carboxamido)-4-fluoropiperidine-1-carboxylate (Cpd 255)

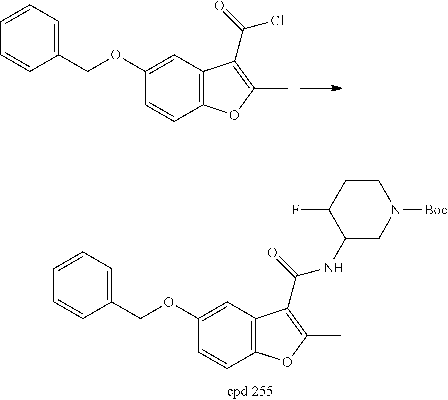 [SMILES: Cc1oc2ccc(OCc3ccccc3)cc2c1C(=O)Cl ; Cc1oc2ccc(OCc3ccccc3)cc2c1C(=O)NC1CN(C(=O)OC(C)(C)C)CCC1F]

A solution of 5-(benzyloxy)-2-methylbenzofuran-3-carbonyl chloride (200 mg; 0.66 mmol) in DCM (2 mL) was slowly added to tert-butyl 3-amino-4-fluoropiperidine-1-carboxylate (174 mg; 0.8 mmol), DIPEA (0.46 mL; 2.7 mmol) and DCM (2 mL). The RM was stirred at RT for 36 h. The volatiles were removed under reduced pressure and the crude material was purified by FCC on silica gel using a gradient of EtOAc (4% to 40%) in heptane to afford 60 mg (18.75%) of tert-butyl 3-(5-(benzyloxy)-2-methylbenzofuran-3-carboxamido)-4-fluoropiperidine-1-carboxylate (cpd 255—Dia 1) and 150 mg (46.87%) of tert-butyl 3-(5-(benzyloxy)-2-methylbenzofuran-3-carboxamido)-4-fluoropiperidine-1-carboxylate (cpd 255—Dia 2).

### Synthesis of 5-(benzyloxy)-2-methyl-N-(7-azaspiro[3.5]nonan-2-yl)benzofuran-3-carboxamide (Cpd 180)

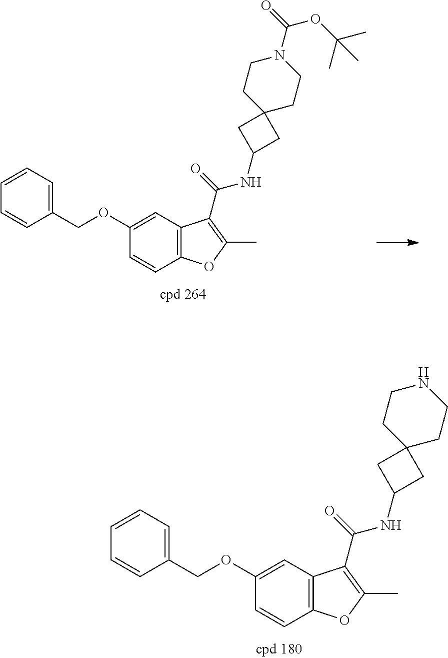 [SMILES: Cc1oc2ccc(OCc3ccccc3)cc2c1C(=O)NC1CC2(CCN(C(=O)OC(C)(C)C)CC2)C1 ; Cc1oc2ccc(OCc3ccccc3)cc2c1C(=O)NC1CC2(CCNCC2)C1]

A solution of hydrogen chloride in 1,4-dioxane (4 M; 4 mL; 16 mmol) was added to a solution of tert-butyl 2-(5-(benzyloxy)-2-methylbenzofuran-3-carboxamido)-7-azaspiro[3.5]nonane-7-carboxylate (cpd 264) (0.124 g; 0.077 mmol) in DCM (2 mL). The mixture was stirred overnight at RT and was concentrated under reduced pressure. The residue was then purified by FCC on silica gel using a gradient of a solution of 3N ammonia in MeOH (1-12%) in DCM to afford 0.035 g (35.4%) of 5-(benzyloxy)-2-methyl-N-(7-azaspiro[3.5]nonan-2-yl)benzofuran-3-carboxamide (cpd 180).

The following compounds were prepared in a manner similar (use of appropriate reagents and purification methods known to those skilled in the art) to cpd 180:
Cpd 042, 048, 049, 073, 074, 075, 077, 078, 082, 083, 084, 085, 093, 094, 095, 096, 097, 103, 114, 115, 116, 119, 120, 121, 126, 127, 130—Dia 1, 130—Dia 2, 131, 135, 136, 137, 140, 141, 143, 157, 173, 184, 190, 191, 201, 202, 203, 204, 211, 220, 223, 226, 227, 231, and 240.

### Synthesis of (S)-5-(benzyloxy)-2-methyl-N-(1-(methylsulfonyl)pyrrolidin-3-yl)benzofuran-3-carboxamide (Cpd 215)

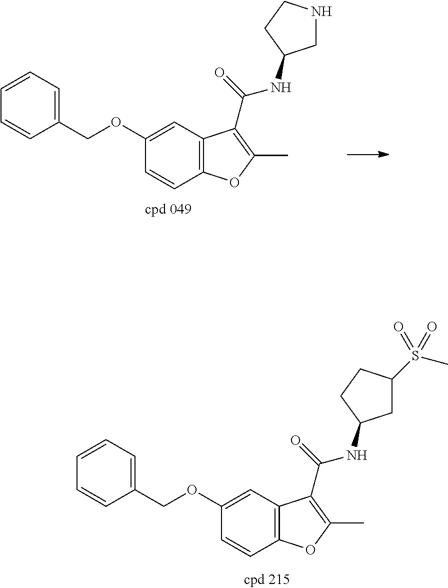 [SMILES: Cc1oc2ccc(OCc3ccccc3)cc2c1C(=O)N[C@H]1CCNC1 ; Cc1oc2ccc(OCc3ccccc3)cc2c1C(=O)N[C@H]1CCC(S(C)(=O)=O)C1]

Step 1: To a solution of (S)-5-(benzyloxy)-2-methyl-N-(pyrrolidin-3-yl)benzofuran-3-carboxamide (cpd 049) (75 mg; 0.21 mmol) in DCM (4 mL) was added methanesulfonyl chloride (18 μL; 0.23 mmol), and TEA (33 μL; 0.23 mmol) at RT. The RM was stirred at RT until the consumption of cpd 049. The volatiles were removed under reduced pressure and the crude material was purified by FCC on silica gel using a gradient of EtOAc (50% to 100%) in heptane to afford 51 mg (56%) of (S)-5-(benzyloxy)-2-methyl-N-(1-(methylsulfonyl)pyrrolidin-3-yl)benzofuran-3-carboxamide (cpd 215).

### Synthesis of (S)—N-(1-acetylpyrrolidin-3-yl)-5-(benzyloxy)-2-methylbenzofuran-3-carboxamide (Cpd 145)

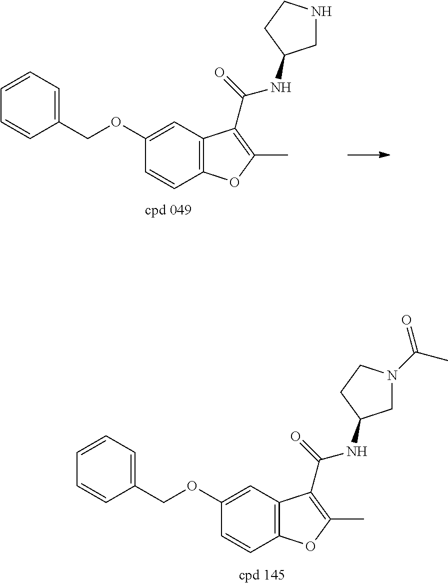 [SMILES: Cc1oc2ccc(OCc3ccccc3)cc2c1C(=O)N[C@H]1CCNC1 ; CC(=O)N1CC[C@H](NC(=O)c2c(C)oc3ccc(OCc4ccccc4)cc23)C1]

To a solution of (S)-5-(benzyloxy)-2-methyl-N-(pyrrolidin-3-yl)benzofuran-3-carboxamide (Cpd 049) (75 mg; 0.21 mmol) in DCM (4 mL) was added acetyl chloride (13 μL; 0.23 mmol) and TEA (33 μL; 0.23 mmol) at RT. The RM was stirred at RT overnight. The volatiles were removed under reduced pressure and the crude material was purified by FCC on silica gel using a gradient of EtOAc (100%) first then MeOH (2%) in DCM to afford 50 mg (59%) of (S)—N-(1-acetylpyrrolidin-3-yl)-5-(benzyloxy)-2-methylbenzofuran-3-carboxamide (cpd 145).

### Synthesis 5-(benzyloxy)-2-methyl-N-(methylsulfonyl)benzofuran-3-carboxamide (Cpd 063)

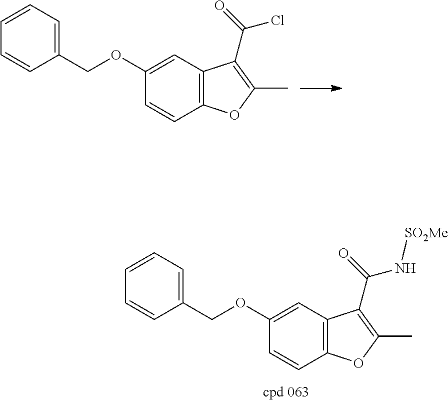 [SMILES: Cc1oc2ccc(OCc3ccccc3)cc2c1C(=O)Cl ; Cc1oc2ccc(OCc3ccccc3)cc2c1C(=O)NS(C)(=O)=O]

Step 1: Sodium hydride (60% in oil; 70.84 mg; 1.77 mmol) was added to a cold (0° C.) solution of methanesulfonamide (40.4 mg; 0.425 mmol) in THF (3 mL). The resulting suspension was stirred at RT for 1.5 h and a solution of 5-(benzyloxy)-2-methylbenzofuran-3-carbonyl chloride (previously described) (106 mg; 0.35 mmol) in THF (3 mL) was added dropwise over 10 min and then, the RM was stirred 18 h at RT. The RM was then cooled down to 0° C., quenched with water and stirred at RT for 20 min. The mixture was extracted with DCM and EtOAc. The combined organic layers were dried over sodium sulfate, filtered and concentrated under reduced pressure. The residue was purified by FCC on silica gel using a gradient of EtOAc (20% to 100%) in heptane then MeOH (0% to 25%) in EtOAc to afford 101.5 mg (74%) of 5-(benzyloxy)-2-methyl-N-(methylsulfonyl)benzofuran-3-carboxamide (cpd 063).

Cpd 133, cpd 169, cpd 170, cpd 175, cpd 176, cpd 177, cpd 195 and cpd 199 were prepared in a manner similar (use of appropriate reagents and purification methods known to the person skilled in the art) to cpd 063.

### Synthesis of 5-((2-(hydroxymethyl)benzyl)oxy)-2-methyl-N-(1-methylpiperidin-4-yl)benzofuran-3-carboxamide (Cpd 192)

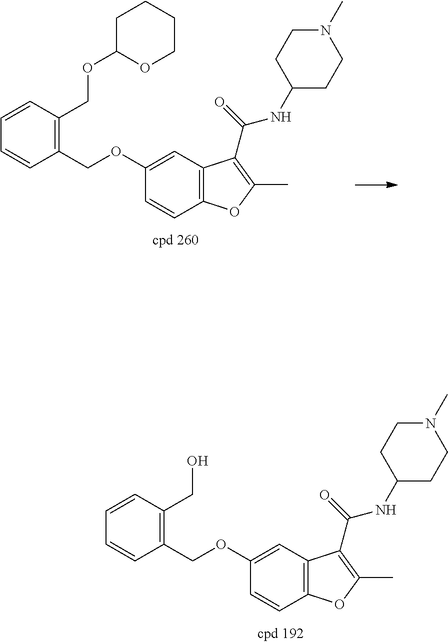 [SMILES: Cc1oc2ccc(OCc3ccccc3COC3CCCCO3)cc2c1C(=O)NC1CCN(C)CC1 ; Cc1oc2ccc(OCc3ccccc3CO)cc2c1C(=O)NC1CCN(C)CC1]

A solution of HCl (2N in water, 3 mL; 6 mmol) was added to a solution of 2-methyl-N-(1-methylpiperidin-4-yl)-5-((2-(((tetrahydro-2H-pyran-2-yl)oxy)methyl)benzyl)oxy)benzofuran-3-carboxamide (cpd 260) (135 mg; 0.27 mmol) in MeOH (3 mL). The RM was stirred at RT for 2 h. Then, 20 mL of a solution of ammonia (10%) in MeOH was added to the RM. The volatiles were removed under reduced pressure. The residue was purified first by FCC on silica gel using a gradient of MeOH (with ammonia) (2-10%) in DCM. The crude product was purified by preparative HPLC (method H1) to afford 10 mg (8%) of the desired product (cpd 192).

### Synthesis of 5-(benzyloxy)-N-(1-ethyl-2-oxopyrrolidin-3-yl)-2-methylbenzofuran-3-carboxamide (Cpd 147)

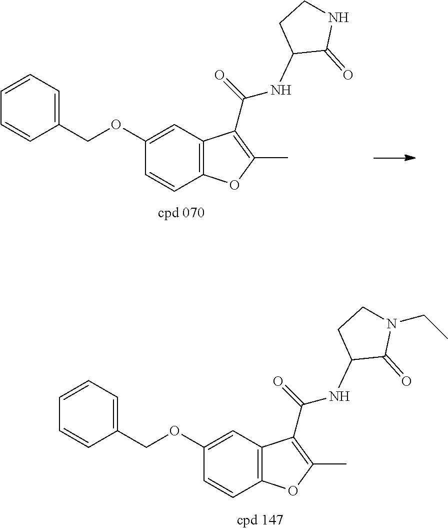 [SMILES: Cc1oc2ccc(OCc3ccccc3)cc2c1C(=O)NC1CCNC1=O ; CCN1CCC(NC(=O)c2c(C)oc3ccc(OCc4ccccc4)cc23)C1=O]

Cesium hydroxide (46 mg; 0.27 mmol) was added to 5-(benzyloxy)-2-methyl-N-(2-oxopyrrolidin-3-yl)benzofuran-3-carboxamide (cpd 070) (100 mg; 0.27 mmol) in DMF (2 mL). Then ethyl iodide (0.05 mL; 0.55 mmol) was added to the reaction. The mixture was stirred at RT for 2 h, quenched with ice and acidified with an aq. solution of HCl 1N. The mixture was extracted with EtOAc and concentrated under reduced pressure. The crude mixture was purified by FCC on silica gel using a gradient of MeOH (0-20%) in DCM. The residual oil was purified by preparative HPLC (method H1) to afford 39 mg (36.2%) of the desired compound (cpd 147).

### Synthesis of 4-(5-(benzyloxy)-2-methylbenzofuran-3-carboxamido)tetrahydro-2H-pyran-4-carboxylic Acid (Cpd 193)

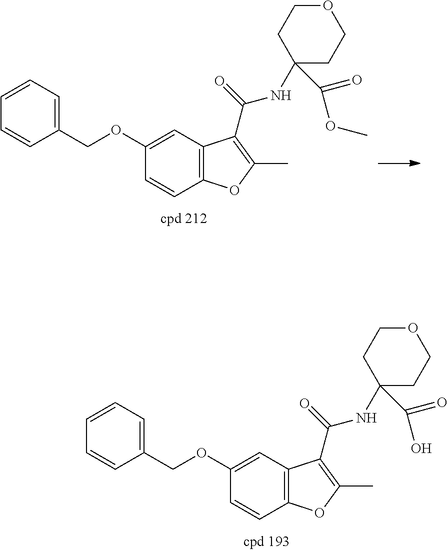 [SMILES: COC(=O)C1(NC(=O)c2c(C)oc3ccc(OCc4ccccc4)cc23)CCOCC1 ; Cc1oc2ccc(OCc3ccccc3)cc2c1C(=O)NC1(C(=O)O)CCOCC1]

An aq. solution of NaOH (2 N; 1 mL) was added to methyl 4-(5-(benzyloxy)-2-methylbenzofuran-3-carboxamido)tetrahydro-2H-pyran-4-carboxylate (cpd 212) in MeOH (1 mL) and the mixture was stirred at RT for 5 days. The RM was concentrated under reduced pressure, dissolved in water and acidified dropwise with an aq. solution of HCl (6 N). The solid formed was filtered and the residue was purified by FCC on silica gel using a gradient of EtOAc (20-100%) in heptane to afford 0.036 g (28%) of 4-(5-(benzyloxy)-2-methylbenzofuran-3-carboxamido)tetrahydro-2H-pyran-4-carboxylic acid (cpd 193).

Cpd 160, cpd 161 and cpd 162 were prepared in a manner similar (use of appropriate reagents and purification methods known to the person skilled in the art) to cpd 193.

### Synthesis of (2S,4R)-4-(5-(benzyloxy)-2-methylbenzofuran-3-carboxamido)pyrrolidine-2-carboxamide (Cpd 158)

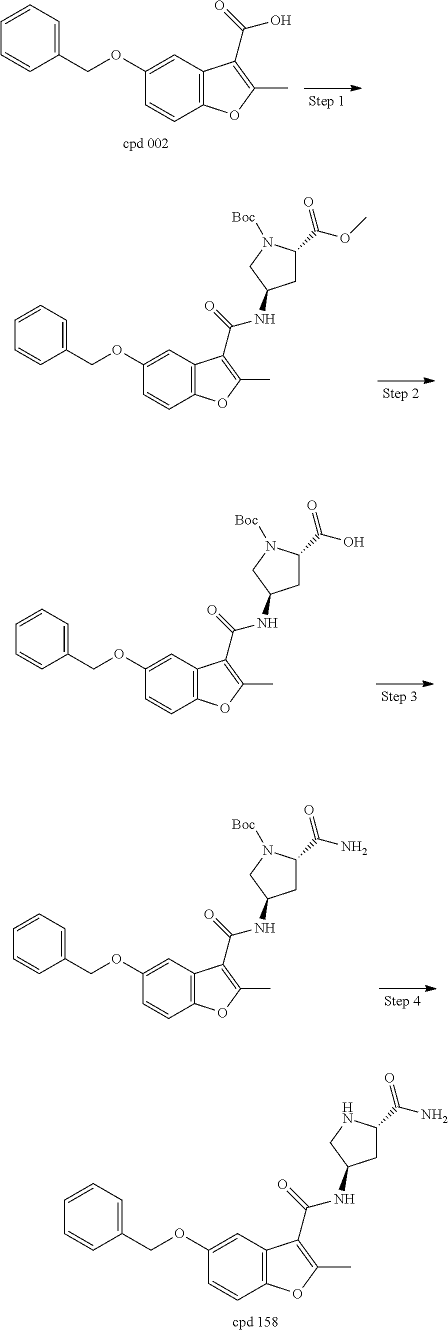 [SMILES: Cc1oc2ccc(OCc3ccccc3)cc2c1C(=O)O ; COC(=O)[C@@H]1C[C@@H](NC(=O)c2c(C)oc3ccc(OCc4ccccc4)cc23)CN1C(=O)OC(C)(C)C ; Cc1oc2ccc(OCc3ccccc3)cc2c1C(=O)N[C@@H]1C[C@@H](C(=O)O)N(C(=O)OC(C)(C)C)C1 ; Cc1oc2ccc(OCc3ccccc3)cc2c1C(=O)N[C@@H]1C[C@@H](C(N)=O)N(C(=O)OC(C)(C)C)C1 ; Cc1oc2ccc(OCc3ccccc3)cc2c1C(=O)N[C@H]1CN[C@H](C(N)=O)C1]

Step 1: DIPEA (0.93 mL; 5.3 mmol) was added to a mixture of 5 5-(benzyloxy)-2-methylbenzofuran-3-carboxylic acid (cpd 002) (0.500 g; 1.77 mmol), HATU (0.808 g; 2.13 mmol), and 1-(tert-butyl) 2-methyl (2S,4R)-4-aminopyrrolidine-1,2-dicarboxylate (0.519 g; 2.13 mmol) in DMF (5 mL). The mixture was stirred at RT for 36 h and was concentrated under reduced pressure. The residue was partitioned between a sat. aq. solution of sodium bicarbonate and DCM, and after separation, the organic layer was concentrated under reduced pressure. The residue was purified by FCC on silica gel using a gradient of EtOAc (20-100%) in heptane to afford 0.708 g (79%) of 1-(tert-butyl) 2-methyl (2S,4R)-4-(5-(benzyloxy)-2-methylbenzofuran-3-carboxamido)pyrrolidine-1,2-dicarboxylate as a white solid. ¹H NMR (300 MHz, CDCl₃) δ ppm: 7.18-7.57 (m, 6H); 7.06 (d, 1H); 6.96 (dd, 1H); 5.77 (br. s., 1H); 5.12 (s, 2H); 4.65-4.84 (m, 1H); 4.25-4.56 (m, 1H); 3.85-4.04 (m, 1H); 3.77 (s, 3H); 3.23-3.60 (m, 1H); 2.68 (s, 3H); 2.11-2.55 (m, 2H); 1.32-1.53 (m, 9H); 1.19-1.32 (m, 1H).

Step 2: An aq. solution of NaOH (2 N; 0.28 mL; 0.56 mmol) was added to a solution of 1-(tert-butyl) 2-methyl (2S,4R)-4-(5-(benzyloxy)-2-methylbenzofuran-3-carboxamido)pyrrolidine-1,2-dicarboxylate (0.286 g; 0.56 mmol) in MeOH (4 mL). The mixture was stirred for 2 h at RT and the volume of the mixture was reduced to the half under reduced pressure. An aq. solution of HCl (1 N) was added and the formed precipitate was collected by filtration and dried to afford 0.278 g (100%) of (2S,4R)-4-(5-(benzyloxy)-2-methylbenzofuran-3-carboxamido)-1-(tert-butoxycarbonyl)pyrrolidine-2-carboxylic acid. ¹H NMR (300 MHz, DMSO-d₆) δ ppm: 8.27 (t, 1H); 7.28-7.58 (m, 5H); 7.21 (s, 1H); 6.97 (dd, 1H); 5.13 (s, 2H); 4.20-4.40 (m, 1H); 4.31-4.28 (m, 1H); 3.59-3.82 (m, 1H); 2.56 (s, 3H); 1.98-2.45 (m, 3H); 1.37 (d, 9H).

Step 3: DIPEA (0.09 mL; 0.5 mmol) was added to a solution of (2S,4R)-4-(5-(benzyloxy)-2-methylbenzofuran-3-carboxamido)-1-(cert-butoxycarbonyl)pyrrolidine-2-carboxylic acid (0.09 g; 0.18 mmol), HATU (0.1 g; 0.27 mmol), and ammoniac in dioxane (0.5 M; 0.54 mL; 0.27 mmol) in DMF (3 mL). The mixture was stirred at RT for 60 h. The mixture was partitioned between an aq. solution of KHSO₄ (2 N) and EtOAc, the organic phase was washed with sat. sodium bicarbonate, dried and concentrated under reduced pressure. The crude residue was purified by FCC on silica using a gradient of MeOH (0-20%) in DCM to afford 0.081 g (90%) of tert-butyl(2S,4R)-4-(5-(benzyloxy)-2-methylbenzofuran-3-carboxamido)-2-calbamoylpyrrolidine-1-carboxylate. M/Z(+): 494 (M+H). M/Z(−): 492 (M−H).

Step 4: A solution of HCl in 1,4-dioxane (4 M; 2 mL) was added to tert-butyl (2S,4R)-4-(5-(benzyloxy)-2-methylbenzofuran-3-carboxamido)-2-carbamoylpyrrolidine-1-carboxylate (81 mg; 0.16 mmol). The mixture was stirred at RT for 4 h and was concentrated under reduced pressure. The residue was purified by a column of supported SiliaBond-PropylSulfonic Acid. First, the column was washed with a gradient of DCM (0% to 100%) in MeOH and finally with a 3N solution of ammonia in MeOH to afford 48 mg (73%) of (2S,4R)-4-(5-(benzyloxy)-2-methylbenzofuran-3-carboxamido)pyrrolidine-2-carboxamide (cpd 158).

Cpd 188 was prepared in a manner similar (use of appropriate reagents and purification methods known to the person skilled in the art) to cpd 158.

### Diastereoisomer Separation of 5-(benzyloxy)-N-(hexahydro-1H-pyrrolizin-1-yl)-2-methylbenzofuran-3-carboxamide (Cpd 144) Leading to (CPD 144—Dia 1) and (Cpd 144—Dia 2)

5-(Benzyloxy)-N-(hexahydro-1H-pyrrolizin-1-yl)-2-methylbenzofuran-3-carboxamide (cpd 144) (0.090 g) was separated into its diastereoisomers by preparative HPLC (Method: H1) to afford 38.7 mg of the faster eluting diastereoisomer (Cpd 144—Dia 1) and 27.8 mg of the slower eluting diastereoisomer (Cpd 144—Dia 2). The shown absolute stereochemistry of all compounds was only randomized but not confirmed.

### Synthesis of 5-(benzyloxy)-4-cyano-N-(4,4-difluoropyrrolidin-3-yl)-2-methylbenzofuran-3-carboxamide (Cpd 297)

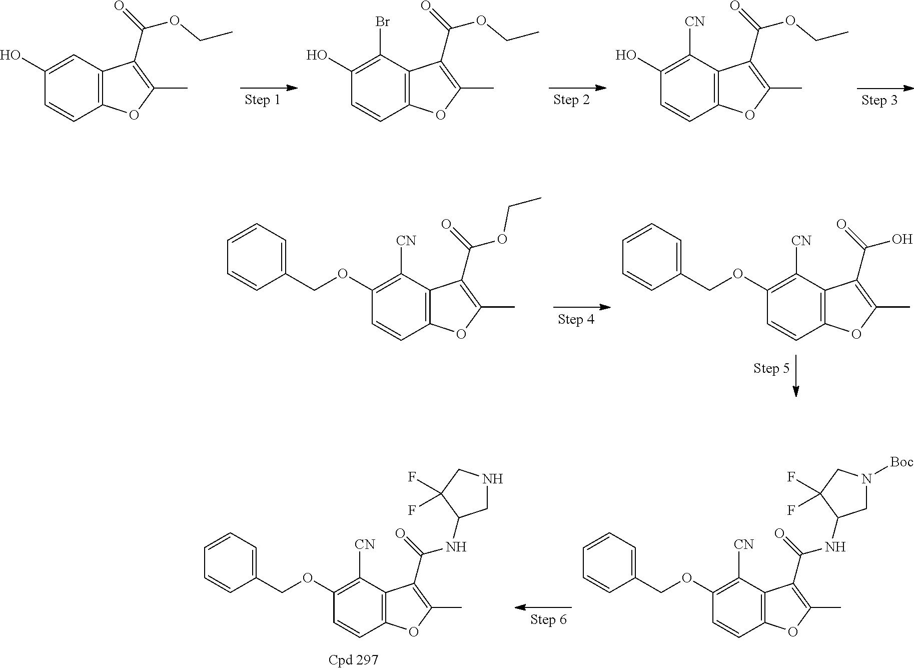

Step 1: NBS (24.2 g, 136.22 mmol) was added to a solution of ethyl 5-hydroxy-2-methylbenzofuran-3-carboxylate (20.0 g, 90.81 mmol) in MeCN (600 mL) at RT under argon atmosphere. The RM was stirred for 16 h at RT. The reaction progress was monitored by TLC. The RM was diluted with water (300 mL), acidified with 1N—HCl to a pH-2. The crude product was extracted with EtOAc (2×300 mL). The combined organic layer was washed with water (200 mL) followed by brine (200 mL). The organic layer was dried over anhydrous Na₂SO₄ and concentrated under reduced pressure. The crude product was purified by FCC over silica gel using 0-20% EtOAc and pet-ether as an eluent to afford ethyl 4-bromo-5-hydroxy-2-methylbenzofuran-3-carboxylate (5.0 g, 19%).

Step 2: CuCN (0.74 g, 8.36 mmol) was added to a solution of ethyl 4-bromo-5-hydroxy-2-methylbenzofuran-3-carboxylate (1.0 g, 3.44 mmol) in DMF (25 mL) at RT under argon atmosphere. The resulting mixture was heated to 160° C. and maintained for 3 h. The reaction progress was monitored by TLC. The RM was cooled to RT, poured into water (100 mL) and extracted with EtOAc (2×100 mL). The combined organic layer was washed with water (2×50 mL) followed by with brine (50 mL). The organic layer was dried over anhydrous Na₂SO₄ and concentrated under reduced pressure. The crude product was purified by FCC over silica gel using 0-20% EtOAc in pet-ether as an eluent to afford ethyl 4-cyano-5-hydroxy-2-methylbenzofuran-3-carboxylate (0.45 g, 55%).

Step 3: Phenylmethanol (0.36 mL, 2.75 mmol), ADDP (0.646 g, 2.56 mmol) and tri-n-butylphosphine (0.63 mL, 2.56 mmol) were added sequentially to a pre-stirred solution of ethyl 4-cyano-5-hydroxy-2-methylbenzofuran-3-carboxylate (0.45 g, 1.83 mmol) in THF (20 mL) at 0° C. under argon atmosphere. The RM was allowed to attain RT and stirred for 3 h. The reaction progress was monitored by TLC. The RM was poured into water (50 mL) and extracted with EtOAc (2×50 mL). The combined organic layer was washed with water (50 mL), brine (50 mL), dried over anhydrous Na₂SO₄ and concentrated under reduced pressure. The crude product was passed through a column of silica gel using 0-20% EtOAc in pet-ether as an eluent to afford ethyl 5-(benzyloxy)-4-cyano-2-methylbenzofuran-3-carboxylate (0.36 g, 58%).

Step 4: A solution of NaOH (0.166 g, 4.16 mmol) in water (5.0 mL) was added to a pre-stirred solution of ethyl 5-(benzyloxy)-4-cyano-2-methylbenzofuran-3-carboxylate (0.36 g, 1.04 mmol) in a mixture of MeOH (10 mL) and THF (10 mL) at RT. The resulting RM was heated to 60° C. and maintained for 3 h. The reaction progress was monitored by TLC. The RM was cooled to RT and poured into ice cold water (50 mL), acidified with 1N HCl (pH˜2). The crude product was extracted with EtOAc (2×50 mL). The combined organic layer was washed with water (2×50 mL) followed by brine (50 mL), dried over anhydrous Na₂SO₄ and concentrated under reduced pressure to afford 5-(benzyloxy)-4-cyano-2-methylbenzofuran-3-carboxylic acid (0.29 g, 87%). The crude product thus obtained was used for next step without further purification.

Step 5: To a pre-stirred solution of mixture of 5-(benzyloxy)-4-cyano-2-methylbenzofuran-3-carboxylic acid (0.29 g, 0.94 mmol) and tert-butyl 4-amino-3,3-difluoropyrrolidine-1-carboxylate (0.251 g, 1.13 mmol) in DMF (10 mL) were added DIPEA (0.46 mL, 2.83 mmol) followed by HATU (0.717 g, 1.88 mmol) at 0° C. under argon atmosphere. The RM was allowed to attain RT and stirred for 3 h. The reaction progress was monitored by TLC. The RM was diluted with water (50 mL), the crude product was extracted with EtOAc (2×50 mL). The combined organic layer was washed with water (50 mL), brine (50 mL), dried over anhydrous Na₂SO₄ and concentrated under reduced pressure. The crude product, tert-butyl 4-(5-(benzyloxy)-4-cyano-2-methylbenzofuran-3-carboxamido)-3,3-difluoropyrrolidine-1-carboxylate (0.25 g, 51%), obtained as an off-white solid, was used further without purification.

Step 6: To a pre-stirred solution of tert-butyl 4-(5-(benzyloxy)-4-cyano-2-methylbenzofuran-3-carboxamido)-3,3-difluoropyrrolidine-1-carboxylate (0.25 g) in DCM (2.5 mL) was added TFA (2.5 mL) dropwise at 0° C. under argon atmosphere. The RM was allowed to attain RT and stirred for 16 h. The reaction progress was monitored by TLC. The RM was concentrated under reduced pressure, basified with NaHCO₃ (pH-8). The crude product was extracted with EtOAc (2×50 mL). The combined organic layer was washed with water (50 mL), then brine (50 mL), dried over anhydrous Na₂SO₄ and concentrated under reduced pressure. The crude product was purified by GRACE flash chromatography using 0-50% acetonitrile in 0.1% FA in water as an eluent to afford racemic Cpd 297 (0.18 g, 89%). A preparative chiral SFC was performed on racemic Cpd 297 to afford Cpd 297—En 1 and Cpd 297—En 2.

### Synthesis of 5-(benzyloxy)-N-(4,4-difluoropyrrolidin-3-yl)-4-fluoro-2-methylbenzofuran-3-carboxamide (Cpd 298) and 5-(benzyloxy)-N-(4,4-difluoropyrrolidin-3-yl)-6-fluoro-2-methylbenzofuran-3-carboxamide (Cpd 302)

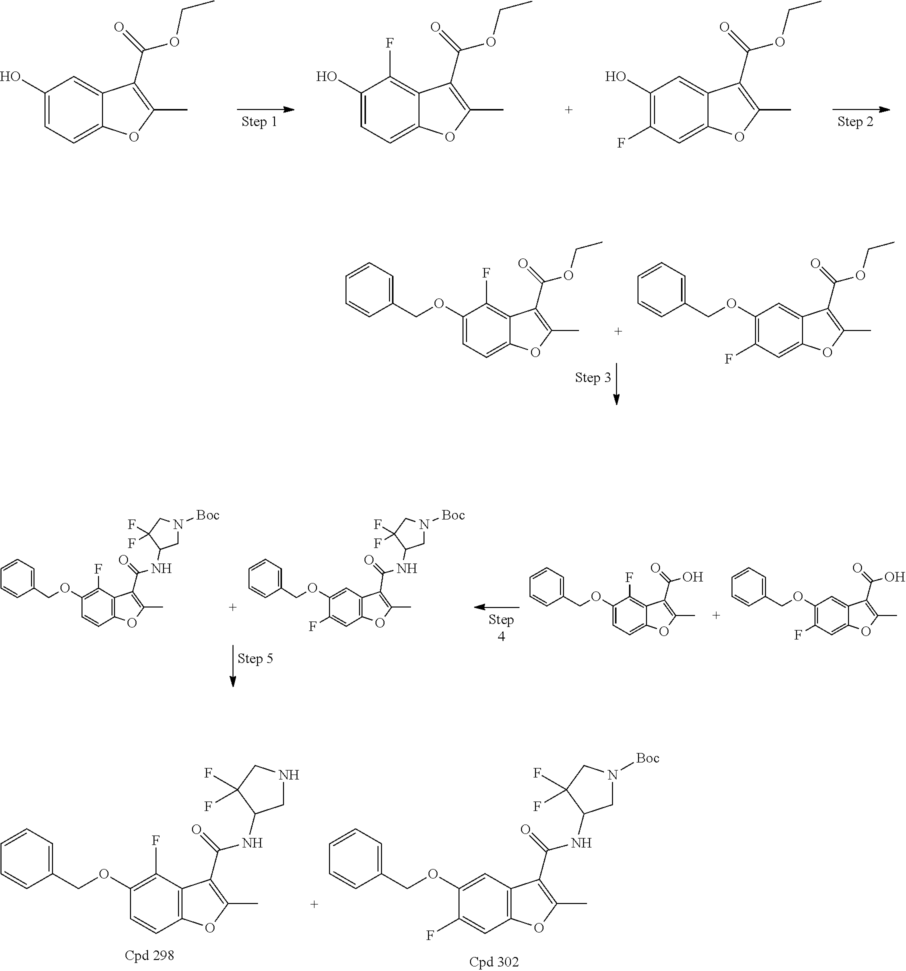

Step 1: To a pre-stirred solution of ethyl 5-hydroxy-2-methylbenzofuran-3-carboxylate (5.0 g, 22.7 mmol) in MeCN (300 mL) was added selectfluor (9.65 g, 27.2 mmol) at RT under argon atmosphere. The resulting RM was stirred for 16 h at RT. The reaction progress was monitored by TLC. The excess solvent was removed under reduced pressure and the crude compound was dissolved in EtOAc (500 mL). The above solution was washed with water (2×250 mL), brine (250 mL), dried over anhydrous Na₂SO₄ and concentrated under reduced pressure. The crude product was purified by FCC over silica using 0-20% EtOAc in pet-ether as an eluent followed by GRACE flash chromatography using 0-47% of acetonitrile in 0.1% FA in water as an eluent to afford mixture of ethyl 4-fluoro-5-hydroxy-2-methylbenzofuran-3-carboxylate and ethyl 6-fluoro-5-hydroxy-2-methylbenzofuran-3-carboxylate (1.0 g, 19%).

Step 2: Phenylmethanol (2 mL, 18.9 mmol), ADDP (4.45 g, 17.6 mmol) and tri-n-butylphosphine (3.56 g, 17.6 mmol) were added sequentially to a pre-stirred solution of mixture of ethyl 4-fluoro-5-hydroxy-2-methylbenzofuran-3-carboxylate and ethyl 6-fluoro-5-hydroxy-2-methylbenzofuran-3-carboxylate (3.0 g, 12.6 mmol) in THF (100 mL) at 0° C. under argon atmosphere. The RM was allowed to attain RT and stirred for 3 h. The reaction progress was monitored by TLC. The RM was poured into water (250 mL) and extracted with EtOAc (3×200 mL). The combined organic layer was washed with brine (2×100 mL), dried over anhydrous Na₂SO₄ and concentrated under reduced pressure. The crude product was purified by FCC over silica using 10-20% EtOAc in pet-ether as an eluent to afford a mixture of ethyl 5-(benzyloxy)-4-fluoro-2-methylbenzofuran-3-carboxylate and ethyl 5-(benzyloxy)-6-fluoro-2-methylbenzofuran-3-carboxylate (2.1 g, 50%).

Step 3: 2N NaOH (30 mL) was added to a pre-stirred solution of mixture of ethyl 5-(benzyloxy)-4-fluoro-2-methylbenzofuran-3-carboxylate and ethyl 5-(benzyloxy)-6-fluoro-2-methylbenzofuran-3-carboxylate (2.0 g, 6.0 mmol) in a mixture of MeOH (50 mL) and THF (20 mL) at RT. The resulting RM was heated to 60° C. and maintained for 3 h. The reaction progress was monitored by TLC. The RM was cooled to RT, poured into ice cold water (250 mL) and acidified with 1N HCl (pH-2). The crude product was extracted with EtOAc (3×200 mL). The combined organic layer was washed with water (2×200 mL) followed by brine (200 mL), dried over anhydrous Na₂SO₄ and concentrated under reduced pressure to afford a mixture of 5-(benzyloxy)-4-fluoro-2-methylbenzofuran-3-carboxylic acid and 5-(benzyloxy)-6-fluoro-2-methylbenzofuran-3-carboxylic acid (1.3 g, 71%). The obtained mixture of crude product was used for next step without further purification.

Step 4: To a pre-stirred solution of mixture of 5-(benzyloxy)-4-fluoro-2-methylbenzofuran-3-carboxylic acid and 5-(benzyloxy)-6-fluoro-2-methylbenzofuran-3-carboxylic acid (1.3 g, 4.33 mmol), and tert-butyl 4-amino-3,3-difluoropyrrolidine-1-carboxylate (1.15 g, 5.19 mmol) in DMF (25 mL) was added DIPEA (1.6 mL, 8.6 mmol) followed by HATU (3.29 g, 8.6 mmol) at 0° C. under argon atmosphere. The RM was allowed to attain RT and stirred for 1 h. The reaction progress was monitored by TLC. The RM was diluted with ice cold water (50 mL) and filtered. The solid thus obtained was washed with water (200 mL), dried under reduced pressure to afford a mixture of tert-butyl 4-(5-(benzyloxy)-4-fluoro-2-methylbenzofuran-3-carboxamido)-3,3-difluoropyrrolidine-1-carboxylate and tert-butyl 4-(5-(benzyloxy)-6-fluoro-2-methylbenzofuran-3-carboxamido)-3,3-difluoropyrrolidine-1-carboxylate (1.2 g, 57%).

Step 5: 4M HCl in dioxane (6 mL) was added dropwise to a solution of mixture of tert-butyl 4-(5-(benzyloxy)-4-fluoro-2-methylbenzofuran-3-carboxamido)-3,3-difluoropyrrolidine-1-carboxylate and tert-butyl 4-(5-(benzyloxy)-6-fluoro-2-methylbenzofuran-3-carboxamido)-3,3-difluoropyrrolidine-1-carb oxy late (1.2 g, 2.3 mmol) in DCM (25 mL) at 0° C. under argon atmosphere. The RM was warmed to RT and stirred for 5 h. The reaction progress was monitored by TLC. The excess solvents were evaporated in vacuo and the residue was cooled to 0° C., basified with sat. NaHCO₃ (pH ˜9) and extracted with EtOAc (3×50 mL). The combined organic layer was washed with water (100 mL), brine (100 mL), dried over anhydrous Na₂SO₄ and concentrated under reduced pressure. The crude product was purified by GRACE flash chromatography using 0-50% acetonitrile and 0.1% FA in water as an eluent to afford a mixture of racemic Cpd 298 and racemic Cpd 302 (0.6 g, 62%). A preparative chiral SFC was performed on the mixture of Cpd 298 and Cpd 302 to afford Cpd 298—En 1, Cpd 298—En 2, Cpd 302—En 1 and Cpd 302—En 2.

### Synthesis of 5-(benzyloxy)-N-(4,4-difluoropyrrolidin-3-yl)-2,4-dimethylbenzofuran-3-carboxamide (Cpd 299)

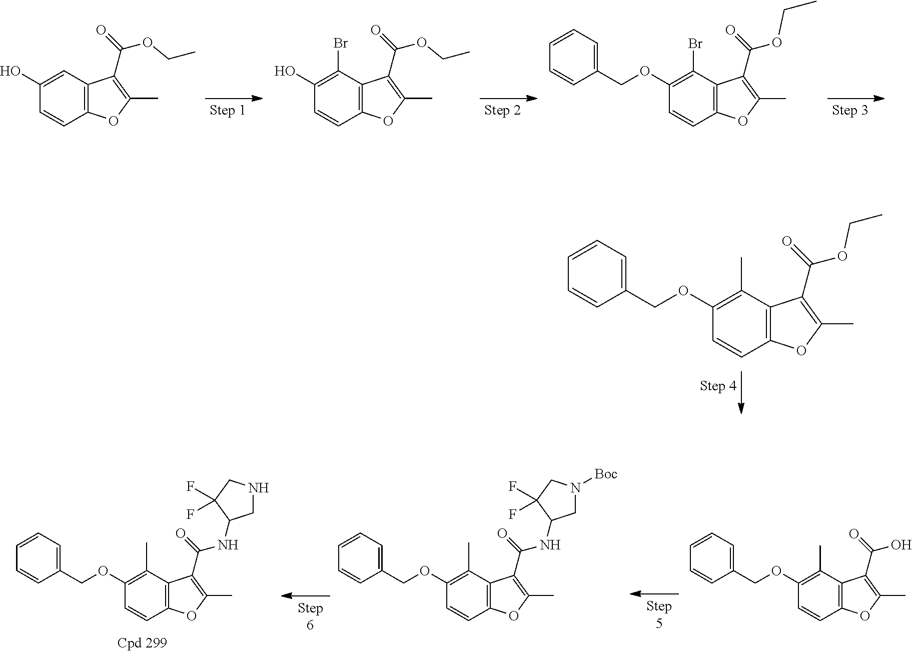

Step 1: NBS (24.2 g, 136.22 mmol) was added to a solution of ethyl 5-hydroxy-2-methylbenzofuran-3-carboxylate (20.0 g, 90.81 mmol) in acetonitrile (600 mL) at RT under an argon atmosphere. The RM was stirred for 16 h at same temperature. The reaction progress was monitored by TLC. The RM was diluted with water (300 mL) and acidified with 1N—HCl to pH-2. The crude product was extracted with EtOAc (2×300 mL). The combined organic layer was washed with water (200 mL) followed by brine (200 mL). The organic layer was dried over anhydrous Na₂SO₄ and concentrated under reduced pressure. The crude product was purified by column chromatography over silica gel using 0-20% EtOAc and pet-ether as an eluent to afford ethyl 4-bromo-5-hydroxy-2-methylbenzofuran-3-carboxylate (5.0 g, 19%).

Step 2: Phenylmethanol (0.678 mL, 6.52 mmol), ADDP (1.77 g, 7.02 mmol) and tri-n-butylphosphine (1.42 g, 7.02 mmol) were added sequentially to a pre-stirred solution of ethyl 4-bromo-5-hydroxy-2-methylbenzofuran-3-carboxylate in THF (50 mL) at 0° C. under argon atmosphere. The RM was warmed to RT and stirred for 2 h. The reaction progress was monitored by TLC. The RM was poured into water (80 mL) and extracted with EtOAc (2×60 mL). The combined organic layer was sequentially washed with water (30 mL), brine (30 mL), dried over anhydrous Na₂SO₄ and concentrated under reduced pressure. The crude product was purified by FCC over silica gel using 0-20% EtOAc in pet-ether as an eluent to afford ethyl 5-(benzyloxy)-4-bromo-2-methylbenzofuran-3-carboxylate (0.95 g, 48%).

Step 3: In a sealed tube, K₃PO₄ (1.81 g, 8.54 mmol) was added to a solution of ethyl 5-(benzyloxy)-4-bromo-2-methylbenzofuran-3-carboxylate (0.95 g, 2.44 mmol) and methylboronic acid (292.1 mg, 4.88 mmol) in a mixture of toluene (9 mL) and water (1 mL) at RT under argon atmosphere. The resulting RM was degassed with argon for 10 min and then (Cy)₃P (60 mg, 0.22 mmol) followed by Pd(OAc)₂ (65.73 mg, 0.29 mmol) was added. The RM was further degassed for 10 min. The RM heated to 120° C. and maintained for 18 h. The reaction progress was monitored by LC-MS. The RM was cooled to RT and filtered through celite pad. The celite pad was washed with EtOAc (2×30 mL). The clear filtrate was dried over anhydrous Na₂SO₄ and evaporated in vacuo. The crude product was purified by FCC over silica gel using 0-20% EtOAc in pet-ether as an eluent to afford ethyl 5-(benzyloxy)-2,4-dimethylbenzofuran-3-carboxylate (0.59 g, 74%).

Step 4: A solution of NaOH (0.287 g, 7.18 mmol) in water (4 mL) was added dropwise to a pre-stirred solution of ethyl 5-(benzyloxy)-2,4-dimethylbenzofuran-3-carboxylate (0.58 g, 1.79 mmol) in a mixture of MeOH (7 mL) and THF (7 mL) at RT. The resulting RM was heated to 60° C. and maintained for 4 h. The reaction progress was monitored by TLC. The RM was cooled to RT and poured into ice cold water (30 mL), acidified with 1N HCl to a pH-2. The crude product was extracted with EtOAc (2×50 mL). The combined organic layer was washed with water (30 mL) followed by brine (30 mL), dried over anhydrous Na₂SO₄ and concentrated under reduced pressure to afford 5-(benzyloxy)-2,4-dimethylbenzofuran-3-carboxylic acid (0.52 g, 98%).

Step 5: To a pre-stirred solution of 5-(benzyloxy)-2,4-dimethylbenzofuran-3-carboxylic acid (0.52 g, 1.76 mmol) and tert-butyl 4-amino-3,3-difluoropyrrolidine-1-carboxylate (0.508 g, 2.28 mmol) in DMF (20 mL) was added DIPEA (2.4 mL, 14.08 mmol) followed by HATU (1.34 g, 3.52 mmol) at 0° C. under argon atmosphere. The RM was allowed to attain RT and stirred for 2 h. The reaction progress was monitored by LC-MS. The RM was diluted with water (80 mL) and the organic compound was extracted with EtOAc (2×50 mL). The combined organic layer was washed with water (30 mL), brine (30 mL), dried over anhydrous Na₂SO₄ and concentrated under reduced pressure. The crude product was purified by GRACE flash chromatography using 0-80% acetonitrile and 0.1% FA in water as an eluent to afford tert-butyl 4-(5-(benzyloxy)-2,4-dimethylbenzofuran-3-carboxamido)-3,3-difluoropyrrolidine-1-carboxylate (0.56 g, 63%).

Step 6: A solution of TFA (5 mL) in DCM (5 mL) was added dropwise to a pre-stirred solution of tert-butyl 4-(5-(benzyloxy)-2,4-dimethylbenzofuran-3-carboxamido)-3,3-difluoropyrrolidine-1-carboxylate (0.55 g, 1.10 mmol) in DCM (1 mL) at 0° C. under argon atmosphere. The RM was allowed to attain RT and stirred for 16 h. The reaction progress was monitored by TLC. The RM was concentrated under reduced pressure, the residue was basified with sat. NaHCO₃ (60 mL) and the organic compound was extracted with 10% MeOH in DCM (3×mL). The combined organic layer was washed with water (40 mL), brine (40 mL), dried over anhydrous Na₂SO₄ and concentrated under reduced pressure. The crude product was purified by GRACE flash chromatography using acetonitrile and 0.1% FA in water as an eluent to afford racemic Cpd 299 (0.12 g, 26%). A preparative chiral SFC was performed on racemic Cpd 299 to afford Cpd 299—En 1 and Cpd 299—En 2.

### Synthesis of 5-(benzyloxy)-6-cyano-N-(4,4-difluoropyrrolidin-3-yl)-2-methylbenzofuran-3-carboxamide (Cpd 301) and 5-(benzyloxy)-7-cyano-N-(4,4-difluoropyrrolidin-3-yl)-2-methylbenzofuran-3-carboxamide (Cpd 305)

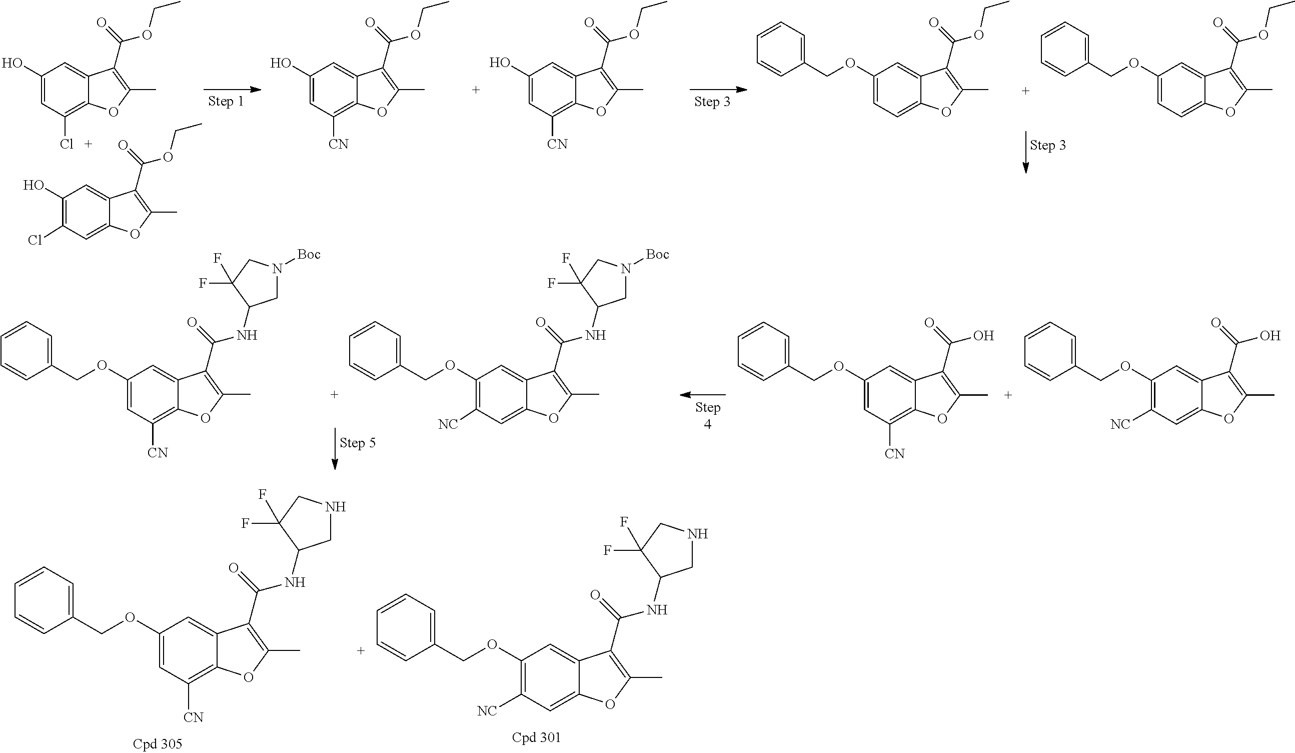

Step 1: A solution of mixture of ethyl 6-chloro-5-hydroxy-2-methylbenzofuran-3-carboxylate and ethyl 7-chloro-5-hydroxy-2-methylbenzofuran-3-carboxylate (1.0 g, 3.93 mmol) and Zn(CN)₂ (2.01 g, 17.71 mmol) in dimethylacetamide (12 mL) was degassed with argon for 10 min. To the above RM, Pd(P(t-Bu)₃)₂ (0.60 g, 1.18 mmol) was added in one portion and degassed for 5 min. The resulting mixture was heated at 160° C. and maintained for 2 h under microwaves. The reaction progress was monitored by TLC. The RM was poured into water (100 mL) and extracted with EtOAc (2×100 mL). The combined organic layer was washed with water (2×50 mL) and brine (50 mL). The organic layer was dried over anhydrous Na₂SO₄ and concentrated under reduced pressure. The crude product was purified by column chromatography over silica gel using 0-20% EtOAc in pet-ether as an eluent to afford a mixture of ethyl 6-cyano-5-hydroxy-2-methylbenzofuran-3-carboxylate and ethyl 7-cyano-5-hydroxy-2-methylbenzofuran-3-carboxylate (0.6 g, 62.5%).

Step 2: Benzyl alcohol (0.72 g, 6.73 mmol), ADDP (1.58 g, 6.28 mmol) and tri-n-butyl phosphine (1.47 mL, 6.28 mmol) were added sequentially to a pre-stirred solution of mixture of ethyl 6-cyano-5-hydroxy-2-methylbenzofuran-3-carboxylate and ethyl 7-cyano-5-hydroxy-2-methylbenzofuran-3-carboxylate (1.1 g, 4.48 mmol) in THF (30 mL) at RT under argon atmosphere. The resulting RM was stirred at RT for 2 h. The reaction progress was monitored by TLC. The RM was poured into water (100 mL) and extracted with EtOAc (2×100 mL). The combined organic layer was washed with water (50 mL), brine (50 mL), dried over anhydrous Na₂SO₄ and concentrated under reduced pressure. The crude product was purified by column chromatography over silica gel using a gradient mixture of 0-20% EtOAc in pet-ether as an eluent to afford a mixture of ethyl 5-(benzyloxy)-6-cyano-2-methylbenzofuran-3-carboxylate and ethyl 5-(benzyloxy)-7-cyano-2-methylbenzofuran-3-carboxylate (1.2 g, 73%).

Step 3: A solution of NaOH (0.57 g, 14.32 mmol) in water (10 mL) was added dropwise to a pre-stirred solution of mixture of ethyl 5-(benzyloxy)-6-cyano-2-methylbenzofuran-3-carboxylate and ethyl 5-(benzyloxy)-7-cyano-2-methylbenzofuran-3-carboxylate (1.2 g, 3.58 mmol) in a mixture of MeOH (20 mL) and THF (20 mL) at RT. The resulting RM was heated to 60° C. and maintained under stirring for 4 h. The reaction progress was monitored by TLC. The RM was cooled to RT and poured into ice cold water (50 mL), acidified with 1N HCl (pH˜2). The crude product was extracted with EtOAc (2×50 mL). The combined organic layer was washed with water (50 mL) followed by brine (50 mL), dried over anhydrous Na₂SO₄ and concentrated under reduced pressure to afford a mixture of 5-(benzyloxy)-6-cyano-2-methylbenzofuran-3-carboxylic acid and 5-(benzyloxy)-7-cyano-2-methylbenzofuran-3-carboxylic acid (1.0 g, 91%).

Step 4: To a pre-stirred solution mixture of 5-(benzyloxy)-6-cyano-2-methylbenzofuran-3-carboxylic acid and 5-(benzyloxy)-7-cyano-2-methylbenzofuran-3-carboxylic acid (1.0 g, 3.25 mmol), and tert-butyl 4-amino-3,3-difluoropyrrolidine-1-carboxylate (1.08 g, 4.88 mmol) in DMF (15 mL) were added DIPEA (1.68 mL, 9.77 mmol) followed by HATU (2.47 g, 6.51 mmol) at 0° C. under argon atmosphere. The resulting RM was allowed to attain RT and stirred for 16 h. The reaction progress was monitored by TLC. The RM was diluted with water (70 mL) and the organic compound was extracted with EtOAc (2×70 mL). The combined organic layer was washed with water (50 mL), brine (50 mL), dried over anhydrous Na₂SO₄ and concentrated under reduced pressure. The crude product was purified by column chromatography over silica gel using a gradient mixture of 0-55% EtOAc and pet-ether as an eluent to afford a mixture of tert-butyl 4-(5-(benzyloxy)-6-cyano-2-methylbenzofuran-3-carboxamido)-3,3-difluoropyrrolidine-1-carboxylate and tert-butyl 4-(5-(benzyloxy)-7-cyano-2-methylbenzofuran-3-carboxamido)-3,3-difluoropyrrolidine-1-carboxylate (1.0 g, 60%).

Step 5: 4.0 M HCl in dioxane (10 mL) was added drop-wise to a pre-stirred solution mixture of tert-butyl 4-(5-(benzyloxy)-6-cyano-2-methylbenzofuran-3-carboxamido)-3,3-difluoropyrrolidine-1-carboxylate and tert-butyl 4-(5-(benzyloxy)-7-cyano-2-methylbenzofuran-3-carboxamido)-3,3-difluoropyrrolidine-1-carboxylate (1.0 g, 1.95 mmol) in DCM (20 mL) at 0° C. The resulting RM was stirred at room temperature for 5 h. The reaction progress was monitored by TLC. The RM was concentrated under reduced pressure. The residue was partitioned between sat. NaHCO₃ solution (100 mL) and 10% MeOH in DCM (3×50 mL). The combined organic extracts were washed with water (50 mL), brine (50 mL), dried over anhydrous Na₂SO₄ and concentrated under reduced pressure. The crude product was purified by GRACE flash chromatography using 0.1% FA in water and acetonitrile as eluent to afford a mixture of Cpd 301 and Cpd 305 (0.6 g, 75%). A preparative chiral SFC was performed on the mixture of racemic Cpd 301 and racemic Cpd 305 to afford Cpd 301—En 1, Cpd 301—En 2, Cpd 305—En 1 and Cpd 305—En 2.

### Synthesis of 5-(benzyloxy)-6-chloro-N-(4,4-difluoropyrrolidin-3-yl)-2-methylbenzofuran-3-carboxamide (Cpd 300) and 5-(benzyloxy)-7-chloro-N-(4,4-difluoropyrrolidin-3-yl)-2-methylbenzofuran-3-carboxamide (Cpd 304)

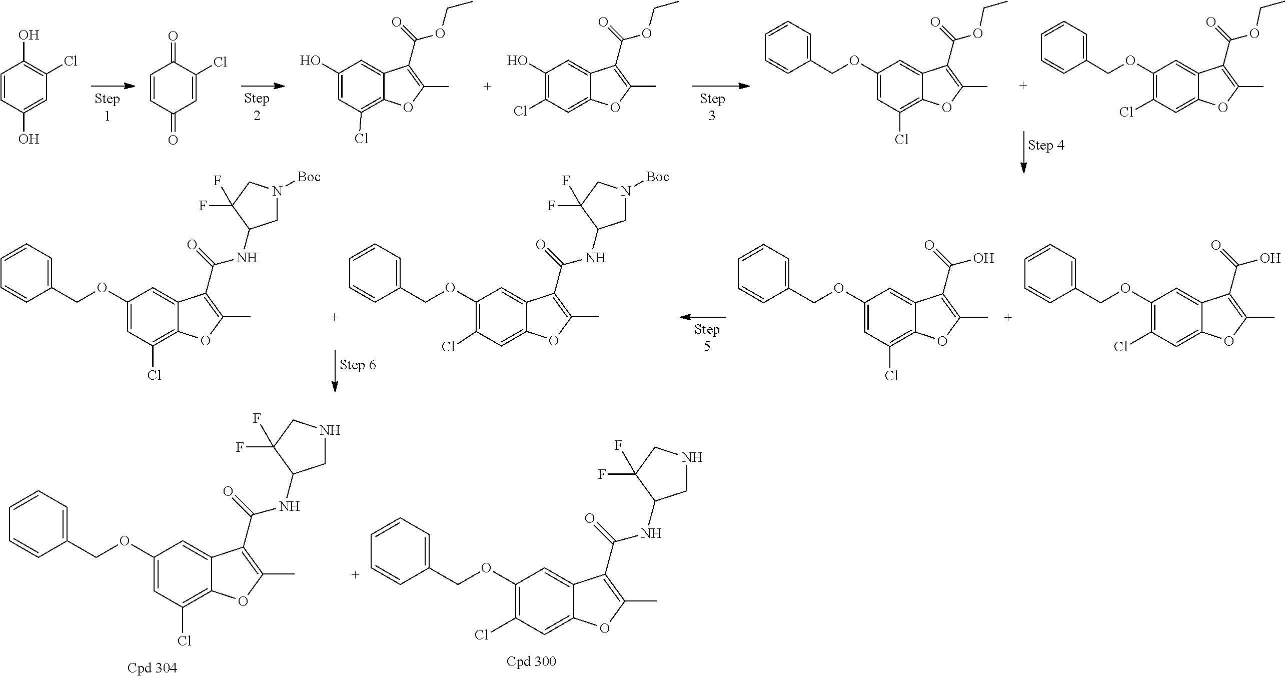

Step 1: To a pre-stirred solution of CAN (199.18 g, 363.32 mmol) in water (500 mL) was added 2-chlorobenzene-1,4-diol (25.0 g, 173.01 mmol) at 0° C. The resulting RM was stirred at RT for 4 h. The reaction progress was monitored by TLC. The organic compound was extracted with diethyl ether (3×200 mL). The combined organic layer was washed with brine (50 mL) and dried over anhydrous Na₂SO₄. The dried organic layer was passed through a silica gel column using diethyl ether as eluant and thus collected fractions were concentrated under reduced pressure to afford 2-chlorocyclohexa-2,5-diene-1,4-dione (20 g, 83%).

Step 2: To a pre-stirred solution of 2-chlorocyclohexa-2,5-diene-1,4-dione (20 g, 140.84 mmol) in toluene (300 mL) was added ethyl 3-oxobutanoate (54.92 g, 422.53 mmol) followed by anhydrous ZnCl₂ (23.0 g, 169.0 mmol) at RT under argon atmosphere. The resulting RM was heated to reflux and maintained for 16 h using Dean-Stark apparatus. The reaction progress was monitored by TLC. The RM was cooled to RT, filtered through celite pad and the celite pad was washed with EtOAc (500 mL). The combined clear filtrate was concentrated under reduced pressure. The crude product was purified by FCC over silica gel and using 0-10% EtOAc in pet-ether as an eluent to afford mixture of ethyl 6-chloro-5-hydroxy-2-methylbenzofuran-3-carboxylate and ethyl 7-chloro-5-hydroxy-2-methylbenzofuran-3-carboxylate (7.0 g, 20%).

Step 3: Phenylmethanol (0.36 g, 5.90 mmol), ADDP (1.38 g, 5.51 mmol) and tri-n-butyl phosphine (1.35 mL, 5.51 mmol) were added sequentially to a pre-stirred solution of a mixture of ethyl 6-chloro-5-hydroxy-2-methylbenzofuran-3-carboxylate compound and ethyl 7-chloro-5-hydroxy-2-methylbenzofuran-3-carboxylate (1.0 g, 3.93 mmol) in THF (30 mL) at RT under argon atmosphere. The RM was stirred for 2 h. The reaction progress was monitored by TLC. The RM was poured into water (100 mL) and extracted with EtOAc (2×100 mL). The combined organic layer was washed with water (50 mL), brine (50 mL) and dried over anhydrous Na₂SO₄ and concentrated under reduced pressure. The crude product was purified by column chromatography over silica using 0-20% EtOAc in pet-ether as an eluent to afford a mixture of ethyl 5-(benzyloxy)-6-chloro-2-methylbenzofuran-3-carboxylate and ethyl 5-(benzyloxy)-7-chloro-2-methylbenzofuran-3-carboxylate (1.2 g, 89%).

Step 4: A solution of NaOH (0.46 g, 11.62 mmol) in water (8 mL) was added drop-wise to a pre-stirred solution of mixture of ethyl 5-(benzyloxy)-6-chloro-2-methylbenzofuran-3-carboxylate and ethyl 5-(benzyloxy)-7-chloro-2-methylbenzofuran-3-carboxylate (1.0 g, 2.90 mmol) in a mixture of MeOH (15 mL) and THF (7 mL) at RT. The resulting RM was heated to 60° C. and maintained for 4 h. The reaction progress was monitored by TLC. The RM was cooled to RT and poured into ice cold water (50 mL), acidified with 1N HCl (pH-2). The crude product was extracted with EtOAc (2×50 mL). The combined organic layer was washed with water (50 mL) followed by brine (50 mL), dried over anhydrous Na₂SO₄ and concentrated under reduced pressure to afford a mixture of 5-(benzyloxy)-6-chloro-2-methylbenzofuran-3-carboxylic acid and 5-(benzyloxy)-7-chloro-2-methylbenzofuran-3-carboxylic acid (0.8 g, 87%).

Step 5: To a pre-stirred solution of mixture of 5-(benzyloxy)-6-chloro-2-methylbenzofuran-3-carboxylic acid and 5-(benzyloxy)-7-chloro-2-methylbenzofuran-3-carboxylic acid (0.8 g, 2.53 mmol), and tert-butyl 4-amino-3,3-difluoropyrrolidine-1-carboxylate (0.85 g, 3.84 mmol) in DMF (15 mL) was added DIPEA (1.35 mL, 7.59 mmol) followed by HATU (1.92 g, 5.06 mmol) at 0° C. under argon atmosphere. The RM was allowed to attain RT and stirred for 16 h. The reaction progress was monitored by TLC. The RM was diluted with water (70 mL) and the organic compound was extracted with EtOAc (2×70 mL). The combined organic layer was washed with water (50 mL), brine (50 mL) dried over anhydrous Na₂SO₄ and concentrated under reduced pressure. The crude product was purified by column chromatography over silica using 0-55% EtOAc and pet-ether as an eluent to afford a mixture of tert-butyl 4-(5-(benzyloxy)-6-chloro-2-methylbenzofuran-3-carboxamido)-3,3-difluoropyrrolidine-1-carboxylate and tert-butyl 4-(5-(benzyloxy)-7-chloro-2-methylbenzofuran-3-carboxamido)-3,3-difluoropyrrolidine-1-carboxylate (0.75 g, 57%).

Step 6: 4.0 M HCl in dioxane (2.88 mL, 11.53 mmol) was added drop-wise to a pre-stirred solution of mixture of tert-butyl 4-(5-(benzyloxy)-6-chloro-2-methylbenzofuran-3-carboxamido)-3,3-difluoropyrrolidine-1-carboxylate and tert-butyl 4-(5-(benzyloxy)-7-chloro-2-methylbenzofuran-3-carboxamido)-3,3-difluoro-pyrrolidine-1-carboxylate (0.75 g, 1.44 mmol) in DCM (10 mL) at 0° C. The RM was allowed to attain RT and stirred for 5 h. The reaction progress was monitored by TLC. The RM was concentrated under reduced pressure and basified with sat. NaHCO₃ solution (100 mL). The organic compound was extracted with 10% MeOH in DCM (3×50 mL), washed with water (50 mL) and brine (50 mL). The organic layer was dried over anhydrous Na₂SO₄ and concentrated under reduced pressure. The crude product was purified by GRACE flash chromatography using acetonitrile and 0.1% FA in water as an eluent to afford a mixture of Cpd 300 and Cpd 304. A preparative chiral SFC was performed on the mixture of racemic Cpd 300 and racemic Cpd 304 to afford Cpd 300—En 1, Cpd 300—En 2, Cpd 304—En 1 and Cpd 304—En 2.

### Synthesis of 5-(benzyloxy)-N-(4,4-difluoropyrrolidin-3-yl)-6-fluoro-2-methylbenzofuran-3-carboxamide (Cpd 302) and 6-fluoro-N-(4-(hydromethyl)tetrahydro-2H-pyran-4-yl)-2-methyl-5-((4-methylthiazol-5-yl)-methoxy)benzofuran-3-carboxamide (Cpd 306)

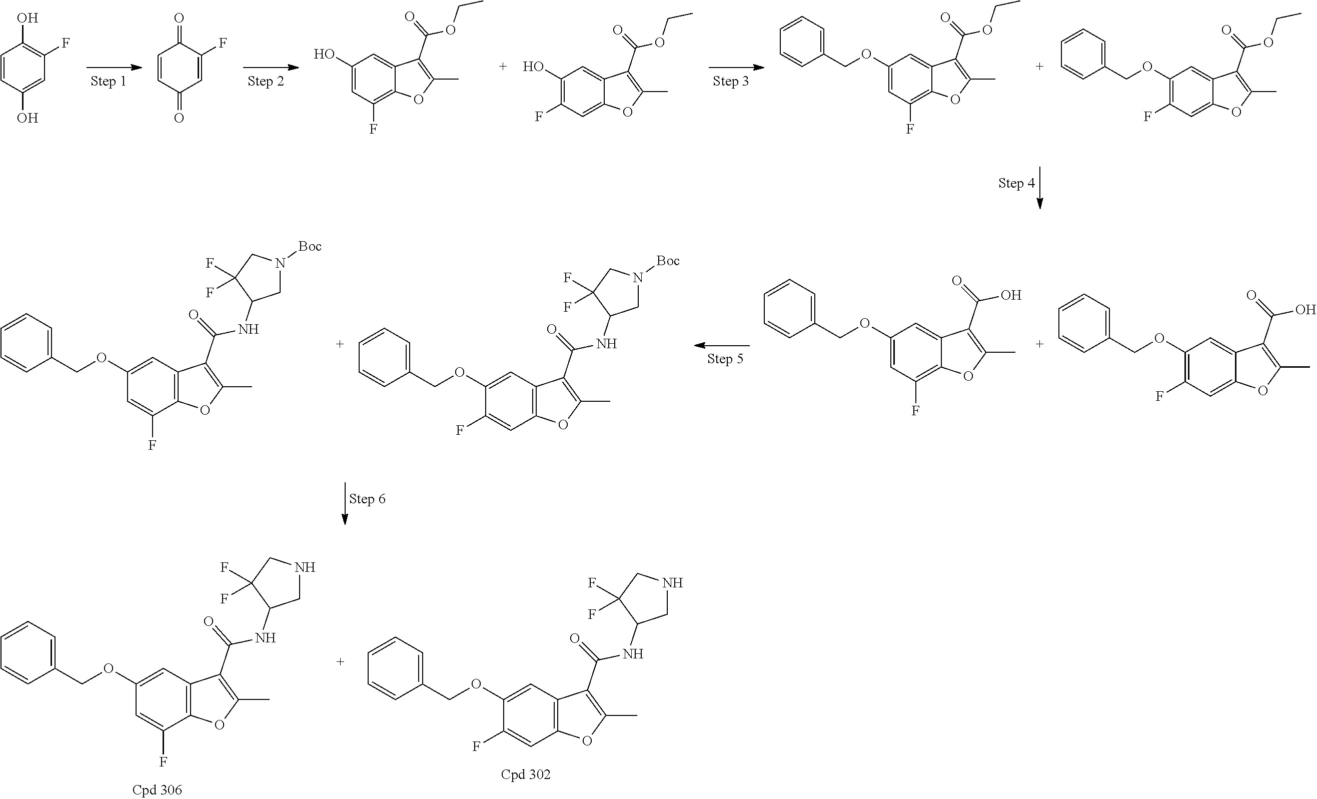

Step 1: To a pre-stirred solution of CAN (27.41 g, 50.00 mmol) in water (60 mL) was added 2-fluorobenzene-1,4-diol (3.0 g, 23.80 mmol) at 0° C. The resulting RM was stirred at RT for 4 h. The reaction progress was monitored by TLC. The organic compound was extracted with diethyl ether (3×50 mL). The combined organic layer was washed with brine (50 mL), dried over anhydrous Na₂SO₄ and filtered. The clear filtrate was passed over a silica gel column, eluted with diethyl ether and the collected fractions were evaporated under vacuo to afford 2-fluorocyclohexa-2,5-diene-1,4-dione (2.5 g, 84%).

Step 2: To a pre-stirred solution of 2-fluorocyclohexa-2,5-diene-1,4-dione (2.5 g, 20.16 mmol) in toluene (30 mL) was added ethyl 3-oxobutanoate (7.86 g, 60.48 mmol) followed by anhydrous ZnCl₂ (3.29 g, 24.19 mmol) at RT under argon atmosphere. The resulting RM was heated to reflux and maintained for 16 h using a Dean-Stark apparatus. The reaction progress was monitored by TLC. The RM was cooled to RT, filtered through celite pad and the celite pad was washed with EtOAc (70 mL). The combined clear filtrate was concentrated under reduced pressure. The crude product was purified by FCC over silica gel using 0-10% EtOAc in pet-ether as an eluent to afford a mixture of ethyl 7-fluoro-5-hydroxy-2-methylbenzofuran-3-carboxylate and ethyl 6-fluoro-5-hydroxy-2-methylbenzofuran-3-carboxylate (0.8 g, 21%).

Step 3: Phenylmethanol (0.47 mL, 3.3 mmol), ADDP (1.18 g, 4.7 mmol) and tri-n-butylphosphine (0.95 g, 4.7 mmol) were added sequentially to a pre-stirred solution of mixture of ethyl 7-fluoro-5-hydroxy-2-methylbenzofuran-3-carboxylate and ethyl 6-fluoro-5-hydroxy-2-methylbenzofuran-3-carboxylate (800 mg, 3.3 mmol) in THF (25 mL) at 0° C. under argon atmosphere. The RM was allowed to attain RT and stirred for 3 h. The reaction progress was monitored by TLC. The RM was poured into water (100 mL) and extracted with EtOAc (3×100 mL). The combined organic layer was washed with brine (100 mL), dried over anhydrous Na₂SO₄ and concentrated under reduced pressure. The crude product was purified by column chromatography over silica using 10-20% EtOAc in pet ether as an eluent to afford a mixture of ethyl 5-(benzyloxy)-7-fluoro-2-methylbenzofuran-3-carboxylate and ethyl 5-(benzyloxy)-6-fluoro-2-methylbenzofuran-3-carboxylate (600 mg, 54%).

Step 4: 2 N NaOH (20 mL) was added dropwise to a pre-stirred solution of mixture of ethyl 5-(benzyloxy)-7-fluoro-2-methylbenzofuran-3-carboxylate and ethyl 5-(benzyloxy)-6-fluoro-2-methylbenzofuran-3-carboxylate (600 mg, 1.82 mmol) in a mixture of MeOH (20 mL) and THF (5 mL) at RT. The resulting RM was heated to 60° C. and maintained for 3 h. The reaction progress was monitored by TLC. The RM was cooled to RT, poured into ice cold water (100 mL) and acidified with 1N HCl (pH-2). The crude product was extracted with EtOAc (3×50 mL). The combined organic layer was washed with water (100 mL) followed by brine (100 mL), dried over anhydrous Na₂SO₄ and concentrated under reduced pressure to afford a mixture of 5-(benzyloxy)-7-fluoro-2-methylbenzofuran-3-carboxylic acid and 5-(benzyloxy)-6-fluoro-2-methylbenzofuran-3-carboxylic acid (450 mg, 82%). The obtained crude product mixture was used for next step without further purification.

Step 5: To a pre-stirred solution of mixture of 5-(benzyloxy)-7-fluoro-2-methylbenzofuran-3-carboxylic acid and 5-(benzyloxy)-6-fluoro-2-methylbenzofuran-3-carboxylic acid (400 mg, 1.33 mmol) and tert-butyl 4-amino-3,3-difluoropyrrolidine-1-carboxylate (355 mg, 1.59 mmol) in DMF (20 mL) was added DIPEA (0.49 mL, 2.66 mmol) followed by HATU (1.01 g, 2.66 mmol) at 0° C. under argon atmosphere. The RM was allowed to attain RT and stirred for 1 h. The reaction progress was monitored by TLC. The RM was diluted with ice cold water (100 mL) and filtered. The resulting solid was washed with water (50 mL), dried under reduced pressure to afford a mixture of tert-butyl 4-(5-(benzyloxy)-7-fluoro-2-methylbenzofuran-3-carboxamido)-3,3-difluoropyrrolidine-1-carboxylate and tert-butyl 4-(5-(benzyloxy)-6-fluoro-2-methylbenzofuran-3-carboxamido)-3,3-difluoropyrrolidine-1-carboxylate (550 mg, 82%).

Step 6: 4M HCl in dioxane (6 mL) was added dropwise to a solution of mixture of tert-butyl 4-(5-(benzyloxy)-7-fluoro-2-methylbenzofuran-3-carboxamido)-3,3-difluoropyrrolidine-1-carboxylate and tert-butyl 4-(5-(benzyloxy)-6-fluoro-2-methylbenzofuran-3-carboxamido)-3,3-difluoropyrrolidine-1-carb oxy late (0.55 g, 1.0 mmol) in DCM (20 mL) at 0° C. under argon atmosphere. The RM was warmed to RT and stirred for 5 h. The reaction progress was monitored by TLC. The excess solvents were evaporated in vacuo. The resulting crude product was basified with sat. NaHCO₃ (pH ˜9). The free base product was extracted with EtOAc (3×50 mL). The combined organic layer was washed with water (100 mL), brine (100 mL), dried over anhydrous Na₂SO₄ and concentrated under reduced pressure. The crude product was purified by GRACE flash chromatography using 0-50% acetonitrile in 0.1% FA in water as an eluent to afford a mixture of Cpd 306 and Cpd 302 (0.4 g, 90%). A preparative chiral SFC was performed on the mixture of racemic Cpd 306 and racemic Cpd 302 to afford Cpd 306—En 1, Cpd 306—En 2, Cpd 302—En 1 and Cpd 302—En 2.

### Synthesis of 5-(benzyloxy)-N-(4,4-difluoropyrrolidin-3-yl)-2,6-dimethylbenzofuran-3-carboxamide (Cpd 303) and 5-(benzyloxy)-N-(4,4-difluoropyrrolidin-3-yl)-2,7-dimethylbenzofuran-3-carboxamide (Cpd 307)

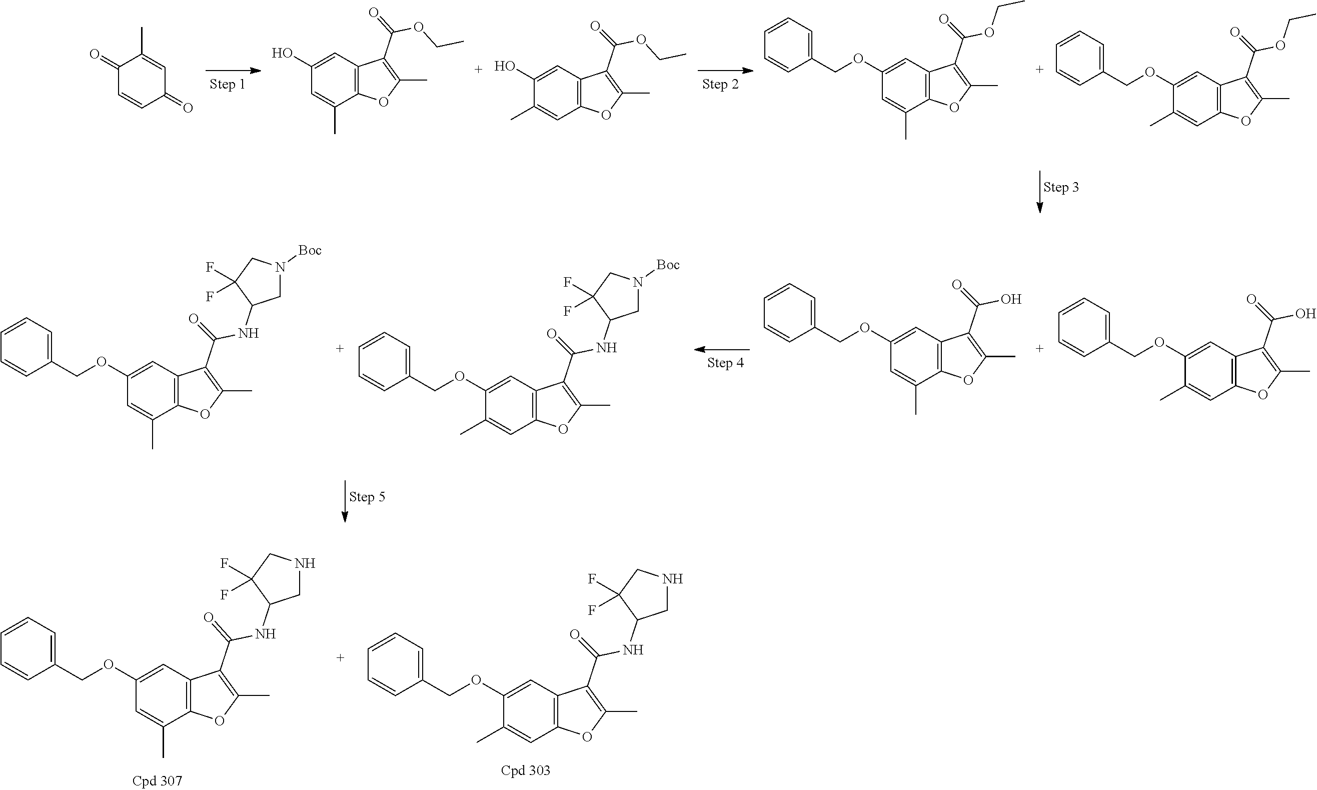

Step 1: To a pre-stirred solution of 2-methylcyclohexa-2,5-diene-1,4-dione (10.0 g, 81.96 mmol) in toluene (250 mL) was added ethyl 3-oxobutanoate (31.9 g, 245.89 mmol) followed by anhydrous ZnCl₂ (13.4 g, 98.35 mmol) at RT under argon atmosphere. The resulting RM was heated to reflux and maintained for 16 h using a Dean-Stark apparatus. The reaction progress was monitored by TLC. The RM was cooled to RT, filtered through celite pad and the celite pad was washed with EtOAc (300 mL). The combined clear filtrate was concentrated under reduced pressure. The crude product was purified by FCC over silica gel using 0-10% EtOAc in pet-ether as an eluent to afford a mixture of ethyl 5-hydroxy-2,7-dimethylbenzofuran-3-carboxylate and ethyl 5-hydroxy-2,6-dimethylbenzofuran-3-carboxylate (10.0 g, 52%).

Step 2: Phenylmethanol (4.7 g, 32.05 mmol), ADDP (7.53 g, 29.91 mmol) and tri-n-butylphosphine (7.36 mL, 29.91 mmol) were added sequentially to a pre-stirred solution of mixture of ethyl 5-hydroxy-2,7-dimethylbenzofuran-3-carboxylate and ethyl 5-hydroxy-2,6-dimethylbenzofuran-3-carboxylate (5.0 g, 21.36 mmol) in THF (250 mL) at 0° C. under argon atmosphere. The RM was allowed to attain RT and stirred for 3 h. The reaction progress was monitored by TLC. The RM was poured into water (150 mL) and extracted with EtOAc (2×100 mL). The combined organic layer was washed with brine (100 mL), dried over anhydrous Na₂SO₄ and concentrated under reduced pressure. The crude product was purified by GRACE flash chromatography using 0-60% acetonitrile and 0.1% FA in water as an eluent to afford a mixture of ethyl 5-(benzyloxy)-2,7-dimethylbenzofuran-3-carboxylate and ethyl 5-(benzyloxy)-2,6-dimethylbenzofuran-3-carboxylate (4.0 g, 41%).

Step 3: A solution of NaOH (2.4 g, 61.7 mmol) in water (32 mL) was added to a pre-stirred solution of mixture of ethyl 5-(benzyloxy)-2,7-dimethylbenzofuran-3-carboxylate and ethyl 5-(benzyloxy)-2,6-dimethylbenzofuran-3-carboxylate (4.0 g, 12.3 mmol) in a mixture of MeOH (40 mL) and THF (40 mL) at RT. The resulting RM was heated to 60° C. and maintained for 3 h. The reaction progress was monitored by TLC. The RM was cooled to RT, poured into ice cold water (200 mL) and acidified with 1N HCl (pH-2). The crude product was extracted with EtOAc (2×100 mL). The combined organic layer was washed with water (2×100 mL) followed by brine (100 mL), dried over anhydrous Na₂SO₄ and concentrated under reduced pressure to afford a mixture of 5-(benzyloxy)-2,7-dimethylbenzofuran-3-carboxylic acid and 5-(benzyloxy)-2,6-dimethylbenzofuran-3-carboxylic acid (2.0 g, 54%). The obtained crude product mixture was used for next step without further purification.

Step 4: To a pre-stirred solution of mixture of 5-(benzyloxy)-2,7-dimethylbenzofuran-3-carboxylic acid and 5-(benzyloxy)-2,6-dimethylbenzofuran-3-carboxylic acid (1.5 g, 5.1 mmol) and tert-butyl 4-amino-3,3-difluoropyrrolidine-1-carboxylate (1.12 g, 5.1 mmol) in DMF (20 mL) was added DIPEA (2.7 mL, 15.2 mmol) followed by HATU (3.85 g, 10.1 mmol) at 0° C. under argon atmosphere. The RM was allowed to attain RT and stirred for 3 h. The reaction progress was monitored by TLC. The RM was diluted with ice cold water (50 mL) and filtered. The obtained solid was washed with water (200 mL), dried under reduced pressure to afford a mixture of tert-butyl 4-(5-(benzyloxy)-2,7-dimethylbenzofuran-3-carboxamido)-3,3-difluoropyrrolidine-1-carboxylate and tert-butyl·4-(5-(benzyloxy)-2,6-dimethylbenzofuran-3-carboxamido)-3,3-difluoropyrrolidine-1-carboxylate (0.9 g, 65%).

Step 5: TFA (1.0 mL) was added to a solution of mixture of tert-butyl 4-(5-(benzyloxy)-2,7-dimethylbenzofuran-3-carboxamido)-3,3-difluoropyrrolidine-1-carboxylate and tert-butyl·4-(5-(benzyloxy)-2,6-dimethylbenzofuran-3-carboxamido)-3,3-difluoropyrrolidine-1-carboxylate (0.9 g, 1.79 mmol) in DCM (10 mL) at 0° C. under argon atmosphere. The RM was warmed to RT and stirred for 16 h. The reaction progress was monitored by TLC. The excess solvents were evaporated in vacuo. The crude product was basified with sat. NaHCO₃ (pH ˜9). The free base product was extracted with EtOAc (2×100 mL). The organic layer was dried over anhydrous Na₂SO₄ and solvent was removed under reduced pressure. The crude product was purified by GRACE flash chromatography using 0-60% acetonitrile and 0.1% FA in water as an eluent to afford a mixture of Cpd 307 and Cpd 303. A preparative chiral SFC was performed on the mixture of racemic Cpd 307 and racemic Cpd 303 to afford Cpd 307—En 1, Cpd 307—En 2, Cpd 303—En 1 and Cpd 303—En 2.

### Synthesis of N-(4-(hydroxymethyl)tetrahydro-2H-pyran-4-yl)-2-methyl-5-(1-phenylethoxy)benzofuran-3-carboxamide (Cpd 308)

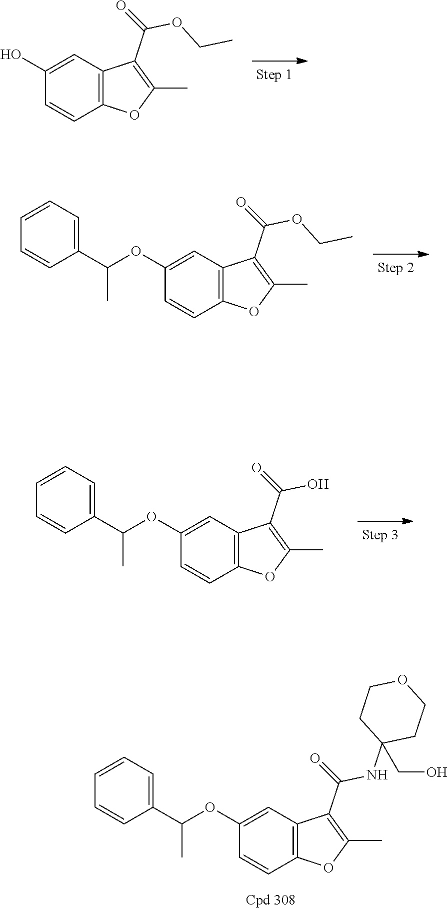 [SMILES: CCOC(=O)c1c(C)oc2ccc(O)cc12 ; CCOC(=O)c1c(C)oc2ccc(OC(C)c3ccccc3)cc12 ; Cc1oc2ccc(OC(C)c3ccccc3)cc2c1C(=O)O ; Cc1oc2ccc(OC(C)c3ccccc3)cc2c1C(=O)NC1(CO)CCOCC1]

Step 1: A suspension of ethyl 5-hydroxy-2-methylbenzofuran-3-carboxylate (1.5 g, 6.818 mmol), (1-bromoethyl)benzene (1.89 g, 10.227 mmol) and K₂CO₃ (2.35 g, 17.045 mmol) in acetone (50 mL) were stirred at for 16 h. The RM was filtered through a celite pad and the bed was washed with EtOAc. The combined filtrate was washed with 2N NaOH solution (2×30 mL), water (50 mL), brine (50 mL), dried over anhydrous Na₂SO₄ and concentrated under reduced pressure to afford ethyl 2-methyl-5-(1-phenylethoxy)benzofuran-3-carboxylate (2.5 g, 52%). The crude product thus obtained was used for next step without further purification.

Step 2: A solution of NaOH (1.23 g, 30.864 mmol) in water (10.0 mL) was added to a pre-stirred solution of ethyl 2-methyl-5-(1-phenylethoxy)benzofuran-3-carboxylate (2.5 g, 7.716 mmol) in MeOH (20 mL) and THF (20 mL) at RT. The resulting RM was heated to 60° C. and maintained for 4 h. The reaction progress was monitored by TLC. The RM was cooled to RT, poured into ice cold water (75 mL), acidified with 1N HCl (pH-2.0) and extracted with EtOAc (2×75 mL). The combined organic layer was washed with water (2×50 mL), brine (50 mL), dried over anhydrous Na₂SO₄ and concentrated under reduced pressure. The crude solid was re-precipitated with DCM and pet-ether, and the resulting solid was dried to afford 2-methyl-5-(1-phenylethoxy)benzofuran-3-carboxylic acid (1.4 g, 70% over two steps).

Step 3: To a pre-stirred solution of 2-methyl-5-(1-phenylethoxy)benzofuran-3-carboxylic acid (300 mg, 1.013 mmol) and (4-aminotetrahydro-2H-pyran-4-yl)methanol (199 mg, 1.520 mmol) in DMF (6 mL) were added DIPEA (0.53 mL, 3.039 mmol) followed by HATU (577 mg, 1.52 mmol) at 0° C. under argon atmosphere. The RM was stirred at RT for 16 h. The reaction progress was monitored by TLC. The RM was diluted with water (50 mL) and extracted with EtOAc (2×50 mL). The combined organic layer was washed with water (50 mL), brine (50 mL), dried over anhydrous Na₂SO₄ and concentrated under reduced pressure. The crude product was re-precipitated with DCM and n-pentane, the solid thus obtained was dried to afford Cpd 308 (250 mg, 60%). A preparative chiral SFC was performed on racemic Cpd 308 to afford Cpd 308—En 1 and Cpd 308—En 2.

The following compounds were prepared in a manner similar (use of appropriate reagents and purification methods known to the person skilled in the art) to Cpd 308—En 1 and Cpd 308—En 2: Cpd 336—Dia 1 and Cpd 336—Dia 2

### Synthesis of N-(1-carbamoylcyclobutyl)-2-methyl-5-(1-phenylethoxy)benzofuran-3-carboxamide (Cpd 343)

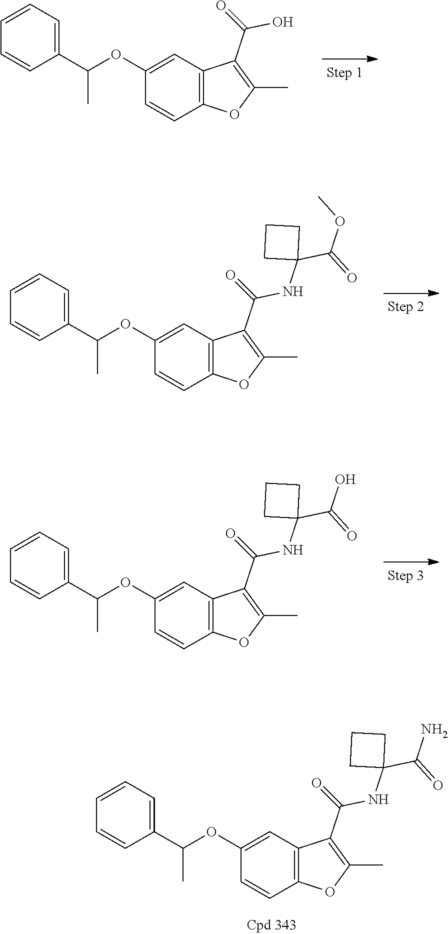 [SMILES: Cc1oc2ccc(OC(C)c3ccccc3)cc2c1C(=O)O ; COC(=O)C1(NC(=O)c2c(C)oc3ccc(OC(C)c4ccccc4)cc23)CCC1 ; Cc1oc2ccc(OC(C)c3ccccc3)cc2c1C(=O)NC1(C(=O)O)CCC1 ; Cc1oc2ccc(OC(C)c3ccccc3)cc2c1C(=O)NC1(C(N)=O)CCC1]

Step 1: Methyl 1-aminocyclobutane-1-carboxylate hydrochloride (334 mg, 2.02 mmol) was added to a stirred solution mixture of 2-methyl-5-(1-phenylethoxy)benzofuran-3-carboxylic acid (400 mg, 1.35 mmol), HATU (1.00 g, 2.70 mmol) and DIPEA (0.74 mL, 4.00 mmol) in DMF (10 mL) at RT. The RM was stirred at RT under argon atmosphere for 1 h. The reaction progress was monitored by TLC. The RM was diluted with water (25 mL) and stirred for 15 min. The precipitated solid was filtered and dried under vacuum to afford 400 mg (72%) of methyl 1-(2-methyl-5-(1-phenylethoxy)benzofuran-3-carboxamido)cyclobutane-1-carboxylate as an off-white solid. TLC system: 30% Ethyl acetate in pet ether; RF: 0.4.

Step 2: 2N NaOH (5 mL) was added to a stirred solution of methyl 1-(2-methyl-5-(1-phenylethoxy)benzofuran-3-carboxamido)cyclobutane-1-carboxylate (0.4 g, 0.98 mmol) in methanol (5 mL) and THF (5 mL) at RT and the RM was stirred at RT for 16 h. The reaction progress was monitored by TLC. The RM was diluted with water (25 mL), acidified to pH ˜2 with 1N aqueous HCl solution (10 mL) and stirred for 15 min. The precipitated solid was filtered and dried under vacuum to afford 300 mg (79%) of 1-(2-methyl-5-(1-phenylethoxy)benzofuran-3-carboxamido)cyclobutane-1-carboxylic acid as an off-white solid. TLC system: 50% Ethyl acetate in pet ether; RF: 0.1.

Step 3: NH₄Cl (202 mg, 3.81 mmol) was added to a stirred solution of 1-(2-methyl-5-(1-phenylethoxy)benzofuran-3-carboxamido)cyclobutane-1-carboxylic acid (300 mg, 0.76 mmol), HATU (580 mg, 1.52 mmol) and DIPEA (0.42 mL, 2.29 mmol) in DMF (10 mL) at RT and the RM was stirred at RT for 1 h. The RM was diluted with water (25 mL) and stirred for 30 minutes. The precipitated solid was filtered and dried under vacuum to afford N-(1-carbamoylcyclobutyl)-2-methyl-5-(1-phenylethoxy)benzofuran-3-carboxamide (Cpd 343) (290 mg, 96%). A preparative chiral SFC was performed on racemic Cpd 343 to afford Cpd 343—En 1 and Cpd 343—En 2.

The following compound was prepared in a similar manner (use of appropriate reagents and purification methods (including chiral HPLC or chiral SFC) known to the person skilled in the art) as described for Cpd 343: Cpd 344.

### Synthesis of N-(4-(hydroxymethyl)tetrahydro-2H-pyran-4-yl)-2-methyl-5-((2-phenylpropan-2-yl)oxy)benzofuran-3-carboxamide (Cpd 345)

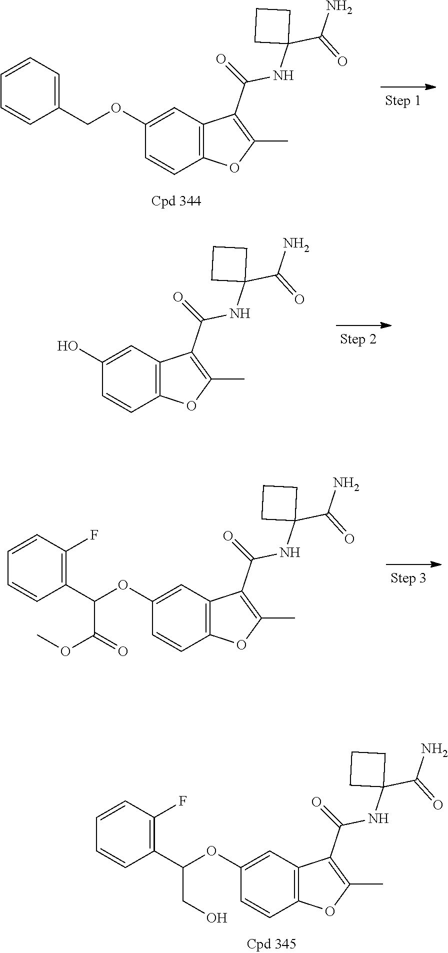 [SMILES: Cc1oc2ccc(OCc3ccccc3)cc2c1C(=O)NC1(C(N)=O)CCC1 ; Cc1oc2ccc(O)cc2c1C(=O)NC1(C(N)=O)CCC1 ; COC(=O)C(Oc1ccc2oc(C)c(C(=O)NC3(C(N)=O)CCC3)c2c1)c1ccccc1F ; Cc1oc2ccc(OC(CO)c3ccccc3F)cc2c1C(=O)NC1(C(N)=O)CCC1]

Step 1: To a suspension of 10% Pd/C (300 mg) in ethyl acetate (10 mL) was added 5-(benzyloxy)-N-(1-carbamoylcyclobutyl)-2-methylbenzofuran-3-carboxamide (Cpd 344) (0.8 g, 2.11 mmol) in ethyl acetate (10 mL) at RT and the resulting reaction mixture was stirred at RT under hydrogen gas balloon pressure for 16 h. The reaction progress was monitored by TLC. The RM was filtered. The solid was washed with ethyl acetate (50 mL). Combined organic layers were concentrated under reduced pressure to afford N-(1-carbamoylcyclobutyl)-5-hydroxy-2-methylbenzofuran-3-carboxamide (600 mg, crude) as a pale yellow solid. TLC system: 100% Ethyl acetate; RF: 0.2.

Step 2: Cesium carbonate (1.35 g, 4.16 mmol) was added to a stirred solution of N-(1-carbamoylcyclobutyl)-5-hydroxy-2-methylbenzofuran-3-carboxamide (600 mg, 2.08 mmol) and methyl-2-bromo-2-(2-fluorophenyl)acetate (617 mg, 2.49 mmol) in acetonitrile (20 mL) at RT. The RM was stirred at RT for 4 h and the reaction progress was monitored by TLC. The RM was filtered and concentrated under reduced pressure to afford methyl 2-((3-((1-carbamoylcyclobutyl)carbamoyl)-2-methylbenzofuran-5-yl)oxy)-2-(2-fluorophenyl)acetate (700 mg) as a pale yellow solid. TLC system: 70% Ethyl acetate in pet-ether; RF: 0.4.

Step 3: NaBH₄ (150 mg, 3.96 mmol) was added to a stirred solution of methyl 2-((3-((1-carbamoylcyclobutyl)carbamoyl)-2-methylbenzofuran-5-yl)oxy)-2-(2-fluorophenyl)acetate (0.6 g, 1.32 mmol) in methanol (20 mL) at 0° C. and the RM was stirred at RT for 2 h. The reaction progress was monitored by TLC. The RM was diluted with water (50 mL) and extracted with ethyl acetate (3×50 mL). Combined organic layers were washed with water (100 mL), brine (100 mL), dried over anhydrous Na₂SO₄ and concentrated under reduced pressure. The residue was purified by grace flash chromatography using 0.1% formic acid in water and acetonitrile as an eluent to afford N-(1-carbamoylcyclobutyl)-5-(1-(2-fluorophenyl)-2-hydroxyethoxy)-2-methylbenzofuran-3-carboxamide (240 mg, 16% over 6 steps)(Cpd 345). A preparative chiral SFC was performed on racemic Cpd 345 to afford Cpd 345—En 1 and Cpd 345—En 2.

### Synthesis of N—((S)-1-amino-3-hydroxy-1-oxopropan-2-yl)-5-(2-methoxy-1-phenylethoxy)-2-methylbenzofuran-3-carboxamide (Cpd 346)

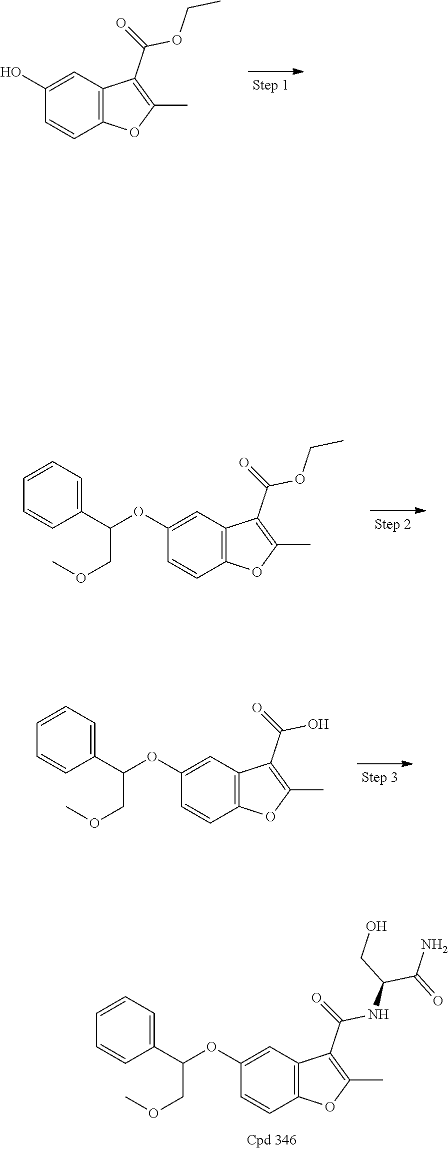 [SMILES: CCOC(=O)c1c(C)oc2ccc(O)cc12 ; CCOC(=O)c1c(C)oc2ccc(OC(COC)c3ccccc3)cc12 ; COCC(Oc1ccc2oc(C)c(C(=O)O)c2c1)c1ccccc1 ; COCC(Oc1ccc2oc(C)c(C(=O)N[C@@H](CO)C(N)=O)c2c1)c1ccccc1]

Step 1: To a stirred solution of ethyl 5-hydroxy-2-methylbenzofuran-3-carboxylate (3) (500 mg, 2.27 mmol) and 1-bromo-2-methoxyethyl)benzene (732.9 mg, 3.40 mmol) in acetonitrile (10 mL) was added Cs₂CO₃ (2.60 g, 6.81 mmol) at RT. The RM was warmed to 80° C. and stirred for 16 h. The reaction progress was monitored by TLC. The RM was filtered and filtrate was concentrated under reduced pressure to get ethyl 5-(2-methoxy-1-phenylethoxy)-2-methylbenzofuran-3-carboxylate (140 mg, 17.4%) as brown liquid. TLC system: 20% Ethyl acetate in pet ether; RF: 0.6.

Step 2: A solution of NaOH (158.19 g, 3.954 mmol) in water (10 mL) was added to a stirred solution of ethyl 5-(2-methoxy-1-phenylethoxy)-2-methylbenzofuran-3-carboxylate (140 mg, 0.3954 mmol) in methanol (5 mL) and THF (5 mL) at RT. The RM was stirred for 16 h at RT. The reaction progress was monitored by TLC. The RM was concentrated under reduced pressure, diluted with water (15 mL) and pH was adjusted to −6 with 1N aqueous HCl solution. The precipitated solid was filtered and dried under vacuum to afford 5-(2-methoxy-1-phenylethoxy)-2-methylbenzofuran-3-carboxylic acid (5) (110 mg, 85%) as a brown solid. TLC system: 100% Ethylacetate; RF: 0.2.

Step 3: To a stirred solution of 5-(2-methoxy-1-phenylethoxy)-2-methylbenzofuran-3-carboxylic acid (110 mg, 0.551 mmol), HATU (314.6 mg, 0.8281 mmol) and DIPEA (214.07 mg, 1.6563 mmol) in DMF (5 mL) was added L-serinamide·HCl (116.4 g, 0.8281 mmol) at RT under argon atmosphere. The RM was stirred at RT for 2 h. The reaction progress was monitored by TLC. The RM was poured into ice water (50 mL) and extracted with ethyl acetate (3×50 mL). Combined organic layers were washed with water (2×100 mL), brine (100 mL), dried over anhydrous Na₂SO₄ and concentrated under reduced pressure. The residue was purified by column chromatography using silica-gel (60-120) and 30-60% ethylacetate in pet ether as an eluent to afford N—((S)-1-amino-3-hydroxy-1-oxopropan-2-yl)-5-(2-methoxy-1-phenylethoxy)-2-methylbenzofuran-3-carboxamide (Cpd 346) (100 mg, 71%). A preparative chiral SFC was performed on racemic Cpd 346 to afford Cpd 346—En 1 and Cpd 346—En 2.

### Synthesis of N—((S)-1-Amino-3-hydroxy-1-oxopropan-2-yl)-5-(2-(dimethylamino)-1-phenylethoxy)-2-methylbenzofuran-3-carboxamide (Cpd 347)

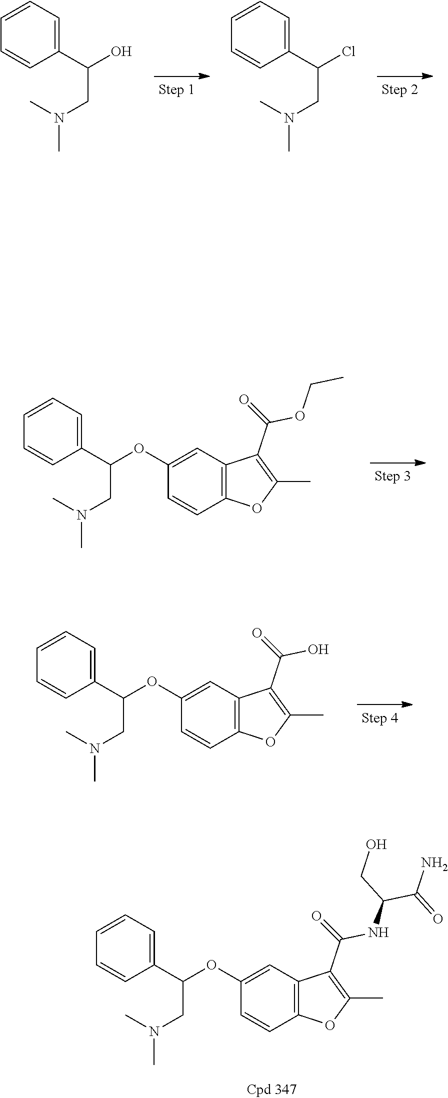 [SMILES: CN(C)CC(O)c1ccccc1 ; CN(C)CC(Cl)c1ccccc1 ; CCOC(=O)c1c(C)oc2ccc(OC(CN(C)C)c3ccccc3)cc12 ; Cc1oc2ccc(OC(CN(C)C)c3ccccc3)cc2c1C(=O)O ; Cc1oc2ccc(OC(CN(C)C)c3ccccc3)cc2c1C(=O)N[C@@H](CO)C(N)=O]

Step 1: Thionyl chloride (6.05 g, 50.88 mmol) was slowly added to a stirred solution of 2-(dimethylamino)-1-phenylethan-1-ol (6.0 g, 36.36 mmol) in chloroform (30 mL) at RT. The RM stirred for 1 h at RT. Filtered the solid and the filter cake was washed with ethyl acetate (20 mL). Combined filtrate was dried under reduced pressure to afford 2-chloro-N,N-dimethyl-2-phenylethan-1-amine (3.1 g, 46.6%) as a white solid.

Step 2: To a pre-stirred solution of ethyl 5-hydroxy-2-methylbenzofuran-3-carboxylate (1.0 g, 4.54 mmol) and 2-chloro-N,N-dimethyl-2-phenylethan-1-amine (1.672 g, 9.09 mmol) in MeCN (20 mL) was added Cs₂CO₃ (5.202 g, 13.62 mmol) at RT. The RM was warmed to 70° C. and stirred for 16 h. The reaction progress was monitored by TLC. The RM was filtered and filtrate was concentrated under reduced pressure to afford ethyl 5-(2-(dimethylamino)-1-phenylethoxy)-2-methylbenzofuran-3-carboxylate (1.2 g, 75%) as a brown gummy liquid. TLC system: 20% ethyl acetate in pet ether; RF: 0.4.

Step 3: A solution of NaOH (1.31 g, 32.96 mmol) in water (3.0 mL) was added to a stirred solution of ethyl 5-(2-(dimethylamino)-1-phenylethoxy)-2-methylbenzofuran-3-carboxylate (1.2 g, 3.296 mmol) in MeOH (10 mL) and THF (10 mL) at RT. The RM was stirred for 16 h at RT. The reaction progress was monitored by TLC. The RM was concentrated under reduced pressure, diluted with water (15 mL) and pH was adjusted to ˜6 with 1N aqueous HCl solution. The precipitated solid was filtered and dried under vacuum to afford 5-(2-(dimethylamino)-1-phenylethoxy)-2-methylbenzofuran-3-carboxylic acid (1.0 g, 90%) as a brown solid. Crude was used in the next step without purification. TLC system: 10% MeOH in dichloromethane; RF: 0.2.

Step 4: To a solution of 5-(2-(dimethylamino)-1-phenylethoxy)-2-methylbenzofuran-3-carboxylic acid (1.0 g, 2.94 mmol), HATU (1.67 g, 4.42 mmol) and DIPEA (1.142 g, 8.82 mmol) in DMF (10 mL) was added L-serinamide·HCl (621.6 g, 4.42 mmol) at 0° C. under argon atmosphere. The RM was stirred at RT for 2 h. The reaction progress was monitored by TLC. The RM was poured into ice water (50 mL) and extracted with ethyl acetate (3×50 mL). Combined organic layers were washed with water (2×100 mL), brine (100 mL), dried over anhydrous Na₂SO₄ and concentrated under reduced pressure. The residue was washed with dichloromethane, n-pentane and dried under reduced pressure to afford N—((S)-1-amino-3-hydroxy-1-oxopropan-2-yl)-5-(2-(dimethylamino)-1-phenylethoxy)-2-methylbenzofuran-3-carboxamide (Cpd 347) (572 mg, 45.76%). A preparative chiral SFC was performed on racemic Cpd 347 to afford Cpd 347—En 1 and Cpd 347—En 2.

### Synthesis of N-(4-(hydroxymethyl)tetrahydro-2H-pyran-4-yl)-2-methyl-5-((2-phenylpropan-2-yl)oxy)benzofuran-3-carboxamide (Cpd 316)

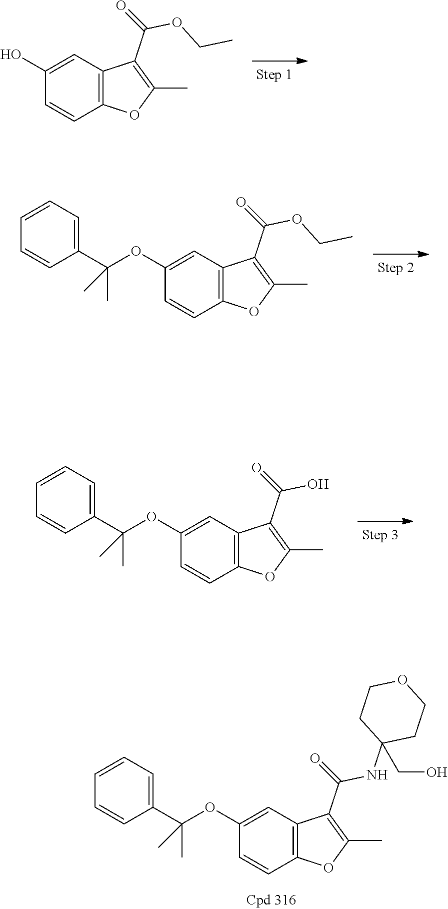 [SMILES: CCOC(=O)c1c(C)oc2ccc(O)cc12 ; CCOC(=O)c1c(C)oc2ccc(OC(C)(C)c3ccccc3)cc12 ; Cc1oc2ccc(OC(C)(C)c3ccccc3)cc2c1C(=O)O ; Cc1oc2ccc(OC(C)(C)c3ccccc3)cc2c1C(=O)NC1(CO)CCOCC1]

Step 1: 2-phenylpropan-2-ol (927 mg, 6.82 mmol), ADDP (1.72 g, 6.82 mmol) and tri-n-butylphosphine (1.6 mL, 6.82 mmol) were added sequentially to a pre-stirred solution of ethyl 5-hydroxy-2-methylbenzofuran-3-carboxylate (1 g, 4.54 mmol) in THF (50 mL) at RT under argon atmosphere. The RM was allowed to attain RT and stirred for 18 h. The reaction progress was monitored by TLC. After 18 h, solvent was evaporated under vacuum and dried. The crude was purified by FCC using 12% EtOAc in pet-ether as eluent to get ethyl 2-methyl-5-(2-phenylpropan-2-yloxy)benzofuran-3-carboxylate (450 mg, 30%)

Step 2: To a stirred solution of ethyl 2-methyl-5-(2-phenylpropan-2-yloxy)benzofuran-3-carboxylate (450 mg, 1.33 mmol) in EtOH:THF:H2O (1:1:1), (21 mL), NaOH (213 mg, 5.32 mmol) was added at 0° C. RM was stirred for 18 h at 80° C. Reaction progress was monitored by TLC. After completion of the reaction, RM was cooled to RT and solvent was evaporated under reduced pressure. The crude was diluted with ice water (10 mL), acidified to pH˜1 using 1N aq. HCl (10 mL), and extracted with DCM (3×100 mL). Combined organic layers were washed with brine solution (100 mL), dried over anhydrous Na₂SO₄, filtered and concentrated under vacuo to get 2-methyl-5-(2-phenylpropan-2-yloxy)benzofuran-3-carboxylic acid (250 mg, 61%).

Step 3: To a stirred solution of 2-methyl-5-(2-phenylpropan-2-yloxy)benzofuran-3-carboxylic acid (250 mg, 0.81 mmol) in DMF (5 mL), HATU (460 mg, 1.21 mmol), DIPEA (0.3 mL, 1.61 mmol), and (4-aminotetrahydro-2H-pyran-4-yl)methanol (158 mg, 1.21 mmol) at 0° C. The RM was stirred for 18 h at RT. Reaction progress was monitored by LCMS. After completion of the reaction, RM was diluted with ice water (50 ml), and extracted with DCM (4×100 mL). Combined organic layers were washed with brine solution (50 mL), dried over anhydrous Na₂SO₄, filtered and concentrated to afford crude. The crude product was purified by reverse phase Prep-HPLC purification to afford Cpd 316 (101 mg, 30%).

The following compounds were prepared in a similar manner (use of appropriate reagents and purification methods (including chiral HPLC or chiral SFC) known to the person skilled in the art) as described for Cpd 316: Cpd 338, Cpd 339, Cpd 340, Cpd 341, Cpd 342.

### Synthesis of 5-(2-hydroxy-1-phenylethoxy)-N-(4-(hydroxymethyl)tetrahydro-2H-pyran-4-yl)-2-methylbenzofuran-3-carboxamide (Cpd 317)

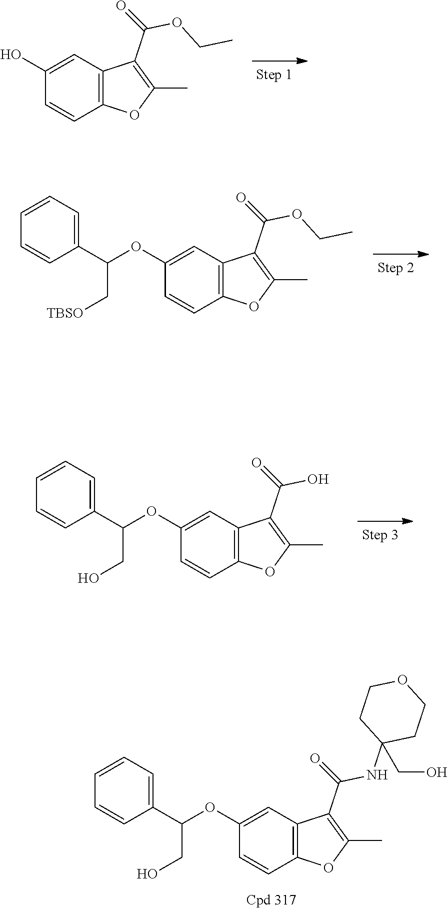 [SMILES: CCOC(=O)c1c(C)oc2ccc(O)cc12 ; CCOC(=O)c1c(C)oc2ccc(OC(CO[Si](C)(C)C(C)(C)C)c3ccccc3)cc12 ; Cc1oc2ccc(OC(CO)c3ccccc3)cc2c1C(=O)O ; Cc1oc2ccc(OC(CO)c3ccccc3)cc2c1C(=O)NC1(CO)CCOCC1]

Step 1: 2-((tert-butyldimethylsilyl)oxy)-1-phenylethan-1-ol (3.2 g, 12.73 mmol), ADDP (3.43 g, 13.64 mmol) and tri-n-butylphosphine (3.3 mL, 13.64 mmol) were added sequentially to a pre-stirred solution of ethyl 5-hydroxy-2-methylbenzofuran-3-carboxylate (2 g, 9.09 mmol) in THF (100 mL) at RT under argon atmosphere. The RM was allowed to attain RT and stirred for 18 h. The reaction progress was monitored by TLC. After completion of reaction, solvent was evaporated and dried. The crude was purified by FCC with silica using 12% EtOAc in pet-ether as eluent to afford ethyl 5-(2-((tert-butyldimethylsilyl)oxy)-1-phenylethoxy)-2-methylbenzofuran-3-carboxylate (480 mg, 11%)

Step 2: To a stirred solution of ethyl 5-(2-((tert-butyldimethylsilyl)oxy)-1-phenylethoxy)-2-methylbenzofuran-3-carboxylate (450 mg, 0.99 mmol) in EtOH:THF:H2O (1:1:1, 20 mL), LiOH·H₂O (333 mg, 7.93 mmol) was added at 0° C. The RM was stirred for 24 h at RT. Reaction progress was monitored by TLC. After completion of the reaction, The RM was poured into ice water (20 mL) and acidified to pH ˜1 using 1N aq. HCl (25 mL) and extracted with DCM (5×100 mL), Combined organic layers were washed with brine solution (100 mL), dried over anhydrous Na₂SO₄, filtered and concentrated under reduced pressure to afford 5-(2-hydroxy-1-phenylethoxy)-2-methylbenzofuran-3-carboxylic acid (380 mg, crude).

Step 3: To a stirred solution of 5-(2-hydroxy-1-phenylethoxy)-2-methylbenzofuran-3-carboxylic acid (380 mg, 1.22 mmol) in DMF (5 mL) was added HATU (694 mg, 1.83 mmol), DIPEA (0.4 mL, 2.43 mmol), and (4-aminotetrahydro-2H-pyran-4-yl)methanol (239 mg, 1.83 mmol) at 0° C. The RM was stirred for 18 h at RT. Reaction progress was monitored by LCMS. After completion of the reaction, The RM was diluted with EtOAc (400 ml), the organic layer was washed with water (6×100 mL), brine solution (100 mL), dried over anhydrous Na₂SO₄, and concentrated under reduced pressure. The crude was purified by reverse phase prep-HPLC purification to afford Cpd 317 (130 mg, 31% over 2 steps). A preparative chiral SFC was performed on racemic Cpd 317 to afford Cpd 317—En 1 and Cpd 317—En 2.

### 5-((2-Fluorobenzyl)oxy)-N-(1-(2-hydroxyethyl)-2-oxopyrrolidin-3-yl)-2-methylbenzofuran-3-carboxamide (Cpd 311)

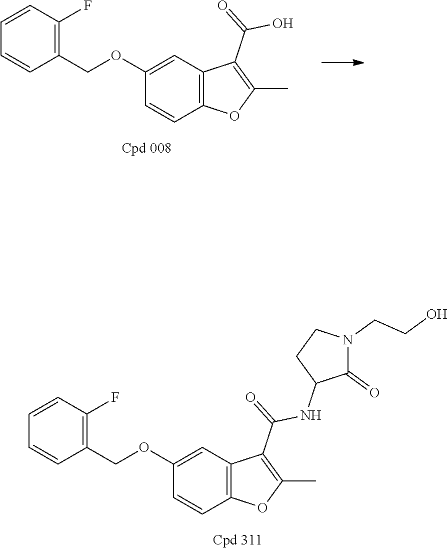 [SMILES: Cc1oc2ccc(OCc3ccccc3F)cc2c1C(=O)O ; Cc1oc2ccc(OCc3ccccc3F)cc2c1C(=O)NC1CCN(CCO)C1=O]

To a solution mixture of 5-((2-fluorobenzyl)oxy)-2-methylbenzofuran-3-carboxylic acid (600 mg, 2.0 mmol) and 3-amino-1-(2-hydroxyethyl)pyrrolidin-2-one hydrochloride (396 mg, 2.2 mmol) in DMF (20 mL) were added DIPEA (0.92 mL, 5.0 mmol) followed by HATU (1.52 g, 4.0 mmol) at 0° C. under argon atmosphere. The resulting reaction mixture was stirred at room temperature for 2 h. The reaction progress was monitored by TLC. The RM was diluted with water (100 mL), extracted with EtOAc (2×100 mL). The combined organic layer was washed with water (100 mL), brine (100 mL), dried over anhydrous Na₂SO₄ and concentrated under reduced pressure. The crude product was purified by GRACE flash chromatography using 0.1% formic acid in water and MeCN as an eluent to afford Cpd 311 (330 mg, 39%). A preparative chiral SFC was performed on racemic Cpd 311 to afford Cpd 311—En 1 and Cpd 311—En 2.

The following compounds were prepared in a manner similar (use of appropriate reagents and purification methods known to the person skilled in the art) to Cpd 311—En 1 and Cpd 311—En 2: Cpd 314—En 1, 314—En 2, 320—En 1, 320—En 2, 329—En 1, 329—En 2, 330—En 1, 330—En 2, 331—En 1, 331—En 2

### N-(1-amino-3-hydroxy-1-oxopropan-2-yl)-5-((2-fluorophenoxy)methyl)-2-methylbenzofuran-3-carboxamide (Cpd 321)

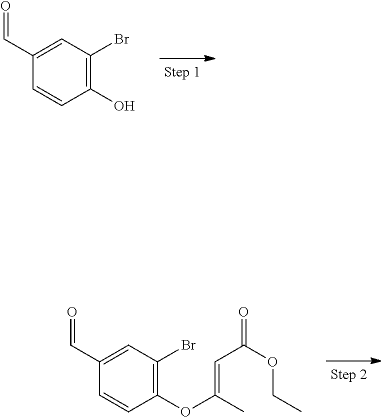 [SMILES: O=Cc1ccc(O)c(Br)c1 ; CCOC(=O)/C=C(\C)Oc1ccc(C=O)cc1Br]

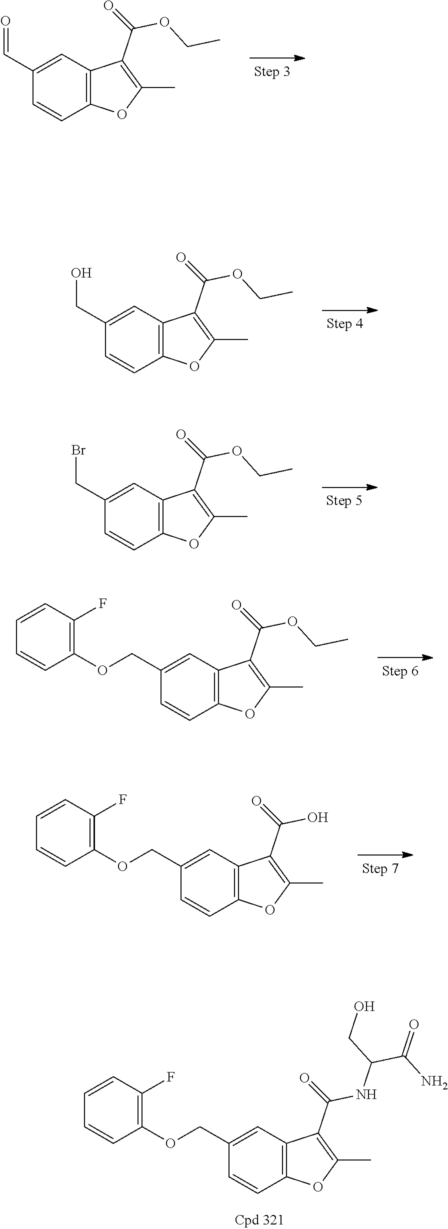 [SMILES: CCOC(=O)c1c(C)oc2ccc(C=O)cc12 ; CCOC(=O)c1c(C)oc2ccc(CO)cc12 ; CCOC(=O)c1c(C)oc2ccc(CBr)cc12 ; CCOC(=O)c1c(C)oc2ccc(COc3ccccc3F)cc12 ; Cc1oc2ccc(COc3ccccc3F)cc2c1C(=O)O ; Cc1oc2ccc(COc3ccccc3F)cc2c1C(=O)NC(CO)C(N)=O]

Step 1: DABCO (2.78 g, 24.75 mmol) was added to a stirred solution mixture of 3-bromo-4-hydroxybenzaldehyde (5.0 g, 24.75 mmol), ethyl but-2-ynoate (3.32 g, 29.70 mmol) in acetonitrile (100 mL) at RT and the RM was stirred at reflux temperature for 4 h. The solvent was removed under reduced pressure. The residue was diluted with EtOAc (300 mL) and was sequentially washed with 1N HCl (100 mL), aq. 1N NaOH (2×75 mL), water (100 mL), brine (150 mL) and dried over anhydrous Na₂SO₄ and concentrated under reduced pressure. The crude was purified by FCC with silica-gel using 10% EtOAc in pet-ether as an eluent to afford ethyl 3-(2-bromo-4-formylphenoxy)but-2-enoate (1.5 g, 29%).

Step 2: A solution mixture of ethyl 3-(2-bromo-4-formylphenoxy)but-2-enoate (1.5 g, 4.75 mmol) and TEA (0.26 mL, 1.91 mmol) in acetonitrile (25 mL) was de-gassed with argon for 5 min and then bis(tri-tert-butylphosphine)palladium(0)] (195 mg, 0.382 mmol) was added. The RM was maintained at reflux for 2 h. The reaction progress was monitored by TLC. The RM was directly concentrated under reduced pressure. The crude was partitioned between water (100 mL) and EtOAc (2×100 mL). The combined organic layer was washed with brine (150 mL), dried over anhydrous Na₂SO₄ and concentrated under reduced pressure to afford ethyl 5-formyl-2-methylbenzofuran-3-carboxylate (1.0 g, 90%).

Step 3: NaBH4 (398 mg, 10.8 mmol) was added portion wise to a solution mixture of ethyl 5-formyl-2-methylbenzofuran-3-carboxylate (1.0 g, 4.31 mmol) in EtOH: THF (1:1) (40 mL) at 0° C. and the RM was stirred for 30 min. Then water (5.0 mL) was added to the RM and concentrated under reduced pressure. The crude was diluted with EtOAc (50 mL), washed with water (2×50 mL), brine (100 mL), dried over anhydrous Na₂SO₄ and concentrated under reduced pressure to afford ethyl 5-(hydroxymethyl)-2-methylbenzofuran-3-carboxylate (900 mg, crude).

Step 4: A solution mixture of ethyl 5-(hydroxymethyl)-2-methylbenzofuran-3-carboxylate (700 mg, 2.99 mmol), PPh₃ (1.17 g, 4.49 mmol) and CBr₄ (1.48 g, 4.49 mmol) in THF (40 mL) was stirred at 0° C. and stirred at RT for 2 h. The reaction progress was monitored by TLC. The RM was diluted with EtOAc (100 mL), washed with NaHCO₃ solution (50 mL), water (50 mL), brine (50 mL), dried over anhydrous Na₂SO₄ and concentrated under reduced pressure to afford ethyl 5-(bromomethyl)-2-methylbenzofuran-3-carboxylate (1.25 g, crude).

Step 5: A suspension of 2-fluorophenol (700 mg, 6.25 mmol), ethyl 5-(bromomethyl)-2-methylbenzofuran-3-carboxylate (2.2 g, 7.50 mmol) and K₂CO₃ (1.72 g, 12.50 mmol) in acetonitrile (20 mL) were stirring at 60° C. for 2 h. The reaction progress was monitored by TLC. The RM was cooled and filtered off the solids. The filtrate was diluted with EtOAc (100 mL), washed with water, brine, dried over anhydrous Na₂SO₄ and concentrated under reduced pressure. The crude was purified by FCC with silica with a solvent gradient mixture of 2% EtOAc in pet-ether as an eluent to afford ethyl 5-((2-fluorophenoxy)methyl)-2-methylbenzofuran-3-carboxylate (1.5 g, 73%).

Step 6: A solution of NaOH (395 mg, 9.9 mmol) dissolved in water (10 mL) was added to a solution of ethyl 5-((2-fluorophenoxy)methyl)-2-methylbenzofuran-3-carboxylate (1.3 g, 1.09 mmol) in a mixture of ethanol: THF (1:1) (24.0 mL) at RT and the RM was maintained under stirring at 70° C. for 4 h. The reaction progress was monitored by TLC. The RM was concentrated under reduced pressure and the residue was dissolved in water, adjusted the pH to ˜4.0 with 1N HCl. The resulting precipitated was collected by filtration, washed with water and dried to afford 5-((2-fluorophenoxy)methyl)-2-methylbenzofuran-3-carboxylic acid (1.0 g, 84%).

Step 7: To a solution mixture of 5-((2-fluorophenoxy)methyl)-2-methylbenzofuran-3-carboxylic acid (500 mg, 1.66 mmol), HATU (823 g, 2.17 mmol) and DIPEA (0.58 mL, 3.33 mmol) in DMF (10 mL) was added 2-amino-3-hydroxypropanamide·HCl (932.9 mg, 6.664 mmol) at RT. The RM was stirred at RT for 2 h. The reaction progress was monitored by TLC. The RM was diluted with ice water (50 mL), the precipitated was collected by filtration and dried. The solid was dissolved in EtOAc (50 mL), treated with activated charcoal and filtered through celite pad. The filtrate was concentrated under reduced pressure to afford Cpd 321 (320 mg, 54%). A preparative chiral SFC was performed on racemic Cpd 321 to afford Cpd 321—En 1 and Cpd 321—En 2.

The following compounds were prepared in a manner similar (use of appropriate reagents and purification methods known to the person skilled in the art) to Cpd 321-En 1 and Cpd 321—En 2: Cpd 318 and 319

### N-(3-carbamoyltetrahydrofuran-3-yl)-5-((2-fluorobenzyl)oxy)-2-methylbenzofuran-3-carboxamide (Cpd 323)

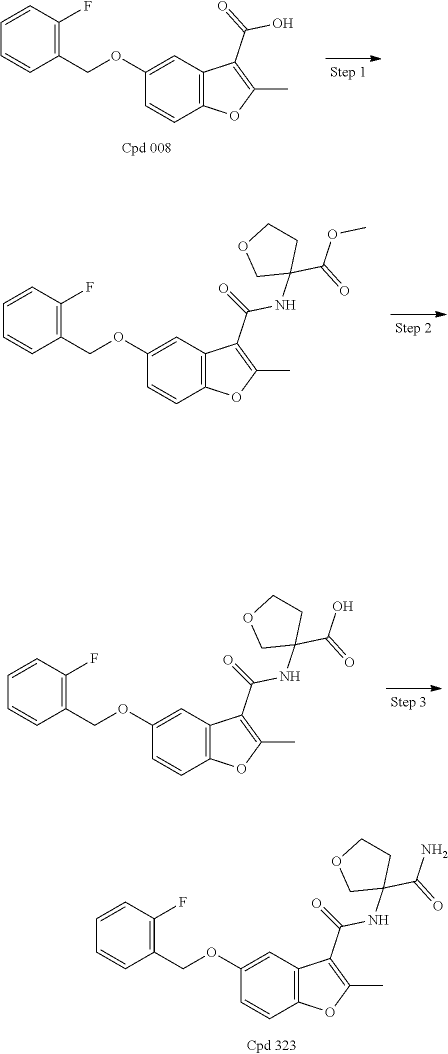 [SMILES: Cc1oc2ccc(OCc3ccccc3F)cc2c1C(=O)O ; COC(=O)C1(NC(=O)c2c(C)oc3ccc(OCc4ccccc4F)cc23)CCOC1 ; Cc1oc2ccc(OCc3ccccc3F)cc2c1C(=O)NC1(C(=O)O)CCOC1 ; Cc1oc2ccc(OCc3ccccc3F)cc2c1C(=O)NC1(C(N)=O)CCOC1]

Step 1: To a solution mixture of 5-((2-fluorobenzyl)oxy)-2-methylbenzofuran-3-carboxylic acid (0.5 g, 1.66 mmol), HATU (1.26 g, 3.33 mmol) and DIPEA (0.61 mL, 3.33 mmol) in DMF (15 mL) was added methyl 3-aminotetrahydrofuran-3-carboxylate hydrochloride (0.363 g, 1.99 mmol) at 0° C. and the RM was stirred at RT for 2 h. The reaction progress was monitored by TLC. The reaction mixture was diluted with ice cold water (50 mL) and extracted with EtOAc (3×50 mL). The combined organic layers were washed with water (2×25 mL), brine (25 mL) and dried over anhydrous Na₂SO₄ and concentrated under reduced pressure to afford methyl 3-(5-((2-fluorobenzyl)oxy)-2-methylbenzofuran-3-carboxamido)tetrahydrofuran-3-carboxylate (650 mg, crude).

Step 2: 2N NaOH (25 mL) was added to a solution of methyl 3-(5-((2-fluorobenzyl)oxy)-2-methylbenzofuran-3-carboxamido)tetrahydrofuran-3-carboxylate (650 mg, 1.52 mmol) in MeOH (20 mL) and THF (10 mL) and the RM was stirred at RT for 16 h. The reaction progress was monitored by TLC. The RM was diluted with ice cold water (50 mL), acidified with 1N HCl and extracted with EtOAc (3×50 mL). The combined organic layer was washed with water (2×25 mL), brine (25 mL) and dried over anhydrous Na₂SO₄ and concentrated under reduced pressure to afford 3-(5-((2-fluorobenzyl)oxy)-2-methylbenzofuran-3-carboxamido)tetrahydrofuran-3-carboxylic acid (450 mg, crude).

Step 3: To a solution mixture of 3-(5-((2-fluorobenzyl)oxy)-2-methylbenzofuran-3-carboxamido)tetrahydrofuran-3-carboxylic acid (0.45 g, 1.08 mmol), HATU (0.828 g, 2.17 mmol) and DIPEA (0.4 mL, 2.17 mmol) in DMF (15 mL) was added ammonium chloride (0.290 g, 5.4 mmol) at 0*C and the RM was stirred at RT for 2 h. The reaction progress was monitored by TLC. The RM was diluted with ice cold water (50 mL) and extracted with EtOAc (3×50 mL). The combined organic layer was washed with water (2×50 mL), brine (50 mL) and dried over anhydrous Na₂SO₄ and concentrated under reduced pressure. The crude was purified by GRACE flash chromatography using 0.1% formic acid in water and acetonitrile as an eluent to afford Cpd 323 (320 mg, 47% over 3 steps). A preparative chiral SFC was performed on racemic Cpd 323 to afford Cpd 323— En 1 and Cpd 323—En 2.

The following compounds were prepared in a manner similar (use of appropriate reagents and purification methods known to the person skilled in the art) to Cpd 323—En 1 and Cpd 323—En 2: Cpd 322

### 5-((2-fluorobenzyl)oxy)-N-(3-(hydroxymethyl)tetrahydrofuran-3-yl)-2-methylbenzofuran-3-carboxamide (Cpd 327)

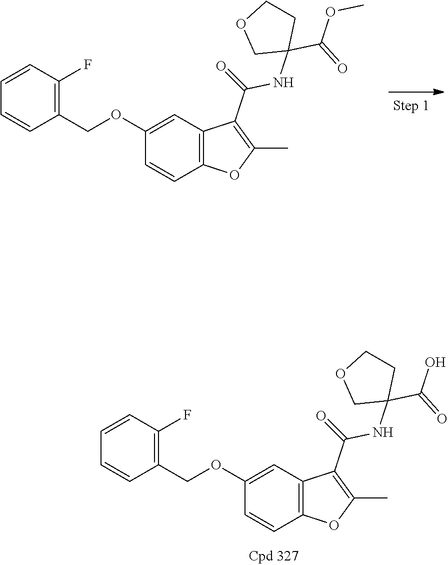 [SMILES: COC(=O)C1(NC(=O)c2c(C)oc3ccc(OCc4ccccc4F)cc23)CCOC1 ; Cc1oc2ccc(OCc3ccccc3F)cc2c1C(=O)NC1(C(=O)O)CCOC1]

Step 1: 1M LAH in THF (2.34 mL, 2.34 mmol) was added to a solution of methyl 3-(5-((2-fluorobenzyl)oxy)-2-methylbenzofuran-3-carboxamido)tetrahydrofuran-3-carboxylate (0.500 g, 1.17 mmol) in THF (20 mL) at 0° C. and the RM was stirred at RT for 1 h. The reaction progress was monitored by TLC. The RM was slowly quenched with saturated sodium sulfate solution (5 mL), diluted with EtOAc (50 mL) and filtered. The filtrate was dried over Na₂SO₄ and concentrated. The crude was purified by GRACE flash chromatography using 0.1% formic acid in water and acetonitrile as an eluent to afford Cpd 237 (280 mg, 60%). A preparative chiral SFC was performed on racemic Cpd 327 to afford Cpd 327—En 1 and Cpd 327—En 2.

Cpd 324, Cpd 325, Cpd 334—En 1 and Cpd 334—En 2 were prepared in a manner similar (use of appropriate reagents and purification methods known to the person skilled in the art) to cpd 327—En 1.

### N-(3,3-difluoropiperidin-4-yl)-2-methyl-5-((2-(methylsulfonyl)benzyl)oxy)benzofuran-3-carboxamide (Cpd 333)

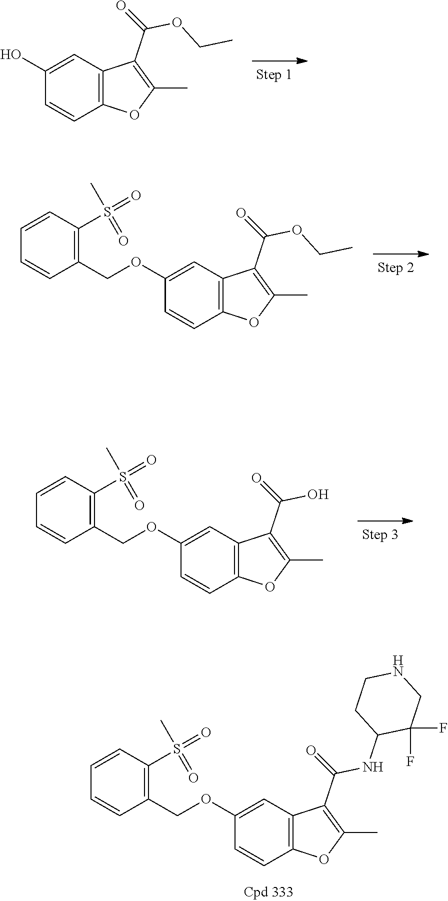 [SMILES: CCOC(=O)c1c(C)oc2ccc(O)cc12 ; CCOC(=O)c1c(C)oc2ccc(OCc3ccccc3S(C)(=O)=O)cc12 ; Cc1oc2ccc(OCc3ccccc3S(C)(=O)=O)cc2c1C(=O)O ; Cc1oc2ccc(OCc3ccccc3S(C)(=O)=O)cc2c1C(=O)NC1CCNCC1(F)F]

Step 1: (2-(methylsulfonyl) phenyl) methanol (1.8 g, 9.54 mmol), ADDP (2.8 g, 11.13 mmol) and PBu₃ (2.7 ml, 11.13 mmol), were added sequentially to a solution of ethyl 5-hydroxy-2-methylbenzofuran-3-carboxylate (1.75 g, 7.95 mmol) in THF (60 mL) at 0° C. under Ar atmosphere. The RM was stirred at RT for 16 h. The reaction progress was monitored by TLC. The RM was diluted with water (50 mL) and extracted with EtOAc (2×100 mL). The combined organic layer was washed with water (1×50 mL), brine (1×50 mL), dried over anhydrous Na₂SO₄ and concentrated under reduced pressure. The crude was purified by column chromatography over silica gel (100-200 mesh) using 0-30% EtOAc in pet-ether as an eluent to afford ethyl 2-methyl-5-((2-(methylsulfonyl) benzyl) oxy) benzofuran-3-carboxylate (1.2 g, 39%).

Step 2: To a solution of ethyl 2-methyl-5-((2-(methylsulfonyl) benzyl) oxy) benzofuran-3-carboxylate (1.2 g, 3.092 mmol), in THF: MeOH (1:1) (40 ml), was added 2N NaOH (8 ml) at RT, and heated to 60° C. The RM was stirred for 6 h at 60° C., and reaction progress was monitored by TLC. The RM was concentrated, then diluted with ice water and pH was adjusted to with 1N HCl solution to give a solid. The solid was filtered and dried under reduced pressure to afford 2-methyl-5-((2-(methylsulfonyl) benzyl) oxy) benzofuran-3-carboxylic acid (1 g, crude).

Step 3: To a stirred solution of 2-methyl-5-((2-(methylsulfonyl)benzyl)oxy)benzofuran-3-carboxylic acid (700 mg, 1.944 mmol) in DCM (30 ml), were added DIPEA (1.3 ml, 7.7 mmol), HATU (1.0 g, 2.721 mmol) at 0° C. and followed by addition of tert-butyl 4-amino-3,3-difluoropiperidine-1-carboxylate (550 mg, 2.332 mmol), then the RM was stirred for 16 h at RT. Reaction progress was monitored by TLC. The RM was diluted with water (100 mL), and extracted with EtOAc (3×100 mL). The combined extracts were washed with brine solution (20 ml), dried over Na₂SO₄, and concentrated under reduced pressure. The crude was purified by FCC with silica using 40% EtOAc in pet ether as eluent to afford Cpd 333 (800 mg, 71%). A preparative chiral SFC was performed on racemic Cpd 333 to afford Cpd 333—En 1 and Cpd 333—En 2.

Cpd 332 was prepared in a manner similar (use of appropriate reagents and purification methods known to the person skilled in the art) to Cpd 333.

### N—((S)-1-amino-3-hydroxy-1-oxopropan-2-yl)-5-(1-(2-fluorophenyl)-2-hydroxyethoxy)-2-methylbenzofuran-3-carboxamide (Cpd 335)

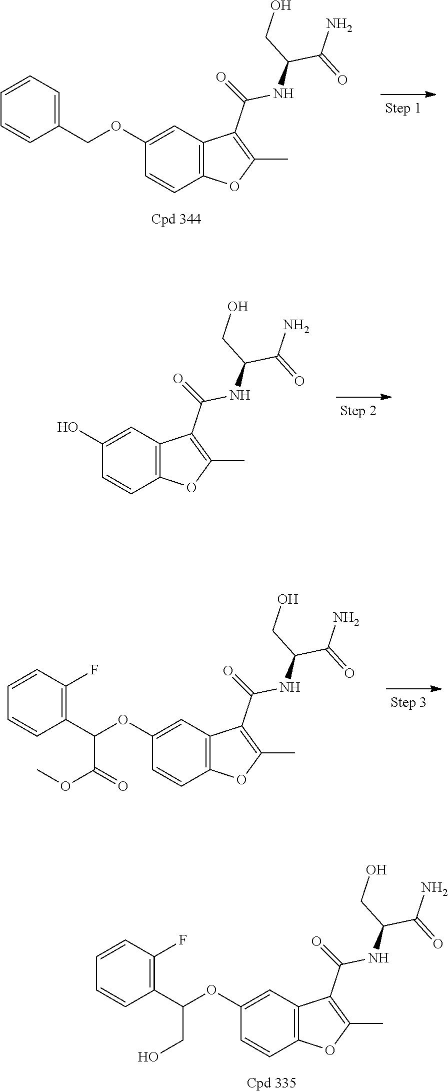 [SMILES: Cc1oc2ccc(OCc3ccccc3)cc2c1C(=O)N[C@@H](CO)C(N)=O ; Cc1oc2ccc(O)cc2c1C(=O)N[C@@H](CO)C(N)=O ; COC(=O)C(Oc1ccc2oc(C)c(C(=O)N[C@@H](CO)C(N)=O)c2c1)c1ccccc1F ; Cc1oc2ccc(OC(CO)c3ccccc3F)cc2c1C(=O)N[C@@H](CO)C(N)=O]

Step 1: Parr hydrogenator flask was charged with (S)—N-(1-amino-3-hydroxy-1-oxopropan-2-yl)-5-(benzyloxy)-2-methylbenzofuran-3-carboxamide (800 mg, 2.17 mmol) in ethanol (20 mL) and was added 10% Pd/C (250 mg) at RT. The RM was stirred under hydrogen gas pressure (70 psi) at RT for 16 h. The reaction progress was monitored by TLC. The RM was diluted with EtOAc (50 mL) and filtered through celite, washed the celite with EtOAc (20 mL). The combined filtrate was concentrated under reduced pressure to afford (S)—N-(1-amino-3-hydroxy-1-oxopropan-2-yl)-5-(benzyloxy)-2-methylbenzofuran-3-carboxamide (0.45 g, 92%).

Step 2: A suspension of (S)—N-(1-amino-3-hydroxy-1-oxopropan-2-yl)-5-hydroxy-2-methyl benzofuran-3-carboxamide (250 mg, 0.89 mmol), methyl 2-bromo-2-(2-fluorophenyl)acetate (222.1 mg, 0.89 mmol) and K₂CO₃ (0.148, 1.07 mmol) in DMF (5 mL) was stirred at RT for 1 h. The reaction progress was monitored by TLC. The RM was diluted with ice water (30 mL) and extracted with EtOAc (2×30 mL). The combined organic layer was washed with brine (40 mL), dried over anhydrous Na₂SO₄ and concentrated under reduced pressure to afford methyl 2-((3-(((S)-1-amino-3-hydroxy-1-oxopropan-2-yl)carbamoyl)-2-methylbenzofuran-5-yl)oxy)-2-(2-fluorophenyl)acetate (500 mg, crude).

Step 3: LAH (1M in THF) (1.3 mL, 1.3 mmol) was added to a solution of methyl 2-((3-(((S)-1-amino-3-hydroxy-1-oxopropan-2-yl)carbamoyl)-2-methylbenzofuran-5-yl)oxy)-2-(2-fluorophenyl)acetate (500 mg, 1.12 mmol) in THF (10 mL) at 0° C. under Ar. The RM was stirred at RT for 1 h. The reaction progress was monitored by TLC. The RM was quenched with saturated Na₂SO₄ solution (5 mL), diluted with EtOAc (20 mL) and filtered through celite pad. The filtrate was dried over anhydrous Na₂SO₄ and concentrated under reduced pressure to afford Cpd 335 (0.250 g, 34% over 2 steps)A preparative chiral SFC was performed on racemic Cpd 335 to afford Cpd 335—En 1 and Cpd 335—En 2.

### Analytical Data

| | [M + H]⁺ | [M − H]⁻ | LC/MS | RT | ¹H NMR |
|---|---|---|---|---|---|
| Cpd | (m/z): | (m/z): | Method | (min.) | (δ ppm) |
| 001 | — | 267 | L2 | 3.0 | (DMSO d6, 300 MHz): 12.99 (br s, 1H); 8.60 (s, 1H); 7.61 |
| | | | | | (d, 1H); 7.55-7.44 (m,3H); 7.44-7.30 (m, 3H); 7.07 (dd, 1H); |
| | | | | | 5.16 (s, 2H). |
| 004 | 297 | 295 | L2 | 5.2 | — |
| 005 | — | 295 | L2 | 4.2 | — |
| 006 | — | 295 | L1 | 8.8 | — |
| 007 | — | 299 | L1 | 9 | — |
| 009 | — | 299 | L1 | 9.0 | — |
| 010 | — | 306 | L1 | 8.2 | (DMSO d6, 300 MHz): 7.92 (d, 1H); 7.80-7.68 (m, 2H); |
| | | | | | 7.62-7.47 (m, 3H); 7.01 (dd, 1H); 5.27 (s, 2H); 2.71 (s, 3H). |
| 011 | — | 307 | L2 | 4.8 | — |
| 012 | 313 | 311 | L2 | 3.5 | — |
| 013 | — | 311 | L2 | 3.7 | — |
| 014 | — | 311 | L1 | 9.1 | — |
| 015 | — | 313 | L1 | 9.4 | (DMSO-d6, 300 MHz): 13.00 (br s, 1H); 7.56-7.40 (m, 3H); |
| | | | | | 7.35-7.25 (m, 2H); 7.16 (ddd, 1H); 7.01 (dd, 1H); 5.17 (s, |
| | | | | | 2H); 3.15 (q, 2H); 1.26 (t, 3H) |
| 019 | 314 | — | — | — | — |
| 020 | — | 329 | L1 | 8.6 | (DMSO, 300 MHz): 7.66 (d, 1H); 7.35 (d, 1H); 7.30-7.16 |
| | | | | | (m, 2H); 7.09-6.95 (m, 2H); 5.14 (s, 2H); 4.98 (s, 2H); 3.53 |
| | | | | | (s, 3H). |
| 021 | — | 332 | L1 | 9.1 | — |
| 023 | — | 335 | L2 | 5.8 | 1H NMR (DMSO d6, 300 MHz) 8 ppm 12.90 (br s, 1H); |
| | | | | | 7.54-7.44 (m, 4H); 7.44-7.28 (m, 3H); 6.98 (dd, 1H); 5.13 (s, |
| | | | | | 2H); 4.14-3.97 (m, 1H); 2.07-1.44 (m, 2H); 1.90-1.62 (m, |
| | | | | | 6H) |
| 024 | — | 349 | L1 | 9.0 | (DMSO d6, 300 MHz): 13.50-12.52 (br s, 1H); 7.85-7.65 (m, |
| | | | | | 3H); 7.65-7.42 (m, 3H); 7.00 (d, 1H); 5.27 (s, 2H); 2.71 (s, |
| | | | | | 3H) |
| 025 | 351 | 349 | L2 | 4.7 | — |
| 026 | — | 349 | L1 | 9.1 | — |
| 028 | — | 365 | L2 | 4.9 | — |
| 035 | 282 | — | L2 | 2.60 | — |
| 036 | 298 | 296 | L3 | 5.3 | — |
| 037 | 300 | 298 | L3 | 6.80 | (CDCl3, 300 MHz): 7.42-7.31 (m, 2H); 7.26-7.15 (m, 3H); |
| | | | | | 7.07-6.90 (m, 2H); 5.75-5.55 (m, 2H); 5.11 (s, 2H); 2.73 (s, |
| | | | | | 3H) |
| 038 | 310 | — | L3 | 7.80 | — |
| 039 | 322 | 320 | L3 | 7.60 | — |
| 040 | 323 | — | L3 | 8.40 | — |
| 041 | 334 | — | L5 | 9.20 | — |
| 042 | 337 | — | L3 | 5.90 | — |
| 043 | 338 | — | L3 | 6.70 | — |
| 044 | 340 | 338 | L2 | 3.30 | — |
| 045 | 348 | — | L1 | 8.80 | — |
| 046 | 350 | 348 | L3 | 8.90 | — |
| 047 | 350 | — | L1 | 9.30 | — |
| 048 | 351 | 349 | L3 | 7.10 | — |
| 049 | 351 | 349 | L3 | 5.90 | — |
| 050 | 352 | — | L1 | 8.10 | — |
| 051 | 352 | 350 | L1 | 7.80 | — |
| 052 | 352 | — | U4 | 3.70 | — |
| 053 | 352 | 350 | U5 | 2.96 | (DMSO d6, 300 MHz): 8.09-8.29 (2 H, m); 8.09-8.29 (2 |
| | | | | | H, m); 7.29-7.42 (4 H, m); 7.26 (1 H, d); 6.92 (1 H, dd), |
| | | | | | 5.12 (2 H, s); 5.12 (2 H, s); 4.96 (1H, d), 4.48-4.50 (1 H, m), |
| | | | | | 4.30-4.40 (1H, m), 2.59 (3 H, s), 2.15-2.40 (4H, m) |
| 054 | 352 | — | U5 | 2.95 | — |
| 055 | 353 | 351 | L3 | 7.40 | — |
| 056 | 354 | 352 | L3 | 7.90 | — |
| 057 | 356 | — | L3 | 5.60 | (DMSO d6, 300 MHz):7.81 (t, 1H); 7.51-7.30 (m, 7H); 6.96 |
| | | | | | (dd, 1H); 5.12 (s, 2H); 3.70-3.40 (m, 3H); 3.27-3.12 (m, 4H); |
| | | | | | 2.60 (s, 3H) |
| 058 | 358 | — | L3 | 9.2 | — |
| 059 | 359 | — | L3 | 7.6 | — |
| 060 | 359 | — | L3 | 7.70 | — |
| 061 | 360 | 358 | L3 | 8.1 | — |
| 062 | 360 | 358 | L3 | 7.6 | — |
| 063 | 360 | 358 | L3 | 4.1 | (DMSO d6, 300 MHz): 7.67 (br s, 1H); 7.49 (d, 2H); 7.45- |
| | | | | | 7.28 (m, 4H); 6.91 (dd, 1H); 5.10 (s, 2H); 3.04 (br s; 3H); |
| | | | | | 2.69 (s, 3H). |
| 064 | 362 | 360 | L1 | 8.50 | — |
| 065 | 362 | — | L1 | 8.90 | — |
| 066 | 364 | 362 | L3 | 9.30 | — |
| 067 | 364 | — | L3 | 9.20 | — |
| 068 | 365 | 363 | U4 | 3.45 | — |
| 069 | 365 | — | U2 | 2.96 | (300 MHz, DMSO-d6): 8.06-8.26 (1 H, m); 7.89 (1 H, s); |
| | | | | | 7.22-7.59 (7H, m); 6.97 (1H, dd); 5.13 (2H, s); 4.46-4.72 |
| | | | | | (1H, m); 3.24 (2 H, dd); 2.61 (3H, s); 2.30-2.43 (1H, m); |
| | | | | | 1.76-2.14 (1 H, m) |
| 070 | 365 | 363 | U3 | 0.6 | — |
| 071 | 365 | 363 | L5 | 8.10 | (DMSO d6, 300 MHz): 8.14 (d, 1H); 7.89 (br s, 1H); 7.52- |
| | | | | | 7.29 (m, 7H); 6.97 (dd, 1H); 5.13 (s, 2H); 4.60 (dd, 1H); 3.24 |
| | | | | | (dd, 2H); 2.61 (s, 3H); 2.45-2.32 (m, 1H); 2.11-1.94 (m, 1H). |
| 072 | 365 | — | L3 | 7.30 | — |
| 073 | 365 | — | L3 | 6.00 | — |
| 074 | 365 | 363 | L3 | 6.30 | — |
| 075 | 365 | — | L3 | 6.50 | — |
| 076 | 365 | 363 | L3 | 6.90 | — |
| 077 | 365 | 363 | L3 | 6.30 | — |
| 078 | 365 | 363 | L3 | 6.80 | — |
| 079 | 365 | 363 | L3 | 7.00 | — |
| 080 | 365 | 363 | L3 | 7.00 | — |
| 081 | 365 | 363 | L3 | 6.80 | (DMSO d6, 300 MHz): 8.53 (s, 1H); 8.13 (d, 1H); 7.63 (d, |
| | | | | | 1H); 7.55 (d, 1H); 7.53-7.44 (m,2H); 7.44-7.29 (m, 3H); 7.04 |
| | | | | | (dd, 1H); 5.13 (s, 2H); 3.85-3.65 (m, 1H); 2.77 (br d, 2H); |
| | | | | | 2.17 (s, 3H); 1.95 (ddd, 2H); 1.85-1.74 (m, 2H); 1.62-1.45 |
| | | | | | (m, 2H). |
| 082 | 365 | — | U6 | 0.90 | — |
| 083 | 365 | 363 | L3 | 6.90 | (DMSO d6, 300 MHz): 7.50 (d, 1H); 7.42 (d, 1H); 7.36-7.20 |
| | | | | | (m, 4H); 7.04 (dd, 1H); 5.16 (s, 2H); 3.56 (dd, 1H); 3.49-3.16 |
| | | | | | (m, 5H); 2.48-2.29 (m, 1H); 2.37 (s, 3H); 2.15-2.00 (m, 1H). |
| 084 | 365 | 363 | L3 | 6.90 | (DMSO d6, 300 MHz): 7.44 (1 H, d); 7.08-7.34 (5 H, m); |
| | | | | | 6.96 (1 H, d); 5.05 (2 H, s); 4.55-4.44 (1 H, t); 3.10-3.61 (4 |
| | | | | | H, m), 2.55 (3 H, s), 2.16-2.37 (4 H, m) 1.90-2.10 (1 H, m) |
| 085 | 365 | 363 | L3 | 7.00 | — |
| 086 | 366 | 364 | U5 | 3.03 | — |
| 087 | 366 | — | L3 | 7.90 | — |
| 088 | 366 | 364 | L1 | 8.00 | — |
| 089 | 366 | — | L3 | 7.80 | — |
| 090 | 366 | — | L3 | 8.80 | — |
| 091 | 367 | 365 | L3 | 7.50 | — |
| 092 | 368 | 366 | U4 | 3.67 | — |
| 093 | 369 | — | U5 | 3.16 | (CDCl3, 300 MHz): 7.20-7.56 (5 H, m); 7.09 (1 H, d); 6.93 |
| | | | | | (1 H, dd); 6.10 (1 H, d); 4.87-5.35 (3 H, m); 4.42-4.62 (1 |
| | | | | | H, m); 3.42-3.62 (1 H, m); 2.95-3.32 (2 H, m); 2.84 (1 H, |
| | | | | | dd); 2.65 (3 H, s); 2.21-2.50 (1 H, m) |
| 094 | 369 | — | U5 | 3.21 | (DMSO d6, 300 MHz): 8.02 (1 H, d); 7.21-7.62 (7 H, m); |
| | | | | | 6.81-7.05 (1 H, m); 4.91-5.34 (3 H, m); 4.25-4.5 (1 H, m); |
| | | | | | 2.75-3.27 (4H, m); 2.59 (3 H, s) |
| 095 | 369 | 367 | L3 | 6.30 | — |
| 096 | 369 | 367 | L3 | 6.50 | (DMSO d6-D20, 300 MHz): 7.40-7.55 (m, 2H); 7.24-7.37 |
| | | | | | (m, 3H); 7.12-7.23 (m, 1H); 6.95-7.10 (m, 1H); 5.17 (s, |
| | | | | | 2H); 4.44-4.68 (m, 1H); 3.16-3.54 (m, 4H); 2.60 (s, 3H); |
| | | | | | 2.21-2.33 (m, 1H); 2.10-1.99 (m, 1H) |
| 097 | 369 | 367 | L3 | 6.90 | (DMSO d6, 300 MHz): 7.40-7.55 (m, 3H); 7.24-7.37 (m, |
| | | | | | 3H); 7.12-7.23 (m, 1H); 6.95-7.10 (m, 1H); 5.17 (s, 2H); |
| | | | | | 4.44-4.68 (m, 1H); 3.16-3.54 (m, 5H); 2.60 (s, 3H); 2.21- |
| | | | | | 2.33 (m, 1H); 2.10-1.99 (m, 1H) |
| 098 | 372 | 370 | L3 | 9.10 | — |
| 099 | 373 | 371 | L3 | 7.60 | — |
| 100 | 373 | 371 | L3 | 7.20 | — |
| 101 | 373 | 371 | L3 | 8.30 | — |
| 102 | 374 | — | L3 | 7.00 | (DMSO d6, 300 MHz) :. 68-8.55 (m, 4H); 7.50-7.36 (m, 7H); |
| | | | | | 6.98 (dd, 1H); 5.13 (s, 2H); 4.67 (d, 2H); 2.64 (s, 3H) |
| 103 | 376 | 374 | U5 | 2.83 | (CDCl3, 300 MHZ):7.21-7.63 (6 H, m); 6.93 (1 H, dd, |
| | | | | | J = 8.67, 1.88 Hz); 6.10 (1 H, d, J-6.03 Hz); 4.86-5.42 (3 H, |
| | | | | | m); 4.38-4.67 (1 H, m); 3.42-3.68 (1 H, m); 2.96-3.34 (2 |
| | | | | | H, m); 2.84 (1 H, dd); 2.65 (3 H, s); 2.29-2.52 (1 H, m) |
| 104 | 376 | — | L3 | 4.20 | — |
| 105 | 377 | — | L3 | 8.20 | (DMSO-d6, 300 MHz): 8.48 (t, 1H); 7.49-7.30 (m, 7H); 6.98 |
| | | | | | (dd, 1H); 6.21 (s, 1H); 5.14 (s, 2H); 4.49 (d, 2H); 2.62 (s, |
| | | | | | 3H); 2.38 (s, 3H) |
| 106 | 378 | — | L3 | 9.60 | — |
| 107 | 379 | — | U5 | 3.08 | — |
| 108 | 379 | 377 | U5 | 2.66 | — |
| 109 | 379 | — | U5 | 2.99 | — |
| 110 | 379 | 377 | U5 | 2.9 | — |
| 111 | 379 | 377 | U4 | 3.55 | (DMSO d6, 300 MHz): 8.08 (1 H, d); 7.30-7.57 (7 H, m); |
| | | | | 3.63 | 7.22 (1 H, d); 6.97 (1 H, dd); 5.13 (2 H, s); 4.10-4.25 (1 H, |
| | | | | | m); 3.35-3.44 (1 H, m); 3.11-3.23 (1 H, m); 2.57 (3 H, s); |
| | | | | | 2.26-2.38 (2 H, m); 1.84-2.07 (2 H, m) |
| 112 | 379 | — | L3 | 6 | — |
| 113 | 379 | — | L3 | 6.3 | — |
| 114 | 379 | 377 | U5 | 3.12 | — |
| 115 | 379 | 377 | U5 | 3.15 | (DMSO d6, 300 MHz): 7.89 (d, 1H), 7.29-7.58 (m, 6H), |
| | | | | | 7.22 (d, 1H), 6.97 (dd, 1H), 5.15 (s, 2H), 3.76-3.93 (m, 1H), |
| | | | | | 2.98 (d,, 1H), 2.55-2.70 (m, 5H), 1.80 (t, 2H), 1.37 (td, 1H), |
| | | | | | 0.91-1.24 (m, 4H). |
| 116 | 379 | 377 | U6 | 1.16 | — |
| 117 | 379 | 377 | L1 | 6.80 | — |
| 118 | 379 | — | L3 | 7.40 | — |
| 119 | 379 | 377 | L3 | 7.5 | — |
| 120 | 379 | 377 | L5 | 8.70 | (DMSO d6, 300 MHz): 7.91 (d, 1H); 7.88-7.61 (m, 2H); |
| | | | | | 7.51-7.28 (m, 6H); 7.20 (d, 1H); 6.97 (dd, 1H); 5.12 (s, 2H); |
| | | | | | 3.80-3.67 (m, 1H); 3.05-2.91 (m, 1H); 2.56 (s, 3H); 2.08-1.85 |
| | | | | | (m, 4H); 1.52-1.31 (m, 4H). |
| 121 | 379 | 377 | L6 | 8.20 | — |
| 122 | 380 | — | L3 | 7.50 | (DMSO d6, 300 MHz): 7.98 (t, 1H); 7.48-7.27 (m, 7H); 6.97 |
| | | | | | (dd, 1H); 5.13 (s, 2H); 3.27-3.16 (m, 4H); 2.69 (s, 2H); 2.58 |
| | | | | | (s, 3H); 1.97-1.73 (m, 1H); 1.63-1.58 (m, 2H); 1.30-1.17 (m, |
| | | | | | 2H) |
| 123 | 380 | 378 | U5 | 3.1 | (300 MHz, DMSO-d6): 7.81 (1 H, d); 7.27-7.64 (6 H, m); |
| | | | | | 7.13-7.25 (1 H, m); 6.96 (1 H, d); 5.12 (2 H, s); 4.61 (1 H, |
| | | | | | d); 3.60-3.90 (1 H, m); 2.55 (3 H, s); 1.86 (4 H, d); 0.85- |
| | | | | | 1.54 (4 H, m) |
| 124 | 380 | 378 | U5 | 3.19 | (DMSO d6, 300 MHz): 7.20-7.77 (6 H, m); 7.10 (1 H, d); |
| | | | | | 6.94 (1 H, dd); 5.61 (1 H, d); 5.03-5.20 (2 H, m); 3.84-4.12 |
| | | | | | (1 H, m); 3.48-3.76 (1 H, m); 2.57-2.78 (3 H, m); 1.94- |
| | | | | | 2.27 (4 H, m); 1.83 (1 H, br. s.); 1.41-1.53 (2 H, m); 1.22- |
| | | | | | 1.34 (2H, m) |
| 125 | 380 | 378 | U5 | 3.26 | (CDCl3, 300 MHz): 7.29-7.59 (6 H, m); 7.29-7.59 (6 H, |
| | | | | | m); 7.08-7.27 (1 H, m); 6.94 (1 H, dd); 5.77 (1 H, d); 5.11 |
| | | | | | (2 H, s); 4.09 (br. s., 1 H); 3.99 (1 H, br. s.); 2.68 (3 H,s); |
| | | | | | 1.70-2.04 (8 H, m) |
| 126 | 381 | 379 | U5 | 2.81 | — |
| 127 | 381 | 379 | L3 | 6.30 | (DMSO d6, 300 MHz): 7.49 (1 H, d); 7.20-7.39 (2 H, m); |
| | | | | | 6.83-7.11 (4 H, m); 5.12 (2 H, s); 4.60-4.52 (m, 1H); 3.03- |
| | | | | | 3.57 (4 H, m); 2.58-2.68 (3 H, s); 1.90-2.37 (2 H, m) |
| 128 | 382 | 380 | U6 | 1.10 | (DMSO d6, 300 MHz): 8.25 (1 H, t); 7.25-7.58 (7 H, m); |
| | | | | | 6.77-7.04 (1 H, m); 5.12 (2 H, s); 5.04 (1 H, t,); 4.45 (2 H, |
| | | | | | d); 4.35 (2 H, d);3.64 (2 H, d); 3.53 (2 H, d); 2.60 (3 H, s) |
| 129 | 383 | 381 | L3 | 6.2 | — |
| 130- | 383 | 381 | U5 | 3.08 | — |
| Dia 1 | | | | | |
| 130- | 382 | — | U5 | 3.29 | — |
| Dia 2 | | | | | |
| 131 | 383 | — | U5 | 3.19 | (DMSO d6, 300 MHz): 8.20-8.54 (1 H, m); 7.28-7.56 (6 |
| | | | | | H, m); 7.22 (1 H, d); 6.90-7.04 (1 H, m); 4.97-5.23 (3 H, |
| | | | | | m); 4.22-4.49 (1 H, m); 3.46-3.73 (2 H, m); 2.99-3.18 (2 |
| | | | | | H, m); 2.58 (3 H, s); 1.56-2.30 (2 H, m) |
| 132 | 384 | 382 | L3 | 8.10 | — |
| 133 | 386 | — | L3 | 8.9 | — |
| 134 | 386 | 384 | L2 | 5.40 | — |
| 135 | 387 | 385 | U5 | 3.24 | — |
| 136 | 387 | 385 | U5 | 3.12 | (DMSO d6, 300 MHz): 8.00 (1 H, d); 7.37-7.64 (2 H, m); |
| | | | | | 7.27 (1 H, d); 7.18 (2 H, t); 6.92 (1 H, dd); 5.16 (2 H, s); 4.23- |
| | | | | | 4.45 (1 H, m); 3.43-3.49 (2 H,m) 3.01-3.20 (2 H, m); 2.57 |
| | | | | | (3H, s); 1.69-2.12 (2 H, m) |
| 137 | 387 | — | U5 | 3.43 | — |
| 138 | 387 | — | L3 | 7.70 | — |
| 139 | 387 | — | L3 | 7.10 | — |
| 140 | 389 | — | U5 | 3.35 | — |
| 141 | 389 | — | U5 | 3.29 | — |
| 142 | 391 | 389 | U5 | 3.39 | (DMSO d6, 300 MHz): 8.18 (1H, d); 7.50-7.35 (6H, m); 7.22 |
| | | | | | (1H, d); 6.99 (1H, dd); 5.15 (2H, s); 4.09-4.07 (1H, m); 3.32 |
| | | | | | (1H, t); 2.98-2.80 (4H, m); 2.59 (3H, s); 2.02-1.91 (2H, m); |
| | | | | | 1.73-1.69 (2H, m); 1.54-1.46 (2H, m). |
| 143 | 391 | — | U5 | 3.32 | (CDCl3, 300 MHz): 7.31-7.57 (7 H, m); 6.99 (1 H, d); 6.16 |
| | | | | | (1 H, d); 5.12 (2 H, s); 4.40 (1 H, d); 3.44-3.91 (2 H, m); |
| | | | | | 2.71 (3 H, s); 2.33 (2 H, d); 1.09-1.42 (4 H, m) 0.63-1.00 |
| | | | | | (2 H, m) |
| 144 | 391 | — | U6 | 1.27 | — |
| | | | | 1.42 | |
| 144- | 391 | — | U5 | 3.36 | — |
| Dia 1 | | | | | |
| 144- | 391 | — | U5 | 3.51 | — |
| Dia 2 | | | | | |
| 145 | 393 | — | L3 | 6.2 | (DMSO d6, 300 MHZ): 8.27-8.21 (m, 1H); 7.40-7.31 (m, |
| | | | | | 6H); 7.20 (s, 1H); 6.96 (dd, 1H); 5.13 (s, 2H); 4.54-4.42 (m, |
| | | | | | 1H); 4.04-3.40 (m, 4H); 2.56 (d, 3H); 2.27-2.02 (m, 2H); |
| | | | | | 1.95 (s, 3H) |
| 146 | 393 | 391 | L3 | 6.2 | (300 MHz; CDCl3): 7.50-7.29 (m, 6H); 7.08 (d, 1H); 6.96 |
| | | | | | (dd, 1H); 5.67 (br d, 1H); 5.11 (s, 2H); 4.60-4.35 (m, 1H); |
| | | | | | 3.55-3.35 (m, 2H); 2.99 (s, 3H); 2.86 (ddd, 1H); 2.68 (s, 3H); |
| | | | | | 2.40-2.35 (m, 2H); 1.97-1.82 (m, 1H). |
| 147 | 396 | 391 | U5 | 3.36 | (CDCl3, 300 MHZ):7.22-7.69 (8 H, m); 6.93 (1 H, d); 6.58 |
| | | | | | (1 H, br. s.) ; 4.96-5.40 (2 H, m); 4.31-4.69 (1 H, m); 3.21- |
| | | | | | 3.79 (4 H, m); 2.81-2.98 (1 H, m); 2.70 (4 H, s); 1.68- |
| | | | | | 2.15 (2 H, m); 0.95-1.43 (4 H, m) |
| 148 | 393 | 391 | U5 | 3.00 | (DMSO d6, 300 MHz): 8.20-8.64 (1 H, m); 7.12-7.68 (8 |
| | | | | | H, m); 6.97 (1 H, d); 4.92-5.26 (2 H, m); 4.19-4.81 (5 H, |
| | | | | | m); 3.63-3.85 (1 H, m); 3.44-3.63 (2 H, m); 2.99-3.21 (2 |
| | | | | | H, m); 2.58 (3 H, s) |
| 149 | 393 | — | L3 | 7.90 | — |
| 150 | 393 | 391 | L4 | 3.10 | — |
| 151 | 393 | — | L3 | 7.50 | — |
| 152 | 393 | 391 | L4 | 4.40 | — |
| 153 | 393 | 391 | L4 | 5.00 | — |
| 154 | 393 | 391 | L3 | 7.40 | — |
| 155 | 393 | — | L3 | 7.3 | — |
| 156 | 393 | — | L3 | 7.00 | — |
| 157 | 394 | 392 | U5 | 1.87 | — |
| 158 | 394 | — | U5 | 2.75 | — |
| 159 | 394 | — | U6 | 1.4 | (300 MHz; CDCl3): 7.31-7.55 (m, 6 H) 7.11 (d, 1 H) 6.87- |
| | | | | | 7.02 (m, 1 H) 5.59 (br. s., 1 H) 5.12 (s, 2 H) 3.38 (s, 3 H) 2.68 |
| | | | | | (s, 3 H) 1.97-2.29 (m, 4 H) 1.34-1.48 (m, 2 H) 1.26 (s, 5 |
| | | | | | H) 0.71-0.97 (m, 3 H) |
| 160 | 395 | 393 | U5 | 1.90 | — |
| 161 | 395 | 393 | U4 | 2.85 | — |
| 162 | 395 | 393 | U4 | 2.88 | — |
| 163 | 395 | — | U4 | 2.94 | — |
| 164 | 395 | 393 | L3 | 6.4 | — |
| 165 | 395 | 393 | L3 | 7.10 | — |
| 166 | 396 | 394 | U5 | 2.65 | — |
| 167 | 396 | 394 | U5 | 3.01 | (300 MHz, CDCl3): 7.22-7.64 (7 H, m); 7.13 (1 H, br. s.); |
| | | | | | 6.83-7.05 (1 H, m); 5.80 (1 H, br. s.); 5.13 (2 H, s); 3.55- |
| | | | | | 4.00 (6 H, m); 2.52-2.88 (3 H, m) 1.96 (4 H, br. s.) |
| 168 | 397 | 395 | L3 | 6.90 | (CDCl3, 300 MHz): 7.42-7.32 (m, 2H); 7.26-7.11 (m, 3H); |
| | | | | | 7.06-6.98 (m, 1H); 6.94 (dd, 1H); 5.69-5.65 (m, 1H); 5.11 (s, |
| | | | | | 2H); 4.98 (s, 2H); 4.05-3.95 (m, 1H); 3.00-2.85 (m, 2H); 2.68 |
| | | | | | (s, 3H); 2.39 (s, 3H); 2.29 (br t, 2H); 2.11 (br d, 2H); 1.80- |
| | | | | | 1.75 m, 2H). |
| 169 | 400 | 398 | L3 | 5.3 | — |
| 170 | 400 | 398 | L2 | 4.3 | (DMSO d6, 300 MHz): 7.67 (1 H, br. s.); 7.39 (1 H, d); 7.27 |
| | | | | | (3 H, d); 7.14 (1 H, br. s) 6.91 (1 H, d); 6.79 (1H, br. s.); 5.05 |
| | | | | | (2 H, br. s.); 2.96 (1 H, br. s.); 2.69 (3 H, br. s.); 2.32 (3 H, |
| | | | | | br. s.); 0.89 (4 H, br. s.) |
| 171 | 401 | 399 | U5 | 3.02 | — |
| 172 | 401 | 399 | U5 | 3.25 | — |
| 173 | 401 | 399 | U5 | 3.19 | (CDCl3, 400 MHZ):7.13-7.61 (7 H, m); 6.98 (1 H, dd); 6.07 |
| | | | | | (1 H, d); 5.05-5.23 (2 H, m); 4.48-4.78 (1 H, m); 3.37 (1 |
| | | | | | H, t); 3.17 (1 H, d); 2.86-3.06 (1 H, m); 2.65-2.85 (4 H, |
| | | | | | m); 2.22-1.19 (1 H, m); 1.53-1.68 (1 H, m) |
| 174 | 403 | — | L3 | 6.3 | — |
| 175 | 404 | 402 | L3 | 5.4 | — |
| 176 | 404 | 402 | L3 | 5.0 | (DMSO d6, 300 MHz): 11.8 (1H, br s); 7.54 (1 H, d) 7.40- |
| | | | | | 7.50 (1 H, m) 7.25-7.39 (3 H, m) 7.14-7.22 (1 H, m) 7.04 (1 |
| | | | | | H, dd) 5.17 (2 H, s) 3.00-3.18 (1 H, m) 2.66 (3 H, s) 1.08- |
| | | | | | 1.21 (4 H, m) |
| 177 | 404 | 402 | L3 | 5.0 | (DMSO d6, 300 MHz): 11.80 (1 H, br. s.); 7.42-7.64 (3 H, |
| | | | | | m) 7.13-7.34 (3 H, m) 7.02 (1 H, dd); 5.13 (2 H, s); 3.09- |
| | | | | | 3.21 (1 H, m); 2.66 (3 H, s); 1.10-1.21 (4 H, m) |
| 178 | 405 | — | L3 | 8.1 | (300 MHz, DMSO-d6): 8.00 (t, 1H); 7.50-7.34 (m, 6H); 7.20 |
| | | | | | (d, 1H); 6.97 (dd, 1H); 5.10 (s, 2H); 3.38 (m, 2H); 2.61 (m, |
| | | | | | 2H); 2.56 (s, 3H); 2.26 (s, 3H); 2.18 (s, 3H) |
| 179 | 405 | — | L3 | 6.30 | — |
| 180 | 405 | 403 | U6 | 1.08 | (CDCl3, 300 MHz): 7.30-7.52 (m, 6H), 7.14 (d, 1H), 6.96 |
| | | | | | (dd, 1H), 5.87 (d, 1H), 5.13 (s, 2H), 4.51-4.65 (m, 1H), 2.87- |
| | | | | | 2.95 (m, 2H), 2.79-2.87 (m, 2H), 2.69 (s, 3H), 2.39-2.51 |
| | | | | | (m, 2H), 1.57-1.78 (m, 6H). |
| 181 | 405 | — | U5 | 3.51 | — |
| | | | | 3.63 | |
| 182 | 405 | 403 | U5 | 3.50 | (CDCl3, 300 MHz): 7.30-7.55 (6 H, m); 7.14 (1 H, d); 6.94 |
| | | | | | (1 H, dd); 5.58 (1 H, d); 5.09 (2 H, s); 4.30-4.50 (1 H, m); |
| | | | | | 3.24 (2 H, br. s.); 2.67 (3 H, s); 2.32 (3 H, s); 2.06-2.18 (2 |
| | | | | | H, m); 1.90-2.10 (2 H, m,); 1.71-1.88 (2 H, m) |
| 183 | 404 | — | U5 | 3.59 | (CDCl3, 300 MHz): 7.30-7.51 (6 H, m); 7.23 (1 H, d); 6.87- |
| | | | | | 7.07 (1 H, m); 6.07-6.36 (1 H, m); 5.12 (2 H, s); 4.26-4.36 |
| | | | | | (1 H,m); 3.21 (2 H, br. s.); 2.71 (3 H, s); 2.27-2.40 (5 H, m) |
| | | | | | 2.08-2.25 (2 H, m) 1.70-1.92 (4 H, m) |
| 184 | 405 | 403 | U5 | 3.52 | — |
| 185 | 407 | 405 | L3 | 8.50 | — |
| 186 | 407 | — | L3 | 7.6 | — |
| | | | | 7.8 | |
| 187 | 407 | — | L3 | 5.00 | — |
| 188 | 408 | — | U5 | 2.89 | — |
| 189 | 408 | 406 | L3 | 6.40 | — |
| 190 | 408 | — | U5 | 2.31 | — |
| 191 | 409 | 407 | U4 | 4.04 | — |
| 192 | 410 | 407 | U5 | 2.56 | (CDCl3, 300 MHz): 7.92 (1H, d); 7.49-7.44 (3H, m); 7.37- |
| | | | | | 7.25 (2H, m); 7.22 (1H, d); 6.96 (1H, dd); 5.21-516 (3H, m); |
| | | | | | 4.63 (2H, s); 3.79-3.69 (1H, m); 2.77-2.73 (2H, m); 2.56 |
| | | | | | (3H, s); 2.17 (3H, s); 2.01-1.94 (2H, m); 1.82-1.79 (2H, m); |
| | | | | | 1.66-1.53 (2H, m). |
| 193 | 410 | 408 | U5 | 1.78 | — |
| 194 | 411 | 409 | L3 | 7.70 | (DMSO d6, 300 MHz):7.41-7.31 (m, 2H); 7.25-7.11 (m, |
| | | | | | 3H); 7.02 (ddd, 1H); 6.94 (dd, 1H); 5.67 (br d; 1H); 5.11 (s, |
| | | | | | 2H); 4.12-3.97(m, 1H); 3.08 (d, 2H); 2.89 (br d, 2H); 2.36 (s, |
| | | | | | 3H); 2.32-2.18 (m, 2H); 2.18-2.02 (m, 2H); 1.72-1.55 (m, |
| | | | | | 2H); 1.34 (t, 3H). |
| 195 | — | 412 | L3 | 6.2 | (DMSO d6, 300 MHz): 7.73 (d, 1H); 7.48 (d, 2H); 7.43-7.28 |
| | | | | | (m, 4H); 6.88 (dd, 1H); 5.08 (s, 2H); 2.69 (s, 3H). |
| 196 | 414 | — | L3 | 6.30 | (DMSO d6, 300 MHz): 9.38 (dd, 1H); 8.86 (dd, 1H); 8.59 (t, |
| | | | | | 1H); 8.16 (bs, 1H); 7.57-7.32 (m, 7H); 6.98 (dd, 1H); 5.15 (s, |
| | | | | | 2H); 4.78 (d, 2H); 2.66 (s, 3H) |
| 197 | 414 | 412 | U5 | 3.20 | — |
| 198 | 414 | — | U5 | 3.28 | — |
| 199 | 416 | 414 | L3 | 5.0 | — |
| 200 | 417 | — | L3 | 7.00 | — |
| 201 | 419 | 417 | U5 | 2.80 | — |
| 202 | 419 | 417 | L3 | 7.70 | — |
| 203 | 419 | 417 | L3 | 7.40 | — |
| 204 | 419 | 417 | L3 | 7.50 | — |
| 205 | 421 | — | U5 | 3.30 | (CDCl3, 300 MHz): 7.17-7.61 (7 H, m); 6.94 (1 H, dd); 6.29 |
| | | | | | (1 H, d); 5.12 (2 H, s); 4.51-4.77 (1 H, m); 2.89-3.13 (3 H, |
| | | | | | m), 2.80 (1 H, d); 2.68 (3 H, s); 2.39-2.62 (2 H, m); 2.08- |
| | | | | | 2.37 (7 H, m); 1.81-1.97 (1 H, m); 1.13-1.38 (1 H, m) |
| 206 | 421 | 419 | U5 | 3.51 | (DMSO d6, 300 MHz): 7.78 (1H, d); 7.48-7.31 (6H, m); 7.24 |
| | | | | | (1H, d); 6.98 (1H, dd); 5.13 (2H, s); 4.51 (4H, ddd); 3.99- |
| | | | | | 3.94 (1H, m); 3.43 (1H, t); 2.69 (2H, m); 2.58 (3H, s); 1.99- |
| | | | | | 1.91 (2H, m); 1.83-1.72 (2H, m); 1.57-1.46 (2H, m). |
| 207 | 421 | 419 | U5 | 3.47 | (DMSO d6, 300 MHz): 7.80 (1H, d); 7.49-7.35 (6H, m); 7.26 |
| | | | | | (1H, d); 6.99 (1H, dd); 5.15 (2H, s); 4.50 (4H, ddd); 4.02- |
| | | | | | 3.99 (1H, m); 3.46 (1H, t); 2.70 (2H, m); 2.52 (3H, s); 1.99- |
| | | | | | 1.91 (2H, m); 1.83-1.72 (2H, m); 1.57-1.46 (2H, m). |
| 208 | 421 | 419 | U5 | 3.11 | — |
| 209 | 421 | 419 | U6 | 1.43 | — |
| 210 | 422 | 420 | U5 | 3.89 | (CDCl3, 300 MHz): 7.21-7.64 (6 H, m); 7.13 (1 H, d); 6.84- |
| | | | | | 7.02 (1 H, m); 5.51-5.74 (1 H, m); 5.11 (2 H, s); 3.77- |
| | | | | | 4.09 (1 H, m); 2.67 (3 H, s); 2.1-2.2 (2 H, m); 1.93 (2 H, d); |
| | | | | | 1.75 (1 H, br. s.) 1.04-1.44 (12 H, m) |
| 211 | 423 | — | U4 | 4.21 | — |
| 212 | 424 | 422 | U5 | 3.60 | |
| 213 | 429 | — | L3 | 6.80 | (DMSO d6, 300 MHz): 9.38 (dd, 1H); 8.86 (dd, 1H); 8.57 (d, |
| | | | | | 1H); 7.50-7.29 (m, 8H); 6.97 (dd, 1H); 5.54-5.43 (m, 1H); |
| | | | | | 5.15 (s, 2H); 2.65 (s, 3H); 1.66 (d, 3H) |
| 214 | 428 | — | L3 | 8.70 | — |
| 215 | 429 | — | L3 | 7.2 | — |
| 216 | 429 | — | L3 | 9 | — |
| 217 | 432 | 430 | U5 | 3.29 | — |
| 218 | 432 | 430 | U5 | 3.25 | — |
| 219 | 433 | 431 | L4 | 2.3 | — |
| 220 | 434 | 432 | U5 | 3.78 | — |
| 221 | 433 | 431 | L3 | 9.10 | — |
| 222 | 434 | 432 | U4 | 3.48 | (DMSO d6, 400 MHZ): 8.04-8.26 (2 H, m); 7.19-7.57 (7 |
| | | | | | H, m); 6.82-7.05 (1 H, m); 5.14 (2 H, s); 4.55-4.65 (1 H, m); |
| | | | | | 4.26-4.39 (1 H, m); 4.03 (1 H, d); 3.69 (1 H, dd); 3.55 (1 H, |
| | | | | | dd); 3.45 (1 H, dd); 2.58 (3 H, s); 1.99-2.25 (1 H, m) |
| 223 | 435 | 433 | L3 | 7.50 | — |
| 224 | 436 | 434 | U5 | 2.6 | — |
| 225 | 436 | — | L3 | 6.60 | — |
| 226 | 437 | 435 | U5 | 3.42 | — |
| 227 | 437 | 435 | U5 | 3.35 | — |
| 228 | 437 | — | L3 | 8.90 | — |
| 229 | 437 | 435 | L3 | 7.30 | — |
| 230 | 443 | 441 | U6 | 2.03 | (CDCl3, 300 MHz): 8.24-8.39 (2 H, m); 7.22-7.58 (6 H, |
| | | | | | m); 7.08 (1 H, d); 6.94 (1 H, d); 6.49 (1 H, t); 5.66 (1 H, d); |
| | | | | | 5.10 (2 H, s); 4.73 (2 H, d); 4.19-4.38 (1 H, m); 3.06-3.25 |
| | | | | | (2 H, m); 2.67 (3 H, s); 2.03-2.23 (2 H, m); 1.43-1.52 (2 |
| | | | | | H,m) |
| 231 | 447 | — | U6 | 2.08 | — |
| 232 | 447 | 445 | L3 | 7.90 | — |
| 233 | 447 | 445 | L3 | 7.80 | — |
| 234 | 447 | 445 | L3 | 8.10 | — |
| 235 | 448 | 446 | U4 | 3.62 | (DMSO d6, 300 MHz): 8.16 (1 H, d); 7.28-7.56 (6 H, m); |
| | | | | | 7.24 (1 H, d); 6.97 (1 H, dd); 5.14 (2 H, s); 4.53-4.83 (1 H, |
| | | | | | m); 4.30-4.44 (1 H, m); 4.24 (1 H, d); 3.62-3.84 (2 H, m); |
| | | | | | 3.41-3.57 (1 H, m); 2.81-3.00 (4 H, m);2.69 (1H.s); 2.57 |
| | | | | | (4 H, s); 1.98-2.28 (1 H, m) |
| 236 | 449 | 447 | L3 | 8.30 | — |
| 237 | 450 | 448 | L4 | 4.90 | — |
| 238 | 451 | 449 | L3 | 8.90 | — |
| 239 | 451 | 449 | L4 | 3.80 | — |
| 240 | 455 | — | U5 | 3.97 | — |
| 241 | 455 | 453 | L3 | 9.20 | — |
| 242 | 457 | 455 | U5 | 2.7 | (300 MHz, DMSO-d6) : 8.03 (1H, d); 7.98 (1H, d); 7.85 (2H, |
| | | | | | m); 7.67 (1H, t); 7.50 (1H, d); 7.26 (1H, d); 7.00 (1H, dd); |
| | | | | | 5.53 (2H, s); 3.86-3.71 (1H, m);3.32 (3H, s); 2.88-2.85 (2H, |
| | | | | | m); 2.57 (3H, s); 2.27 (3H, s); 2.22-2.15 (2H, m); 1.87-1.83 |
| | | | | | (2H, m); 1.66-1.57 (2H, m). |
| 243 | 460 | 458 | L4 | 4.50 | — |
| 244 | 463 | 461 | L3 | 8.30 | — |
| 245 | 463 | — | U5 | 2.8 | (300 MHz, DMSO-d6) 8.17 (d,, 1H), 7.88 (s, 1H), 7.40- |
| | | | | | 7.54 (m, 2H), 7.25-7.38 (m, 2H), 7.16 (d, 1H), 7.09 (t, 1H), |
| | | | | | 6.93 (dd, 1H), 5.14 (s, 2H), 4.52-4.65 (m, 1H), 3.20-3.30 |
| | | | | | (m, 2H), 2.91 (br. s., 4H), 2.60 (s, 3H), 2.30-2.48 (m, 6H), |
| | | | | | 2.21 (s, 3H), 1.93-2.11 (m, 1H). |
| 246 | 465 | — | U6 | 2.21 | — |
| 247 | — | 463 | L4 | 5.50 | — |
| 248 | 465 | 463 | L4 | 4.80 | — |
| 249 | — | 463 | L4 | 5.20 | — |
| 250 | 466 | — | U5 | 3.3 | — |
| 251 | 479 | 477 | U6 | 2.14 | — |
| 252 | 479 | 477 | U6 | 2.19 | — |
| 253 | — | 477 | L4 | 5.40 | — |
| 254 | — | 481 | U5 | 4.48 | — |
| 255 | 483 | — | U6 | 2.36 | — |
| Dia 1 | | | | | |
| 255- | 483 | — | U6 | 2.4 | — |
| Dia 2 | | | | | |
| 256 | 487 | 485 | U6 | 1.8 | — |
| 257 | — | 485 | U6 | 2.0 | — |
| 258 | 491 | — | U6 | 2.42 | — |
| 259 | — | 489 | U6 | 2.22 | — |
| 260 | 493 | — | U4 | 1.5 | — |
| 261 | 394 | — | U5 | 3.2 | — |
| 262 | — | 493 | U6 | 1.60 | — |
| 263 | — | 499 | U6 | 2.35 | — |
| 264 | 505 | 503 | U6 | 2.36 | (CDCl3, 300 MHz): 7.30-7.51 (m, 6H), 7.15 (s, 1H), 6.96 |
| | | | | | (d, 1H), 5.87 (d, 1H), 5.13 (s, 2H), 4.50-4.68 (m, 1H), 3.39 |
| | | | | | (t, 2H), 3.32 (t, 2H), 2.69 (s, 3H), 2.44 (t, 2H), 1.63-1.74 |
| | | | | | (m, 4H), 1.50-1.57 (m, 2H), 1.46 (s, 9H) |
| 265 | 508 | 506 | U6 | 2.51 | — |
| 267 | 533 | 531 | U6 | 2.7 | — |

| | [M + H]⁺ | ¹H NMR |
|---|---|---|
| Cpd | (m/z): | (δ ppm) |
| 297-En 1 | 412.1 | (DMSO-d₆): 8.70 (d, 1H), 7.88 (d, 1H), 7.50-7.48 (m, 2H), 7.44-7.40 (m, 2H), 7.37- |
| | | 7.33 (m, 1H), 7.28 (d, 1H), 5.33 (s, 2H), 4.58-4.49 (m, 1H), 3.32-3.20 (m, 2H), 3.10- |
| | | 2.82 (m, 3H), 2.52 (m, 3H). |
| 298-En 1 | 405.2 | (MeOD, aliphatic-NH proton not seen in H-NMR): 7.46-7.44 (m, 2H), 7.38-7.34 |
| | | (m, 2H), 7.33-7.28 (m, 1H), 7.21-7.18 (m, 1H), 7.15-7.11 (m, 1H), 5.16 (s, 2H), |
| | | 4.78-4.69 (m, 1H), 3.55-3.48 (m, 1H), 3.41-3.32 (m, 1H), 3.19-3.10 (m, 1H), 2.94- |
| | | 2.85 (m, 1H), 2.60 (s, 3H). |
| 299-En 1 | 401.2 | (DMSO-d6): 8.65 (d, 1H), 7.49-7.45 (m, 2H), 7.39 (t, 2H), 7.33-7.28 (m, 2H), 7.04 |
| | | (d, 1H), 5.12 (s, 2H), 4.62-4.54 (m, 1H), 3.27-3.20 (m, 2H), 3.02-2.71 (m, 3H), 2.49 |
| | | (s, 3H), 2.28 (s, 3H). |
| 300-En 1 | 421.1 | (DMSO-d6): 8.24 (d, 1H), 7.81 (s, 1H), 7.51-7.48 (m, 2H), 7.43-7.32 (m, 4H), 5.22 |
| | | (s, 2H), 4.64-4.58 (m, 1H), 3.29-3.26 (m, 2H), 3.10-2.80 (m, 3H), 2.59 (s, 3H). |
| 301-En 1 | 412.1 | (DMSO): 8.35 (d, 1H), 8.14 (s, 1H), 7.53-7.48 (m, 2H), 7.45-7.41 (m, 3H), 7.39-7.33 |
| | | (m, 1H), 5.29 (s, 2H), 4.64-4.56 (m, 1H), 3.10-3.26 (m, 1H), 3.08-2.82 (m, 3H), 2.63 |
| | | (s, 3H). |
| 302-En 1 | 405.2 | (MeOD): 7.48-7.45 (m, 2H), 7.41-7.36 (m, 2H), 7.35-7.31 (m, 1H), 7.29 (d, 1H), |
| | | 7.23 (d, 1H), 6.00 (d, 1H), 5.18 (s, 2H), 4.75-4.66 (m, 1H), 3.70-3.64 (m, 1H), 3.50- |
| | | 3.40 (m, 1H), 3.31-3.22 (m, 1H), 2.87-2.80 (m, 1H), 2.68 (s, 3H), 1.85 (br s, 1H). |
| 303-En 1 | 401.2 | (DMSO-d6): 8.13 (d, 1H), 7.52-7.46 (m, 2H), 7.44-7.39 (m, 3H), 7.37-7.30 (m, 1H), |
| | | 7.23 (s, 1H), 5.14 (s, 2H), 4.69-4.57 (m, 1H), 3.30-3.20 (m, 2H), 3.10-2.80 (m, 3H), |
| | | 2.57 (s, 3H), 2.29 (s, 3H). |
| 304-En 1 | 421.1 | (DMSO-d6): 8.29 (d, 1H), 7.47-7.32 (m, 5H), 7.19 (dd, 2H), 5.15 (s, 2H), 4.68-4.59 |
| | | (m, 1H), 3.95-3.49 (m, 1H), 3.37-3.26 (m, 2H), 3.11-3.00 (m, 1H), 2.92-2.86 (m, |
| | | 1H), 2.59 (s, 3H). |
| 305-En 1 | 412.1 | (DMSO-d6): 8.36 (d, 1H), 7.58-7.52 (dd, 2H), 7.49-7.46 (m, 2H), 7.42-7.38 (m, 2H), |
| | | 7.37-7.32 (m, 1H), 5.19 (s, 2H), 4.67-4.57 (m, 1H), 3.30-3.25 (m, 1H), 3.07-2.84 (m, |
| | | 3H), 2.65 (s, 3H). |
| 306-En 1 | 405.2 | (CDCl3): 7.48-7.38 (m, 4H), 7.37-7.31 (m, 1H), 7.01-6.99 (m, 1H), 6.75 (dd, 1H), |
| | | 6.11 (d, 1H), 5.10 (s, 2H), 4.78-4.65 (m, 1H), 3.70-3.62 (m, 1H), 3.51-3.40 (m, 1H), |
| | | 3.31-3.20 (m, 1H), 2.90-2.82 (m, 1H), 2.73 (s, 3H), 1.88 (br s, 1H). |
| 307-En 1 | 401.2 | (DMSO-d6): 8.12 (d, 1H), 7.50-7.43 (m, 2H), 7.40 (t, 2H), 7.35-7.30 (m, 1H), 7.07 |
| | | (d, 1H), 6.85 (d, 1H), 5.13 (s, 2H), 4.70-4.57 (m, 1H), 3.29-3.20 (m, 2H), 3.10-2.98 |
| | | (m, 1H), 2.95-2.81 (m, 2H), 2.59 (s, 3H), 2.42 (s, 3H). |
| 308-En 1 | 410.1 | (DMSO-d6): 7.41-7.31 (m, 6H), 7.26-7.22 (m, 1H), 7.10 (d, 1H), 6.87 (dd, 1H), 5.45 |
| | | (q, 1H), 4.87 (t, 1H), 3.75-3.52 (m, 6H), 2.53 (s, 3H), 2.13 (t, 2H), 1.67-1.60 (m, 2H), |
| | | 1.59 (d, 3H). |
| 309 | 399.1 | (400 MHZ, DMSO-d6): δ 8.70 (s, 1H), 7.59 (td, 1H), 7.51-7.40 (m, 3H), 7.29-7.16 |
| | | (m, 4H), 7.01 (dd, 1H), 5.19 (s, 2H), 4.86 (d, 2H), 4.67 (d, 2H), 2.66 (s, 3H). |
| 310 | 386.1 | (500 MHz, DMSO-d6): δ 8.34 (s, 1H), 7.58 (td, 1H), 7.48-7.40 (m, 2H), 7.34 (d, |
| | | 1H), 7.28-7.23 (m, 2H), 6.97 (dd, 1H), 5.20-5.17 (m, 3H), 4.66 (d, 2H), 4.54 (d, 2H), |
| | | 3.77 (d, 2H), 2.60 (s, 3H). |
| 311-En 1 | 427.1 | (400 MHZ, DMSO-d6): δ 8.18 (d, 1H), 7.58 (td, 1H), 7.50-7.36 (m, 3H), 7.29-7.22 |
| | | (m, 2H), 6.97 (dd, 1H), 5.16 (s, 2H), 4.75-4.65 (m, 2H), 3.55-3.50 (m, 2H), 3.48-3.36 |
| | | (m, 3H), 3.20-3.13 (m, 1H), 2.61 (s, 3H), 2.39-2.31 (m, 1H), 2.02-1.91 (m, 1H). |
| 311-En 2 | 427.0 | (400 MHZ, DMSO-d6): δ 8.18 (d, 1H), 7.58 (td, 1H), 7.50-7.36 (m, 3H), 7.29-7.22 |
| | | (m, 2H), 6.97 (dd, 1H), 5.16 (s, 2H), 4.75-4.65 (m, 2H), 3.55-3.50 (m, 2H), 3.48- |
| | | 3.36 (m, 3H), 3.20-3.13 (m, 1H), 2.61 (s, 3H), 2.39-2.31 (m, 1H), 2.02-1.91 (m, 1H). |
| 312 | 388.1 | (400 MHZ, DMSO-d6): δ 7.62-7.56 (m, 1H), 7.49-7.40 (m, 2H) 7.31 (d, 1H), 7.29- |
| | | 7.22 (m, 2H), 7.05 (s, 1H), 6.96 (dd, 1H), 5.14 (s, 2H), 4.96 (t, 2H), 3.67-3.61 (m, |
| | | 2H), 3.58-3.52 (m, 2H), 2.59 (s, 3H), 1.31 (s, 3H). |
| 313 | 372.2 | (400 MHZ, DMSO-d6): δ 7.61-7.55 (m, 1H), 7.47-7.39 (m, 2H) 7.29-7.20 (m, 4H), |
| | | 6.96 (dd, 1H), 5.15 (s, 2H), 5.04 (t, 1H), 3.49 (d, 2H), 2.57 (s, 3H), 1.31 (s, 3H). |
| 314 | 387.1 | (400 MHz, DMSO-d6): δ 7.66-7.57 (m, 2H), 7.51-7.40 (m, 4H), 7.29-7.22 (m, 2H), |
| | | 7.18 (brs, 1H), 6.98 (dd, 1H), 5.16 (s, 2H), 5.01 (s, 1H), 4.50-4.44 (m, 1H), 3.75 (s, |
| | | 2H), 2.64 (s, 3H). |
| 315 | 385.1 | (500 MHz, DMSO-d6): δ 7.83 (s, 1H), 7.62-7.56 (m, 1H), 7.48 (d, 1H), 7.45-7.40 |
| | | (m, 2H) 7.34 (brs, 1H), 7.29-7.22 (m, 2H), 7.15 (brs, 1H), 6.98 (dd, 1H), 5.17 (s, |
| | | 2H), 2.63 (s, 3H), 1.55 (s, 6H). |
| 316 | 424.1 | (400 MHZ, DMSO-d6): δ 7.51-7.49 (m, 2H), 7.38-7.34 (m, 2H), 7.34-7.25 (m, |
| | | 3H), 6.92 (d, 1H), 6.65 (dd, 1H), 4.83 (t, 1H), 3.68-3.66 (m, 2H), 3.65 (d, 2H), 3.59- |
| | | 3.48 (m, 2H), 2.53 (s, 3H), 2.07 (brd, 2H), 1.64-1.57 (m, 8H). |
| 317 | 426.1 | (400 MHZ, DMSO-d6): δ 7.39-7.30 (m, 6H), 7.26-7.22 (m, 1H), 7.09 (d, 1H), 6.89- |
| | | 6.86 (dd, 1H), 5.27-5.24 (m, 1H), 5.13 (t, 1H), 4.86 (t, 1H), 3.75-3.52 (m, 8H), |
| | | 2.53 (s, 3H), 2.16-2.08 (m, 2H), 1.65-1.61 (m, 2H). |
| 318 | 385.1 | (400 MHZ, DMSO-d6): δ 7.58 (s, 1H), 7.41 (d, 1H), 7.33-7.17 (m, 3H), 7.16-7.05 |
| | | (m, 2H), 6.95-6.92 (m, 1H), 2.52 (s, 2H), 2.65 (s, 3H), 1.54 (s, 6H). |
| 319 | 372.1 | (400 MHZ, DMSO-d6): δ 7.76 (s, 1H), 7.58 (d, 1H), 7.38 (dd, 1H), 7.32-7.25 (m, |
| | | 2H), 7.24-7.18 (m, 1H), 7.14-7.08 (m, 1H), 6.97-6.92 (m, 1H), 5.25 (s, 2H), 4.98 (t, |
| | | 1H), 3.51 (d, 2H), 2.59 (s, 3H), 1.34 (s, 6H). |
| 320 | 371.1 | (500 MHz, DMSO-d6): δ 7.88 (d, 1H), 7.60-7.56 (m, 1H), 7.48-7.44 (m, 2H), 7.43- |
| | | 7.39 (m, 1H), 7.37 (d, 1H), 7.28-7.22 (m, 2H), 7.09 (s, 1H), 6.97 (dd, 1H), 5.19-5.14 |
| | | (m, 2H), 4.50-4.43 (m, 1H), 2.60 (s, 3H), 1.35 (d, 3H). |
| 321 | 387.1 | (400 MHZ, DMSO-d6): δ 7.87 (d, 1H), 7.72 (d, 1H), 7.59 (d, 1H), 7.46 (brs, 1H), |
| | | 7.41 (dd, 1H), 7.29 (td, 1H), 7.24-7.10 (m, 3H), 6.67-6.93 (m, 1H), 5.24 (s, 2H), 5.00 |
| | | (t, 1H), 4.50-4.45 (m, 1H), 3.74 (t, 2H), 2.67 (s, 3H). |
| 322 | 397.1 | (400 MHZ, DMSO-d6): δ 8.26 (s, 1H), 7.62-7.55 (m, 1H), 7.49-7.36 (m, 3H), 7.29- |
| | | 7.21 (m, 2H), 7.02-6.95 (m, 2H), 6.87 (brs, 1H), 5.18 (s, 2H), 2.63 (s, 3H), 2.60-2.52 |
| | | (m, 2H), 2.29-2.20 (m, 2H), 2.00-1.80 (m, 2H). |
| 323 | 413 | (400 MHZ, DMSO-d6): δ 8.24 (s, 1H), 7.58 (td, 1H), 7.50-7.40 (m, 2H), 7.33 (d, |
| | | 1H), 7.29-7.21 (m, 2H), 7.12 (brs, 1H), 7.07 (brs, 1H), 6.97 (dd, 1H), 5.17 (s, 2H), |
| | | 4.18 (d, 1H), 3.93 (d, 1H), 3.84 (t, 2H), 2.61 (s, 3H), 2.40-2.31 (m, 2H). |
| 324 | 384.1 | (400 MHZ, DMSO-d6): δ 7.76 (s, 1H), 7.57 (t, 1H), 7.48-7.34 (m, 2H), 7.30-7.22 (m, |
| | | 3H), 6.96 (dd, 1H), 5.16 (s, 2H), 4.90 (t, 1H), 3.65 (d, 2H), 2.58 (s, 3H), 2.38-2.26 |
| | | (m, 2H), 2.19-2.09 (m, 2H), 1.89-1.71 (m, 2H). |
| 325 | 370.1 | (400 MHZ, DMSO-d6): δ 8.15 (s, 1H), 7.57 (td, 1H), 7.46-7.39 (m, 2H), 7.29-7.21 |
| | | (m, 3H), 6.95 (dd, 1H), 5.16 (s, 2H), 4.78 (bs, 1H), 3.55 (s, 2H), 2.56 (s, 3H), 0.80- |
| | | 0.71 (m, 4H). |
| 326 | 383.1 | (400 MHZ, DMSO-d6): δ 8.31 (s, 1H), 7.57 (td, 1H), 7.48-7.39 (m, 2H), 7.36 (d, |
| | | 1H), 7.29-7.22 (m, 2H), 7.19 (brs, 1H), 7.05 (brs, 1H), 6.95 (dd, 1H), 5.18 (s, 2H), |
| | | 2.60 (s, 3H), 1.37-1.30 (m, 2H), 1.05-1.00 (m, 2H). |
| 327 | 400.1 | (400 MHZ, DMSO-d6): δ 7.85 (s, 1H), 7.58 (t, 1H), 7.48-7.40 (m, 2H), 7.28-7.22 (m, |
| | | 3H), 6.96 (dd, 1H), 5.15 (s, 2H), 5.07 (t, 1H), 3.92 (d, 1H), 3.83-3.75 (m, 3H), 3.72- |
| | | 3.56 (m, 2H), 2.57 (s, 3H), 2.27-2.20 (m, 1H). 2.10-2.03 (m, 1H). |
| 328 | 358.1 | (400 MHZ, DMSO-d6): δ 7.69-7.63 (m, 1H), 7.57 (td, 1H), 7.47-7.39 (m, 2H), 7.29- |
| | | 7.21 (m, 3H), 6.96 (dd, 1H), 5.16 (s, 2H), 4.80 (brs, 1H), 4.07-3.99 (m, 1H), 3.52- |
| | | 3.46 (m, 1H), 3.41-3.35 (m, 1H), 2.61 (s, 3H), 1.15 (d, 3H). |
| 329 | 386.1 | (400 MHZ, DMSO-d6): δ 7.62-7.55 (m, 1H), 7.50-7.40 (m, 3H), 7.37 (d, 1H), 7.29- |
| | | 7.21 (m, 2H), 6.98 (dd, 1H), 5.58 (d, 1H), 5.15 (s, 2H), 4.44-4.37 (m, 1H), 4.33-4.27 |
| | | (m, 1H), 4.00-3.90 (m, 2H), 3.67-3.56 (m, 2H), 2.63 (s, 3H). |
| 330 | 386.1 | (500 MHZ, DMSO-d6): δ 8.14 (d, 1H), 7.57 (td, 1H), 7.46 (d, 1H), 7.45-7.40 (m, |
| | | 1H), 7.30-7.21 (m, 3H), 6.97 (dd, 1H), 5.30 (d, 1H), 5.16 (s, 2H), 4.25-4.20 (m, 2H), |
| | | 4.03-3.98 (m, 1H), 3.92-3.88 (m, 1H), 3.67-3.64 (m, 1H), 3.57-3.54 (m, 1H), 2.56 (s, |
| | | 3H). |
| 331 | 406.1 | (400 MHZ, DMSO-d6): δ 8.40 (d, 1H), 7.61-7.54 (m, 1H), 7.50 (d, 1H), 7.46-7.39 |
| | | (m, 1H), 7.30-7.22 (m, 3H), 6.99 (dd, 1H), 5.16 (s, 2H), 4.91-4.80 (m, 1H), 4.32 (t, |
| | | 1H), 4.19-4.08 (m, 1H), 3.96-3.82 (m, 2H), 2.59 (s, 3H). |
| 332 | 474.1 | (400 MHz, DMSO-d6): δ 8.02 (m, 1H), 7.82 (d, 1H), 7.78 (t, 1H), 7.65-7.68 |
| | | (m,1H), 7.49 (d, 1H), 7.50 (d, 1H), 7.48 (s, 1H), 7.28 (s, 1H), 6.98 (dd, 1H), 5.52 (m, |
| | | 2H), 4.86 (bs, 1H), 3.57-3.7 (m, 6H), 3.30 (s,3H), 2.61 (s, 2H), 2.16 (d, 2H), 1.59- |
| | | 1.65 (m, 2H). |
| 333 | 479.2 | (400 MHZ, DMSO-d6): δ 8.23 (d, 1H), 8.02 (d, 1H), 7.76-7.83 (m, 2H), 7.65-7.68 |
| | | (m, 1H), 7.50 (d, 1H), 7.24 (d, 1H), 6.99-7.02 (d, 1H), 5.51 (s, 2H), 4.44-4.52 (m, |
| | | 1H), 3.36 (s, 3H), 3.07-3.30 (m, 1H), 2.77-2.93 (m, 2H), 2.58-2.63 (s, 4 H), 2.47 |
| | | (bs, 1H), 1.78-1.81 (m, 2H). |
| 334 | 413.1 | (400 MHz, DMSO-d6): δ 7.78 (s, 1H), 7.59 (td, 1H), 7.53-7.39 (m, 3H), 7.30-7.21 |
| | | (m, 3H), 6.97 (dd, 1H), 5.23-5.10 (m, 3H), 3.65-3.55 (m, 2H), 3.30-3.22 (m, 2H), |
| | | 2.51 (s, 3H), 2.50-2.35 (m, 2H). |
| 335 Dia 1 | 417.1 | (400 MHz, DMSO-d6): δ 7.53 (d, 1H), 7.50-7.44 (m, 2H), 7.41 (d, 1H), 7.36-7.27 |
| | | (m, 2H), 7.23-7.12 (m, 3H), 6.87 (dd, 1H), 5.55-5.51 (m, 1H), 5.24 (t, 1H), 5.00 (t, |
| | | 1H), 4.50-4.42 (m, 1H), 3.87-3.80 (m, 1H), 3.78-3.65 (m, 3H), 2.60 (s, 3H) |
| 335 Dia 2 | 417.1 | (400 MHZ, DMSO-d6): δ 7.53 (d, 1H), 7.50-7.44 (m, 2H), 7.41 (d, 1H), 7.36-7.27 |
| | | (m, 2H), 7.23-7.12 (m, 3H), 6.87 (dd, 1H), 5.55-5.51 (m, 1H), 5.24 (t, 1H), 5.00 (t, |
| | | 1H), 4.50-4.42 (m, 1H), 3.87-3.80 (m, 1H), 3.78-3.65 (m, 3H), 2.60 (s, 3H). |
| 336 Dia 1 | 381.2 | (400 MHZ, DMSO-d6): δ 7.54-7.42 (m, 4H), 7.39 (d, 1H), 7.34-7.30 (m, 3H), 7.26- |
| | | 7.21 (m, 2H), 6.88 (dd, 1H), 5.50 (q, 1H), 5.02 (t, 1H), 4.49-4.45 (m, 1H), 3.75- |
| | | 3.72 (m, 2H), 2.59 (s, 3H), 1.57 (d, 3H). |
| 336 Dia 2 | 381.2 | (400 MHZ, DMSO-d6): δ 7.54-7.42 (m, 4H), 7.39 (d, 1H), 7.34-7.30 (m, 3H), 7.26- |
| | | 7.20 (m, 2H), 6.88 (dd, 1H), 5.50 (q, 1H), 5.04 (t, 1H), 4.49-4.44 (m, 1H), 3.78- |
| | | 3.72 (m, 2H), 2.60 (s, 3H), 1.57 (d, 3H). |
| 337 | 369.1 | (500 MHz, DMSO-d6): δ 7.61 (d, 1H), 7.50-7.45 (m, 4H), 7.43-7.36 (m, 3H), 7.35- |
| | | 7.34 (m, 1H), 7.19 (bs, 1H), 6.98 (dd, 1H), 5.12 (s, 2H), 5.04 (bs, 1H), 4.49-4.46 |
| | | (m, 1H), 3.78-3.71 (m, 2H), 2.63 (s, 3H). |
| 338 | 401.1 | (500 MHz, DMSO-d6): δ 7.66-7.62 (m, 2H), 7.54-7.45 (m, 3H), 7.44 (d, 1H), 7.43- |
| | | 7.37 (m, 2H), 7.19 (bs, 1H), 7.00 (dd, 1H), 5.18 (s, 2H), 5.01 (t, 1H), 4.50-4.44 |
| | | (m, 1H), 3.75-3.72 (m, 2H), 2.64 (s, 3H). |
| 339 | 399.2 | (500 MHZ, DMSO-d6): δ 7.63 (d, 2H), 7.48-7.45 (m, 2H), 7.45-7.41 (m, 1H), 7.40 |
| | | (m, 1H), 7.36-7.31 (m, 1H), 7.18 (s,1H), 7.01 (d, 1H), 6.99-6.93 (m, 2H), 5.07 (s, |
| | | 2H), 5.01 (t, 1H), 4.50-4.44 (m, 1H), 3.83 (s, 3H), 3.76-3.72 (m, 2H), 2.64 (s, 3H). |
| 340 | 405.2 | (500 MHz, DMSO-d6): δ 7.69-7.61 (m, 2H), 7.52-7.45 (m, 2H), 7.43 (d, 1H), 7.30 |
| | | (td, 1H), 7.19 (s, 1H), 7.12 (td, 1H), 6.98 (dd, 1H), 5.13 (s, 2H), 5.01 (t, 1H), 4.50- |
| | | 4.45 (m, 1H), 3.79-3.71 (m, 2H), 2.64 (s, 3H). |
| 341 | 394.2 | (500 MHz, DMSO-d6): δ 7.91 (d, 1H), 7.78-7.72 (m, 2H), 7.65 (d, 1H), 7.60-7.56 |
| | | (m, 1H), 7.52-7.44 (m, 3H), 7.18 (s, 1H), 7.00 (dd, 1H), 5.26 (s, 2H), 5.00 (t, 1H), |
| | | 4.50-4.45 (m, 1H), 3.77-3.72 (m, 2H), 2.64 (s, 3H). |
| 342 | 401.2 | (500 MHz, DMSO-d6): δ 7.62 (d, 1H), 7.49-7.41 (m, 4H), 7.18 (s, 1H), 7.08 (d, |
| | | 1H), 7.04 (d, 1H), 6.96 (dd, 1H), 5.10 (s, 2H), 5.01 (t, 1H), 4.50-4.45 (m, 1H), 3.78- |
| | | 3.72 (m, 2H), 2.63 (s, 3H), 2.32 (s, 3H). |
| 343-En 1 | 393.2 | (500 MHz, DMSO-d6): δ 8.16 (s, 1H), 7.45-7.41 (m, 2H), 7.38 (d, 1H), 7.35- |
| | | 7.31 (m, 2H), 7.26-7.21 (m, 2H), 6.93 (s, 1H), 6.90-6.85 (m, 2H), 5.50 (q, 1H), |
| | | 2.60-2.51 (m, 5H), 2.28-2.14 (m, 2H), 1.98-1.84 (m, 2H), 1.56 (d, 3H). |
| 345-En 1 | 427.2 | (500 MHz, DMSO-d6): δ 8.24 (s, 1H), 7.48-7.40 (m, 2H), 7.35-7.29 (m, 1H), 7.22- |
| | | 7.13 (m, 3H), 6.91-6.86 (m, 3H), 5.57-5.54 (m, 1H), 5.26 (bs, 1H), 3.84-3.78 |
| | | (m, 1H), 3.70-3.65 (m, 1H), 2.61-2.55 (m, 5H), 2.25-2.10 (m, 2H), 1.98-1.80 |
| | | (m, 2H). |
| 346-En 1 | 413.2 | (500 MHz, DMSO-d6): δ 7.53 (d, 1H), 7.48-7.44 (m, 3H), 7.38 (d, 1H), 7.34-7.24 |
| | | (m, 4H), 7.20 (s, 1H), 6.88 (dd, 1H), 5.53-5.50 (m, 1H), 5.06 (s, 1H), 4.46-4.43 |
| | | (m, 1H), 3.78-3.70 (m, 3H), 3.59-3.54 (m, 1H), 3.35 (s, 3H), 2.59 (s, 3H). |
| 347-En 1 | 426.2 | (500 MHz, DMSO-d6): δ 7.54-7.36 (m, 5H), 7.33-7.27(m, 3H), 7.25-7.20 (m, |
| | | 2H), 6.88 (dd, 1H), 5.47-5.43 (m, 1H), 5.08 (bs, 1H), 4.47-4.42 (m, 1H), 3.79- |
| | | 3.70 (m, 2H), 2.87-2.82 (m, 1H), 2.59 (s, 3H), 2.55-2.52 (m, 1H), 2.29 (s, 6H). |

In all above cases the analytical LCMS and ¹H NMR data collected for En2 matched the data for En1.

Chiral Analytical Data

Chiral SFC analysis

| | | | Co- | | | |
|---|---|---|---|---|---|---|
| | | | solvent | Flow | RT | Purity |
| Cpd | Column Name | Co-solvent | [%] | [g/min] | [min] | [%] |
| Cpd 297-En 1 | CHIRALCEL OX-3 | 0.5% DEA | 40 | 3 | 1.87 | 99.6 |
| | (4.6*150 mm) 3 μm | in MeOH | | | | |
| Cpd 297-En 2 | CHIRALCEL OX-3 | 0.5% DEA | 40 | 3 | 3.60 | 99.2 |
| | (4.6*150 mm) 3 μm | in MeOH | | | | |
| Cpd 298-En 1 | CHIRALPAK AD-3 | 0.5% DEA | 40 | 3 | 9.24 | 99.9 |
| | (4.6*150 mm) 3 μm | in MeOH | | | | |
| Cpd 298-En 2 | CHIRALPAK AD-3 | 0.5% DEA | 40 | 3 | 3.71 | 99.7 |
| | (4.6*150 mm) 3 μm | in MeOH | | | | |
| Cpd 299-En 1 | CHIRALCEL OX-3 | MeOH | 30 | 3 | 2.43 | 98.9 |
| | (4.6*150 mm) 3 μm | | | | | |
| Cpd 299-En 2 | CHIRALCEL OX-3 | MeOH | 30 | 3 | 4.66 | 98.1 |
| | (4.6*150 mm) 3 μm | | | | | |
| Cpd 300-En 1 | CHIRACEL OD-H | MeOH | 25 | 3 | 7.55 | 98.4 |
| | (250 mm × 4.6), 5 μm | | | | | |
| Cpd 300-En 2 | CHIRACEL OD-H | MeOH | 25 | 3 | 4.43 | 98.6 |
| | (250 mm × 4.6), 5 μm | | | | | |
| Cpd 301-En 1 | CHIRALCEL OZ-3 | MeOH | 40 | 3 | 1.68 | 96.7 |
| | (4.6*150 mm) 3 μm | | | | | |
| Cpd 301-En 2 | CHIRALCEL OZ-3 | MeOH | 40 | 3 | 3.14 | 95.8 |
| | (4.6*150 mm) 3 μm | | | | | |
| Cpd 302-En 1 | CHIRALPAK AD-3 | 0.5% DEA | 30 | 3 | 4.45 | 99.8 |
| | (4.6*150 mm) 3 μm | in MeOH | | | | |
| Cpd 302-En 2 | CHIRALPAK AD-3 | 0.5% DEA | 30 | 3 | 1.86 | 99.9 |
| | (4.6*150 mm) 3 μm | in MeOH | | | | |
| Cpd 303-En 1 | CHIRALCEL OD-3 | MeOH | 30 | 3 | 1.90 | 94.5 |
| | (4.6*150 mm) 3 μm | | | | | |
| Cpd 303-En 2 | CHIRALCEL OD-3 | MeOH | 30 | 3 | 1.32 | 97.7 |
| | (4.6*150 mm) 3 μm | | | | | |
| Cpd 304-En 1 | CHIRACEL OD-H | MeOH | 25 | 3 | 11.03 | 98.7 |
| | (250 mm x 4.6), 5 μm | | | | | |
| Cpd 304-En 2 | CHIRACEL OD-H | MeOH | 25 | 3 | 5.46 | 97.5 |
| | (250 mm × 4.6), 5 μm | | | | | |
| Cpd 305-En 1 | CHIRALCEL OZ-3 | MeOH | 40 | 3 | 2.37 | 96.6 |
| | (4.6*150 mm) 3 μm | | | | | |
| Cpd 305-En 2 | CHIRALCEL OZ-3 | MeOH | 40 | 3 | 6.83 | 99.6 |
| | (4.6*150 mm) 3 μm | | | | | |
| Cpd 306-En 1 | CHIRALPAK AD-3 | 0.5% DEA | 40 | 4 | 1.38 | 99.5 |
| | (4.6*150 mm) 3 μm | in MeOH | | | | |
| Cpd 306-En 2 | CHIRALPAK AD-3 | 0.5% DEA | 40 | 4 | 5.48 | 99.9 |
| | (4.6*150 mm) 3 μm | in MeOH | | | | |
| Cpd 307-En 1 | CHIRALCEL OD-3 | MeOH | 30 | 3 | 2.84 | 96.7 |
| | (4.6*150 mm) 3 μm | | | | | |
| Cpd 307-En 2 | CHIRALCEL OD-3 | MeOH | 30 | 3 | 1.61 | 96.0 |
| | (4.6*150 mm) 3 μm | | | | | |
| Cpd 308-En 1 | CHIRALPAK AD-3 | IPA | 30 | 3 | 1.22 | 99.9 |
| | (4.6*150 mm) 3 μm | | | | | |
| Cpd 308-En 2 | CHIRALPAK AD-3 | IPA | 30 | 3 | 1.94 | 99.5 |
| | (4.6*150 mm) 3 μm | | | | | |
| Cpd 311-En 1 | CHIRALPAK IC-3 | MeOH | 40 | 3 | 2.32 | 99.9 |
| | (4.6*150 mm) 3 μm | | | | | |
| Cpd 311-En 2 | CHIRALPAK IC-3 | MeOH | 40 | 3 | 3.19 | 99.8 |
| | (4.6*150 mm) 3 μm | | | | | |
| Cpd 314-En 1 | Chiralpak IG-3 | MeOH | 40 | 3 | 1.91 | 99.9 |
| | (4.6 × 150 mm), 3 μm | | | | | |
| Cpd 314-En 2 | Chiralpak IG-3 | MeOH | 40 | 3 | 3.09 | 99.8 |
| | (4.6 × 150 mm), 3 μm | | | | | |
| Cpd 317-En 1 | CHIRALPAK AD-3 | EtOH | 40 | 3 | 1.94 | 99.9 |
| | (4.6*150 mm) 3 μm | | | | | |
| Cpd 317-En 2 | CHIRALPAK AD-3 | EtOH | 40 | 3 | 2.83 | 99.5 |
| | (4.6*150 mm) 3 μm | | | | | |
| Cpd 320-En 1 | Chiralpak IG-3 | 0.5% DEA | 40 | 3 | 3.12 | 99.9 |
| | (4.6 × 150 mm), 3 μm | in MeOH | | | | |
| Cpd 320-En 2 | Chiralpak IG-3 | 0.5% DEA | 40 | 3 | 7.14 | 99.9 |
| | (4.6 × 150 mm), 3 μm | in MeOH | | | | |
| Cpd 321-En 1 | CHIRALPAK AD-3 | MeOH | 40 | 3 | 2.66 | 98.7 |
| | (4.6*150 mm) 3 μm | | | | | |
| Cpd 321-En 2 | CHIRALPAK AD-3 | MeOH | 40 | 3 | 3.59 | 97.5 |
| | (4.6*150 mm)3um | | | | | |
| Cpd 323-En 1 | CHIRALPAK AD-3 | MeOH | 40 | 3 | 3.37 | 100.0 |
| | (4.6*150 mm) 3 μm | | | | | |
| Cpd 323-En 2 | CHIRALPAK AD-3 | MeOH | 40 | 3 | 4.84 | 99.9 |
| | (4.6*150 mm) 3 μm | | | | | |
| Cpd 327-En 1 | CHIRALCEL OX-3 | MeOH | 20 | 3 | 3.02 | 98.8 |
| | (4.6*150 mm) 3 μm | | | | | |
| Cpd 327-En 2 | CHIRALCEL OX-3 | MeOH | 20 | 3 | 4.11 | 97.8 |
| | (4.6*150 mm) 3 μm | | | | | |
| Cpd 329-En 1 | CHIRALPAK AD-3 | MeOH | 40 | 3 | 2.32 | 100.0 |
| | (4.6*150 mm) 3 μm | | | | | |
| Cpd 329-En 2 | CHIRALPAK AD-3 | MeOH | 40 | 3 | 5.53 | 100.0 |
| | (4.6*150 mm) 3 μm | | | | | |
| Cpd 330-En 1 | CHIRALPAK AD-3 | MeOH | 40 | 3 | 3.39 | 100.0 |
| | (4.6*150 mm) 3 μm | | | | | |
| Cpd 330- En 2 | CHIRALPAK AD-3 | MeOH | 40 | 3 | 7.39 | 99.9 |
| | (4.6*150 mm) 3 μm | | | | | |
| Cpd 331-En 1 | CHIRALPAK AD-3 | MeOH | 40 | 3 | 1.48 | 100.0 |
| | (4.6*150 mm) 3 μm | | | | | |
| Cpd 331-En 2 | CHIRALPAK AD-3 | MeOH | 40 | 3 | 1.87 | 99.6 |
| | (4.6*150 mm) 3 μm | | | | | |
| Cpd 333-En 1 | CHIRALPAK AD-3 | 0.5% DEA | 40 | 3 | 2.45 | 99.8 |
| | (4.6*150 mm) 3 μm | in MeOH | | | | |
| Cpd 333-En 2 | CHIRALPAK AD-3 | 0.5% DEA | 40 | 3 | 4.09 | 99.9 |
| | (4.6*150 mm) 3 μm | in MeOH | | | | |
| Cpd 334-En 1 | CHIRALPAK IE-3 | MeOH | 40 | 4 | 4.48 | 99.7 |
| | (4.6*150 mm) 3 μm | | | | | |
| Cpd 334-En 2 | CHIRALPAK IE-3 | MeOH | 40 | 4 | 6.91 | 99.7 |
| | (4.6*150 mm) 3 μm | | | | | |
| Cpd 335-Dia 1 | CHIRALCEL OX-3 | MeOH | 40 | 3 | 1.80 | 99.4 |
| | (4.6*150 mm) 3 μm | | | | | |
| Cpd 335-Dia 2 | CHIRALCEL OX-3 | MeOH | 40 | 3 | 2.88 | 99.4 |
| | (4.6*150 mm) 3 μm | | | | | |
| Cpd 336-Dia 1 | CHIRALCEL OX-3 | MeOH | 15 | 3 | 2.63 | 99.8 |
| | (4.6*150 mm) 3 μm | | | | | |
| Cpd 336-Dia 2 | CHIRALCEL OX-3 | MeOH | 15 | 3 | 3.18 | 99.1 |
| | (4.6*150 mm) 3 μm | | | | | |
| Cpd 343-En 1 | CHIRALCEL OJ-3 | MeOH | 40 | 3 | 1.62 | 99.97 |
| | (4.6*150 mm) 3 μm | | | | | |
| Cpd 343-En 2 | CHIRALCEL OJ-3 | MeOH | 40 | 3 | 2.94 | 98.79 |
| | (4.6*150 mm) 3 μm | | | | | |
| Cpd 345-En 1 | CHIRALPAK AD-3 | MeOH | 40 | 3 | 1.83 | 99.74 |
| | (4.6*150 mm) 3 μm | | | | | |
| Cpd 345-En 2 | CHIRALPAK AD-3 | MeOH | 40 | 3 | 3.20 | 99.73 |
| | (4.6*150 mm) 3 μm | | | | | |
| Cpd 346-En 1 | CHIRALPAK AD-3 | MeOH | 40 | 3 | 2.53 | 99.25 |
| | (4.6*150 mm) 3 μm | | | | | |
| Cpd 346-En 2 | CHIRALPAK AD-3 | MeOH | 40 | 3 | 6.00 | 99.89 |
| | (4.6*150 mm) 3 μm | | | | | |
| Cpd 347-En 1 | CHIRALPAK IG | 0.5% DEA | 40 | 3 | 2.46 | 99.89 |
| | (4.6*150 mm) 5 μm | in MeOH | | | | |
| Cpd 347-En 2 | CHIRALPAK IG | 0.5% DEA | 40 | 3 | 5.58 | 99.75 |
| | (4.6*150 mm) 5 μm | in MeOH | | | | |

Part B

### Monitoring the TRPM3 Ion Channel Driven Ca²⁺ Uptake.

In order to monitor the inhibition of the mouse TRPM3α2 (mTRPM3) ion channel by the compounds of the invention, a cellular system making use of an mTRPM3alpha2 or hTRPM3 overexpressing cell line (flip-in HEK293) was used. The TRPM3 channel was stimulated/opened with Pregnenolone sulfate (PS) (50 μM) which results in Ca²⁺ influx.

For mTRPM3, the intracellular Ca²⁺ was measured with a Calcium responsive dye, Fluor-4 AM ester (Invitrogen). Cells were cultured until a confluence of 80-90%, washed with Versene (Invitrogen) and detached from the surface by a short incubation with 0.05% Trypsin (Invitrogen). The trypsination process was stopped by the addition of complete cell culture medium (DMEM, glutamax, 10% FCS, NEAA, Pen-Strep). Cells were collected and resuspended in Krebs buffer without Calcium at RT.

Prior the cell seeding (±2000 cells/well into a black, 384 well plate (Greiner)) the diluted compound was added in the assay plate, together with the PS dissolved in Krebs buffer containing Calcium. This resulted in a 2.4 mM Ca²⁺ assay solution. Directly after cell addition the plates were read on an Envision fluorescence reader (Perkin Elmer) by an Excitation of 485 nM and emission at 535 nM.

Channel inhibition was calculated compared to a non-PS stimulated control versus a condition stimulated with PS (50 μM) with vehicle. The ability of the compounds of the invention to inhibit this activity was determined as: Percentage inhibition=[1−((RFU determined for sample with test compound present−RFU determined for sample with positive control inhibitor) divided by (RFU determined in the presence of vehicle−RFU determined for sample with positive control inhibitor))]*100.

The activities of the Example compounds tested are depicted in the table below. The activity ranges A, B and C refer to IC50 values in the Fluo-4 AM assay as follows: “A”: IC₅₀<1 μM; “B”: 1 μM≤IC₅₀≤20 μM and “C”: IC₅₀>20 μM

| CPD CODE | IC₅₀ |
|---|---|
| Cpd 001 | B |
| Cpd 002 | A |
| Cpd 003 | A |
| Cpd 004 | B |
| Cpd 005 | A |
| Cpd 006 | B |
| Cpd 008 | A |
| Cpd 009 | B |
| Cpd 010 | B |
| Cpd 011 | B |
| Cpd 012 | A |
| Cpd 013 | B |
| Cpd 014 | B |
| Cpd 015 | B |
| Cpd 016 | B |
| Cpd 017 | B |
| Cpd 018 | B |
| Cpd 020 | B |
| Cpd 021 | B |
| Cpd 022 | B |
| Cpd 023 | C |
| Cpd 024 | A |
| Cpd 025 | B |
| Cpd 026 | C |
| Cpd 027 | C |
| Cpd 028 | C |
| Cpd 029 | C |
| Cpd 035 | A |
| Cpd 036 | A |
| Cpd 037 | C |
| Cpd 038 | B |
| Cpd 039 | B |
| Cpd 040 | B |
| Cpd 041 | A |
| Cpd 042 | C |
| Cpd 043 | A |
| Cpd 044 | B |
| Cpd 045 | A |
| Cpd 046 | A |
| Cpd 047 | C |
| Cpd 048 | A |
| Cpd 049 | A |
| Cpd 050 | C |
| Cpd 051 | A |
| Cpd 052 | A |
| Cpd 053 | A |
| Cpd 054 | A |
| Cpd 055 | B |
| Cpd 056 | B |
| Cpd 057 | A |
| Cpd 058 | A |
| Cpd 059 | A |
| Cpd 060 | A |
| Cpd 061 | A |
| Cpd 062 | C |
| Cpd 063 | B |
| Cpd 064 | A |
| Cpd 065 | B |
| Cpd 066 | A |
| Cpd 067 | B |
| Cpd 068 | B |
| Cpd 069 | A |
| Cpd 070 | A |
| Cpd 071 | A |
| Cpd 072 | C |
| Cpd 073 | C |
| Cpd 074 | A |
| Cpd 075 | A |
| Cpd 076 | A |
| Cpd 077 | B |
| Cpd 078 | A |
| Cpd 079 | A |
| Cpd 080 | A |
| Cpd 081 | B |
| Cpd 082 | A |
| Cpd 083 | A |
| Cpd 084 | A |
| Cpd 085 | A |
| Cpd 086 | A |
| Cpd 087 | A |
| Cpd 088 | A |
| Cpd 089 | B |
| Cpd 090 | A |
| Cpd 091 | B |
| Cpd 092 | A |
| Cpd 093 | A |
| Cpd 094 | A |
| Cpd 095 | A |
| Cpd 096 | A |
| Cpd 097 | A |
| Cpd 098 | C |
| Cpd 099 | B |
| Cpd 100 | B |
| Cpd 101 | B |
| Cpd 102 | B |
| Cpd 103 | B |
| Cpd 104 | A |
| Cpd 105 | A |
| Cpd 106 | C |
| Cpd 107 | A |
| Cpd 108 | B |
| Cpd 109 | A |
| Cpd 110 | B |
| Cpd 111 | B |
| Cpd 112 | A |
| Cpd 113 | A |
| Cpd 114 | A |
| Cpd 115 | A |
| Cpd 116 | A |
| Cpd 117 | A |
| Cpd 118 | C |
| Cpd 119 | A |
| Cpd 120 | B |
| Cpd 121 | B |
| Cpd 122 | B |
| Cpd 123 | A |
| Cpd 124 | A |
| Cpd 125 | A |
| Cpd 126 | A |
| Cpd 127 | A |
| Cpd 128 | A |
| Cpd 129 | A |
| Cpd 130-Dia 1 | A |
| Cpd 130-Dia 2 | A |
| Cpd 131 | A |
| Cpd 132 | A |
| Cpd 133 | A |
| Cpd 134 | C |
| Cpd 135 | A |
| Cpd 136 | A |
| Cpd 137 | A |
| Cpd 138 | B |
| Cpd 139 | B |
| Cpd 140 | A |
| Cpd 141 | A |
| Cpd 142 | A |
| Cpd 143 | A |
| Cpd 144 | A |
| Cpd 144-Dia 1 | A |
| Cpd 144-Dia 2 | A |
| Cpd 145 | B |
| Cpd 146 | B |
| Cpd 147 | A |
| Cpd 148 | A |
| Cpd 149 | C |
| Cpd 150 | C |
| Cpd 151 | A |
| Cpd 152 | B |
| Cpd 153 | A |
| Cpd 154 | B |
| Cpd 155 | A |
| Cpd 156 | B |
| Cpd 157 | C |
| Cpd 158 | A |
| Cpd 159 | B |
| Cpd 163 | B |
| Cpd 164 | A |
| Cpd 165 | B |
| Cpd 166 | A |
| Cpd 167 | A |
| Cpd 168 | B |
| Cpd 169 | B |
| Cpd 170 | B |
| Cpd 171 | A |
| Cpd 172 | A |
| Cpd 173 | A |
| Cpd 174 | B |
| Cpd 175 | A |
| Cpd 176 | A |
| Cpd 177 | B |
| Cpd 178 | B |
| Cpd 179 | B |
| Cpd 180 | A |
| Cpd 181 | A |
| Cpd 182 | A |
| Cpd 183 | A |
| Cpd 184 | A |
| Cpd 185 | A |
| Cpd 186 | A |
| Cpd 187 | A |
| Cpd 188 | B |
| Cpd 189 | B |
| Cpd 190 | C |
| Cpd 191 | A |
| Cpd 192 | B |
| Cpd 193 | B |
| Cpd 194 | B |
| Cpd 195 | C |
| Cpd 196 | B |
| Cpd 197 | A |
| Cpd 198 | A |
| Cpd 199 | A |
| Cpd 200 | C |
| Cpd 201 | A |
| Cpd 202 | B |
| Cpd 203 | A |
| Cpd 204 | A |
| Cpd 205 | B |
| Cpd 206 | B |
| Cpd 207 | A |
| Cpd 208 | B |
| Cpd 209 | B |
| Cpd 210 | B |
| Cpd 211 | A |
| Cpd 212 | A |
| Cpd 214 | B |
| Cpd 215 | A |
| Cpd 216 | B |
| Cpd 217 | A |
| Cpd 218 | A |
| Cpd 219 | A |
| Cpd 220 | A |
| Cpd 221 | C |
| Cpd 222 | B |
| Cpd 223 | B |
| Cpd 224 | C |
| Cpd 225 | B |
| Cpd 226 | A |
| Cpd 227 | A |
| Cpd 228 | B |
| Cpd 229 | B |
| Cpd 230 | A |
| Cpd 231 | A |
| Cpd 232 | B |
| Cpd 233 | B |
| Cpd 234 | B |
| Cpd 235 | B |
| Cpd 236 | C |
| Cpd 237 | C |
| Cpd 238 | C |
| Cpd 239 | B |
| Cpd 240 | A |
| Cpd 241 | B |
| Cpd 242 | C |
| Cpd 243 | C |
| Cpd 244 | B |
| Cpd 245 | C |
| Cpd 246 | B |
| Cpd 247 | C |
| Cpd 248 | C |
| Cpd 249 | B |
| Cpd 250 | C |
| Cpd 251 | A |
| Cpd 252 | B |
| Cpd 253 | C |
| Cpd 255-Dia 1 | A |
| Cpd 256 | B |
| Cpd 257 | B |
| Cpd 258 | A |
| Cpd 259 | B |
| Cpd 260 | B |
| Cpd 261 | C |
| Cpd 262 | B |
| Cpd 263 | A |
| Cpd 265 | C |
| Cpd 267 | C |
| Cpd 297-En 1 | A |
| Cpd 297-En 2 | B |
| Cpd 298-En 1 | A |
| Cpd 298-En 2 | B |
| Cpd 299-En 1 | A |
| Cpd 299-En 2 | A |
| Cpd 300-En 1 | B |
| Cpd 300-En 2 | C |
| Cpd 301-En 1 | C |
| Cpd 301-En 2 | C |
| Cpd 302-En 1 | A |
| Cpd 302-En 2 | A |
| Cpd 303-En 1 | B |
| Cpd 303-En 2 | C |
| Cpd 304-En 1 | B |
| Cpd 304-En 2 | C |
| Cpd 305-En 1 | C |
| Cpd 305-En 2 | C |
| Cpd 306-En 1 | A |
| Cpd 306-En 2 | B |
| Cpd 307-En 1 | B |
| Cpd 307-En 2 | C |
| Cpd 308-En 1 | A |
| Cpd 308-En 2 | A |
| Cpd 309 | A |
| Cpd 310 | A |
| Cpd 311-En 1 | A |
| Cpd 311-En 2 | A |
| Cpd 312 | A |
| Cpd 313 | A |
| Cpd 314-En 1 | A |
| Cpd 314-En 2 | A |
| Cpd 315 | A |
| Cpd 316 | A |
| Cpd 317-En 1 | A |
| Cpd 317-En 2 | B |
| Cpd 318 | B |
| Cpd 319 | B |
| Cpd 320-En 1 | A |
| Cpd 320-En 2 | A |
| Cpd 321-En 1 | B |
| Cpd 321-En 2 | A |
| Cpd 322 | A |
| Cpd 323-En 1 | A |
| Cpd 323-En 2 | A |
| Cpd 324 | A |
| Cpd 325 | A |
| Cpd 326 | A |
| Cpd 327-En 1 | A |
| Cpd 327-En 2 | A |
| Cpd 328-En 1 | A |
| Cpd 328-En 2 | A |
| Cpd 329-En 1 | A |
| Cpd 329-En 2 | A |
| Cpd 330-En 1 | A |
| Cpd 330-En 2 | A |
| Cpd 331-En 1 | C |
| Cpd 331-En 2 | A |
| Cpd 332 | B |
| Cpd 333-En 1 | B |
| Cpd 333-En 2 | A |
| Cpd 334-En 1 | A |
| Cpd 334-En 2 | A |
| Cpd 335-Dia 1 | A |
| Cpd 335-Dia 2 | A |
| Cpd 336-Dia 1 | A |
| Cpd 336-Dia 2 | A |
| Cpd 337 | A |
| Cpd 338 | A |
| Cpd 339 | A |
| Cpd 340 | A |
| Cpd 342 | A |
| Cpd 343-En 1 | A |
| Cpd 343-En 2 | A |
| Cpd 344 | |
| Cpd 345-En 1 | A |
| Cpd 345-En 2 | A |
| Cpd 346-En 1 | A |
| Cpd 346-En 2 | A |
| Cpd 347-En 1 | B |
| Cpd 347-En 2 | A |

1. A compound of formula (I), a stereo-isomeric form, a physiologically acceptable salt, solvate and/or polymorph thereof 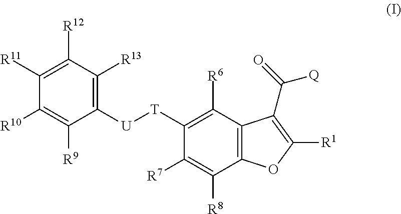
wherein
R¹ represents —F, —Cl, —Br, —I, —CN, —OC(═O)R^{W}, —NR^{W}R^{X}, —NR w C(═O)R^{X}, —SR^{W}, —S(═O)R^{W}, —S(═O)₂R^{W}, —C(═O)R^{W}, —C(═O)OR^{W}, or —C(═O)NR^{W}R^{X};
Q represents —NR³R⁴;
R³ represents —OH or —R^{Y};
R⁴ represents —R^{Y} or —S(═O)₂R^{Y};
or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —CR⁵R⁵′—; or T represents —CR⁵R⁵′— and U represents —O—;
R⁵ and R⁵′ independently of one another represent —R^{Y};
or, provided that T represents —O— and U represents —CR⁵R⁵′—, alternatively R⁵ and R⁹ together form a 4-8-membered carbocycle, saturated or unsaturated, unsubstituted or mono- or polysubstituted; or a 4-8 membered heterocycle, saturated or unsaturated, containing 1 to 3 heteroatoms selected from N, O and S, unsubstituted or mono- or polysubstituted;
R⁶, R⁷ and R⁸ independently of one another represent —F, —Cl, —Br, —I, —CN, —NO₂, —SF₅, —R^{W}—OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
R⁹, R¹⁰, R¹¹, R¹² and R¹³ independently of one another represent —F, —Cl, —Br, —I, —CN, —NO, —NO₂, ═O, ═S, —SF₅, —R^{Y}, —OR^{Y}, —OC(═O)R^{Y}, —NR^{Y}R_{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z};
wherein
R^{W} and R^{X} independently of one another in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted;
wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y} and R^{Z} independently of one another in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted;
wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, —S(═O)₂-(5 to 14-membered heteroaryl).

2. A compound of formula (I), a stereo-isomeric form, a physiologically acceptable salt, solvate and/or polymorph thereof, as defined in claim 1, wherein
R³ represent —H;
R⁴ represents
—C₁-C₆-alkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted; and
at least one of R⁹, R¹⁰, R¹¹, R¹² and R¹³ does not represent —H.

3. A compound of formula (I), a stereo-isomeric form, a physiologically acceptable salt, solvate and/or polymorph thereof, as defined in claims 1 and 2, wherein
at least one of R⁹, R¹⁰, R¹¹, R¹² and R¹³ does not represent —H; and
with the proviso that the following compounds are excluded:

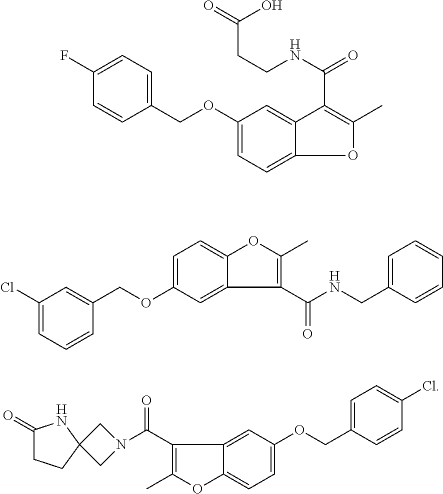 [SMILES: Cc1oc2ccc(OCc3ccc(F)cc3)cc2c1C(=O)NCCC(=O)O ; Cc1oc2ccc(OCc3cccc(Cl)c3)cc2c1C(=O)NCc1ccccc1 ; Cc1oc2ccc(OCc3ccc(Cl)cc3)cc2c1C(=O)N1CC2(CCC(=O)N2)C1]

4. A compound of formula (I), a stereo-isomeric form, a physiologically acceptable salt, solvate and/or polymorph thereof, as defined in claim 1, wherein
at least one of R⁶, R⁷ and R⁸ does not represent —H; and
with the proviso that the following compound is excluded:

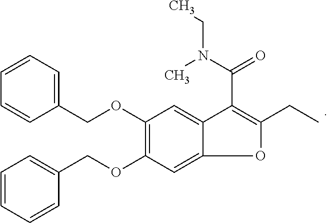 [SMILES: CCc1oc2cc(OCc3ccccc3)c(OCc3ccccc3)cc2c1C(=O)N(C)CC]

5. The compound according to claim 1, wherein T represents —O— and U represents —CR⁵R⁵′—.

6. The compound according to claim 1, wherein R¹ represents —H, —F, —Cl, —Br, —I, —C₁₋₆-alkyl, —O—C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NHC₁₋₆-alkyl, —C(═O)N(C₁₋₆-alkyl)₂, —S(═O)—C₁₋₆-alkyl, —S(═O)₂—C₁₋₆-alkyl, —OC₁₋₆ alkyl, -cyclopropyl unsubstituted, cyclobutyl unsubstituted, cyclopentyl unsubstituted or cyclohexyl unsubstituted.

7. The compound according to claim 1, wherein R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.

8. The compound according to claim 1, wherein R⁴ represents
—H;
—S(═O)₂C₁₋₆-alkyl, saturated, unsubstituted, monosubstituted or polysubstituted with —F;
—S(═O)₂(3-14-membered cycloalkyl), saturated, unsubstituted;
—C₁₋₆-alkyl, saturated, unsubstituted, monosubstituted or disubstituted with substituents independently of one another selected from the group consisting of —OH, ═O, —OC₁₋₆-alkyl, —NH₂, —NHC₁₋₆-alkyl, —N(C₁₋₆-alkyl)₂, —C₁₋₆-alkylene-NH₂, —C₁₋₆-alkylene-NH—C₁₋₆-alkyl, —C(═O)NH₂, —C(═O)—NH—C₁₋₃-alkyl, —C(═O)—N(C₁₋₃-alkyl)₂, or -phenyl unsubstituted;
3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is saturated, unsubstituted, monosubstituted or disubstituted with substituents independently of one another selected from the group consisting of —C₁₋₆-alkyl, —C₁₋₆-alkylene-NH₂, —C₁₋₆-alkylene-NH—C₁₋₆-alkylene-CF₃, —C₁₋₆-alkylene-OH, —C₁₋₆-alkylene-NHC(═O)O—C₁₋₆-alkyl, —OH, —OC₁₋₆-alkyl, —NH₂, —N(C₁₋₆-alkyl)₂, —NHC(═O)O—C₁₋₆-alkyl;
3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from azetane, 1,4-oxazepane, pyrrolidine, piperidine, azepane, diazepane, tetrahydrofuran, tetrahydropyrane, oxetane, morpholine, piperazine, hexahydrocyclopenta[c]pyrrole, octahydrocyclopenta[c]pyrrole, octahydropyrrolo[1,2-a]pyrazin, 8-azabicyclo[3.2.1]octane, 9-azabicyclo[3.3.1]nonane, quinuclidine, hexahydro-1H-pyrrolizine, 2-oxaspiro[3 0.3]heptane, 2-azaspiro[3 0.3]heptane, 7-azaspiro[3.5]nonane, 1,1-dioxothiacyclohexane, in each case unsubstituted, mono- or polysubstituted with substituents independently of one another selected from the group consisting of —F, —OH, ═O, —C₁₋₆-alkyl, —C₁₋₆-alkylene-CF₃, —C₁₋₆-alkylene-OH, —C₁₋₆-alkylene-O—C₁₋₆-alkyl, —NH₂, —N(C₁₋₆-alkyl)₂, —C₁₋₆-alkylene-NH₂, —C₁₋₆-alkylene-N(C₁₋₆-alkyl)₂, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —S(═O)₂C₁₋₆-alkyl, oxetanyl, pyrimidinyl, —C₁₋₆-alkylene-phenyl;
-phenyl unsubstituted;
5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —C₁₋₆-alkyl, —OH.

9. The compound according to claim 1, wherein R³ and R⁴ together form a heterocycle selected from the group consisting of pyrrolidine, piperidine, morpholine, and piperazine, in each case unsubstituted, mono- or polysubstituted with substituents independently of one another selected from the group consisting of —C₁₋₆-alkyl, —NH₂, —NHCH₃, —N(CH₃)₂, —C(═O)NH—C₁₋₆-alkyl, —C(═O)N(C₁₋₆-alkyl)₂, —C(═O)O—C₁₋₆-alkyl, —NHC(═O)O—C₁₋₆-alkyl, -pyridyl unsubstituted, and 1,2,4-oxadiazole unsubstituted or monosubstituted with —C₁₋₆-alkyl.

10. The compound according to claim 1, wherein R⁵ and R⁵′ independently of one another represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.

11. The compound according to claim 1, wherein R⁶, R⁷ and R⁸ independently of one another represent
—H, —F, —Cl, —Br, —I, —OH, —SH, —SF₅, —CN, —NO₂, —C(═O)OH, —NH₂,
—C₁₋₆-alkyl, —CF₃, —CHF₂, —CH₂F,
—O—C₁₋₆-alkyl, —OCF₃, —OCHF₂, —OCH₂F,
—NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—C(═O)OC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—OC(═O)C₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂; or
—C₁₋₆-heteroalkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂.

12. The compound according to claim 1, wherein R⁹, R¹⁰, R¹¹, R¹² and R¹³ independently of one another represent —H, —F, —Cl, —CN, —OH, ═O, —C₁₋₆-alkyl, —CHF₂, —CF₃, —C₁₋₆-alkylene-NH₂, —C₁₋₆-alkylene-NHC(═O)O—C₁₋₆-alkyl, —C₁₋₆-alkylene-OH, —C₁₋₆-alkylene-NHC(═O)—O—C₁₋₆-alkyl, —C(═O)O—C₁₋₆-alkyl, —N(C₁₋₆-alkyl)₂, —OC₁₋₆-alkyl, —OCF₃, —S(═O)₂—C₁₋₆-alkyl, -azetidine, —C₁₋₆-alkylene-O-tetrahydropyran, or -piperazine substituted with —C₁₋₆-alkyl.

13. The compound according to claim 1, which is selected from the group consisting of
Cpd 001 5-(benzyloxy)benzofuran-3-carboxylic acid
Cpd 002 5-(benzyloxy)-2-methylbenzofuran-3-carboxylic acid
Cpd 003 2-methyl-5-((2-methylbenzyl)oxy)benzofuran-3-carboxylic acid
Cpd 004 2-methyl-5-(1-phenylethoxy)benzofuran-3-carboxylic acid
Cpd 005 2-methyl-5-((3-methylbenzyl)oxy)benzofuran-3-carboxylic acid
Cpd 006 2-methyl-5-((4-methylbenzyl)oxy)benzofuran-3-carboxylic acid
Cpd 007 5-((3-fluorobenzyl)oxy)-2-methylbenzofuran-3-carboxylic acid
Cpd 008 5-((2-fluorobenzyl)oxy)-2-methylbenzofuran-3-carboxylic acid
Cpd 009 5-((4-fluorobenzyl)oxy)-2-methylbenzofuran-3-carboxylic acid
Cpd 010 5-((2-cyanobenzyl)oxy)-2-methylbenzofuran-3-carboxylic acid
Cpd 011 5-((2,3-dihydro-1H-inden-1-yl)oxy)-2-methylbenzofuran-3-carboxylic acid
Cpd 012 5-((3-methoxybenzyl)oxy)-2-methylbenzofuran-3-carboxylic acid
Cpd 013 5-((2-methoxybenzyl)oxy)-2-methylbenzofuran-3-carboxylic acid
Cpd 014 5-((4-methoxybenzyl)oxy)-2-methylbenzofuran-3-carboxylic acid
Cpd 015 2-ethyl-5-((3-fluorobenzyl)oxy)benzofuran-3-carboxylic acid
Cpd 016 5-((4-chlorobenzyl)oxy)-2-methylbenzofuran-3-carboxylic acid
Cpd 017 5-((3-chlorobenzyl)oxy)-2-methylbenzofuran-3-carboxylic acid
Cpd 018 5-((2-chlorobenzyl)oxy)-2-methylbenzofuran-3-carboxylic acid
Cpd 019 5-((2,6-difluorobenzyl)oxy)-2-methylbenzofuran-3-carboxylic acid
Cpd 020 5-((3-fluorobenzyl)oxy)-2-(methoxymethyl)benzofuran-3-carboxylic acid
Cpd 021 5-((2-chloro-6-fluorobenzyl)oxy)-2-methylbenzofuran-3-carboxylic acid
Cpd 022 5-((2-chloro-4-fluorobenzyl)oxy)-2-methylbenzofuran-3-carboxylic acid
Cpd 023 5-(benzyloxy)-2-cyclopentylbenzofuran-3-carboxylic acid
Cpd 024 2-methyl-5-((2-(trifluoromethyl)benzyl)oxy)benzofuran-3-carboxylic acid
Cpd 025 2-methyl-5-((3-(trifluoromethyl)benzyl)oxy)benzofuran-3-carboxylic acid
Cpd 026 2-methyl-5-((4-(trifluoromethyl)benzyl)oxy)benzofuran-3-carboxylic acid
Cpd 027 5-((2,4-dichlorobenzyl)oxy)-2-methylbenzofuran-3-carboxylic acid
Cpd 028 2-methyl-5-((3-(trifluoromethoxy)benzyl)oxy)benzofuran-3-carboxylic acid
Cpd 029 ethyl 5-(benzyloxy)benzofuran-3-carboxylate
Cpd 030 methyl 2-ethyl-5-((3-fluorobenzyl)oxy)benzofuran-3-carboxylate
Cpd 031 methyl 5-((2-chlorobenzyl)oxy)-2-methylbenzofuran-3-carboxylate
Cpd 032 ethyl 5-((2,3-dihydro-1H-inden-1-yl)oxy)-2-methylbenzofuran-3-carboxylate
Cpd 033 ethyl 5-((2-chloro-6-fluorobenzyl)oxy)-2-methylbenzofuran-3-carboxylate
Cpd 034 5-((2,3-difluorobenzyl)oxy)-2-methylbenzofuran-3-carboxylic acid
Cpd 035 5-(benzyloxy)-2-methylbenzofuran-3-carboxamide
Cpd 036 5-(benzyloxy)-N-hydroxy-2-methylbenzofuran-3-carboxamide
Cpd 037 5-((3-fluorobenzyl)oxy)-2-methylbenzofuran-3-carboxamide
Cpd 038 5-(benzyloxy)-N-ethyl-2-methylbenzofuran-3-carboxamide
Cpd 039 5-(benzyloxy)-N-cyclopropyl-2-methylbenzofuran-3-carboxamide
Cpd 040 5-(benzyloxy)-2-methyl-N-propylbenzofuran-3-carboxamide
Cpd 041 5-((2-chloro-6-fluorobenzyl)oxy)-2-methylbenzofuran-3-carboxamide
Cpd 042 N-(azetidin-3-yl)-5-(benzyloxy)-2-methylbenzofuran-3-carboxamide
Cpd 043 5-(benzyloxy)-2-methyl-N-(oxetan-3-yl)benzofuran-3-carboxamide
Cpd 044 5-(benzyloxy)-N-(2-methoxyethyl)-2-methylbenzofuran-3-carboxamide
Cpd 045 5-((2-chloro-6-fluorobenzyl)oxy)-N,2-dimethylbenzofuran-3-carboxamide
Cpd 046 5-(benzyloxy)-N-cyclopentyl-2-methylbenzofuran-3-carboxamide
Cpd 047 (5-(benzyloxy)-2-methylbenzofuran-3-yl)(piperidin-1-yl)methanone
Cpd 048 (R)-5-(benzyloxy)-2-methyl-N-(pyrrolidin-3-yl)benzofuran-3-carboxamide
Cpd 049 (S)-5-(benzyloxy)-2-methyl-N-(pyrrolidin-3-yl)benzofuran-3-carboxamide
Cpd 050 (5-(benzyloxy)-2-methylbenzofuran-3-yl)(morpholino)methanone
Cpd 051 5-(benzyloxy)-2-methyl-N-(tetrahydrofuran-3-yl)benzofuran-3-carboxamide
Cpd 052 5-(benzyloxy)-N-(3-hydroxycyclobutyl)-2-methylbenzofuran-3-carboxamide
Cpd 053 5-(benzyloxy)-N-(trans-3-hydroxycyclobutyl)-2-methylbenzofuran-3-carboxamide
Cpd 054 5-(benzyloxy)-N-(cis-3-hydroxycyclobutyl)-2-methylbenzofuran-3-carboxamide
Cpd 055 5-(benzyloxy)-N-(2-(dimethylamino)ethyl)-2-methylbenzofuran-3-carboxamide
Cpd 056 5-(benzyloxy)-N-(3-methoxypropyl)-2-methylbenzofuran-3-carboxamide
Cpd 057 5-(benzyloxy)-N-(2,3-dihydroxypropyl)-2-methylbenzofuran-3-carboxamide
Cpd 058 5-(benzyloxy)-2-methyl-N-phenylbenzofuran-3-carboxamide
Cpd 059 5-(benzyloxy)-2-methyl-N-(pyridin-3-yl)benzofuran-3-carboxamide
Cpd 060 5-(benzyloxy)-2-methyl-N-(pyridin-4-yl)benzofuran-3-carboxamide
Cpd 061 5-(benzyloxy)-2-methyl-N-(pyrazin-2-yl)benzofuran-3-carboxamide
Cpd 062 5-(benzyloxy)-2-methyl-N-(pyridazin-3-yl)benzofuran-3-carboxamide
Cpd 063 5-(benzyloxy)-2-methyl-N-(methylsulfonyl)benzofuran-3-carboxamide
Cpd 064 5-(benzyloxy)-2-methyl-N-(1-methyl-1H-pyrazol-3-yl)benzofuran-3-carboxamide
Cpd 065 5-((2-chloro-6-fluorobenzyl)oxy)-N,N,2-trimethylbenzofuran-3-carboxamide
Cpd 066 5-(benzyloxy)-N-cyclohexyl-2-methylbenzofuran-3-carboxamide
Cpd 067 (5-(benzyloxy)-2-methylbenzofuran-3-yl)(4-methylpiperidin-1-yl)methanone
Cpd 068 5-(benzyloxy)-2-methyl-N-(5-oxopyrrolidin-3-yl)benzofuran-3-carboxamide
Cpd 069 (S)-5-(benzyloxy)-2-methyl-N-(2-oxopyrrolidin-3-yl)benzofuran-3-carboxamide
Cpd 070 (R)-5-(benzyloxy)-2-methyl-N-(2-oxopyrrolidin-3-yl)benzofuran-3-carboxamide
Cpd 071 5-(benzyloxy)-2-methyl-N-(2-oxopyrrolidin-3-yl)benzofuran-3-carboxamide
Cpd 072 (5-(benzyloxy)-2-methylbenzofuran-3-yl)(4-methylpiperazin-1-yl)methanone
Cpd 073 (4-aminopiperidin-1-yl)(5-(benzyloxy)-2-methylbenzofuran-3-yl)methanone
Cpd 074 5-(benzyloxy)-2-methyl-N-(piperidin-4-yl)benzofuran-3-carboxamide
Cpd 075 5-(benzyloxy)-2-methyl-N-(piperidin-3-yl)benzofuran-3-carboxamide
Cpd 076 5-(benzyloxy)-2-methyl-N-(1-methylpyrrolidin-3-yl)benzofuran-3-carboxamide
Cpd 077 5-(benzyloxy)-2-methyl-N-(pyrrolidin-3-ylmethyl)benzofuran-3-carboxamide
Cpd 078 5-(benzyloxy)-2-methyl-N-(pyrrolidin-2-ylmethyl)benzofuran-3-carboxamide
Cpd 079 (S)-5-(benzyloxy)-2-methyl-N-(1-methylpyrrolidin-3-yl)benzofuran-3-carboxamide
Cpd 080 (R)-5-(benzyloxy)-2-methyl-N-(1-methylpyrrolidin-3-yl)benzofuran-3-carboxamide
Cpd 081 5-(benzyloxy)-N-(1-methylpiperidin-4-yl)benzofuran-3-carboxamide
Cpd 082 N-(1-(aminomethyl)cyclobutyl)-5-(benzyloxy)-2-methylbenzofuran-3-carboxamide
Cpd 083 (S)-2-methyl-5-((2-methylbenzyl)oxy)-N-(pyrrolidin-3-yl)benzofuran-3-carboxamide
Cpd 084 (S)-2-methyl-5-((3-methylbenzyl)oxy)-N-(pyrrolidin-3-yl)benzofuran-3-carboxamide
Cpd 085 (S)-2-methyl-5-((4-methylbenzyl)oxy)-N-(pyrrolidin-3-yl)benzofuran-3-carboxamide
Cpd 086 5-(benzyloxy)-N-(3-hydroxycyclopentyl)-2-methylbenzofuran-3-carboxamide
Cpd 087 5-(benzyloxy)-2-methyl-N-((tetrahydrofuran-2-yl)methyl)benzofuran-3-carboxamide
Cpd 088 5-(benzyloxy)-2-methyl-N-(tetrahydro-2H-pyran-4-yl)benzofuran-3-carboxamide
Cpd 089 (R)-5-(benzyloxy)-2-methyl-N-((tetrahydrofuran-2-yl)methyl)benzofuran-3-carboxamide
Cpd 090 (S)-5-(benzyloxy)-2-methyl-N-((tetrahydrofuran-2-yl)methyl)benzofuran-3-carboxamide
Cpd 091 5-(benzyloxy)-N-(3-(dimethylamino)propyl)-2-methylbenzofuran-3-carboxamide
Cpd 092 5-(benzyloxy)-N-(3-(hydroxymethyl)oxetan-3-yl)-2-methylbenzofuran-3-carboxamide
Cpd 093 rac-5-(benzyloxy)-N-(trans-4-fluoropyrrolidin-3-yl)-2-methylbenzofuran-3-carboxamide
Cpd 094 rac-5-(benzyloxy)-N-(cis-4-fluoropyrrolidin-3-yl)-2-methylbenzofuran-3-carboxamide
Cpd 095 (S)-5-((2-fluorobenzyl)oxy)-2-methyl-N-(pyrrolidin-3-yl)benzofuran-3-carboxamide
Cpd 096 (S)-5-((3-fluorobenzyl)oxy)-2-methyl-N-(pyrrolidin-3-yl)benzofuran-3-carboxamide
Cpd 097 (S)-5-((4-fluorobenzyl)oxy)-2-methyl-N-(pyrrolidin-3-yl)benzofuran-3-carboxamide
Cpd 098 N-benzyl-5-(benzyloxy)-2-methylbenzofuran-3-carboxamide
Cpd 099 5-(benzyloxy)-2-methyl-N-(pyridin-2-ylmethyl)benzofuran-3-carboxamide
Cpd 100 5-(benzyloxy)-2-methyl-N-(pyridin-3-ylmethyl)benzofuran-3-carboxamide
Cpd 101 5-(benzyloxy)-2-methyl-N-(pyridin-4-ylmethyl)benzofuran-3-carboxamide
Cpd 102 5-(benzyloxy)-2-methyl-N-(pyrazin-2-ylmethyl)benzofuran-3-carboxamide
Cpd 103 (S)-5-((4-cyanobenzyl)oxy)-2-methyl-N-(pyrrolidin-3-yl)benzofuran-3-carboxamide
Cpd 104 5-(benzyloxy)-2-methyl-N-((1-methyl-1H-pyrazol-5-yl)methyl)benzofuran-3-carboxamide
Cpd 105 5-(benzyloxy)-2-methyl-N-((5-methylisoxazol-3-yl)methyl)benzofuran-3-carboxamide
Cpd 106 5-(benzyloxy)-2-methyl-N-(4-methylcyclohexyl)benzofuran-3-carboxamide
Cpd 107 (R)-5-(benzyloxy)-2-methyl-N-(2-oxopiperidin-3-yl)benzofuran-3-carboxamide
Cpd 108 5-(benzyloxy)-2-methyl-N-(2-oxopiperidin-4-yl)benzofuran-3-carboxamide
Cpd 109 (S)-5-(benzyloxy)-2-methyl-N-(2-oxopiperidin-3-yl)benzofuran-3-carboxamide
Cpd 110 5-(benzyloxy)-2-methyl-N-(1-methyl-5-oxopyrrolidin-3-yl)benzofuran-3-carboxamide
Cpd 111 5-(benzyloxy)-2-methyl-N-(6-oxopiperidin-3-yl)benzofuran-3-carboxamide
Cpd 112 5-(benzyloxy)-2-methyl-N-((5-oxopyrrolidin-2-yl)methyl)benzofuran-3-carboxamide
Cpd 113 5-(benzyloxy)-2-methyl-N-(2-oxopiperidin-3-yl)benzofuran-3-carboxamide
Cpd 114 rac-5-(benzyloxy)-2-methyl-N-(trans-2-methylpiperidin-4-yl)benzofuran-3-carboxamide
Cpd 115 rac-5-(benzyloxy)-2-methyl-N-(cis-2-methylpiperidin-4-yl)benzofuran-3-carboxamide
Cpd 116 N-(cis-4-aminocyclohexyl)-5-(benzyloxy)-2-methylbenzofuran-3-carboxamide
Cpd 117 5-(benzyloxy)-2-methyl-N-(1-methylpiperidin-4-yl)benzofuran-3-carboxamide
Cpd 118 (R)-(5-(benzyloxy)-2-methylbenzofuran-3-yl)(3-(dimethylamino)pyrrolidin-1-yl)methanone
Cpd 119 N-(azepan-4-yl)-5-(benzyloxy)-2-methylbenzofuran-3-carboxamide
Cpd 120 N-(trans-4-aminocyclohexyl)-5-(benzyloxy)-2-methylbenzofuran-3-carboxamide
Cpd 121 5-(benzyloxy)-2-methyl-N-(piperidin-4-ylmethyl)benzofuran-3-carboxamide
Cpd 122 5-(benzyloxy)-2-methyl-N-((tetrahydro-2H-pyran-4-yl)methyl)benzofuran-3-carboxamide
Cpd 123 5-(benzyloxy)-N-(4-hydroxycyclohexyl)-2-methylbenzofuran-3-carboxamide
Cpd 124 5-(benzyloxy)-N-(trans-4-hydroxycyclohexyl)-2-methylbenzofuran-3-carboxamide
Cpd 125 5-(benzyloxy)-N-(cis-4-hydroxycyclohexyl)-2-methylbenzofuran-3-carboxamide
Cpd 126 5-(benzyloxy)-N-((3S,5S)-5-(hydroxymethyl)pyrrolidin-3-yl)-2-methylbenzofuran-3-carboxamide
Cpd 127 (S)-5-((3-methoxybenzyl)oxy)-2-methyl-N-(pyrrolidin-3-yl)benzofuran-3-carboxamide
Cpd 128 5-(benzyloxy)-N-43-(hydroxymethyl)oxetan-3-yl)methyl)-2-methylbenzofuran-3-carboxamide
Cpd 129 5-((2-fluorobenzyl)oxy)-2-methyl-N-(2-oxopyrrolidin-3-yl)benzofuran-3-carboxamide
Cpd 130 5-(benzyloxy)-N-(4-fluoropiperidin-3-yl)-2-methylbenzofuran-3-carboxamide
Cpd 131 5-(benzyloxy)-N-(3-fluoropiperidin-4-yl)-2-methylbenzofuran-3-carboxamide
Cpd 132 5-((3-fluorobenzyl)oxy)-2-methyl-N-((tetrahydrofuran-2-yl)methyl)benzofuran-3-carboxamide
Cpd 133 5-(benzyloxy)-N-(cyclopropylsulfonyl)-2-methylbenzofuran-3-carboxamide
Cpd 134 5-(benzyloxy)-2-methyl-N-phenethylbenzofuran-3-carboxamide
Cpd 135 (S)-5-((2,3-difluorobenzyl)oxy)-2-methyl-N-(pyrrolidin-3-yl)benzofuran-3-carboxamide
Cpd 136 (S)-5-((2,6-difluorobenzyl)oxy)-2-methyl-N-(pyrrolidin-3-yl)benzofuran-3-carboxamide
Cpd 137 5-(benzyloxy)-N-(4,4-difluoropyrrolidin-3-yl)-2-methylbenzofuran-3-carboxamide
Cpd 138 5-(benzyloxy)-2-methyl-N-(2-(pyridin-2-yl)ethyl)benzofuran-3-carboxamide
Cpd 139 5-(benzyloxy)-2-methyl-N-(2-(pyridin-4-yl)ethyl)benzofuran-3-carboxamide
Cpd 140 5-(benzyloxy)-2-methyl-N-(trans-octahydrocyclopenta[c]pyrrol-5-yl)benzofuran-3-carboxamide
Cpd 141 5-(benzyloxy)-N-((1R,3s,5S)-8-azabicyclo[3.2.1]octan-3-yl)-2-methylbenzofuran-3-carboxamide
Cpd 142 5-(benzyloxy)-2-methyl-N-(quinuclidin-3-yl)benzofuran-3-carboxamide
Cpd 143 5-(benzyloxy)-N-((1R,3r,5S)-8-azabicyclo[3.2.1]octan-3-yl)-2-methylbenzofuran-3-carboxamide
Cpd 144 5-(benzyloxy)-N-(hexahydro-1H-pyrrolizin-1-yl)-2-methylbenzofuran-3-carboxamide
Cpd 145 (S)—N-(1-acetylpyrrolidin-3-yl)-5-(benzyloxy)-2-methylbenzofuran-3-carboxamide
Cpd 146 5-(benzyloxy)-2-methyl-N-(1-methyl-2-oxopiperidin-4-yl)benzofuran-3-carboxamide
Cpd 147 5-(benzyloxy)-N-(1-ethyl-2-oxopyrrolidin-3-yl)-2-methylbenzofuran-3-carboxamide
Cpd 148 5-(benzyloxy)-2-methyl-N-(1-(oxetan-3-yl)azetidin-3-yl)benzofuran-3-carboxamide
Cpd 149 (5-(benzyloxy)-2-methylbenzofuran-3-yl)(4-ethyl-1,4-diazepan-1-yl)methanone
Cpd 150 (5-(benzyloxy)-2-methylbenzofuran-3-yl)(3-(dimethylamino)piperidin-1-yl)methanone
Cpd 151 5-(benzyloxy)-N-(1-ethylpiperidin-4-yl)-2-methylbenzofuran-3-carboxamide
Cpd 152 (S)-5-(benzyloxy)-N-((1-ethylpyrrolidin-2-yl)methyl)-2-methylbenzofuran-3-carboxamide
Cpd 153 (R)-5-(benzyloxy)-N-((1-ethylpyrrolidin-2-yl)methyl)-2-methylbenzofuran-3-carboxamide
Cpd 154 2-methyl-5-((4-methylbenzyl)oxy)-N-(1-methylpiperidin-4-yl)benzofuran-3-carboxamide
Cpd 155 5-(benzyloxy)-N-(1,2-dimethylpiperidin-4-yl)-2-methylbenzofuran-3-carboxamide
Cpd 156 5-(benzyloxy)-N,2-dimethyl-N-(1-methylpiperidin-4-yl)benzofuran-3-carboxamide
Cpd 157 5-((3-(aminomethyl)benzyl)oxy)-2-methyl-N-(2-oxopyrrolidin-3-yl)benzofuran-3-carboxamide
Cpd 158 (2S,4R)-4-(5-(benzyloxy)-2-methylbenzofuran-3-carboxamido)pyrrolidine-2-carboxamide
Cpd 159 5-(benzyloxy)-N-(4-methoxycyclohexyl)-2-methylbenzofuran-3-carboxamide
Cpd 160 (2S,4S)-4-(5-(benzyloxy)-2-methylbenzofuran-3-carboxamido)pyrrolidine-2-carboxylic acid
Cpd 161 (2S,4R)-4-(5-(benzyloxy)-2-methylbenzofuran-3-carboxamido)pyrrolidine-2-carboxylic acid
Cpd 162 (2R,4R)-4-(5-(benzyloxy)-2-methylbenzofuran-3-carboxamido)pyrrolidine-2-carboxylic acid
Cpd 163 (2R,4S)-4-(5-(benzyloxy)-2-methylbenzofuran-3-carboxamido)pyrrolidine-2-carboxylic acid
Cpd 164 5-((2-methoxybenzyl)oxy)-2-methyl-N-(2-oxopyrrolidin-3-yl)benzofuran-3-carboxamide
Cpd 165 5-(benzyloxy)-2-methyl-N-(2-morpholinoethyl)benzofuran-3-carboxamide
Cpd 166 5-(benzyloxy)-N-(3,3-bis(hydroxymethyl)cyclobutyl)-2-methylbenzofuran-3-carboxamide
Cpd 167 5-(benzyloxy)-N-(4-(hydroxymethyl)tetrahydro-2H-pyran-4-yl)-2-methylbenzofuran-3-carboxamide
Cpd 168 5-((3-fluorobenzyl)oxy)-2-methyl-N-(1-methylpiperidin-4-yl)benzofuran-3-carboxamide
Cpd 169 N-(cyclopropylsulfonyl)-2-methyl-5-((4-methylbenzyl)oxy)benzofuran-3-carboxamide
Cpd 170 N-(cyclopropylsulfonyl)-2-methyl-5-((3-methylbenzyl)oxy)benzofuran-3-carboxamide
Cpd 171 5-((2,3-difluorobenzyl)oxy)-2-methyl-N-(2-oxopyrrolidin-3-yl)benzofuran-3-carboxamide
Cpd 172 5-((2,6-difluorobenzyl)oxy)-2-methyl-N-(2-oxopyrrolidin-3-yl)benzofuran-3-carboxamide
Cpd 173 5-(benzyloxy)-N-(3,3-difluoropiperidin-4-yl)-2-methylbenzofuran-3-carboxamide
Cpd 174 5-(benzyloxy)-N-(2-hydroxy-2-(pyridin-3-yl)ethyl)-2-methylbenzofuran-3-carboxamide
Cpd 175 N-(cyclopropylsulfonyl)-5-((2-fluorobenzyl)oxy)-2-methylbenzofuran-3-carboxamide
Cpd 176 N-(cyclopropylsulfonyl)-5-((3-fluorobenzyl)oxy)-2-methylbenzofuran-3-carboxamide
Cpd 177 N-(cyclopropylsulfonyl)-5-((4-fluorobenzyl)oxy)-2-methylbenzofuran-3-carboxamide
Cpd 178 5-(benzyloxy)-N-(2-(3,5-dimethylisoxazol-4-yl)ethyl)-2-methylbenzofuran-3-carboxamide
Cpd 179 5-(benzyloxy)-N-(2-(3,5-dimethyl-1H-1,2,4-triazol-1-yl)ethyl)-2-methylbenzofuran-3-carboxamide
Cpd 180 5-(benzyloxy)-2-methyl-N-(7-azaspiro[3.5]nonan-2-yl)benzofuran-3-carboxamide
Cpd 181 5-(benzyloxy)-2-methyl-N-(8-methyl-8-azabicyclo[3.2.1]octan-3-yl)benzofuran-3-carboxamide
Cpd 182 5-(benzyloxy)-2-methyl-N-((1R,3s,5S)-8-methyl-8-azabicyclo[3.2.1]octan-3-yl)benzofuran-3-carboxamide
Cpd 183 5-(benzyloxy)-2-methyl-N-((1R,3r,5S)-8-methyl-8-azabicyclo[3.2.1]octan-3-yl)benzofuran-3-carboxamide
Cpd 184 5-(benzyloxy)-N-((1R,3s,5S)-9-azabicyclo[3.3.1]nonan-3-yl)-2-methylbenzofuran-3-carboxamide
Cpd 185 5-(benzyloxy)-N-(1-isopropylpiperidin-4-yl)-2-methylbenzofuran-3-carboxamide
Cpd 186 5-(benzyloxy)-N-(4-(dimethylamino)cyclohexyl)-2-methylbenzofuran-3-carboxamide
Cpd 187 5-(benzyloxy)-N-41-(dimethylamino)cyclopentyl)methyl)-2-methylbenzofuran-3-carboxamide
Cpd 188 (2S,4R)-4-(5-(benzyloxy)-2-methylbenzofuran-3-carboxamido)-N-methylpyrrolidine-2-carboxamide
Cpd 189 5-(benzyloxy)-2-methyl-N-(2-(4-methylpiperazin-1-yl)ethyl)benzofuran-3-carboxamide
Cpd 190 5-((4-(aminomethyl)benzyl)oxy)-2-methyl-N-(1-methylpiperidin-4-yl)benzofuran-3-carboxamide
Cpd 191 methyl (2S,4S)-4-(5-(benzyloxy)-2-methylbenzofuran-3-carboxamido)pyrrolidine-2-carboxylate
Cpd 192 5-((2-(hydroxymethyl)benzyl)oxy)-2-methyl-N-(1-methylpiperidin-4-yl)benzofuran-3-carboxamide
Cpd 193 4-(5-(benzyloxy)-2-methylbenzofuran-3-carboxamido)tetrahydro-2H-pyran-4-carboxylic acid
Cpd 194 2-ethyl-5-((3-fluorobenzyl)oxy)-N-(1-methylpiperidin-4-yl)benzofuran-3-carboxamide
Cpd 195 5-(benzyloxy)-2-methyl-N-((trifluoromethyl)sulfonyl)benzofuran-3-carboxamide
Cpd 196 N-([1,2,4]triazolo[4,3-a]pyrimidin-3-ylmethyl)-5-(benzyloxy)-2-methylbenzofuran-3-carboxamide
Cpd 197 5-((2-fluorobenzyl)oxy)-N-(4-(hydroxymethyl)tetrahydro-2H-pyran-4-ye-2-methylbenzofuran-3-carboxamide
Cpd 198 5-(benzyloxy)-N-(1,1-dioxidotetrahydro-2H-thiopyran-4-yl)-2-methylbenzofuran-3-carboxamide
Cpd 199 N-(cyclopropylsulfonyl)-5-((2-methoxybenzyl)oxy)-2-methylbenzofuran-3-carboxamide
Cpd 200 5-(benzyloxy)-N-((4,6-dimethyl-2-oxo-1,2-dihydropyridin-3-yl)methyl)-2-methylbenzofuran-3-carboxamide
Cpd 201 N-(3,3-difluoropiperidin-4-yl)-5-((2-fluorobenzyl)oxy)-2-methylbenzofuran-3-carboxamide
Cpd 202 (S)-2-methyl-N-(pyrrolidin-3-yl)-5-((4-(trifluoromethyl)benzyl)oxy)benzofuran-3-carboxamide
Cpd 203 (S)-2-methyl-N-(pyrrolidin-3-yl)-5-((2-(trifluoromethyl)benzyl)oxy)benzofuran-3-carboxamide
Cpd 204 (S)-2-methyl-N-(pyrrolidin-3-yl)-5-((3-(trifluoromethyl)benzyl)oxy)benzofuran-3-carboxamide
Cpd 205 5-(benzyloxy)-2-methyl-N-((7S,8aS)-2-methyloctahydropyrrolo[1,2-a]pyrazin-7-yl)benzofuran-3-carboxamide
Cpd 206 (S)-5-(benzyloxy)-2-methyl-N-(1-(oxetan-3-yl)piperidin-3-yl)benzofuran-3-carboxamide
Cpd 207 (R)-5-(benzyloxy)-2-methyl-N-(1-(oxetan-3-yl)piperidin-3-yl)benzofuran-3-carboxamide
Cpd 208 5-(benzyloxy)-2-methyl-N-(1-(oxetan-3-yl)piperidin-4-yl)benzofuran-3-carboxamide
Cpd 209 5-(benzyloxy)-2-methyl-N-(2,2,6,6-tetramethylpiperidin-4-yl)benzofuran-3-carboxamide
Cpd 210 5-(benzyloxy)-N-(trans-4-(2-hydroxypropan-2-yl)cyclohexyl)-2-methylbenzofuran-3-carboxamide
Cpd 211 5-((2,3-difluorobenzyl)oxy)-N-(4,4-difluoropyrrolidin-3-yl)-2-methylbenzofuran-3-carboxamide
Cpd 212 methyl 4-(5-(benzyloxy)-2-methylbenzofuran-3-carboxamido)tetrahydro-2H-pyran-4-carboxylate
Cpd 213 N-(1-([1,2,4]triazolo[4,3-a]pyrimidin-3-yl)ethyl)-5-(benzyloxy)-2-methylbenzofuran-3-carboxamide
Cpd 214 (5-(benzyloxy)-2-methylbenzofuran-3-yl)(4-(pyridin-2-yl)piperazin-1-yl)methanone
Cpd 215 (S)-5-(benzyloxy)-2-methyl-N-(1-(methylsulfonyl)pyrrolidin-3-yl)benzofuran-3-carboxamide
Cpd 216 5-(benzyloxy)-N-(2-(dimethylamino)-2-phenylethyl)-2-methylbenzofuran-3-carboxamide
Cpd 217 5-((2,3-difluorobenzyl)oxy)-N-(4-(hydroxymethyl)tetrahydro-2H-pyran-4-yl)-2-methylbenzofuran-3-carboxamide
Cpd 218 5-((2,6-difluorobenzyl)oxy)-N-(4-(hydroxymethyl)tetrahydro-2H-pyran-4-yl)-2-methylbenzofuran-3-carboxamide
Cpd 219 2-methyl-N-(2-oxopyrrolidin-3-yl)-5-((2-(trifluoromethyl)benzyl)oxy)benzofuran-3-carboxamide
Cpd 220 rac-2-methyl-N—((R)-pyrrolidin-3-yl)-5-(1-(2-(trifluoromethyl)phenyl)ethoxy)benzofuran-3-carboxamide
Cpd 221 5-(benzyloxy)-2-cyclopentyl-N-(1-methylpiperidin-4-yl)benzofuran-3-carboxamide
Cpd 222 5-(benzyloxy)-N-((7S,8aS)-1,4-dioxooctahydropyrrolo[1,2-a]pyrazin-7-yl)-2-methylbenzofuran-3-carboxamide
Cpd 223 (S)-2-methyl-N-(pyrrolidin-3-yl)-5-((3-(trifluoromethoxy)benzyl)oxy)benzofuran-3-carboxamide
Cpd 224 5-(3-(dimethylamino)-1-phenylpropoxy)-2-methyl-N-(2-oxopyrrolidin-3-yl)benzofuran-3-carboxamide
Cpd 225 5-(benzyloxy)-N-(1-(2-(dimethylamino)ethyl)piperidin-4-yl)-2-methylbenzofuran-3-carboxamide
Cpd 226 5-((2,6-difluorobenzyl)oxy)-N-(3,3-difluoropiperidin-4-yl)-2-methylbenzofuran-3-carboxamide
Cpd 227 5-((2,3-difluorobenzyl)oxy)-N-(3,3-difluoropiperidin-4-yl)-2-methylbenzofuran-3-carboxamide
Cpd 228 tert-butyl 3-(5-(benzyloxy)-2-methylbenzofuran-3-carboxamido)azetidine-1-carboxylate
Cpd 229 5-(benzyloxy)-N-(1-(3-methoxypropyl)piperidin-4-yl)-2-methylbenzofuran-3-carboxamide
Cpd 230 5-(benzyloxy)-2-methyl-N-(1-(pyrimidin-2-yl)piperidin-4-yl)benzofuran-3-carboxamide
Cpd 231 rac-2-methyl-N—((R)-pyrrolidin-3-yl)-5-(1-(2-(trifluoromethyl)phenyl)propoxy)benzofuran-3-carboxamide
Cpd 232 2-methyl-N-(1-methylpiperidin-4-yl)-5-((3-(trifluoromethyl)benzyl)oxy)benzofuran-3-carboxamide
Cpd 233 2-methyl-N-(1-methylpiperidin-4-yl)-5-((2-(trifluoromethyl)benzyl)oxy)benzofuran-3-carboxamide
Cpd 234 2-methyl-N-(1-methylpiperidin-4-yl)-5-((4-(trifluoromethyl)benzyl)oxy)benzofuran-3-carboxamide
Cpd 235 5-(benzyloxy)-2-methyl-N-((7S,8aS)-2-methyl-1,4-dioxooctahydropyrrolo[1,2-a]pyrazin-7-yl)benzofuran-3-carboxamide
Cpd 236 1-(5-(benzyloxy)-2-methylbenzofuran-3-carbonyl)-N-isobutylpiperidine-3-carboxamide
Cpd 237 tert-butyl 1-(5-(benzyloxy)-2-methylbenzofuran-3-carbonyl)piperidine-4-carboxylate
Cpd 238 tert-butyl (R)-3-(5-(benzyloxy)-2-methylbenzofuran-3-carboxamido)pyrrolidine-1-carboxylate
Cpd 239 tert-butyl (S)-3-(5-(benzyloxy)-2-methylbenzofuran-3-carboxamido)pyrrolidine-1-carboxylate
Cpd 240 N-(4,4-difluoropyrrolidin-3-yl)-2-methyl-5-((2-(trifluoromethyl)benzyl)oxy)benzofuran-3-carboxamide
Cpd 241 5-(benzyloxy)-N-(1-benzylpiperidin-4-yl)-2-methylbenzofuran-3-carboxamide
Cpd 242 2-methyl-N-(1-methylpiperidin-4-yl)-5-((2-(methylsulfonyl)benzyl)oxy)benzofuran-3-carboxamide
Cpd 243 (5-(benzyloxy)-2-methylbenzofuran-3-yl)(3-(3-isopropyl-1,2,4-oxadiazol-5-yl)piperidin-1-yl)methanone
Cpd 244 2-methyl-N-(1-methylpiperidin-4-yl)-5-((3-(trifluoromethoxy)benzyl)oxy)benzofuran-3-carboxamide
Cpd 245 2-methyl-5-((2-(4-methylpiperazin-1-yl)benzyl)oxy)-N-(2-oxopyrrolidin-3-yl)benzofuran-3-carboxamide
Cpd 246 tert-butyl ((1-(5-(benzyloxy)-2-methylbenzofuran-3-carboxamido)cyclobutyl)methyl)carbamate
Cpd 247 tert-butyl 3-(5-(benzyloxy)-2-methylbenzofuran-3-carboxamido)piperidine-1-carboxylate
Cpd 248 tert-butyl (1-(5-(benzyloxy)-2-methylbenzofuran-3-carbonyl)piperidin-4-yl)carbamate
Cpd 249 tert-butyl 4-(5-(benzyloxy)-2-methylbenzofuran-3-carboxamido)piperidine-1-carboxylate
Cpd 250 5-((2-(2-(dimethylamino)ethoxy)benzyl)oxy)-2-methyl-N-(1-methylpiperidin-4-yl)benzofuran-3-carboxamide
Cpd 251 tert-butyl trans-4-(5-(benzyloxy)-2-methylbenzofuran-3-carboxamido)-2-methylpiperidine-1-carboxylate
Cpd 252 tert-butyl cis-4-(5-(benzyloxy)-2-methylbenzofuran-3-carboxamido)-2-methylpiperidine-1-carboxylate
Cpd 253 tert-butyl 4-((5-(benzyloxy)-2-methylbenzofuran-3-carboxamido)methyl)piperidine-1-carboxylate
Cpd 254 tert-butyl 4-(5-(benzyloxy)-2-methylbenzofuran-3-carboxamido)-3-fluoropiperidine-1-carboxylate
Cpd 255 tert-butyl 3-(5-(benzyloxy)-2-methylbenzofuran-3-carboxamido)-4-fluoropiperidine-1-carboxylate
Cpd 256 tert-butyl (S)-3-(5-((2,3-difluorobenzyl)oxy)-2-methylbenzofuran-3-carboxamido)pyrrolidine-1-carboxylate
Cpd 257 tert-butyl (S)-3-(5-((2,6-difluorobenzyl)oxy)-2-methylbenzofuran-3-carboxamido)pyrrolidine-1-carboxylate
Cpd 258 tert-butyl (1R,3s,5S)-3-(5-(benzyloxy)-2-methylbenzofuran-3-carboxamido)-8-azabicyclo[3.2.1]-octane-8-carboxylate
Cpd 259 tert-butyl trans-5-(5-(benzyloxy)-2-methylbenzofuran-3-carboxamido)hexahydrocyclopenta[c]pyrrole-2(1H)-carboxylate
Cpd 260 2-methyl-N-(1-methylpiperidin-4-yl)-5-((2-(((tetrahydro-2H-pyran-2-yl)oxy)methyl)-benzyl)oxy)benzofuran-3-carboxamide
Cpd 261 tert-butyl (3-4(2-methyl-3-((2-oxopyrrolidin-3-yl)carbamoyl)benzofuran-5-yl)oxy)methyl)-benzyl)carbamate
Cpd 262 tert-butyl 4-(5-(benzyloxy)-2-methylbenzofuran-3-carboxamido)-4-(hydroxymethyl)piperidine-1-carboxylate
Cpd 263 tert-butyl 4-(5-(benzyloxy)-2-methylbenzofuran-3-carboxamido)-3,3-difluoropiperidine-1-carboxylate
Cpd 264 tert-butyl 2-(5-(benzyloxy)-2-methylbenzofuran-3-carboxamido)-7-azaspiro[3.5]nonane-7-carboxylate
Cpd 265 tert-butyl (1R,5S,7r)-7-(5-(benzyloxy)-2-methylbenzofuran-3-carboxamido)-3-oxa-9-azabicyclo-[3.3.1]nonane-9-carboxylate
Cpd 266 tert-butyl 4-(5-((2,3-difluorobenzyl)oxy)-2-methylbenzofuran-3-carboxamido)-3,3-difluoro-pyrrolidine-1-carboxylate
Cpd 267 rac-tert-butyl (3R)-3-(2-methyl-5-(1-(2-(trifluoromethyl)phenyl)ethoxy)benzofuran-3-carboxamido)pyrrolidine-1-carboxylate
Cpd 268 tert-butyl 4-(5-((2,6-difluorobenzyl)oxy)-2-methylbenzofuran-3-carboxamido)-3,3-difluoropiperidine-1-carboxylate
Cpd 269 tert-butyl 3-((5-(benzyloxy)-2-methylbenzofuran-3-carboxamido)methyl)pyrrolidine-1-carboxylate
Cpd 270 tert-butyl 2-((5-(benzyloxy)-2-methylbenzofuran-3-carboxamido)methyl)pyrrolidine-1-carboxylate
Cpd 271 tert-butyl trans-3-(5-(benzyloxy)-2-methylbenzofuran-3-carboxamido)-4-fluoropyrrolidine-1-carboxylate
Cpd 272 tert-butyl cis-3-(5-(benzyloxy)-2-methylbenzofuran-3-carboxamido)-4-fluoropyrrolidine-1-carboxylate
Cpd 273 tert-butyl (cis-4-(5-(benzyloxy)-2-methylbenzofuran-3-carboxamido)cyclohexyl)carbamate
Cpd 274 tert-butyl 4-(5-(benzyloxy)-2-methylbenzofuran-3-carboxamido)azepane-1-carboxylate
Cpd 275 tert-butyl (2R,4R)-4-(5-(benzyloxy)-2-methylbenzofuran-3-carboxamido)-2-(hydroxy-methyl)pyrrolidine-1-carboxylate
Cpd 276 tert-butyl 4-(5-(benzyloxy)-2-methylbenzofuran-3-carboxamido)-3,3-difluoropyrrolidine-1-carboxylate
Cpd 277 tert-butyl (1R,5R)-3-(5-(benzyloxy)-2-methylbenzofuran-3-carboxamido)-9-azabicyclo-[3.3.1]nonane-9-carboxylate
Cpd 278 1-(tert-butyl) 2-methyl (2S,4S)-4-(5-(benzyloxy)-2-methylbenzofuran-3-carboxamido)pyrrolidine-1,2-dicarboxylate
Cpd 279 tert-butyl 3,3-difluoro-4-(5-((2-fluorobenzyl)oxy)-2-methylbenzofuran-3-carboxamido)piperidine-1-carboxylate
Cpd 280 tert-butyl (R)-3-(2-methyl-5-((3-(trifluoromethoxy)benzyl)oxy)benzofuran-3-carboxamido)-pyrrolidine-1-carboxylate
Cpd 281 tert-butyl 3,3-difluoro-4-(2-methyl-5-((2-(trifluoromethyl)benzyl)oxy)benzofuran-3-carboxamido)pyrrolidine-1-carboxylate
Cpd 282 tert-butyl (R)-3-(2-methyl-5-((3-methylbenzyl)oxy)benzofuran-3-carboxamido)pyrrolidine-1-carboxylate
Cpd 283 tert-butyl (R)-3-(2-methyl-5-((4-methylbenzyl)oxy)benzofuran-3-carboxamido)pyrrolidine-1-carboxylate
Cpd 284 rac-tert-butyl (R)-3-(2-methyl-5-((2-methylbenzyl)oxy)benzofuran-3-carboxamido)pyrrolidine-1-carboxylate
Cpd 285 tert-butyl (R)-3-(5-((2-fluorobenzyl)oxy)-2-methylbenzofuran-3-carboxamido)pyrrolidine-1-carboxylate
Cpd 286 tert-butyl (R)-3-(5-((3-fluorobenzyl)oxy)-2-methylbenzofuran-3-carboxamido)pyrrolidine-1-carboxylate
Cpd 287 tert-butyl (R)-3-(5-((4-fluorobenzyl)oxy)-2-methylbenzofuran-3-carboxamido)pyrrolidine-1-carboxylate
Cpd 288 tert-butyl (R)-3-(5-(3-methoxybenzyl)oxy)-2-methylbenzofuran-3-carboxamido)pyrrolidine-1-carboxylate
Cpd 289 tert-butyl (1R,3r,5S)-3-(5-(benzyloxy)-2-methylbenzofuran-3-carboxamido)-8-azabicyclo-[3.2.1]octane-8-carboxylate
Cpd 290 tert-butyl (R)-3-(2-methyl-5-((4-(trifluoromethyl)benzyl)oxy)benzofuran-3-carboxamido)-pyrrolidine-1-carboxylate
Cpd 291 tert-butyl (R)-3-(2-methyl-5-((2-(trifluoromethyl)benzyl)oxy)benzofuran-3-carboxamido)-pyrrolidine-1-carboxylate
Cpd 292 tert-butyl (R)-3-(2-methyl-5-((3-(trifluoromethyl)benzyl)oxy)benzofuran-3-carboxamido)-pyrrolidine-1-carboxylate
Cpd 293 tert-butyl (R)-3-(5-((4-cyanobenzyl)oxy)-2-methylbenzofuran-3-carboxamido)pyrrolidine-1-carboxylate
Cpd 294 rac-tert-butyl (3R)-3-(2-methyl-5-(1-(2-(trifluoromethyl)phenyl)propoxy)benzofuran-3-carboxamido)pyrrolidine-1-carboxylate
Cpd 295 tert-butyl (4-(((2-methyl-3-((1-methylpiperidin-4-yl)carbamoyl)benzofuran-5-yl)oxy)methyl)-benzyl)carbamate
Cpd 296 tert-butyl 4-(5-((2,3-difluorobenzyl)oxy)-2-methylbenzofuran-3-carboxamido)-3,3-difluoro-piperidine-1-carboxylate
Cpd 297 5-(benzyloxy)-4-cyano-N-(4,4-difluoropyrrolidin-3-yl)-2-methylbenzofuran-3-carboxamide
Cpd 298 5-(benzyloxy)-N-(4,4-difluoropyrrolidin-3-yl)-4-fluoro-2-methylbenzofuran-3-carboxamide
Cpd 299 5-(benzyloxy)-N-(4,4-difluoropyrrolidin-3-yl)-2,4-dimethylbenzofuran-3-carboxamide
Cpd 300 5-(benzyloxy)-6-chloro-N-(4,4-difluoropyrrolidin-3-yl)-2-methylbenzofuran-3-carboxamide
Cpd 301 5-(benzyloxy)-6-cyano-N-(4,4-difluoropyrrolidin-3-yl)-2-methylbenzofuran-3-carboxamide
Cpd 302 5-(benzyloxy)-N-(4,4-difluoropyrrolidin-3-yl)-6-fluoro-2-methylbenzofuran-3-carboxamide
Cpd 303 5-(benzyloxy)-N-(4,4-difluoropyrrolidin-3-yl)-2,6-dimethylbenzofuran-3-carboxamide
Cpd 304 5-(benzyloxy)-7-chloro-N-(4,4-difluoropyrrolidin-3-yl)-2-methylbenzofuran-3-carboxamide
Cpd 305 5-(benzyloxy)-7-cyano-N-(4,4-difluoropyrrolidin-3-yl)-2-methylbenzofuran-3-carboxamide
Cpd 306 5-(benzyloxy)-N-(4,4-difluoropyrrolidin-3-yl)-7-fluoro-2-methylbenzofuran-3-carboxamide
Cpd 307 5-(benzyloxy)-N-(4,4-difluoropyrrolidin-3-yl)-2,7-dimethylbenzofuran-3-carboxamide
Cpd 308 N-(4-(hydroxymethyl)tetrahydro-2H-pyran-4-yl)-2-methyl-5-(1-phenylethoxy)benzofuran-3-carboxamide
Cpd 309 N-(3-carbamoyloxetan-3-yl)-5-((2-fluorobenzyl)oxy)-2-methylbenzofuran-3-carboxamide
Cpd 310 5-((2-fluorobenzyl)oxy)-N-(3-(hydroxymethyl)oxetan-3-yl)-2-methylbenzofuran-3-carboxamide
Cpd 311 5-((2-fluorobenzyl)oxy)-N-(1-(2-hydroxyethyl)-2-oxopyrrolidin-3-yl)-2-methylbenzofuran-3-carboxamide
Cpd 312 N-(1,3-dihydroxy-2-methylpropan-2-yl)-5-((2-fluorobenzyl)oxy)-2-methylbenzofuran-3-carboxamide
Cpd 313 5-((2-fluorobenzyl)oxy)-N-(1-hydroxy-2-methylpropan-2-yl)-2-methylbenzofuran-3-carboxamide me
Cpd 314 N-(1-amino-3-hydroxy-1-oxopropan-2-yl)-5-((2-fluorobenzyl)oxy)-2-methylbenzofuran-3-carboxamide
Cpd 315 N-(1-amino-2-methyl-1-oxopropan-2-yl)-5-((2-fluorobenzyl)oxy)-2-methylbenzofuran-3-carboxamide
Cpd 316 N-(4-(hydroxymethyl)tetrahydro-2H-pyran-4-yl)-2-methyl-5-((2-phenylpropan-2-yl)oxy)benzofuran-3-carboxamide
Cpd 317 5-(2-hydroxy-1-phenylethoxy)-N-(4-(hydroxymethyl)tetrahydro-2H-pyran-4-yl)-2-methylbenzofuran-3-carboxamide
Cpd 318 N-(1-amino-2-methyl-1-oxopropan-2-yl)-5-((2-fluorophenoxy)methyl)-2-methylbenzofuran-3-carboxamide
Cpd 319 5-((2-fluorophenoxy)methyl)-N-(1-hydroxy-2-methylpropan-2-yl)-2-methylbenzofuran-3-carboxamide
Cpd 320 N-(1-amino-1-oxopropan-2-yl)-5-((2-fluorobenzyl)oxy)-2-methylbenzofuran-3-carboxamide
Cpd 321 N-(1-amino-3-hydroxy-1-oxopropan-2-yl)-5-((2-fluorophenoxy)methyl)-2-methylbenzofuran-3-carboxamide
Cpd 322 N-(1-carbamoylcyclobutyl)-5-((2-fluorobenzyl)oxy)-2-methylbenzofuran-3-carboxamide
Cpd 323 N-(3-carbamoyltetrahydrofuran-3-yl)-5-((2-fluorobenzyl)oxy)-2-methylbenzofuran-3-carboxamide
Cpd 324 5-((2-fluorobenzyl)oxy)-N-(1-(hydroxymethyl)cyclobutyl)-2-methylbenzofuran-3-carboxamide
Cpd 325 5-((2-fluorobenzyl)oxy)-N-(1-(hydroxymethyl)cyclopropyl)-2-methylbenzofuran-3-carboxamide
Cpd 326 N-(1-carbamoylcyclopropyl)-5-((2-fluorobenzyl)oxy)-2-methylbenzofuran-3-carboxamide
Cpd 327 5-((2-fluorobenzyl)oxy)-N-(3-(hydroxymethyl)tetrahydrofuran-3-yl)-2-methylbenzofuran-3-carboxamide
Cpd 328 5-((2-fluorobenzyl)oxy)-N-(1-hydroxypropan-2-yl)-2-methylbenzofuran-3-carboxamide
Cpd 329 5-((2-fluorobenzyl)oxy)-N-(cis-4-hydroxytetrahydrofuran-3-yl)-2-methylbenzofuran-3-carboxamide
Cpd 330 5-((2-fluorobenzyl)oxy)-N-(trans-4-hydroxytetrahydrofuran-3-yl)-2-methylbenzofuran-3-carboxamide
Cpd 331 N-(4,4-difluorotetrahydrofuran-3-yl)-5-((2-fluorobenzyl)oxy)-2-methylbenzofuran-3-carboxamide
Cpd 332 N-(4-(hydroxymethyl)tetrahydro-2H-pyran-4-yl)-2-methyl-5-((2-(methylsulfonyl)benzyl)oxy)benzofuran-3-carboxamide
Cpd 333 N-(3,3-difluoropiperidin-4-yl)-2-methyl-5-((2-(methylsulfonyl)benzyl)oxy)benzofuran-3-carboxamide
Cpd 334 5-((2-fluorobenzyl)oxy)-N-(3-(hydroxymethyl)-2-oxopyrrolidin-3-yl)-2-methylbenzofuran-3-carboxamide
Cpd 335 N—((S)-1-amino-3-hydroxy-1-oxopropan-2-yl)-5-(1-(2-fluorophenyl)-2-hydroxyethoxy)-2-methylbenzofuran-3-carboxamide
Cpd 336 N—((S)-1-amino-3-hydroxy-1-oxopropan-2-yl)-2-methyl-5-(1-phenylethoxy)benzofuran-3-carboxamide
Cpd 337 (S)—N-(1-amino-3-hydroxy-1-oxopropan-2-yl)-5-(benzyloxy)-2-methylbenzofuran-3-carboxamide
Cpd 338 (S)—N-(1-Amino-3-hydroxy-1-oxopropan-2-yl)-5-((2-chlorobenzyl)oxy)-2-methylbenzofuran-3-carboxamide
Cpd 339 (S)—N-(1-Amino-3-hydroxy-1-oxopropan-2-yl)-5-((2-methoxybenzyl) oxy)-2-methylbenzofuran-3-carboxamide
Cpd 340 (S)—N-(1-amino-3-hydroxy-1-oxopropan-2-yl)-5-((2,4-difluorobenzyl)oxy)-2-methylbenzofuran-3-carboxamide
Cpd 341 (S)—N-(1-Amino-3-hydroxy-1-oxopropan-2-yl)-5-((2-cyanobenzyl)oxy)-2-methylbenzofuran-3-carboxamide
Cpd 342 (S)—N-(1-amino-3-hydroxy-1-oxopropan-2-yl)-5-((2-fluoro-4-methylbenzyl)oxy)-2-methylbenzofuran-3-carboxamide
Cpd 343 N-(1-carbamoylcyclobutyl)-2-methyl-5-(1-phenylethoxy)benzofuran-3-carboxamide
Cpd 344 5-(benzyloxy)-N-(1-carbamoylcyclobutyl)-2-methylbenzofuran-3-carboxamide
Cpd 345 N-(1-carbamoylcyclobutyl)-5-(1-(2-fluorophenyl)-2-hydroxyethoxy)-2-methylbenzofuran-3-carboxamide
Cpd 346 N—((S)-1-Amino-3-hydroxy-1-oxopropan-2-yl)-5-(2-methoxy-1-phenylethoxy)-2-methylbenzofuran-3-carboxamide
Cpd 347 N—((S)-1-Amino-3-hydroxy-1-oxopropan-2-yl)-5-(2-(dimethylamino)-1-phenylethoxy)-2-methylbenzofuran-3-carboxamide
and the physiologically acceptable salts thereof.

14. A pharmaceutical composition comprising a compound according to claim 1.

15. The compound according to claim 1 or the pharmaceutical composition according to claim 14, for use in the treatment of pain.

16. The compound or the pharmaceutical composition for use in the treatment of pain according to claim 15, wherein the pain is selected from nociceptive pain, inflammatory pain, and neuropathic pain; preferably post-operative pain.
